SEQUENCE LISTING (141 sequences)

SEQ ID NO: 1 - DNA, 28 bp, Artificial Sequence
  Primer sequence for SARS CoV-2 Nucleocapsid
ttttgtctgataatggaccccaaaatca

SEQ ID NO: 2 - DNA, 28 bp, Artificial Sequence
  Primer sequence for SARS CoV-2 Nucleocapsid
tttgttctccattctggttactgccagt

SEQ ID NO: 3 - DNA, 28 bp, Artificial Sequence
  Primer sequence for coronavirus Nucleocapsid
tttaggaactaatcagacaaggaactga

SEQ ID NO: 4 - DNA, 28 bp, Artificial Sequence
  Primer sequence for coronavirus Nucleocapsid
tttgttcccgaaggtgtgacttccatgc

SEQ ID NO: 5 - DNA, 28 bp, Artificial Sequence
  Primer sequence for coronavirus Nucleocapsid
tttcggcatcatatgggtygcaactgag

SEQ ID NO: 6 - DNA, 28 bp, Artificial Sequence
  Primer sequence for coronavirus Nucleocapsid
tttccttttggcaatgttgttccttgag

SEQ ID NO: 7 - DNA, 28 bp, Artificial Sequence
  Primer sequence for MERS coronavirus
ttttgtttccactgttttcgtgcctgca

SEQ ID NO: 8 - DNA, 28 bp, Artificial Sequence
  Primer sequence for MERS coronavirus
tttctgttttcgtgcctgcaacgcgcga

SEQ ID NO: 9 - DNA, 28 bp, Artificial Sequence
  Primer sequence for human coronavirus 229E
ttttaatgcaatcactgtcacaaccgtg

SEQ ID NO: 10 - DNA, 28 bp, Artificial Sequence
  Primer sequence for human coronavirus 229E
tttaaaacccagcctgtgctattttgtg

SEQ ID NO: 11 - DNA, 28 bp, Artificial Sequence
  Primer sequence for human coronavirus OC43
tttgtatgttaggccgataattgaggac

SEQ ID NO: 12 - DNA, 28 bp, Artificial Sequence
  Primer sequence for human coronavirus OC43
ttcaaacagcaaaaccactagtatcgct

SEQ ID NO: 13 - DNA, 28 bp, Artificial Sequence
  Primer sequence for human coronavirus NL63
ttattcctcctccttcattttacatgcc

SEQ ID NO: 14 - DNA, 28 bp, Artificial Sequence
  Primer sequence for human coronavirus NL63
tttaatttaaggtccttatgaggtccag

SEQ ID NO: 15 - DNA, 28 bp, Artificial Sequence
  Primer sequence for human coronavirus HKU1
tttacacttctaytccctccgatgtttc

SEQ ID NO: 16 - DNA, 28 bp, Artificial Sequence
  Primer sequence for human coronavirus HKU1
tttaagattagcratctcatcagccata

SEQ ID NO: 17 - DNA, 28 bp, Artificial Sequence
  Primer sequence for Influenza A
tttatggctaaagacaagaccratcctg

SEQ ID NO: 18 - DNA, 28 bp, Artificial Sequence
  Primer sequence for Influenza A
tttttaagggcattytggacaaakcgtc

SEQ ID NO: 19 - DNA, 28 bp, Artificial Sequence
  Primer sequence for Influenza B
tttggatgaagaagatggccatcggatc

SEQ ID NO: 20 - DNA, 28 bp, Artificial Sequence
  Primer sequence for Influenza B
ttttctaattgtctccctcttctggtga

SEQ ID NO: 21 - DNA, 28 bp, Artificial Sequence
  Primer sequence for human RNAse P control
tttacttcagcatggcggtgtttgcaga

SEQ ID NO: 22 - DNA, 28 bp, Artificial Sequence
  Primer sequence for human RNAse P control
ttttgatagcaacaactgaatagccaag

SEQ ID NO: 23 - DNA, 28 bp, Artificial Sequence
  Primer sequence for SARS CoV-2 Nucleocapsid
ttttaatggaccccaaaatcagcgaaat

SEQ ID NO: 24 - DNA, 28 bp, Artificial Sequence
  Primer sequence for SARS CoV-2 Nucleocapsid
  modified_base 1: Thymidine at position 1 is modified with a fluorescent label
tttttctggttactgccagttgaatctg

SEQ ID NO: 25 - DNA, 28 bp, Artificial Sequence
  Primer sequence for coronavirus Nucleocapsid
tttactgattacaaacattggccgcaaa

SEQ ID NO: 26 - DNA, 29 bp, Artificial Sequence
  Primer sequence for coronavirus Nucleocapsid
  modified_base 1: Thymidine at position 1 is modified with a fluorescent label
ttttgccaatgcgycgacattccraagaa

SEQ ID NO: 27 - DNA, 28 bp, Artificial Sequence
  Primer sequence for coronavirus Nucleocapsid
tttagggagccttgaatacaccaaaaga

SEQ ID NO: 28 - DNA, 28 bp, Artificial Sequence
  Primer sequence for coronavirus Nucleocapsid
tttaagttgtagcacgattgcagcattg

SEQ ID NO: 29 - DNA, 28 bp, Artificial Sequence
  Primer sequence for MERS CoV
tttccatatgtccaaagagagactaatg

SEQ ID NO: 30 - DNA, 28 bp, Artificial Sequence
  Primer sequence for MERS CoV
  modified_base 1: Thymidine at position 1 is modified with a fluorescent label
ttttagtagcgcagagctgcttaaacga

SEQ ID NO: 31 - DNA, 28 bp, Artificial Sequence
  Primer sequence for HCoV-229E
tttacatactatcaacccattcaacaag

SEQ ID NO: 32 - DNA, 28 bp, Artificial Sequence
  Primer sequence for HCoV-229E
  modified_base 1: Thymidine at position 1 is modified with a fluorescent label
tttctcggcacggcaactgtcatgtatt

SEQ ID NO: 33 - DNA, 28 bp, Artificial Sequence
  Primer sequence for human coronavirus OC43
ttttcatacyctgacggtcacaataata

SEQ ID NO: 34 - DNA, 28 bp, Artificial Sequence
  Primer sequence for human coronavirus OC43
  modified_base 1: Thymidine at position 1 is modified with a fluorescent label
ttttaaccttagcaacagwcatataagc

SEQ ID NO: 35 - DNA, 28 bp, Artificial Sequence
  Primer sequence for human coronavirus NL63
ttatagttctgataaggcaccatatagg

SEQ ID NO: 36 - DNA, 28 bp, Artificial Sequence
  Primer sequence for human coronavirus NL63
  modified_base 1: Thymidine at position 1 is modified with a fluorescent label
tttgaactttaggaggcaaatcaacacg

SEQ ID NO: 37 - DNA, 28 bp, Artificial Sequence
  Primer sequence for human coronavirus HKU1
tttgatcctactaytcaagaagctatcc

SEQ ID NO: 38 - DNA, 28 bp, Artificial Sequence
  Primer sequence for human coronavirus HKU1
  modified_base 1: Thymidine at position 1 is modified with a fluorescent label
tttcttaatgaacgaktattgggtccac

SEQ ID NO: 39 - DNA, 28 bp, Artificial Sequence
  Primer sequence for Influenza A
tttcaagaccratcctgtcacctctgac

SEQ ID NO: 40 - DNA, 28 bp, Artificial Sequence
  Primer sequence for Influenza A
  modified_base 1: Thymidine at position 1 is modified with a fluorescent label
tttaagggcattytggacaaakcgtcta

SEQ ID NO: 41 - DNA, 28 bp, Artificial Sequence
  Primer sequence for Influenza B
tttgcgtctcaatgaaggacattcaaag

SEQ ID NO: 42 - DNA, 28 bp, Artificial Sequence
  Primer sequence for Influenza B
  modified_base 1: Thymidine at position 1 is modified with a fluorescent label
ttttaatcggtgctcttgaccaaattgg

SEQ ID NO: 43 - DNA, 28 bp, Artificial Sequence
  Primer sequence for human RNAse P control
tttgtttgcagatttggacctgcgagcg

SEQ ID NO: 44 - DNA, 27 bp, Artificial Sequence
  Primer sequence for human RNAse P control
  modified_base 1: Thymidine at position 1 is modified with a fluorescent label
tttaaggtgacggctgtctccacaagt

SEQ ID NO: 45 - DNA, 30 bp, Artificial Sequence
  Probe sequence for SARS CoV-2 Nucleocapsid
tttttttccgcattacgtttggtgtttttt

SEQ ID NO: 46 - DNA, 30 bp, Artificial Sequence
  Probe sequence for SARS CoV-2 Nucleocapsid
tttttttatcagcgaaatgcaccctttttt

SEQ ID NO: 47 - DNA, 30 bp, Artificial Sequence
  Probe sequence for SARS CoV-2 Nucleocapsid
ttttttttttgcccccagcgcttctttttt

SEQ ID NO: 48 - DNA, 30 bp, Artificial Sequence
  Probe sequence for SARS CoV-2 Nucleocapsid
ttttttacaatttgcccccagcgtcttttt

SEQ ID NO: 49 - DNA, 30 bp, Artificial Sequence
  Probe sequence for SARS Nucleocapsid
tttttttttgctccragtgcctcttttttt

SEQ ID NO: 50 - DNA, 30 bp, Artificial Sequence
  Probe sequence for SARS Nucleocapsid
ttttttttgctccragtgcctctgtccttt

SEQ ID NO: 51 - DNA, 32 bp, Artificial Sequence
  Probe sequence for coronavirus Bat precursor
tttttgtttgcacctagtgcttcagccctttt

SEQ ID NO: 52 - DNA, 32 bp, Artificial Sequence
  Probe sequence for coronavirus Pangolin precursor
tttttatttgcwcctagcgcttctgctctttt

SEQ ID NO: 53 - DNA, 32 bp, Artificial Sequence
  Probe sequence for coronavirus Bat precursor- Yunnan 2013
tttttgtttgcacccagtgcttctgctctttt

SEQ ID NO: 54 - DNA, 30 bp, Artificial Sequence
  Probe sequence for coronavirus Bat precursor- Yunnan 2019
ttttttacaattcgctcccagcgtcttttt

SEQ ID NO: 55 - DNA, 30 bp, Artificial Sequence
  Probe sequence for coronavirus Nucleocapsid
tttttctggcacccgcaatcctgtcttttt

SEQ ID NO: 56 - DNA, 30 bp, Artificial Sequence
  Probe sequence for coronavirus Nucleocapsid
ttttttaycacattggcacccgcatctttt

SEQ ID NO: 57 - DNA, 30 bp, Artificial Sequence
  Probe sequence for MERS coronavirus
ttttatctcttcacataatcgccctttttt

SEQ ID NO: 58 - DNA, 30 bp, Artificial Sequence
  Probe sequence for MERS coronavirus
ttttttataatcgccccgagctcgtctttt

SEQ ID NO: 59 - DNA, 32 bp, Artificial Sequence
  Probe sequence for human coronavirus 229E
tttttttgctttacgttgacggacattttttt

SEQ ID NO: 60 - DNA, 32 bp, Artificial Sequence
  Probe sequence for human coronavirus 229E
tttttttcaggtgttcaggttcataatctttt

SEQ ID NO: 61 - DNA, 32 bp, Artificial Sequence
  Probe sequence for human coronavirus OC43
tttttcatctttacattcaaggtataattttt

SEQ ID NO: 62 - DNA, 32 bp, Artificial Sequence
  Probe sequence for human coronavirus OC43
ttttctgctattctttggcagatttgcttttt

SEQ ID NO: 63 - DNA, 32 bp, Artificial Sequence
  Probe sequence for human coronavirus NL63
tttttctaagagcgttggcgtatgcttttttt

SEQ ID NO: 64 - DNA, 32 bp, Artificial Sequence
  Probe sequence for human coronavirus NL63
ttttttaagatgagcagattggttaccttttt

SEQ ID NO: 65 - DNA, 32 bp, Artificial Sequence
  Probe sequence for human coronavirus HKU1
ttttttcaggttcacgttctcaatcatttttt

SEQ ID NO: 66 - DNA, 32 bp, Artificial Sequence
  Probe sequence for human coronavirus HKU1
ttttctgtacgattytgcctcaaggccttttt

SEQ ID NO: 67 - DNA, 30 bp, Artificial Sequence
  Probe sequence for Influenza A
tttttttcgtgcccagtgagcgagtttttt

SEQ ID NO: 68 - DNA, 30 bp, Artificial Sequence; identical to SEQ ID NO: 67
  Probe sequence for Influenza A

SEQ ID NO: 69 - DNA, 30 bp, Artificial Sequence
  Probe sequence for Influenza B
ttttttccaattcgagcagctgaatttttt

SEQ ID NO: 70 - DNA, 30 bp, Artificial Sequence
  Probe sequence for Influenza B
ttttttagcagctgaaactgcggttttttt

SEQ ID NO: 71 - DNA, 34 bp, Artificial Sequence
  Probe sequence for human RNAse P control
ttttttttctgacctgaaggctctgcgcgttttt

SEQ ID NO: 72 - DNA, 30 bp, Artificial Sequence
  Probe sequence for human RNAse P control
tttttcttgacctgaaggctctgctttttt

SEQ ID NO: 73 - DNA, 34 bp, Artificial Sequence
  Probe sequence for Negative Control
ttttttctactacctatgctgattcactcttttt

SEQ ID NO: 74 - DNA, 28 bp, Artificial Sequence
  Primer sequence for ?-coronavirus Nucleocapsid
  modified_base 1: Thymidine at position 1 is modified with a fluorescent label
ttttgccaatgcgcgacattccgaagaa

SEQ ID NO: 75 - DNA, 28 bp, Artificial Sequence
  Primer sequence for SARS-CoV-2 Spike Gene
tttagtgttataacaccaggaacaaata

SEQ ID NO: 76 - DNA, 28 bp, Artificial Sequence
  Primer sequence for SARS-CoV-2 Spike Gene
  modified_base 1: Thymidine at position 1 is modified with a fluorescent label
ttttgcatgaatagcaacagggacttct

SEQ ID NO: 77 - DNA, 30 bp, Artificial Sequence
  Primer sequence for Pan-coronavirus RNA- dependent RNA polymerase
ttttttaataagtattttaagcaytggagt

SEQ ID NO: 78 - DNA, 29 bp, Artificial Sequence
  Primer sequence for Pan-coronavirus RNA- dependent RNA polymerase
  modified_base 1: Thymidine at position 1 is modified with a fluorescent label
tttaagagtgtgttaaaatttgaacaatg

SEQ ID NO: 79 - DNA, 30 bp, Artificial Sequence
  Primer sequence for Pan-coronavirus RNA- dependent RNA polymerase
ttttgtttaagaagtattttaartattggg

SEQ ID NO: 80 - DNA, 29 bp, Artificial Sequence
  Primer sequence for Pan-coronavirus RNA- dependent RNA polymerase
  modified_base 1: Thymidine at position 1 is modified with a fluorescent label
tttaatagtgtattraaattagcacaatg

SEQ ID NO: 81 - DNA, 28 bp, Artificial Sequence
  Primer sequence for Influenza B
tttggatcctcaactcactcttcgagcg

SEQ ID NO: 82 - DNA, 28 bp, Artificial Sequence
  Primer sequence for Influenza A
  modified_base 1: Thymidine at position 1 is modified with a fluorescent label
tttgggcattytggacaaakcgtctacg

SEQ ID NO: 83 - DNA, 28 bp, Artificial Sequence
  Primer sequence for human respiratory syncytial virus
tttaaaratggctcttagcaaagtcaag

SEQ ID NO: 84 - DNA, 28 bp, Artificial Sequence
  Primer sequence for human respiratory syncytial virus
  modified_base 1: Thymidine at position 1 is modified with a fluorescent label
tttcgttgratrgtrtatttgctggatg

SEQ ID NO: 85 - DNA, 30 bp, Artificial Sequence
  Probe sequence for coronavirus Bat precursor
  modified_base 1: Thymidine at position 1 is modified with a fluorescent label
ttttttgtttgcacctagtgcttccttttt

SEQ ID NO: 86 - DNA, 30 bp, Artificial Sequence
  Probe sequence for coronavirus Pangolin precursor
tttttttttgctcctagcgcttcttttttt

SEQ ID NO: 87 - DNA, 34 bp, Artificial Sequence
  Probe sequence for Pan SARS-CoV-2 614
tttttctctttatcaggrtgttaactgctttttt

SEQ ID NO: 88 - DNA, 30 bp, Artificial Sequence
  Probe sequence for SARS-CoV-2 614 variant
ttttttcctatcagggtgttaacttttttt

SEQ ID NO: 89 - DNA, 30 bp, Artificial Sequence
  Probe sequence for SARS-CoV-2 614 variant
tttttcttatcaggatgttaactttttttt

SEQ ID NO: 90 - DNA, 35 bp, Artificial Sequence
  Probe sequence for human coronavirus OC43
tttttatatcatcctaacactgttgattgtttttt

SEQ ID NO: 91 - DNA, 35 bp, Artificial Sequence
  Probe sequence for human coronavirus NL63
tttttttatcatcctaattgtagtgactgtttttt

SEQ ID NO: 92 - DNA, 35 bp, Artificial Sequence
  Probe sequence for human coronavirus HKU1
tttttgtatcatcctaatactgtggattgtttttt

SEQ ID NO: 93 - DNA, 35 bp, Artificial Sequence
  Probe sequence for human coronavirus 229E
tttttttatcatcctgattgtgttgattgcttttt

SEQ ID NO: 94 - DNA, 35 bp, Artificial Sequence
  Probe sequence for MERS
tttttaattgcgttaattgtactgatgaccttttt

SEQ ID NO: 95 - DNA, 36 bp, Artificial Sequence
  Probe sequence for Influenza B
ttttccaattcgagcagctgaaactgcggtgttttt

SEQ ID NO: 96 - DNA, 36 bp, Artificial Sequence
  Probe sequence for human respiratory syncytial virus-A
tttttcacactcaacaaagatcaacttcttcttctt

SEQ ID NO: 97 - DNA, 35 bp, Artificial Sequence
  Probe sequence for human respiratory syncytial virus-B
tttttcgatacattaaataaggatcagcttttttt

SEQ ID NO: 98 - DNA, 48 bp, Artificial Sequence
  Sequence for SARS CoV-2
ttcttcggaatgtcgcgcattggcaaaacatggtcatagctgtttcct

SEQ ID NO: 99 - DNA, 52 bp, Artificial Sequence
  Sequence for discordant SARS CoV-2
  misc_feature 5, 6, 10, 16, 27, 32, 38, 39, 40, 41, 47: n is any nucleotide
ttccnncggnaggccngccattgggcnaaacntggacnnnngcgtgntttcc

SEQ ID NO: 100 - DNA, 50 bp, Artificial Sequence
  Sequence for discordant SARS CoV-2
  misc_feature 16, 26: n is any nucleotide
ttcttcgggagggcgngcattgggcnaaacatgggtcatagctgtttcct

SEQ ID NO: 101 - DNA, 54 bp, Artificial Sequence
  Sequence for discordant SARS CoV-2
  misc_feature 12, 23,48: n is any nucleotide
ttcttcgggaangtcgcggcatnggcaaaacatggggtcataggctgntttcct

SEQ ID NO: 102 - DNA, 55 bp, Artificial Sequence
  Sequence for discordant SARS CoV-2
  misc_feature 17, 37, 46: n is any nucleotide
ttcttcgggaaggtcgnggcattggcaaaacatgggntcataggcntgatttcct

SEQ ID NO: 103 - DNA, 50 bp, Artificial Sequence
  Sequence for discordant SARS CoV-2
  misc_feature 12, 22, 32: n is any nucleotide
ttcttcgggaangtcgcgcatnggcaaaacanggtcatagctggtttcct

SEQ ID NO: 104 - DNA, 53 bp, Artificial Sequence
  Sequence for discordant SARS CoV-2
  misc_feature 44, 47: n is any nucleotide
ttcttcgggaaggtcgcggcattggcaaaacatggatcatagtntgntttcct

SEQ ID NO: 105 - DNA, 49 bp, Artificial Sequence
  Sequence control for Positive SARS CoV-2
  misc_feature 41: n is any nucleotide
ttcttcggaatgtcgcgcattggcaaaacatggtcatagcntgtttcct

SEQ ID NO: 106 - DNA, 28 bp, Artificial Sequence
  Primer sequence for Influenza B.8.univ-RE1.3
ttttccctcttctggtgataatcggtgc

SEQ ID NO: 107 - DNA, 60 bp, Artificial Sequence
  Sequence for human respiratory syncytial virus-A
gatggctcttagcaaagtcaagttgaatgatacactcaacaaagatcaacttctgtcatc

SEQ ID NO: 108 - DNA, 60 bp, Artificial Sequence
  Sequence for human respiratory syncytial virus-B
gatggctcttagcaaagtcaagttaaatgatacattaaataaggatcagctgctgtcatc

SEQ ID NO: 109 - DNA, 60 bp, Artificial Sequence
  Sequence for human respiratory syncytial virus-A
cagcaaatacaccatccaacggagcacaggagatagtattgatactcctaattatgatgt

SEQ ID NO: 110 - DNA, 60 bp, Artificial Sequence
  Sequence for human respiratory syncytial virus-B
cagcaaatacactattcaacgtagtacaggagataatattgacactcccaattatgatgt

SEQ ID NO: 111 - DNA, 26 bp, Artificial Sequence
  Probe sequence for human respiratory syncytial virus
gctcttagcaaagtcaagttgaatga

SEQ ID NO: 112 - DNA, 21 bp, Artificial Sequence
  Probe sequence for human respiratory syncytial virus
tgctccgttggatggtgtatt

SEQ ID NO: 113 - DNA, 33 bp, Artificial Sequence
  Probe sequence for human respiratory syncytial virus
acactcaacaaagatcaacttctgtcatccagc

SEQ ID NO: 114 - DNA, 26 bp, Artificial Sequence
  Probe sequence for human respiratory syncytial virus
gatggctcttagcaaagtcaagttaa

SEQ ID NO: 115 - DNA, 30 bp, Artificial Sequence
  Probe sequence for human respiratory syncytial virus
tgtcaatattatctcctgtactacgttgaa

SEQ ID NO: 116 - DNA, 35 bp, Artificial Sequence
  Probe sequence for human respiratory syncytial virus
tgatacattaaataaggatcagctgctgtcatcca

SEQ ID NO: 117 - DNA, 25 bp, Artificial Sequence
  Probe sequence for human respiratory syncytial virus
aaaratggctcttagcaaagtcaag

SEQ ID NO: 118 - DNA, 25 bp, Artificial Sequence
  Probe sequence for human respiratory syncytial virus
cgttgratrgtrtatttgctggatg

SEQ ID NO: 119 - DNA, 23 bp, Artificial Sequence
  Probe sequence for human respiratory syncytial virus
acactcaacaaagatcaacttct

SEQ ID NO: 120 - DNA, 23 bp, Artificial Sequence
  Probe sequence for human respiratory syncytial virus
acattaaataaggatcagctgct

SEQ ID NO: 121 - DNA, 27 bp, Artificial Sequence
  Primer sequence for human coronavirus NL63 and OC43
tttaataagtattttaagcaytggagt

SEQ ID NO: 122 - DNA, 27 bp, Artificial Sequence
  Primer sequence for MERS and human coronavirus 229E and HKU1
tgtttaagaagtattttaartattggg

SEQ ID NO: 123 - DNA, 26 bp, Artificial Sequence
  Primer sequence for human coronavirus 299E
aagagtgtgttaaaatttgaacaatg

SEQ ID NO: 124 - DNA, 26 bp, Artificial Sequence
  Primer sequence for MERS and human coronavirus NL63, human coronavirus OC43 and human coronavirus HKU 1
aatagtgtattraaattagcacaatg

SEQ ID NO: 125 - DNA, 25 bp, Artificial Sequence
  Probe sequence for human coronavirus NL63
ttatcatcctaattgtagtgactgt

SEQ ID NO: 126 - DNA, 25 bp, Artificial Sequence
  Probe sequence for human coronavirus 229E
ttatcatcctgattgtgttgattgc

SEQ ID NO: 127 - DNA, 25 bp, Artificial Sequence
  Probe sequence for human coronavirus OC43
atatcatcctaacactgttgattgt

SEQ ID NO: 128 - DNA, 25 bp, Artificial Sequence
  Probe sequence for human coronavirus HKU1
gtatcatcctaatactgtggattgt

SEQ ID NO: 129 - DNA, 25 bp, Artificial Sequence
  Probe sequence for MERS
aattgcgttaattgtactgatgacc

SEQ ID NO: 130 - DNA, 27 bp, Artificial Sequence
  Primer sequence for human coronavirus NL63 and human coronavirus OC43
ttyaataagtaytttaagcaytggagt

SEQ ID NO: 131 - DNA, 27 bp, Artificial Sequence
  Primer sequence for MERS and human coronavirus 229E and human coronavirus HKU1
tstttrabaagtaytttaartattggg

SEQ ID NO: 132 - DNA, 26 bp, Artificial Sequence
  Primer sequence for human coronavirus 299E
aagagtgtgttaaaatttgaacaatg

SEQ ID NO: 133 - DNA, 26 bp, Artificial Sequence
  Primer sequence for MERS and human coronavirus NL63, human coronavirus OC43 and human coronavirus HKU1
aahartryrttraaattagcacaatg

SEQ ID NO: 134 - DNA, 25 bp, Artificial Sequence
  Primer sequence for human coronavirus NL63
ttatcatcctaattgtagtgactgt

SEQ ID NO: 135 - DNA, 25 bp, Artificial Sequence
  Primer sequence for human coronavirus 229E
ttatcatcctgattgtgttgattgc

SEQ ID NO: 136 - DNA, 25 bp, Artificial Sequence
  Primer sequence for human coronavirus OC43
atatcatcctaacackgttgattgt

SEQ ID NO: 137 - DNA, 25 bp, Artificial Sequence
  Primer sequence for human coronavirus HKU1
gtatcatcctaatactgtggattgt

SEQ ID NO: 138 - DNA, 24 bp, Artificial Sequence
  Primer sequence for MERS
attgcgttaattgtactgatgacc

SEQ ID NO: 139 - DNA, 24 bp, Artificial Sequence
  Sequence for SARS?CoV?2 Wuhan-Hu-1
  misc_feature 7: n is any nucleotide
ccctagnttcaactggcagtaacc

SEQ ID NO: 140 - DNA, 25 bp, Artificial Sequence
  Sequence for SARS?CoV?2 Wuhan-Hu-1
ccctcagattcaactggcagtaacc

SEQ ID NO: 141 - DNA, 76 bp, Artificial Sequence
  Sequence for SARS?CoV?2 Wuhan-Hu-1
  misc_feature 20,29,61,62,63: n is any nucleotide
ctgactgggactggtgacgncgccctagnttcaactggcagtaacccgtagaaaacatggnnntagctgtttccta

(19) United States
(12) Patent Application Publication
(10) Pub. No.: US 2022/0364157 A1
(43) Pub. Date: Nov. 17, 2022

(54) Methods for Detecting Low Levels of Covid-19 Virus

(71) Applicants: Michael Edward Hogan, Stony Brook, NJ (US); Benjamin Alan Katchman, Tucson, AZ (US); Frederick Henry Eggers, Sahuarita, AZ (US); Cory Scott Newland, Tucson, AZ (US)

(72) Inventors: Michael Edward Hogan, Stony Brook, NJ (US); Benjamin Alan Katchman, Tucson, AZ (US); Frederick Henry Eggers, Sahuarita, AZ (US); Cory Scott Newland, Tucson, AZ (US)

(73) Assignee: PathogenDx, Inc., Scottsdale, AZ (US)

(21) Appl. No.: 16/950,210

(22) Filed: Nov. 17, 2020

Related U.S. Application Data
(60) Provisional application No. 63/078,783, filed on Sep. 15, 2020.
(60) Provisional application No. 63/000,844, filed on Mar. 27, 2020.

(51) Int. Cl. C12Q 1/6816; C12Q 1/686; C12Q 1/6876

(52) U.S. Cl. CPC C12Q 1/6816; C12Q 1/686; C12Q 1/6876; C12Q 2527/101; C12Q 2527/113; C12Q 2527/119; C12Q 2563/107

(57) ABSTRACT

Provided herein is a method for detecting the presence of a COVID-19 virus in a human sample or an environmental sample having one or more viruses and bacterial pathogens. Samples are processed to obtain total nucleic acids. A combined reverse transcription and asymmetric PCR amplification reaction is performed to obtain fluorescent labeled COVID-19 virus specific amplicons. The amplicons are detected by microarray hybridization near the lowest limit of detection. Also provided is a method for detecting concurrently with COVID-19 virus, the presence of respiratory disease-causing pathogens including viruses, bacteria and fungus in a single assay using the above method.

### CROSS-REFERENCE TO RELATED APPLICATIONS

This non-provisional application claims the benefit of priority under 35 U.S.C. § 119(e) of provisional applications U.S. Ser. No. 63/078,783, filed Sep. 15, 2020, and U.S. Ser. No. 63/000,844, filed Mar. 27, 2020, both of which are hereby incorporated by reference in their entireties.

### BACKGROUND OF THE INVENTION

### Field of the Invention

The present invention relates to the field of multiplex based viral pathogen detection and analysis. More particularly, the present invention relates to detecting the presence of COVID-19 virus in patient and environmental samples.

### Description of the Related Art

The COVID-19 pandemic has increased awareness that viral infection can be an existential threat to health, public safety and the US economy. More fundamentally, there is a recognition that the viral risks are more dangerous and more complex than had been thought and will require new approaches to diagnostics and screening.

The next pandemic wave is expected to have more pronounced flu-like symptoms (seasonal influenza A and/or B) coupled with the COVID-19, or COVID-19 variants that will coexist with the Coronavirus already responsible for the common cold. These complexities are expected to pose significant challenges to public health and the healthcare system in diagnosing multi-symptom conditions accurately and efficiently.

The COVID-19 pandemic has also led to the realization of an additional level of complexity that the realization that human health and environmental contamination are linked in a fundamental way that affects collection efficiency and increases risk to the healthcare workers (1, 2). Alternatives to nasopharyngeal collection methods such as for example, saliva collection are needed to enable scalability among millions of individuals.

Q-RT-PCR technology has dominated COVID-19 diagnostics and public health screening. Independent of the test developer, Q-RT-PCR has been shown to have an unusually high false negative rate (15% up to 30%). As of May 2020, the CDC has recorded 613,041 COVID-19 tests. With a 15% false negative rate, approximately 91,956 people would thus be falsely classified as free of infection. Meta-analysis has shown that the false negative rate for Q-RT-PCR is high below day 7 of infection when viral load is still low. This renders Q-RT-PCR ineffective as a tool for early detection of weak symptomatic carriers while also lessening its value in epidemiology.

Thus, there is a need in the art for superior tools to not only administer and stabilize sample collection for respiratory viruses from millions of samples in parallel obtained from diverse locations including, clinic, home, work, school and in transportation hubs, but also to test multiple respiratory markers at the highest levels of sensitivity and specificity.

### SUMMARY OF THE INVENTION

The present invention is directed to a method for detecting Coronavirus disease 2019 (COVID-19) in a sample. A sample is obtained and a total RNA isolated. A combined, reverse transcription reaction and an asymmetric PCR amplification reaction is performed on the isolated total RNA using at least one fluorescent labeled primer pair selective for a target nucleotide sequence in the COVID-19 virus, the fluorescent labeled primer pair comprising an unlabeled primer and a fluorescently labeled primer. Fluorescent labeled COVID-19 virus amplicons thus generated are hybridized to a plurality of nucleic acid probes, each attached to a solid microarray support. Each of the nucleic acid probes have sequence corresponding to a sequence determinant in the COVID-19 virus. The microarray is washed at least once and imaged to detect at least one fluorescent signal from the hybridized fluorescent labeled COVID-19 virus amplicons. The present invention is also directed to a related method further comprising calculating an intensity of the fluorescent signal for the COVID-19 virus amplicons, correlating with the number of COVID-19 virus genomes in the sample. The present invention is further directed to a related method for detecting at least one other non-COVID-19 virus in the sample by employing at least two fluorescent labeled primer pairs selective for the target nucleotide sequence in the COVID-19 virus and in the at least one other non-COVID-19 virus, and nucleic acid probes having a sequence corresponding to sequence determinants in the COVID-19 virus and the at least one of the other non-COVID-19 virus to perform the steps described above.

The present invention is also directed to a method for detecting a respiratory disease-causing pathogen in a sample. A sample is obtained, and total nucleic acids are isolated. A combined, reverse transcription reaction and an asymmetric PCR amplification reaction is performed on the isolated total nucleic acids using at least one fluorescent labeled primer pair selective for a target nucleotide sequence in at least one respiratory disease-causing pathogen, the fluorescent labeled primer pair comprising an unlabeled primer and a fluorescently labeled primer. At last one fluorescent labeled pathogen specific amplicon thus generated are hybridized to a plurality of nucleic acid probes, each attached to a solid microarray support. Each of the nucleic acid probes have sequence corresponding to sequence determinants in the pathogen. The microarray is washed at least once and imaged to detect at least one fluorescent signal from the hybridized fluorescent labeled pathogen specific amplicons. The present invention is also directed to a related method further comprising calculating an intensity of the fluorescent signal for the fluorescent labeled pathogen specific amplicons, correlating with the number of pathogen specific genomes in the sample.

The present invention is further directed to a method for detecting a Coronavirus disease 2019 (COVID-19) virus in a sample. A sample is obtained, and a total nucleic acid is isolated. A combined, reverse transcription reaction and an asymmetric PCR amplification reaction is performed on the isolated total nucleic using at least one fluorescent labeled primer pair selective for a target nucleotide sequence in the COVID-19 virus, the fluorescent labeled primer pair comprising an unlabeled primer and a fluorescently labeled primer in an excess over the unlabeled primer. Fluorescent labeled COVID-19 virus amplicons thus generated are hybridized to a plurality of nucleic acid probes each attached to a solid microarray support. Each of the nucleic acid probes have a sequence corresponding to a sequence determinant in the COVID-19 virus. The microarray is washed at least once and imaged to detect at least one fluorescent signal from the hybridized fluorescent labeled COVID-19 virus amplicons thereby detecting the COVID-19 in the sample. The present invention is also directed to a related method for detecting additionally, at least one non-COVID-19 virus in the sample by performing the combined reverse transcription and the asymmetric PCR amplification reaction on the isolated total nucleic acid using at least two fluorescent labeled primer pairs, each comprising the unlabeled primer and the fluorescently labeled primer selective for a target nucleotide sequence in the COVID-19 virus and the at least one non-COVID 19 virus. Fluorescent labeled COVID-19 and non-COVID-19 virus specific amplicons generated are hybridized to the plurality of nucleic acid probes having a sequence corresponding to the sequence determinant in the COVID-19 virus and the at least one non-COVID-19 virus. The present invention is also directed to a related method for detecting additionally, at least one bacterium in the sample by performing the combined reverse transcription and the asymmetric PCR amplification reaction on the isolated total nucleic acid using at least two fluorescent labeled primer pairs, each comprising the unlabeled primer and the fluorescently labeled primer selective for a target nucleotide sequence in the COVID-19 virus and the at least one bacterium. Fluorescent labeled COVID-19 virus and bacterium specific amplicons generated are hybridized to the plurality of nucleic acid probes having a sequence corresponding to the sequence determinant in the COVID-19 virus and the at least one bacterium. The present invention is further directed to a related method for detecting additionally, at least one fungus in the sample by performing the combined reverse transcription and the asymmetric PCR amplification reaction on the isolated total nucleic acid using at least two fluorescent labeled primer pairs, each comprising the unlabeled primer and the fluorescently labeled primer selective for a target nucleotide sequence in the nucleic acids from the COVID-19 virus and the at least one fungus. Fluorescent labeled COVID-19 virus and fungus specific amplicons generated are hybridized to the plurality of nucleic acid probes having a sequence corresponding to the sequence determinant in the COVID-19 virus and the at least one fungus.

### BRIEF DESCRIPTION OF THE FIGURES

FIG. 1 shows that random fluid aliquot sampling can deliver “positive” and “negative” aliquots and that amplification by tandem PCR for subsequent hybridization testing does not alter lowest limit of detection (LLoD) counting statistics.

FIG. 2 shows that DNA microarray-based hybridization near the lowest limit of detection allows “positive” hybridization signals to be validated on each sample tested based on internal “mismatched” and “sequence specific” controls.

FIGS. 3A-3C shows signal to noise near the lowest limit of detection. FIG. 3A shows Q-RT-PCR signal-to-noise in the limit of (1) vs (0) Genomes per Reaction. FIG. 3B shows the amount of DNA amplicons produced as a function of PCR cycle number. FIG. 3C shows microarray detection limit as a function of copy number of viral genome.

FIGS. 4A and 4B shows the probability of RT-PCR. FIG. 4A shows the probability of RT-PCR positive detection in samples from SARS-CoV2 infected patients. FIG. 4B shows the probability of samples identified as infected when RT-PCR reports negative detection.

FIG. 5 shows that near the lowest limit of detection tandem PCR then microarray hybridization distinguishes “positive” from a “negative” signal relative to internal controls and “binary” over significant dilution.

FIGS. 6A-6C shows relative fluorescent values for hybridization-based SARS-CoV2 detection in nasal samples. FIG. 6A shows a box and whiskers plot of relative fluorescent values for hybridization-based SARS-CoV2 detection in nasal samples. FIG. 6B shows sensitivity of DETECTX-RV in detecting SARS-CoV2 RNA. FIG. 6C shows sensitivity of Q-RT-PCR in detecting SARS-CoV2 RNA.

FIGS. 7A-7C shows the DETECTX-RV-V2 platform. FIG. 7A shows the workflow, based on an Asymmetric, Tandem, Two-Step Labelling PCR reaction, for the automated DETECTX-RV-V2 platform used for detecting SARS-CoV2 RNA. FIG. 7B shows the related workflow, based on the corresponding Asymmetric, One-Step RT-PCR reaction, for the automated DETECTX-RV-V2 platform used for detecting SARS-CoV2 RNA. FIG. 7C shows a 96-well automation-friendly microarray format for DETECTX-RV-V2.

FIG. 8 shows a DETECTX-RV pan respiratory pathogen diagnostic platform roadmap.

FIG. 9 shows the enhanced content DETECTX-RV pan respiratory pathogen diagnostic platform roadmap.

FIG. 10 shows the results of RNA stability analysis during environmental air analysis.

FIG. 11 shows the results of RNA stability analysis during environmental monitoring of surfaces by swabbing.

FIG. 12 shows microarray data for detection of SARS-CoV2 N3 target gene at various time points after spiking into SOW+ (with dye) and SOW− (minus dye).

FIGS. 13A-13B show quality control images for printed microarray plates. FIG. 13A shows a representative image a printed 96-well DETECTX-RV plate. FIG. 13B shows a printed 384-well Mini-RV plate comprising 13,824 probe spots with no printing errors.

FIG. 14 shows a representative DETECTX-RV hybridization data for clinical nasopharyngeal swab samples in 96-well format.

FIG. 15 is a microarray layout for 384-well printing showing triplicates for 12 probes (D=100-110 μm, P=160 μm) and two “make-up” slots, where “D” refers to average spot diameter and “P” refers to the pitch, i.e. the average separation.

FIGS. 16A-16D show hybridization data for a clinical nasopharyngeal swab sample in one well of the 384-well Mini-RV plate, shown magnified. FIG. 16A is a CY5 image showing initial SARS-CoV2 hybridization feasibility. FIG. 16A is a CY3 image showing initial SARS-CoV2 hybridization feasibility. FIG. 16C is a CY5-color analysis of the Cy-5 image shown in FIG. 16A showing probe identification. FIG. 16D is a CY3-color analysis of the Cy-3 image shown in FIG. 17B showing probe identification.

FIGS. 17A-17F shows the effects of parameters such as hybridization time, washing and spin-drying on signal strength. FIG. 17A shows an imaging matrix for 1 hour hybridization with mixing. FIG. 17B shows the imaging matrix in FIG. 17A after spin drying. FIG. 17C shows the benefit of a low salt wash buffer incubation prior to spin-drying on background, where the arrow signifies the benefit associated with the low salt wash prior to spin drying. FIG. 17D shows an imaging matrix for 30 hour hybridization with intermittent pipette mixing of the hybridization solution. FIG. 17E shows the imaging matrix in FIG. 17D after spin drying. FIG. 17F shows Optimization of hybridization in 96-well format.

FIGS. 18A-18B shows optimization data for Asymmetric One-Step RT-PCR reaction. FIG. 18A shows optimization data for SARS-CoV2 containing samples at a primer ratio of 4:1. FIG. 18A shows optimization data for SARS-CoV2 containing samples at a primer ratio of 8:1.

FIGS. 19A-19B show gel analysis for discordant TriCore clinical samples. FIG. 19A shows gel analysis for samples PATHO-003, PATHO-005, PATHO-008 and PATHO-012. FIG. 19B shows gel analysis for samples PATHO-015 and Positive sample-215981.

FIG. 20 shows a representative sequencing chromatograph for N1-M13F sample.

FIG. 21 shows a representative fully automated hybridization and wash in 96-well format.

FIGS. 22A and 22B show a comparison of automated and manual hybridization analysis in 96-well format. FIG. 22A show a representative (well A1) automated hybridization and wash in 96-well format. FIG. 22B show a representative (well G1) manual hybridization and wash in 96-well format.

FIGS. 23A-23C show the results of altering RT-PCR parameters on hybridization analysis. FIG. 23A compares the hybridization analysis for RNA from SARS-COV2-N1-RE1, amplified using 4 different protocols. FIG. 23B compares the hybridization analysis for RNA from SARS-COV2-N2-RE1.4, amplified using 4 different protocols. FIG. 23C compares the hybridization analysis for RNA from SARS-COV2-N3-RE1.1, amplified using 4 different protocols.

FIGS. 24A-24C compares the effect of hybridization conditions on the analysis. FIG. 24A compares static, shaking and pipetting hybridization methods in analysis of SARS-COV2-N1-RE1 samples. FIG. 24B compares static, shaking and pipetting hybridization methods in analysis of SARS-COV2-N2-RE1.4 samples. FIG. 24C compares static, shaking and pipetting hybridization methods in analysis of SARS-COV2-N3-RE1.1 samples.

FIG. 25 shows an illustration of the Ceres NANOTRAP method.

FIG. 26 shows a flowchart for the Ceres NANOTRAP method.

FIGS. 27A-27D shows microarray images from samples processed using the Ceres NANOTRAP method. FIG. 27A shows one microarray images from samples processed using the Ceres NANOTRAP method. FIG. 27B shows a second microarray images from samples processed using the Ceres NANOTRAP method. FIG. 27C shows a third microarray images from samples processed using the Ceres NANOTRAP method. FIG. 27D shows a fourth microarray images from samples processed using the Ceres NANOTRAP method.

FIG. 28 is a graphical representation of hybridization analysis for samples processed using the Ceres NANOTRAP method.

FIG. 29 is a graphical representation of hybridization analysis for samples processed using the Ceres NANOTRAP method.

FIGS. 30A-30D show clinical sensitivity and specificity of the Ceres NANOTRAP Mini-RV technology using the Cobas-Positive TriCore samples. FIG. 30A shows the RFU versus Ct value plot for RNase P probe. FIG. 30B shows the RFU versus Ct value plot for SARS-COV-2 N2-RE1.1 probe. FIG. 30C shows the RFU versus Ct value plot for SARS-COV-2 N2-RE1.4 probe. FIG. 30D shows the RFU versus Ct value plot for SARS-COV-2 N3-RE1.1 probe.

FIGS. 31A-31C show LoD analysis of the samples using Ceres NANOTRAP Mini-RV technology. FIG. 31A LoD analysis for the SARS-COV-2 N1 probe. FIG. 31B LoD analysis for the SARS-COV-2 N2 probe. FIG. 31C LoD analysis for the SARS-COV-2 N3 probe.

FIGS. 32A-32E shows the LoD analysis for contrived samples in VTM. FIG. 32A shows the results of probe signal versus threshold for SARS-COV-2 N1-RE1.1 probe. FIG. 32B shows the results of probe signal versus threshold for SARS-COV-2 N2-RE1.3 probe. FIG. 32C shows the results of probe signal versus threshold for SARS-COV-2 N2-RE1.4 probe. FIG. 32D shows the results of probe signal versus threshold for SARS-COV-2 N3-RE1.1 probe. FIG. 32E is an additional dataset showing the results of probe signal versus threshold for probes SARS-COV-2 N1-RE1.1, SARS-COV-2 N2-RE1.4 and SARS-COV-2 N3-RE1.1.

FIGS. 33A-33B shows LoD analysis for contrived samples in VTM. FIG. 33A shows the results of probe signal versus threshold for SARS-COV-2 N1-RE1.1 probe. FIG. 33B shows the results of probe signal versus threshold for SARS-COV-2 N2-RE1.4 probe.

FIG. 34 shows the results of stability testing for probes SARS-COV-2 N1-RE1.1, SARS-COV-2 N2-RE1.4 and SARS-COV-2 N3-RE1.1.

FIG. 35 shows a checkerboard pattern to evaluate the Ceres run on the Tecan EVO150.

FIG. 36 shows a summary of threshold analysis for clinical matrix samples.

FIGS. 37A-37C show LoD determination in clinical validation for Influenza samples.

FIG. 37A is a background analysis showing low thresholds for Inf A and Inf B. FIG. 37B is a representative LoD analysis for Inf A samples. FIG. 37C is a representative LoD analysis for Inf B samples.

FIGS. 38A-38C show data from an extended clinical threshold analysis for Influenza samples. FIG. 38A is a background analysis showing low thresholds for Inf A and Inf B. FIG. 38B is a representative LoD analysis for Inf A samples. FIG. 38C is a representative LoD analysis for Inf B samples.

FIG. 39 shows a comparison of Zymo and Ceres processing of mouthwash clinical samples on LoD range analysis.

FIGS. 40A-40B show LoD analysis for SARS-CoV-2. FIG. 40A shows a representative plot of LoD threshold determination for SARS-CoV-2 N1 probe. FIG. 40B shows a representative plot of LoD threshold determination for SARS-CoV-2 N2 probe.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein the phrase “lowest limit of detection (LLoD)” corresponds to the lowest number of genome copies capable of generating a measurable signal in the assay under consideration. For example, the LLoD corresponds to an analytical sensitivity of ˜0.3 copies/reaction and post extraction sensitivity of ˜3 copies/reaction.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure. For example, a fold excess of 3.6-fold to 8.8-fold is encompassed by about 4-fold to about 8-fold.

In one embodiment of the present invention, there is provided a method for detecting a Coronavirus disease 2019 (COVID-19) in a sample, comprising, obtaining the sample; isolating from the sample a total RNA; performing a combined reverse transcription and an asymmetric PCR amplification reaction on the isolated total RNA using at least one fluorescent labeled primer pair comprising an unlabeled primer, and a fluorescently labeled primer selective for a target nucleotide sequence in the COVID-19 virus to generate at least one fluorescent labeled COVID-19 virus amplicon; hybridizing the fluorescent labeled COVID-19 virus amplicons to a plurality of nucleic acid probes each having a sequence corresponding to a sequence determinant in the COVID-19 virus, each of said nucleic acid probes attached at a specific position on a solid microarray support; washing the microarray at least once; and imaging the microarray to detect at least one fluorescent signal from the hybridized fluorescent labeled COVID-19 virus amplicons, thereby detecting the COVID-19 in the sample.

In this embodiment, in one aspect, the sample is any sample obtained from a subject including, but not limited to a nasopharyngeal swab, nasal swab, mouth swab, and mouth wash (sample obtained by rinsing the subject's buccal cavity). A pooled sample obtained by combining two or more of these samples or by combining samples from multiple subjects may also be used. In another aspect of this embodiment, the sample is an environmental sample obtained from inanimate sources including but is not limited to an aerosol and a hard surface. In this embodiment, the aerosol samples are obtained using commercial air samplers such as for example a Coriolis Micro Air Sampler. In this embodiment, a sample from a hard surface is obtained using a swab. In either aspect of this embodiment, the viruses from samples obtained on swabs are dispersed in a liquid such as phosphate buffered saline. Aerosol samples are transferred into a volume of a liquid such as phosphate buffered saline.

In this embodiment, the COVID-19 virus is a Severe Acute Respiratory Syndrome Coronavirus 2 (SARS-CoV 2) or a mutated form thereof. In this embodiment, in some aspects, the sample is mixed with an RNA stabilizer such as for example, a chemical stabilizer that would protect the RNA from degradation during storage and transportation, prior to the RNA isolating step.

In this embodiment, total RNA is isolated from the COVID-19 virus and other potential contaminating pathogens and human cells. Any commercially available RNA isolation kits such as for example, a Quick-DNA/RNA Viral MagBead Kit from Zymo Research may be used for this purpose. The RNA thus isolated is used without further purification. Alternatively, an intact SARS-CoV-2 virus may be captured and enriched by binding to magnetic beads, using kits as for example that from Ceres Nanosciences (e.g. Ceres NANOTRAP technology), or by precipitation of the virus with polyethylene glycol (PEG), after which the enriched virus can be lyzed by heating with a “PCR-Friendly” lysis solution such as 1% NP40 in TE buffer and then used without additional purification.

In this embodiment, the COVID-19 virus RNA is used as a template in a combined reverse transcription/amplification reaction (RT-PCR). In this step, the nucleic acid sequences in the COVID-19 virus RNA are transcribed using a reverse transcriptase enzyme to generate COVID-19 complementary DNA (cDNA) that is amplified in the same reaction using COVID-19 virus selective fluorescent labeled primer pairs to generate fluorescent labeled COVID-19 virus amplicons. In this embodiment, fluorescent labeled primer pair comprises an unlabeled primer, and a fluorescently labeled primer in about 4-fold to about 8-fold excess of the unlabeled primer whereby, upon completion of the reaction, the fluorescently labelled amplicon will be primarily single stranded (that is, the reaction is a type of “asymmetric PCR”). In this embodiment, the fluorescent labeled primer pairs have forward (odd numbers) and reverse (even number) sequences shown in SEQ ID: 23 to SEQ ID: 28 (Table 1) and SEQ ID: 74 to SEQ ID: 80 (Table 37). Commercially available reverse transcriptase enzyme and buffers are used in this step. Controls including, but not limited to a RNAse P control having fluorescent labeled primer pair (forward primer SEQ ID: 43, reverse primer SEQ ID: 44) are also used herein. Any fluorescent label may be used, including, but not limited to CY3, a CY5, SYBR Green, a DYLIGHT™ DY647, a ALEXA FLUOR 647, a DYLIGHT™ DY547 and a ALEXA FLUOR 550.

TABLE 1
Primer sequences used for PCR

| | | | |
|---|---|---|---|
| SEQ ID NOS. | Target | Gene | Primer Sequence (5′ to 3′) |

First ampification primers

| | | | |
|---|---|---|---|
| SEQ ID: 1 | SARS CoV-2 | N₁ | TTTTGTCTGATAATGGACCCCAAAATCA |
| | Nucleocapsid | | |
| SEQ ID: 2 | SARS CoV-2 | N₁ | TTTGTTCTCCATTCTGGTTACTGCCAGT |
| | Nucleocapsid | | |
| SEQ ID: 3 | CoV | N₂ | TTTAGGAACTAATCAGACAAGGAACTGA |
| | Nucleocapsid* | | |
| SEQ ID: 4 | CoV | N₂ | TTTGTTCCCGAAGGTGTGACTTCCATGC |
| | Nucleocapsid* | | |
| SEQ ID: 5 | CoV | N₃ | TTTCGGCATCATATGGGTYGCAACTGAG |
| | Nucleocapsid* | | |
| SEQ ID: 6 | CoV | N₃ | TTTCCTTTTGGCAATGTTGTTCCTTGAG |
| | Nucleocapsid* | | |
| SEQ ID: 7 | MERS CoV | upE | TTTTGTTTCCACTGTTTTCGTGCCTGCA |
| SEQ ID: 8 | MERS CoV | upE | TTTCTGTTTTCGTGCCTGCAACGCGCGA |
| SEQ ID: 9 | HCoV-229E | M | TTTTAATGCAATCACTGTCACAACCGTG |
| SEQ ID: 10 | HCoV-229E | M | TTTAAAACCCAGCCTGTGCTATTTTGTG |
| SEQ ID: 11 | HCoV-OC43 | M | TTTGTATGTTAGGCCGATAATTGAGGAC |
| SEQ ID: 12 | HCoV-OC43 | M | TTCAAACAGCAAAACCACTAGTATCGCT |
| SEQ ID: 13 | NHCoV-NL63 | N | TTATTCCTCCTCCTTCATTTTACATGCC |
| SEQ ID: 14 | NHCoV-NL63 | N | TTTAATTTAAGGTCCTTATGAGGTCCAG |
| SEQ ID: 15 | NHCoV-HKU1 | N | TTTACACTTCTAYTCCCTCCGATGTTTC |
| SEQ ID: 16 | NHCoV-HKU1 | N | TTTAAGATTAGCRATCTCATCAGCCATA |
| SEQ ID: 17 | Influenza A | M | TTTATGGCTAAAGACAAGACCRATCCTG |
| SEQ ID: 18 | Influenza A | M | TTTTTAAGGGCATTYTGGACAAAKCGTC |
| SEQ ID: 19 | Influenza B | NS1 | TTTGGATGAAGAAGATGGCCATCGGATC |
| SEQ ID: 20 | Influenza B | NS1 | TTTTCTAATTGTCTCCCTCTTCTGGTGA |
| SEQ ID: 21 | Human RNAse | RNAse P | TTTACTTCAGCATGGCGGTGTTTGCAGA |
| | P control | | |
| SEQ ID: 22 | Human RNAse | RNAse P | TTTTGATAGCAACAACTGAATAGCCAAG |
| | P control | | |

Second amplification primers

| | | | |
|---|---|---|---|
| SEQ ID: 23 | SARS CoV-2 | N₁ | TTTTAATGGACCCCAAAATCAGCGAAAT |
| | Nucleocapsid | | |
| SEQ ID: 24 | SARS CoV-2 | N₁ | (FL)TTTTTCTGGTTACTGCCAGTTGAATCTG |
| | Nucleocapsid | | |
| SEQ ID: 25 | CoV | N₂ | TTTACTGATTACAAACATTGGCCGCAAA |
| | Nucleocapsid* | | |
| SEQ ID: 26 | CoV | N₂ | (FL)TTTTGCCAATGCGYCGACATTCCRAAG |
| | Nucleocapsid* | | AA |
| SEQ ID: 27 | CoV | N₃ | TTTAGGGAGCCTTGAATACACCAAAAGA |
| | Nucleocapsid* | | |
| SEQ ID: 28 | CoV | N₃ | (FL)TTTAAGTTGTAGCACGATTGCAGCATTG |
| | Nucleocapsid* | | |
| SEQ ID: 29 | MERS CoV | upE | TTTCCATATGTCCAAAGAGAGACTAATG |
| SEQ ID: 30 | MERS CoV | upE | (FL)TTTTAGTAGCGCAGAGCTGCTTAAACG |
| | | | A |
| SEQ ID: 31 | HCoV-229E | M | TTTACATACTATCAACCCATTCAACAAG |
| SEQ ID: 32 | HCoV-229E | M | (FL)TTTCTCGGCACGGCAACTGTCATGTATT |
| SEQ ID: 33 | HCoV-OC43 | M | TTTTCATACYCTGACGGTCACAATAATA |
| SEQ ID: 34 | HCoV-OC43 | M | (FL)TTTTAACCTTAGCAACAGWCATATAAGC |
| SEQ ID: 35 | NHCoV-NL63 | N | TTATAGTTCTGATAAGGCACCATATAGG |
| SEQ ID: 36 | NHCoV-NL63 | N | (FL)TTTGAACTTTAGGAGGCAAATCAACAC |
| | | | G |
| SEQ ID: 37 | NHCoV-HKU1 | N | TTTGATCCTACTAYTCAAGAAGCTATCC |
| SEQ ID: 38 | NHCoV-HKU1 | N | (FL)TTTCTTAATGAACGAKTATTGGGTCCAC |
| SEQ ID: 39 | Influenza A | M | TTTCAAGACCRATCCTGTCACCTCTGAC |
| SEQ ID: 40 | Influenza A | M | (FL)TTTAAGGGCATTYTGGACAAAKCGTCTA |
| SEQ ID: 41 | Influenza B | NS1 | TTTGCGTCTCAATGAAGGACATTCAAAG |
| SEQ ID: 42 | Influenza B | NS1 | (FL)TTTTAATCGGTGCTCTTGACCAAATTGG |
| SEQ ID: 43 | Human RNAse | RNAse P | TTTGTTTGCAGATTTGGACCTGCGAGCG |
| | P control | | |
| SEQ ID: 44 | Human RNAse | RNAse P | (FL)TTTAAGGTGAGCGGCTGTCTCCACAAG |
| | P control | | T |

*Amplifies SARS-CoV-2, SARS, Bat_SARS-like CoV, Pangolin CoV (S. China), Bat precursor CoV (Yunnan 2013) and New Bat CoV (Yunnan 2019).
(FL) = fluorescent label.

Further in this embodiment, the fluorescent labeled COVID-19 virus amplicons generated are hybridized to a plurality of nucleic acid probes. The nucleic acid probes have a sequence corresponding to sequence determinants in the COVID-19 virus and have sequences SEQ ID: 45 to SEQ ID: 48 (Table 2) and SEQ ID: 85 to SEQ ID: 94 (Table 38). Controls including, but not limited to a RNAse P control nucleic acid probe (SEQ ID: 71 and SEQ ID: 72) and a negative control nucleic acid probe (SEQ ID: 73) are also used herein (Table 2). The nucleic acid probes are attached to a solid microarray support. The solid support is any microarray including but not limited to a 3-dimensional lattice microarray.

Further in this embodiment, after hybridization, unhybridized amplicons are removed by washing the microarray. Washed microarrays are imaged to detect a fluorescent signal corresponding to the fluorescent labeled COVID-19 virus specific amplicons to detect presence of the COVID-19 virus in the sample.

Further to this embodiment, the method further comprises calculating an intensity for the fluorescent signal. The calculated intensity is correlated with the number of COVID-19 virus specific genomes in the sample. The measured intensity is correlated with the number of COVID-19 virus specific genomes in the sample. Based on analysis of virus-free samples, an experimentally determined intensity threshold is established for the hybridization to each probe on the microarray, such that a fluorescent intensity above that threshold signifies the presence of SARS-CoV-2 viral RNA, while fluorescence intensities below the threshold signifies that SARS-CoV-2 was not detected.

TABLE 2
Nucleic acid probe sequences used for hybridization

| SEQ ID | | | |
|---|---|---|---|
| NOS. | Target | Gene | Primer Sequence |
| SEQ ID: 45 | SARS CoV-2 | N₁ | TTTTTTTCCGCATTACGTTTGGTGTTTTTT |
| | Nucleocapsid | | |
| SEQ ID: 46 | SARS CoV-2 | N₁ | TTTTTTTATCAGCGAAATGCACCCTTTTTT |
| | Nucleocapsid | | |
| SEQ ID: 47 | SARS CoV-2 | N₂ | TTTTTTTTTTGCCCCCAGCGCTTCTTTTTT |
| | Nucleocapsid | | |
| SEQ ID: 48 | SARS CoV-2 | N₂ | TTTTTTACAATTTGCCCCCAGCGTCTTTTT |
| | Nucleocapsid | | |
| SEQ ID: 49 | SARS | N₂ | TTTTTTTTTGCTCCRAGTGCCTCTTTTTTT |
| | Nucleocapsid | | |
| SEQ ID: 50 | SARS | N₂ | TTTTTTTTGCTCCRAGTGCCTCTGTCCTTT |
| | Nucleocapsid | | |
| SEQ ID: 51 | CoV Bat | N₂ | TTTTTGTTTGCACCTAGTGCTTCAGCCCTTTT |
| | precursor | | |
| SEQ ID: 52 | CoV Pangolin | N₂ | TTTTTATTTGCWCCTAGCGCTTCTGCTCTTTT |
| | precursor | | |
| SEQ ID: 53 | CoV Bat | N₂ | TTTTTGTTTGCACCCAGTGCTTCTGCTCTTTT |
| | precursor - | | |
| | Yunnan 2013 | | |
| SEQ ID: 54 | CoV Bat | N₂ | TTTTTTACAATTCGCTCCCAGCGTCTTTTT |
| | precursor - | | |
| | Yunnan 2019 | | |
| SEQ ID: 55 | CoV | N₃ | TTTTTCTGGCACCCGCAATCCTGTCTTTTT |
| | Nucleocapsid* | | |
| SEQ ID: 56 | CoV | N₃ | TTTTTTAYCACATTGGCACCCGCATCTTTT |
| | Nucleocapsid* | | |
| SEQ ID: 57 | MERS CoV | upE | TTTTATCTCTTCACATAATCGCCCTTTTTT |
| SEQ ID: 58 | MERS | upE | TTTTTTATAATCGCCCCGAGCTCGTCTTTT |
| SEQ ID: 59 | HCoV-229E | M | TTTTTTTGCTTTACGTTGACGGACATTTTTTT |
| SEQ ID: 60 | HCoV-229E | M | TTTTTTTCAGGTGTTCAGGTTCATAATCTTTT |
| SEQ ID: 61 | HCoV-OC43 | M | TTTTTCATCTTTACATTCAAGGTATAATTTTT |
| SEQ ID: 62 | HCoV-OC43 | M | TTTTCTGCTATTCTTTGGCAGATTTGCTTTTT |
| SEQ ID: 63 | NHCoV-NL63 | N | TTTTTCTAAGAGCGTTGGCGTATGCTTTTTTT |
| SEQ ID: 64 | NHCoV-NL63 | N | TTTTTTAAGATGAGCAGATTGGTTACCTTTTT |
| SEQ ID: 65 | NHCoV-HKU1 | N | TTTTTTCAGGTTCACGTTCTCAATCATTTTTT |
| SEQ ID: 66 | NHCoV.HKU1 | N | TTTTCTGTACGATTYTGCCTCAAGGCCTTTTT |
| SEQ ID: 67 | Influenza A | NS1 | TTTTTTTCGTGCCCAGTGAGCGAGTTTTTT |
| SEQ ID: 68 | Influenza A | NS1 | TTTTTTAGTGAGCGAGGACTGCATTTTTTT |
| SEQ ID: 69 | Influenza B | M | TTTTTTCCAATTCGAGCAGCTGAATTTTTT |
| SEQ ID: 70 | Influenza B | M | TTTTTTAGCAGCTGAAACTGCGGTTTTTTT |
| SEQ ID: 71 | Human RNAse | RNAse | TTTTTTTTCTGACCTGAAGGCTCTGCGCGTTT |
| | P control | P | TT |
| SEQ ID: 72 | Human RNAse | RNAse | TTTTTCTTGACCTGAAGGCTCTGCTTTTTT |
| | P control | P | |
| SEQ ID: 73 | Negative | — | TTTTTTCTACTACCTATGCTGATTCACTCTTTT |
| | Control | | T |

*Hybridizes with SARS-CoV-2, SARS Bat_SARS-like CoV, Pangolin CoV (S. China), Bat precursor CoV (Yunnan 2013), New Bat CoV (Yunnan 2019)

Further to this embodiment, when the sample comprises in addition to the COVID-19 virus, at last one other non-COVID-19 virus comprising a Respiratory Syncytial Virus, a Middle East Respiratory Syndrome coronavirus (MERS-CoV), a Severe Acute Respiratory Syndrome Coronavirus (SARS-CoV), a 229E Coronavirus, a OC43 Coronavirus, a NL63 Coronavirus, a HKU1 Coronavirus an Influenza A virus or an Influenza B virus, the method further comprises detecting presence of both COVID-19 virus and the at least one non-COVID-19 virus by performing the amplification and hybridization steps described above for these viruses. In this embodiment, the fluorescent labeled primer pairs have forward (odd numbers) and reverse (even number) sequences shown in SEQ ID: 23 to SEQ ID: 42 (Table 1) and SEQ ID: 74 to SEQ ID: 84 (Table 37). and nucleic acid probe sequences SEQ ID: 45 to SEQ ID: 70 (Table 2) and SEQ ID: 85 to SEQ ID: 97 (Table 38). Controls including, but not limited to a RNAse P control having primer pair (forward primer SEQ ID: 43, reverse primer SEQ ID: 44) and nucleic acid probe (SEQ ID: 71 and SEQ ID: 72) and, a negative control nucleic acid probe (SEQ ID: 73) are also used herein.

In another embodiment of the present invention, there is provided a method for detecting a respiratory disease-causing pathogen in a sample, comprising obtaining a sample; isolating total nucleic acids from the sample; performing a combined reverse transcription and an asymmetric PCR amplification reaction on the isolated total nucleic acids using at least one fluorescent labeled primer pair comprising an unlabeled primer, and a fluorescently labeled primer selective for a target nucleotide sequence in the at least one respiratory disease-causing pathogen, to generate at least one fluorescent labeled pathogen specific amplicon; hybridizing the fluorescent labeled pathogen specific amplicons to a plurality of nucleic acid probes each having a sequence corresponding to a sequence determinant in the pathogen, each of said nucleic acid probes attached at a specific position on a solid microarray support; washing the microarray at least once; and imaging the microarray to detect at least one fluorescent signal from the hybridized fluorescent labeled pathogen specific amplicons, thereby detecting the respiratory disease-causing pathogen in the sample.

In this embodiment, in one aspect, the sample is any sample obtained from a subject including, but not limited to a nasopharyngeal swab, nasal swab, mouth swab, and mouthwash (sample obtained by rinsing the subject's buccal cavity). A pooled sample obtained by combining two or more of these samples or by combining samples from multiple subjects may also be used. In another aspect of this embodiment, the sample is an environmental sample obtain from inanimate sources including but is not limited to an aerosol and a hard surface. In this embodiment, the aerosol samples are obtained using commercial air samplers such as for example a Coriolis Micro Air Sampler. In this embodiment, a sample from a hard surface is obtained using a swab. In either aspect of this embodiment, the viruses from samples obtained on swabs are dispersed in a liquid such as phosphate buffered saline. Aerosol samples are transferred into a volume of a liquid such as phosphate buffered saline.

In this embodiment, the respiratory disease-causing pathogen is a virus, a bacteria, a fungi, or a combination of these. The sample may also comprise mutated forms of these pathogens. Examples of respiratory disease-causing viruses include, but are not limited to, Severe Acute Respiratory Syndrome Coronavirus 2 (COVID-19 virus), a Respiratory Syncytial Virus, a Middle East Respiratory Syndrome coronavirus (MERS-CoV), or a Severe Acute Respiratory Syndrome Coronavirus (SARS-CoV), or a 229E Coronavirus, or a OC43 Coronavirus, or a NL63 Coronavirus, or a HKU1 Coronavirus or an Influenza A virus or an Influenza B virus, an adenovirus, a bocavirus, a metapneumovirus, a parainfluenza and a rhinovirus. Examples of respiratory disease-causing bacteria include, but are not limited to, a Mycobacterium species (e.g. Mycobacterium tuberculosis), a Streptococcus species (e.g. Streptococcus pneumoniae), a Mycoplasma species, an Enterococcus species, a Haemophilus species, a Klebsiella species, a Moraxella species and a Corynebacterium species. Examples of respiratory disease-causing fungus include, but are not limited to, a Histoplasma species, a Coccidioides species, a Blastomyces species, a Rhizopus species, an Aspergillus species, a Pneumocystis species and a Cryptococcus species. In this embodiment, in some aspects, the sample is mixed with a nucleic acid stabilizer such as for example, a chemical stabilizer that would protect the nucleic acids from degradation during storage and transportation, prior to the isolating step.

In this embodiment, a total nucleic acids potentially comprising nucleic acids from the pathogen and contaminating human cells is isolated. Commercially available nucleic acid isolation kits such as for example, a Quick-DNA/RNA MagBead Kit from Zymo Research are used for this purpose. The total nucleic acids thus isolated is used without further purification. Alternatively, the pathogens may be captured using hydrogel chemistry (Ceres Nanosciences) or enriched using methods including, but not limited to centrifugation and polyethylene glycol (PEG), followed by lysis of the enriched pathogens by heating with a “PCR-Friendly” lysis solution such as 1% NP40 in TE buffer and the total nucleic acids used without additional purification.

In this embodiment, the isolated total nucleic acids are used as a template in a combined reverse transcription/amplification reaction (RT-PCR). In this step, the nucleic acid sequences in the pathogen are transcribed using a reverse transcriptase enzyme to generate pathogen specific complementary DNA (cDNA) that is amplified in the same reaction using pathogen selective fluorescent labeled primer pairs to generate fluorescent labeled pathogen specific amplicons. In this embodiment, fluorescent labeled primer pair comprises an unlabeled primer, and a fluorescently labeled primer in about 4-fold to about 8-fold excess of the unlabeled primer whereby, upon completion of the reaction, the fluorescently labelled amplicon will be primarily single stranded (that is, the reaction is a type of “asymmetric PCR”). In this embodiment, when the pathogen is a virus, the fluorescent labeled primer pairs have forward (odd numbers) and reverse (even number) sequences shown in SEQ ID: 23 to SEQ ID: 28 (Table 1) and SEQ ID: 74 to SEQ ID: 80 (Table 37). Commercially available reverse transcriptase enzyme and buffers are used in this step. Controls including, but not limited to a RNAse P control having fluorescent labeled primer pair (forward primer SEQ ID: 43, reverse primer SEQ ID: 44) are also used herein. Any fluorescent label may be used, including, but not limited to CY3, a CY5, SYBR Green, a DYLIGHT™ DY647, a ALEXA FLUOR 647, a DYLIGHT™ DY547 and a ALEXA FLUOR 550.

Further in this embodiment, the fluorescent labeled pathogen specific cDNA amplicons generated are hybridized to a plurality of nucleic acid probes. In this embodiment, when the pathogen is a virus, the nucleic acid probes have a sequence corresponding to sequence determinants in the pathogen and have sequences SEQ ID: 45 to SEQ ID: 48 (Table 2) and SEQ ID: 85 to SEQ ID: 94 (Table 38). Controls including, but not limited to a RNAse P control nucleic acid probe (SEQ ID: 71 and SEQ ID: 72) and a negative control nucleic acid probe (SEQ ID: 73) are also used herein (Table 2). The nucleic acid probes are attached at specific positions on a solid support. The solid support is any microarray including but not limited to a 3-dimensional lattice microarray.

Further in this embodiment, after hybridization, unhybridized amplicons are removed by washing the microarray. Washed microarrays are imaged to detect a fluorescent signal corresponding to the fluorescent labeled pathogen specific amplicons to detect presence of the respiratory disease-causing pathogen in the sample.

In this embodiment, the method steps for detecting the respiratory disease-causing virus, bacterium and fungus are concurrently performed in a single assay. This is advantageous since it enables streamlined detection of COVID-19 virus and the other pathogens in a single assay. Further in this embodiment, the methods described above may be used to detect in any combination, a COVID-19 virus, another virus, a bacterium, or a fungus.

Further to this embodiment, the method further comprises calculating an intensity for the fluorescent signal. The calculated intensity is correlated with the number of pathogen specific genomes in the sample. The measured intensity is a function of the number of pathogen specific genomes in the sample. Based on analysis of pathogen-free samples, an experimentally determined intensity threshold is established for the hybridization to each probe on the microarray, such that a fluorescent intensity above that threshold signifies the presence of pathogen nucleic acid, while fluorescence intensities below the threshold signifies that the pathogen was not detected.

In yet another embodiment of the present invention, there is provided a method for detecting a Coronavirus disease 2019 (COVID-19) in a sample, comprising obtaining the sample; isolating from the sample, a total nucleic acid; performing a combined reverse transcription and an asymmetric PCR amplification reaction on the total nucleic to generate fluorescent labeled COVID-19 virus amplicons using at least one fluorescent labeled primer pair selective for a target nucleotide sequence in the COVID-19 virus RNA, said fluorescent labeled primer pair comprising an unlabeled primer and a fluorescently labeled primer in an excess over the unlabeled primer; hybridizing the fluorescent labeled COVID-19 virus amplicons to a plurality of nucleic acid probes each having a sequence corresponding to a sequence determinant in the COVID-19 virus, each of said nucleic acid probes attached at a specific position on a microarray support; washing the microarray at least once; and imaging the microarray to detect at least one fluorescent signal from the hybridized fluorescent labeled COVID-19 virus amplicons, thereby detecting the COVID-19 in the sample.

In this embodiment, in one aspect, the sample is any sample obtained from a subject including, but not limited to a nasopharyngeal swab, nasal swab, mouth swab, and mouthwash (sample obtained by rinsing the subject's buccal cavity). A pooled sample obtained by combining two or more of these samples or by combining samples from multiple subjects may also be used. In another aspect of this embodiment, the sample is an environmental sample obtain from inanimate sources including but is not limited to an aerosol and a hard surface. In this embodiment, the aerosol samples are obtained using commercial air samplers such as for example a Coriolis Micro Air Sampler. In this embodiment, a sample from a hard surface is obtained using a swab. In either aspect of this embodiment, the viruses from samples obtained on swabs are dispersed in a liquid such as phosphate buffered saline. Aerosol samples are transferred into a volume of a liquid such as phosphate buffered saline.

In this embodiment, the COVID-19 virus is a Severe Acute Respiratory Syndrome Coronavirus 2 (SARS-CoV 2) or a mutated form thereof. In this embodiment, in some aspects, the sample is mixed with an RNA stabilizer such as for example, a chemical stabilizer that would protect the RNA from degradation during storage and transportation, prior to the RNA isolating step.

In this embodiment, a total nucleic acids potentially comprising nucleic acids from pathogens including the COVID-19 virus, and contaminating human cells is isolated. Commercially available nucleic acid isolation kits such as for example, a Quick-DNA/RNA MagBead Kit from Zymo Research are used for this purpose. The total nucleic acids thus isolated is used without further purification. Alternatively, the pathogens may be captured using hydrogel chemistry (Ceres Nanosciences) or enriched using methods including, but not limited to centrifugation and polyethylene glycol (PEG), followed by lysis of the enriched pathogens by heating with a “PCR-Friendly” lysis solution such as 1% NP40 in TE buffer and the total nucleic acids used without additional purification.

In this embodiment, the COVID-19 virus RNA is used as a template in a combined reverse transcription/amplification reaction (RT-PCR). In this step, the nucleic acid sequences in the COVID-19 virus RNA are transcribed using a reverse transcriptase enzyme to generate COVID-19 complementary DNA (cDNA) that is amplified in the same reaction using COVID-19 virus selective fluorescent labeled primer pairs to generate fluorescent labeled COVID-19 virus amplicons. In this embodiment, fluorescent labeled primer pair comprises an unlabeled primer, and a fluorescently labeled primer in about 4-fold to about 8-fold excess of the unlabeled primer whereby, upon completion of the reaction, the fluorescently labelled amplicon will be primarily single stranded (that is, the reaction is a type of “asymmetric PCR”). In this embodiment, the fluorescent labeled primer pairs have forward (odd numbers) and reverse (even number) sequences shown in SEQ ID: 23 to SEQ ID: 28 (Table 1) and SEQ ID: 74 to SEQ ID: 84 (Table 37). Commercially available reverse transcriptase enzyme and buffers are used in this step. Controls including, but not limited to a RNAse P control having fluorescent labeled primer pair (forward primer SEQ ID: 43, reverse primer SEQ ID: 44) are also used herein. Any fluorescent label may be used, including, but not limited to CY3, a CY5, SYBR Green, a DYLIGHT™ DY647, a ALEXA FLUOR 647, a DYLIGHT™ DY547 and a ALEXA FLUOR 550.

Further in this embodiment, the fluorescent labeled COVID-19 virus amplicons generated are hybridized to a plurality of nucleic acid probes. The nucleic acid probes have a sequence corresponding to sequence determinants in the COVID-19 virus and have sequences SEQ ID: 45 to SEQ ID: 48 (Table 2) and SEQ ID: 85 to SEQ ID: 94 (Table 38). Controls including, but not limited to a RNAse P control nucleic acid probe (SEQ ID: 71 and SEQ ID: 72) and a negative control nucleic acid probe (SEQ ID: 73) are also used herein (Table 2). The nucleic acid probes are attached to a solid microarray support. The solid support is any microarray including but not limited to a 3-dimensional lattice microarray.

Further in this embodiment, after hybridization, unhybridized amplicons are removed by washing the microarray. Washed microarrays are imaged to detect a fluorescent signal corresponding to the fluorescent labeled COVID-19 virus specific amplicons to detect presence of the COVID-19 virus in the sample.

Further to this embodiment, when the sample additionally comprises at least one virus that is not a COVID-19 virus, the step of performing the combined reverse transcription and the asymmetric PCR amplification reaction on the isolated total nucleic acid comprises using at least two fluorescent labeled primer pairs, each comprising the unlabeled primer and the fluorescently labeled primer selective for a target nucleotide sequence in the COVID-19 virus and the at least one non-COVID-19 virus to generate at least one fluorescent labeled COVID-19 virus specific amplicons and at least one fluorescent labeled non-COVID-19 virus specific amplicon; and the step of hybridizing comprises hybridizing the at least one fluorescent labeled COVID-19 virus specific amplicon and the at least one fluorescent labeled non-COVID-19 virus specific amplicon to the plurality of nucleic acid probes each having a sequence corresponding to the sequence determinant in the COVID-19 virus and the at least one non-COVID-19 virus.

In this embodiment, the non-COVID-19 virus is any virus including, but not limited to a respiratory disease-causing RNA or DNA virus. Examples of RNA viruses include, and are not limited to a Respiratory Syncytial Virus, a Middle East Respiratory Syndrome coronavirus (MERS-CoV), a Severe Acute Respiratory Syndrome Coronavirus (SARS-CoV), a 229E Coronavirus, a OC43 Coronavirus, a NL63 Coronavirus, a HKU1 Coronavirus an Influenza A virus, an Influenza B virus, a metapneumovirus, a parainfluenza, and a rhinovirus. In this embodiment, the fluorescent labeled primer pairs have forward (odd numbers) and reverse (even number) sequences shown in SEQ ID: 23 to SEQ ID: 42 (Table 1) and SEQ ID: 74 to SEQ ID: 84 (Table 37). and nucleic acid probe having sequences SEQ ID: 45 to SEQ ID: 70 (Table 2) and SEQ ID: 85 to SEQ ID: 97 (Table 38). Controls including, but not limited to a RNAse P control having primer pair (forward primer SEQ ID: 43, reverse primer SEQ ID: 44) and nucleic acid probe (SEQ ID: 71 and SEQ ID: 72) and, a negative control nucleic acid probe (SEQ ID: 73) are also used herein. Examples of DNA viruses include and are not limited to an adenovirus and a bocavirus.

Further to this embodiment, when the sample additionally comprises at least one bacterium, the step of performing the combined reverse transcription and the asymmetric PCR amplification reaction on the isolated total nucleic acid comprises using at least two fluorescent labeled primer pairs, each comprising the unlabeled primer and the fluorescently labeled primer selective for a target nucleotide sequence in the COVID-19 virus and the at least one bacterium to generate at least one fluorescent labeled COVID-19 virus specific amplicons and at least one fluorescent labeled bacterium specific amplicon; and the step of hybridizing comprises hybridizing the at least one fluorescent labeled COVID-19 virus specific amplicon and the at least one fluorescent labeled bacterium specific amplicon to the plurality of nucleic acid probes each having a sequence corresponding to the sequence determinant in the COVID-19 virus and the at least one bacterium.

In this embodiment, the bacterium is any bacterium including, but not limited to a respiratory disease-causing bacterium. Examples of bacteria include, and are not limited to a Mycobacterium species (e.g. Mycobacterium tuberculosis), a Streptococcus species (e.g. Streptococcus pneumoniae), a Mycoplasma species, an Enterococcus species, a Haemophilus species, a Klebsiella species, a Moraxella species and a Corynebacterium species.

Further to this embodiment, when the sample additionally comprises at least one fungus, the step of performing the combined reverse transcription and the asymmetric PCR amplification reaction on the isolated total nucleic acid comprises using at least two fluorescent labeled primer pairs, each comprising the unlabeled primer and the fluorescently labeled primer selective for a target nucleotide sequence in the COVID-19 virus and the at least one fungus to generate at least one fluorescent labeled COVID-19 virus specific amplicons and at least one fluorescent labeled fungus specific amplicon; and the step of hybridizing comprises hybridizing the at least one fluorescent labeled COVID-19 virus specific amplicon and the at least one fluorescent labeled fungus specific amplicon to the plurality of nucleic acid probes each having a sequence corresponding to the sequence determinant in the COVID-19 virus and the at least one fungus.

In this embodiment, the fungus is any virus including, but not limited to a respiratory disease-causing fungus. Examples of fungus include, and are not limited to a Histoplasma species, a Coccidioides species, a Blastomyces species, a Rhizopus species, an Aspergillus species, a Pneumocystis species and a Cryptococcus species.

In any of the above embodiments, the method steps for detecting the virus, the bacterium and the fungus are concurrently performed in a single assay with the COVID-19 virus detection steps described above. This is advantageous since it enables streamlined detection of COVID-19 virus and the other pathogens in a one assay. Further in this embodiment, the methods described above may be used to detect in any combination, a COVID-19 virus, another virus, a bacterium, or a fungus.

Also, in any of the above embodiments, the imaging step further comprises calculating an intensity for the fluorescent signal. The calculated intensity is correlated with the number of genomes of the virus, bacterium, and fungus in the sample. The measured intensity is a function of the number of such genomes in the sample. Based on analysis of pathogen-free samples, an experimentally determined intensity threshold is established for the hybridization to each probe on the microarray, such that a fluorescent intensity above that threshold signifies the presence of nucleic acids for the virus, bacterium or fungus, while fluorescence intensities below the threshold signifies that the virus, bacterium or fungus was not detected respectively.

Further to this embodiment, the imaging step further comprises calculating an intensity for the fluorescent signal. The calculated intensity is correlated with the number of genomes in the sample. The measured intensity is correlated with the number of genomes in the sample. Based on analysis of pathogen-free samples, an experimentally determined intensity threshold is established for the hybridization to each probe on the microarray, such that a fluorescent intensity above that threshold signifies the presence of pathogen specific nucleic acids, while fluorescence intensities below the threshold signifies that the pathogen was not detected.

Described herein is a method for detecting a COVID-19 disease in a sample such as a nasopharyngeal swab, a nasal swab, a mouth swab, a mouth wash, an aerosol, or a swab from a hard surface. The sample is mixed with a chemical stabilizer after sample collection. The stabilizer prevents RNA degradation during storage and transportation prior to RNA isolation. The isolated RNA is a total RNA preparation comprising viral and non-viral RNA including COVID-19 virus RNA that is used without further purification. This RNA preparation is used in a combined reverse transcription and asymmetric PCR amplification reaction to generate fluorescent labeled COVID-19 virus amplicons. The fluorescent labeled COVID-19 virus amplicons are hybridized to nucleic acid probes attached to a microarray. This method allows positive hybridization signals to be validated on each sample tested based on internal “mismatched” and “sequence specific” controls. Also described herein is a method for detecting presence of a respiratory virus disease-causing virus, bacterium and fungus in the sample using pathogen specific primers and nucleic acid probes and the same method steps described above. The method steps may be performed concurrently performed in a single assay, which is beneficial since it enables streamlined detection of COVID-19 virus and the other pathogens in a single assay. Any combination of COVID-19 virus, non-COVID-19 virus, bacterium, and fungus may be detected using this method.

In the embodiments described above, the microarray is made of any suitable material known in the art including but not limited to borosilicate glass, a thermoplastic acrylic resin (e.g., poly(methyl methacrylate-VSUVT) a cycloolefin polymer (e.g. ZEONOR 1060R), a metal including, but not limited to gold and platinum, a plastic including, but not limited to polyethylene terephthalate, polycarbonate, nylon, a ceramic including, but not limited to TiO₂, and Indium tin oxide (ITO) and engineered carbon surfaces including, but not limited to graphene. A combination of these materials may also be used. The solid support has a front surface and a back surface and is activated on the front surface by chemically activatable groups for attachment of the nucleic acid probes. In this embodiment, the chemically activatable groups include but are not limited to epoxysilane, isocyanate, succinimide, carbodiimide, aldehyde and maleimide. These materials are well known in the art and one of ordinary skill in this art would be able to readily functionalize any of these supports as desired. In a preferred embodiment, the solid support is epoxysilane functionalized borosilicate glass support.

The nucleic acid probes are attached either directly to the microarray support, or indirectly attached to the support using bifunctional polymer linkers. In this embodiment, the bifunctional polymer linker has a top domain and a bottom end. On the bottom end is attached a first reactive moiety that allows covalent attachment to the chemically activatable groups in the solid support. Examples of first reactive moieties include but are not limited to an amine group, a thiol group and an aldehyde group. In one aspect the first reactive moiety is an amine group. On the top domain of the bifunctional polymer linker is provided a second reactive moiety that allows covalent attachment to the oligonucleotide probe. Examples of second reactive moieties include but are not limited to nucleotide bases like thymidine, adenine, guanine, cytidine, uracil and bromodeoxyuridine and amino acid like cysteine, phenylalanine, tyrosine glycine, serine, tryptophan, cystine, methionine, histidine, arginine and lysine. The bifunctional polymer linker may be an oligonucleotide such as OLIGOdT, an amino polysaccharide such as chitosan, a polyamine such as spermine, spermidine, cadaverine and putrescine, a polyamino acid, with a lysine or histidine, or any other polymeric compounds with dual functional groups which can be attached to the chemically activatable solid support on the bottom end, and the nucleic acid probes on the top domain. Preferably, the bifunctional polymer linker is OLIGOdT having an amine group at the 5′ end.

In this embodiment, the bifunctional polymer linker may be unmodified with a fluorescent label. Alternatively, the bifunctional polymer linker has a fluorescent label attached covalently to the top domain, the bottom end, or internally. The second fluorescent label is different from the fluorescent label in the fluorescent labeled primers. Having a fluorescent label (fluorescent tag) attached to the bifunctional polymer linker is beneficial since it allows the user to image and detect the position of the individual nucleic acid probes (“spot”) printed on the microarray. By using two different fluorescent labels, one for the bifunctional polymer linker and the second for the amplicons generated from the viral RNA being queried, the user can obtain a superimposed image that allows parallel detection of those nucleic acid probes which have been hybridized with amplicons. This is advantageous since it helps in identifying the virus comprised in the sample using suitable computer and software, assisted by a database correlating nucleic acid probe sequence and microarray location of this sequence with a known RNA signature in viruses. Examples of fluorescent labels include, but are not limited to CY5, DYLIGHT™ DY647, ALEXA FLUOR 647, CY3, DYLIGHT™ DY547, or ALEXA FLUOR 550. The fluorescent labels may be attached to any reactive group including but not limited to, amine, thiol, aldehyde, sugar amido and carboxy on the bifunctional polymer linker. In one aspect, the bifunctional polymer linker is CY5-labeled OLIGOdT having an amino group attached at its 3′terminus for covalent attachment to an activated surface on the solid support.

Further in this embodiment, when the bifunctional polymer linker is also fluorescently labeled a second fluorescent signal image is detected in the imaging step. Superimposing the first fluorescent signal image and second fluorescent signal image allows identification of the virus by comparing the sequence of the nucleic acid probe at one or more superimposed signal positions on the microarray with a database of signature sequence determinants for a plurality of viral RNA. This embodiment is particularly beneficial since it allows identification of more than one type of virus in a single assay.

DETECTX-RV enables screening for COVID-19 in nasopharyngeal swabs. The microarray has the capacity to test for multiple viral analytes in parallel DETECTX-RV is based on endpoint PCR (rather than qPCR) and is coupled to concurrent analysis of up to 144 distinct nucleic acid probes (rather than just 4 probes for qPCR). This enhanced test capacity enables concurrent testing of 3 different sites (N1,N2,N3) in the SARS CoV-2 genome and further, include a human RNA control (RNAse P). The testing may be performed in triplicate along with a panel of 8 viral controls, enabling confirmation of COVID-19 at a level of experimental specificity of over 10× compared to q-RT-PCR. The DETECTX-RV-V2 microarray differs from DETECTX-RV in the additional inclusion of the newly discovered S-D614G variant in the same assay and an additional amplification step. This microarray is suitable for fully automated testing capable of processing samples in a 96-well array plate format, or the higher throughput 384-well microarray plate format.

The following examples are given for the purpose of illustrating various embodiments of the invention and are not meant to limit the present invention in any fashion.

### Example 1

Tandem PCR (or RT-PCR, then Asymmetric PCR) Reactions to Enhance the Ability to Accurately Detect the Population Density (i.e. Molecules/μL) Near the Lowest Limit of Detection (LLoD)

The first of the two tandem reactions coverts segments of RNA genome into an abundance of amplified DNA. It is a type of Endpoint PCR reaction, such that the original RNA input is amplified 35 cycles, to form an Endpoint PCR product, wherein the input RNA target segments have been amplified to generate a maximal number of DNA amplicons.

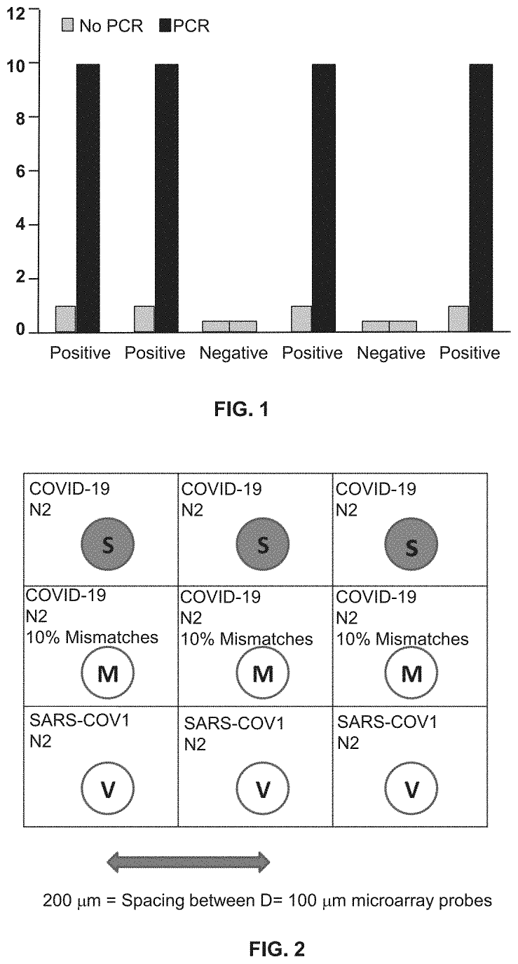

The second PCR reaction, which may be a real-time or an endpoint PCR reaction, builds upon the first reaction such that if one or more molecules of DNA or RNA are input into the first reaction, that first PCR reaction produces an amplified DNA segment which has been amplified to yield a sample that may display up to a 10+⁶ fold increase in strand concentration within the amplicon product (FIG. 1).

The second PCR reaction additionally tags the PCR amplified product with a Dye (e.g. CY-3), which enables amplicon detection after microarray hybridization. The second reaction is performed as an asymmetric PCR reaction, such that upon completion of this second “Endpoint” PCR reaction, the product is >90% single stranded (due to the asymmetry of the PCR reaction) with the single strand of interest being the only strand bearing the CY-3 dye probe. This asymmetry allows the product to be used for hybridization without the need for heat denaturation and avoids hybridization artifacts which are otherwise common.

If no RNA (or DNA) were input into the first reaction, none will be amplified (FIG. 1). Having received that amplified input into the second PCR reaction, the quantitative distinction between (0) copies of original genomic nucleic acid. i.e. a “negative Aliquot”, vs (1, >1) copies of genomic nucleic acid in an aliquot, that is, “positive” aliquot is thus greatly amplified. Thus, in the context of the tandem reactions of the present invention, the first PCR (or RT-PCR) reaction can be thought of as a method of signal amplification to increase signal “gain” (FIG. 1) to be of benefit to the second PCR reaction or similar amplification reaction.

Equation 1. PCR Reaction #1. Amplifies mass distinction between sample aliquots with (0) vs (>0)

DNA Copies in the original:

1 copy of genomic DNA Target→at 1×2^{M} Copies of Product Targets (M=number of PCR Cycles)
0 copy of genomic DNA Target→at 0×2^{M} Copies of Product Targets (M=number of PCR Cycles)

DNA target signal strength increases after PCR. 1 Copy→1×2^{M} copies

Statistical occurrence of “Negative” events (Pr0) in aliquots of the original sample does not change as a result of PCR Reaction #1.

Pr(0)=exp-(<N>) before and after PCR

In medical diagnostics, food safety and other demanding applications, the LLoD is a crucial test parameter, which is defined by, and directly measured, in the context of samples where, for nucleic acids, the number of microbial or viral genomes in fluid solution are introduced as small (typically 1 μL) aliquots into the PCR reaction at levels so dilute that such single 1 μL aliquots will, via ordinary random sampling statistics, be expected to capture a significant number of “negative” aliquots, i.e. (0) copies of the original nucleic acid genome in each (see FIG. 1).

The present invention serves to greatly increase the amplitude of the signal associated with the “positive” events (≥1 genomes per aliquot) relative to the “negative” events where, lacking a template for PCR, PCR does not occur (see Equation 1 and FIG. 1). Thus, without altering the relative frequency of “positive” vs “negative” sampling events, the signal associated with the “positive” signals is greatly amplified, making subsequent analysis of such positives more accurate, while still providing an accurate determination of the original nucleic acid sample density, as manifest in the “positive/”negative” sampling frequency ratio.

For example, in the context of Poisson statistics, LLoD can be measured by counting the statistical likelihood of “negative” signals derived from the “negative” (N=0) aliquot events, relative to statistical occurrence of all “positive” events, i.e. signals obtained when (N=1+N>1). Using Poisson statistics, that (positive/negative) event ratio can be used to calculate the average population density (<N>) of nucleic acid target molecules in the original sample aliquot size. For instance, in an ideal assay near the LLOD, where <N>=1 per sample aliquot (e.g. 1 genome per 1 μL) Poisson statistics specify that the statistical likelihood of “positive” vs “negative” signals on repeat 1 μL aliquoting will approach 1-e¹/e¹≈2. Alternatively, when <N>=3 per aliquot, the ratio of (positive vs “negative) signals on repeat measurement would approach 1-e⁻³/e⁻³≈20 which is the standard definition of the LLoD defined by the FDA and the USDA food safety and medical diagnostics.

Thus, as seen in Equation #1 and FIG. 1, the first PCR reaction of the present invention does not change the statistical likelihood of introducing an aliquot of fluid sample which, by chance had no genomic DNA or RNA in it to support PCR, or RT-PCR, respectively. Thus, determination of original sample density (genomes/μL) is not altered by PCR #1 (FIG. 1). The substantial signal amplification afforded by the use of that first PCR reaction (FIG. 1) greatly increases the number of amplified DNA molecules in those samples which, by chance, contained one or more nucleic target strands (FIG. 1) thus improving the sensitivity of single molecule detection near LLoD.

### Use of a Panel of Multiple DNA Hybridization Reactions to Enhance Authentication of “Bona Fide” PCR-Amplified “Positive” Hybridization Signals

The present invention describes the use of a first PCR or RT-PCR reaction (as in FIG. 1) in the context of a second PCR reaction coupled to DNA hybridization analysis on a microarray, rather than the use of a second real time PCR reaction as the second PCR step. The reason for such a choice is based on the capacity of a microarray to introduce a very large number of control measurements on a microarray, such that any hybridization signal obtained from a “positive” aliquot of amplified RNA or DNA to its cognate surface bound probe, can be verified as being a bona fide (specific) signal by means of direct comparison of that hybridization signal to multiple control probes on the same array (FIG. 2). Such control probes can be readily introduced into each microarray test and can be “mismatched” probes which have been altered by a simple physical change (i.e. to produce mismatched base pairings) or by the use of probes specific to other closely-related organisms, i.e. “species specific” probes. The ability to use a panel of multiple control probes to independently validate the data quality for a “positive” hybridization signal in the LLoD limit, on every sample being analyzed via the microarray test, is a unique property of microarray analysis in the present invention and is not generally possible with real time PCR.

More formally, and in the context of the present invention, when the (n) nucleic acid target sites are distributed throughout the pathogen genome, each can be interrogated by a set (Pₙ) of at least 9 microarray probes, comprising at least three types of probe (in triplicate). The first probe type (sₙ) is perfectly matched to a sequence in target site (n) which is chosen to be unique to the pathogen. A second probe type (mₙ) is identical to (sₙ) but altered to include at least 10% of base changes to induce mismatches. In addition, there is created at least a third probe type (vₙ) which is intentionally made to be identical to a sequence in a closely related species variant and to differ in sequence relative to (sₙ) by at least 10% of base changes (See FIG. 2).

The aggregated signal from all three probe types can be compared to each other to define a numerical value for the certainty that the hybridization signal (S) obtained from the pathogen (on probe sₙ) is statistically different from the hybridization signal (M) obtained on mₙ and also the signal (V) on vₙ; and wherein one such representative numerical value could comprise the relationship “Merit”=[S/(M+V)/2] where based on previous analysis of manufacturing and other sources of variance, “Merit” values at >10 would be significant of validated detection of the pathogen in any sample and values for “Merit” at <2 would indicate that the pathogen signal is not detected.

Nucleic acid-based microarray technology is based on the ability to mass produce DNA microarrays in a low cost a 1″×3″ glass slide format. This platform is used for DETECTX-RV and is scalable to 100,000 DETECTX-RV tests per month.

Briefly, viral RNA is extracted from a swab sample (see below) and taken through two Endpoint PCR reactions performed in tandem. The first PCR performs endpoint RT-PCR reactions on COVID-19 RNA to generate a set of primary DNA amplicons, each directed to one of several important regions of the COVID-19 genome N1, N2, N3. The primary DNA amplicons are used as a template for a second PCR reaction which additionally amplifies the primary product, while also applying a CY-3 fluorescent label to it. The second PCR is set-up as asymmetric PCR, a specialized version of Endpoint PCR, which produces a large excess of the CY-3 dye tagged strand of interest. The second PCR product is single stranded and can be used directly for microarray hybridization without clean-up or thermal denaturation. The workflow enables generation of 576 samples of microarray data/shift, which can be doubled with doubling upfront automation of RNA extraction.

The data is analyzed via AUGURY (Augury Technology company New York N.Y.), cloud-based automated software developed at PathogenDx, which can be implemented with modifications as appropriate. The software uses a basic logarithmic analysis to determine the results and is automatically processed and reported without any user interaction. Further, the cloud-based network capability enables data sharing with any number of testing labs needed to support national screening.

### Example 2

### A Microarray to Measure Very Low Levels of a Virus Such as COVID-19

Based on the general principles described in background, a microarray test is described with a LLoD at about 1 viral genome per assay and as such more than 10× more sensitive than Q-RT-PCR. Such a >10× sensitivity enhancement enables the ability to detect and speciate COVID-19 at 100 virus particles per swab, which according to the literature is roughly 10× greater sensitivity than any known Q-RT-PCR reaction. Such LLoD performance is a direct result of 3 fundamental principles of tandem PCR coupled to microarray analysis.

Two 30 Cycle PCR Reactions Performed Serially, which Deliver, De Facto, 60 Cycles of Endpoint RT-PCR Amplification Prior to Microarray Analysis

RNA template input held constant, such a 2-step tandem RT-PCR+PCR reaction produces DNA amplicon (to support microarray hybridization) at a concentration that is >3 orders of magnitude greater than the amount of PCR amplified DNA which generates the Cq metric in Q-RT-PCR.

### Analysis of Multiple COVID-19 Loci to Reduce the LLoD

Nucleic acid analysis becomes more sensitive when interrogating multiple copy loci, e.g. rDNA in bacteria or fungi, because one genome becomes represented by (n) identical target nucleic acid strands. In the present microarray test, (n)=6 independent COVID-19 test loci were configured, distributed throughout the genome.

Near the LLoD, where aliquot sampling becomes stochastic (see Equations 1 and FIG. 1) ordinary FDA standards specify that the LLOD be defined as the point where the assay generates at least 19/20 positives (due to aliquot sampling statistics). If a single PCR based assay is performed on each COVID-19 genome, such ordinary (Poisson) counting statistics specify that 19/20 positives would result from a population average of N=3 COVID-19 “events” in each sample aliquot being tested, typically 1 μL of an RNA extract. Thus, at n=1 target loci per genome, the practical limit of COVID-19 detection is about 3 genomes of purified RNA per μL.

Equation #2. The Analysis of Multiple Loci per Genome. The Effect on LLoD. Pr(0)=e^{-<N>}, where <N>=the population average of events per sample aliquot=(n×<g>), where <g> is the average number of genomes per sample and n is the number of loci analyzed per genome.

LLoD is defined by the FDA as Pr(0)=1/20=e^{-<N>}, thus at LLoD <N>=3 (3 average events per aliquot).

For 1 Locus analyzed per genome, (n)=1 and <N>=<g> and LLoD=3 genomes per aliquot (<N>=3).

For 6 Loci analyzed independently per genome, (n)=6 and <N>=6×<g> and LLoD=3/6=0.5 genomes per aliquot (n=6).

(n)=6 independent target loci per genome is easily obtained in a microarray test. In that important case, due to the multiple sampling (n=6) per genome, Equation #2 shows that the same 19/20 LLoD limit would be satisfied by a population average of 3/6=0.5 COVID-19 genomes per test. Thus, simultaneous measurement of (n)=6 independent target loci per COVID-19 test (each being an independent assay of the same genome) can be seen to reduce the LLOD 6-fold from about 3 COVID-19 genomes/assay to about 0.5 COVID-19 genomes/assay.

### Multiple, “Built-In” Hybridization Probe Controls to Define “Threshold” Internally for Every Sample

The widely used TaqMan qPCR technology, like all similar Molecular Beacon technologies, is based on deployment of PCR Primers (to amplify the RNA target) and the use of a Probe, to detect by hybridization, the PCR amplicon. Therefore, TaqMan Q-RT-PCR or its various Molecular Beacon equivalents are formally analogous to microarray hybridization analysis, which also relies on deployment of PCR amplification, then detection by hybridization.

Near the LLoD, the sensitivity of all nucleic acid tests (Q-RT-PCR and microarray hybridization included) become limited by the ability to distinguish “positive” signals from background, via the knowledge of a Threshold value to distinguish them from each other. In Q-RT-PCR, the analytical parameter of interest to define a “positive” signal is Cq. In microarrays, the analytical parameter is Relative Fluorescence Units (RFU).

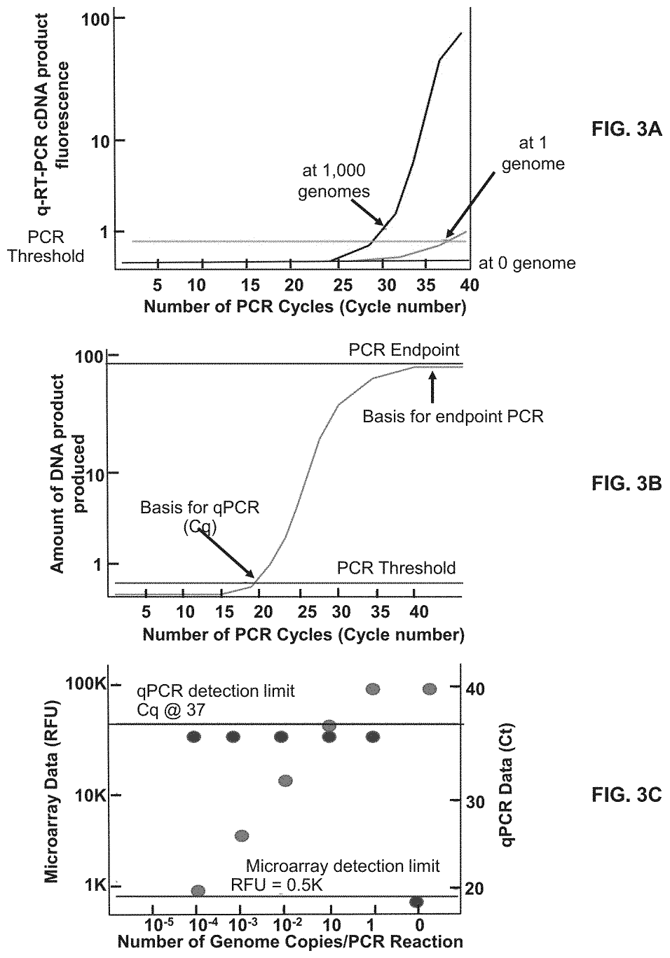

For Q-RT-PCR, background discrimination is based on the setting of a “Threshold” which is based on accumulated historical data, or external “no template controls” run in the same batch, along with actual samples, but external to the sample aliquots themselves. Thus in Q-RT-PCR, in nearly all cases, including the current COVID-19 Q-RT-PCR assays, the crucial calculation of “positive” Cq values vs “negative” signals is performed by extrapolation of an external Threshold and not by direct reference to internal standards within the same sample aliquot (FIG. 3).

As such, the capacity to detect positive signals, near the LLoD, where Cq values are typically at or greater than 35, becomes subject to sample-to-sample variation extrapolated from other measurements, rather than from the sample itself. Such extrapolation is a source of systematic calculational error to reduce the statistical certainty of distinguish “positive” from “negative” signals.

In the present Example, the DNA microarray test performs 144 individual hybridization tests in parallel for each sample aliquot tested. For each of the (n)=6 hybridization tests being used to detect COVID-19, 3 different “specific” probes are used to detect the presence of each of the (n)=6 viral cDNA targets/genome, along with 3 “mismatched” hybridization probes for each of the 6 target loci.

Thus, for the seminal parameter of importance to LLoD in microarray analysis (“positive” probe hybridization) the threshold, which defines the signal as being distinct from a “negative” is obtained for every sample by direct experimental numerical comparison. This is achieved by comparing a set of three “specific” vs three “mismatched” probes vs 3 or more species specific probes (FIG. 2). This set defines the magnitude of a “negative” signal and thus the threshold, via multiple independent methods in the same sample. Consequently, the LLoD for the present DNA microarray based test is much less sensitive to systematic (sample-sample) error in Threshold determination because the crucial comparison between “positive” and “negative” signal is not based on extrapolation, but is based on direct experimental analysis within each sample.

Testing of the microarray in this Example is focused on demonstrating that the LLoD for COVID-19 analysis is superior by an order of magnitude relative to that obtained by any of the known Q-RT-PCR assays. Such demonstration is done by third party testing on matched sample aliquots near the LLoD for microarray analysis relative to multiple commercial Q-RT-PCR COVID-19 tests.

### Lowest Limit of Detection

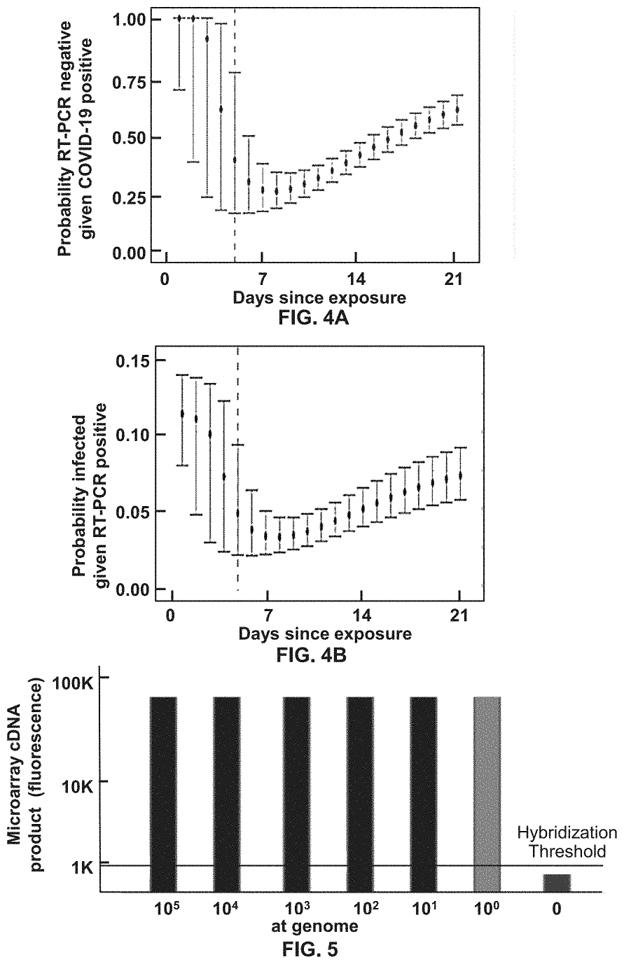

Q-RT-PCR technology has been widely implemented to test for COVID-19 among patients. Q-RT-PCR has been shown to have significantly high false negative rates in the range of 15% up to 48%, requiring re-testing (with the same level of inaccuracy). Therefore, it is challenging to detect low viral loads for patients who are asymptomatic or pre-symptomatic (3,4). FIG. 4A shows the probability of being RT-PCR negative among SARS-CoV2 infected patients and the FIG. 4B shows the probability of being infected, given RT-PCR positive (3).

False negative rates seen for Q-RT-PCR is due in part to the poor signal/noise ratio associated with Q-RT-PCR when it is implemented in the limit of low viral load and may be due to the nature of the principal Q-RT-PCR observable (Cq). It may also be due to poor control of RNA stability during and after collection.

Cq refers to the point at which PCR amplification of the COVID-19 genome produces enough product to be resolved from background (FIG. 3B). In that limit, the signal for (1) genome (Cq≈≈35) is not well-resolved from signal associated with (0) genomes at 40 Cq (FIG. 3A). While that distinction may seem esoteric, in the processing of low viral load samples (swabs or saliva) no more than 10 uL of the RNA extracted from such a sample can be introduced into the Q-RT-PCR reaction. The ability to resolve>1 genome from (0) genomes per PCR reaction is a requirement to set the useful LLoD. If as is ordinarily the case, the processed COVID-19 RNA delivered into Q-RT-PCR constitutes 5% of the viral RNA collected in the original sample to detect viral load of a hundred virion/\swab, the LLoD must approach that nearly-theoretical detection limit of 1 genome/reaction, which may be more than 10× lower than the present LLoD for Q-RT-PCR.

The LLoD (Solution): DETECTX-RV, an Alternative Technology Platform. The problems associated with LLoD is well known in the detection of other pathogens. The nucleic acid-based microarray technology of the present invention obviates LLoD limitations. The nucleic acid-based microarray technology is based on the ability to mass produce DNA microarrays in a low cost a 1″×3″ glass slide format.

### Deployment of Tandem PCR Prior to Microarray Hybridization Increases the Difference Between “Positive” and “Negative” Hybridization Signal Amplitude

By inspection of typical Q-RT-PCR data (FIG. 3) vs typical microarray data (FIG. 5) is can be seen that the signal size which distinguishes a “positive from a “negative signal in Q-RT-PCR (typically Cq=37 vs Cq=40) comprises a signal change that is generally small. For comparison, it can be seen that the signal size that distinguishes a “positive from a “negative” signal after tandem PCR then microarray hybridization (typically RFU 60,000 vs RFU=500) comprises a signal change that is almost 20×greater than that for Q-RT-PCR. Given that the ability to discriminate “positive” vs “negative” signal is the basis for the determination of LLoD for such testing, these data demonstrate that the signal strength (i.e. the positive-negative signal differential) is more than 10× greater for the present microarray technology, than is the case for Q-RT-PCR. Such representative differences are summarized in Table 3.

TABLE 3
Typical Microarray Hybridization Data
vs Q-RT-PCR Data, Limit near 0

| | | Q-RT-PCR | | Microarray |
|---|---|---|---|---|
| | | signal | Tandem PCR + | Signal |
| Copy | Q-RT-PCR | change | Microarray | change |
| Number per | Signal | relative to | Signal | relative to |
| reaction | (Cq) | zero | (RFU/1000) | zero |
| 100,000 | 30 | 20 | 60 | 59.5 |
| 10,000 | 34 | 16 | 60 | 59.5 |
| 1,000 | 27 | 13 | 60 | 59.5 |
| 100 | 30 | 10 | 60 | 59.5 |
| 10 | 33 | 7 | 60 | 59.5 |
| 1 | 36 | 4 | 60 | 59.5 |
| 0 | 40 | 0 | about 0.5 | 0 |

### Epidemiological Pooling is Enabled by Tandem Endpoint PCR

FIG. 5 shows that an additional important attribute of the present invention is that the data of importance, i.e. a positive” vs a “negative” signal in a sample aliquot, is binary in the sense that positive signals quickly converge to a limiting hybridization signal value (about 60,000 in FIG. 5) over about a 4-log dynamic range. Such a binary signal saturation is intentional in the present invention and is a direct result of the fact that both of the tandem PCR reactions (RT-PCR#1 or PCR #1+PCR #2) have been designed to proceed to completion during their execution, and thus are each a type of “Endpoint PCR”. The defining feature of Endpoint PCR (FIG. 3, right) is that the final amount of PCR product obtained after 30 or more cycles of PCR, often reaches a common plateau, independent of the amount of original pathogen input in a sample aliquot. This saturation is used to the benefit of the invention, to create a tandem PCR product, and in turn microarray hybridization data which remains constant (and large) over many factors of sample dilution.

A direct practical result of such saturation is that in many cases, such saturation allows samples to be pooled, as might be useful to expedite very large-scale epidemiological screening. See for instance, representative data as in FIG. 5, where it can be concluded that a sample containing 1,000 genome equivalents could easily be diluted with 10 samples, each lacking any pathogen, to yield a “pooled” sample, at 100 copies per aliquot in the present example, that would still be expected to demonstrate the presence of one or more contaminated samples within the pooled sample cohort.

### Exemplary Microarray Test, to Detect COVID-19 and Other Respiratory Viruses Test Content

In this example, COVID-19 is the primary analyte, plus multiple coronavirus targets [SARS-CoV, MERS-CoV, CoV 229E, CoV OC43, CoV NL63, CoV HKU1] plus Influenza [type A and B] as species variants (Table 4).

TABLE 4
DETECTX-RV Content. PCR Primers and Microarray Probes

| | Target | Microarray | PCR |
|---|---|---|---|
| Viral Target | Sites/Virus | Probes | Primers |
| SARS-CoV2 | N1, N2, N3 | 12 (Sₙ) 12 (mₙ) | 3 sets |
| SARS-CoV | N, 1ab | 4 (vₙ) | |
| SARS-CoV2 (Mutation) | S - D614G | 2 | 1 set |
| MERS-CoV | N, 1ab | 2 (vₙ) | 2 sets |
| CoV 229E | N, 1ab | 2 (vₙ) | 2 sets |
| CoV OC43 | N, 1ab | 2 (vₙ) | 2 sets |
| CoV NL63 | N, 1ab | 2 (vₙ) | 2 sets |
| CoV HKU1 | N, 1ab | 2 (vₙ) | 2 sets |
| Pan Influenza A-type | M, NS1 | 2 | 2 sets |
| Pan Influenza B-type | M, NS1 | 2 | 2 sets |
| Internal RNA Control | RNAse P | 2 | 1 set |

The extra content available in the microarray format allows a very large panel of COVID-19 target sites (n=6) to be measured in parallel and in triplicate. The other six coronavirus targets and two influenza targets are included and are being used as both controls and as a universal screening tool for coronavirus and influenza.

### Specificity

For each of the n=6 unique SARS-CoV2 target loci [N1, N2, N3, ORF1ab, RNA-dependent RNA polymerase (RdRP), E] there are (2) microarray probes (Sₑ), 12 specific probes in total, and 2 mismatched probes (mₙ) for each, with 10% of intentional base mismatching (i.e. there are 12 mismatched specificity probes). Relative to the twelve COVID-19 specific probes (Se), the 14 species specific controls (vₙ) are distributed among other coronavirus (SARS-CoV, MERS-CoV, CoV 229E, CoV OC43, CoV NL63, CoV HKU1). In that format, a Positive COVID-19 signal for any one of the set of six loci, deemed valid if it possesses a fluorescence signal strength of >10× background (>10,000 RFU) while at the same time and in the same microarray, the mismatched specificity probe (mₙ) generates a signal less than 2× background (<2,000 RFU).

### DETECTX-RV Assay Improves the LLoD for Viral Detection

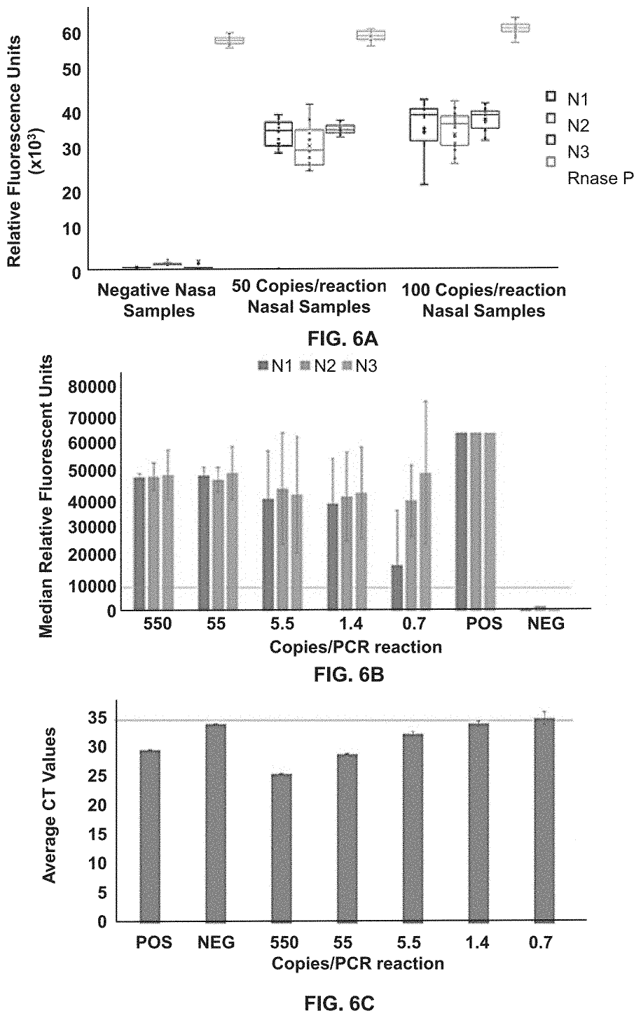

The serial application of two PCR Endpoint reactions (RT-PCR, with Asymmetric PCR) creates a type of analysis which is “Binary” in the sense that, an aliquot of specimen which lacks RNA produces no measurable hybridization signal, under conditions where any input with at least 1 genome copy produces a signal of nearly constant, very large signal amplitude. Such behavior is shown graphically in FIGS. 6A-6C, where during the course of microarray analysis only two classes of hybridization signal are detected namely, “Positives” resulting from samples with one or more copies of viral RNA target (producing fluorescent signals in excess of about 40,000 RFU) vs “Negatives” resulting from samples with (0) copies of viral RNA target, which produce no hybridization signal above background.

### “Binary” Hybridization Principles

For LLoD analysis, the highly characterized COVID-19 standards (BEI) have been doped into N=30 separate pooled human nasal secretion samples (Lee Bioscience) along with 20 matched (negative) nasal secretion controls. Subsequent to RNA purification (Zymo kit) the resulting 50 RNA samples were subjected to PCR-Microarray.

As seen from FIGS. 6A-6C, all three COVID-19 targets N1,N2,N3 and the human RNA internal control (P) each display clustered signals that are independent of viral load and which give a (+)/(−) ratio of about 20-110, which is approximately 20× the signal strength typically obtained by Q-RT-PCR in the same range of viral load (FIGS. 6B and 6C). That log increase in Signal-Background is central to the detection power of the DETECTX-RV technology. It should be noted that only 20% of the original 1 ml sample is subjected to RNA preparation, and in turn only 20% of that is used for microarray analysis. Thus, assuming 100% recovery from RNA extraction, the data shown in FIG. 6A comprise at most, signal from 2 copies and 4 copies per test (that is, 1/25th of the original 50 and 100 copies doped into the pre-processed sample).

To confirm the intrinsic detection limit inferred from the LLoD analysis (FIG. 6A, <<5 copies/reaction) a simple dilution series was performed (FIGS. 6B, 6C), where well-characterized purified SARS-CoV2 RNA (ATCC/BEI) is titrated into PBS followed by DETECTX-RV analysis (FIG. 6B) or in parallel, Q-RT-PCR (FIG. 6C) using kits from RayBiotech (gift from RevolutionDx Labs, Dayton Ohio). In all cases, the data shown comprise n=10 determinations at each dilution level, measured in units of copies added/PCR reaction. The DETECTX-RV data (FIG. 6B) displays the “Binary” Characterization described above, especially for N2 and N3 SARS-COV2 target sequences. Within experimental accuracy, the measured signal strength does not diminish with dilution over the range from 550 to 0.7 genome copies/PCR reaction and retains a signal strength of about 20× to negative controls at (0) copy per reaction. Thus, consistent with the LLoD data (FIG. 6A) the detection limit for DETECTX-RV is <1 genome copy per reaction, becoming limited by the stochastic nature of such “copy counting” rather than by diminishment of signal strength as the 1 genome/reaction limit is approached. By comparison, signal from the N1 target diminishes marginally at the lowest level (0.7 copies/reaction). This observed N1 behavior at about 1 copy/reaction can be mitigated by increase of its PCR primer concentration.

Comparison to matched Q-RT-PCR data (N1 target) shows performance typical of all such Q-RT-PCR tests. The data obtained below 5.5 copies per reaction becomes indistinguishable from the detection threshold (Ct≈35) as defined by negative controls. Thus, the detection limit for DETECTX-RV (<1 genome copy per reaction, is more than 5× lower than that of the present Q-RT-PCR assay. A summary of COVID-19 hybridization statistics is shown in Table 5.

TABLE 5
COVID-19 Hybridization Statistics

| | | | | Signal |
|---|---|---|---|---|
| | | | | Divided by |
| SARS-CoV2 Targets | | | Standard | Negative |
| N1, N2, and N3 RNase P Control | | Average | Deviation | Background |
| Negative | N1 | 273 | 63 | — |
| Nasal Samples | N2 | 1189 | 287 | — |
| | N3 | 1726 | 6601 | — |
| | RNase P | 56479 | 5531 | — |
| 50 copies/reaction | N1 | 31199 | 11194 | 114 |
| Nasal Samples | N2 | 30904 | 5507 | 26 |
| | N3 | 35181 | 1372 | 20 |
| | RNase P | 58614 | 1317 | — |
| 100 copies/reaction | N1 | 34781 | 9650 | 127 |
| Nasal Samples | N2 | 33740 | 8224 | 28 |
| | N3 | 37647 | 3459 | 22 |
| | RNase P | 60586 | 1604 | — |

### Sample Pooling

Based on the substantial Signal/Background ratio obtained with DETECTX-RV near the LLoD (FIGS. 6A-6C), it was determined whether positive samples containing COVID-19 copies near the LLoD could be “pooled” with samples that were also in nasal matrix, but lacked COVID-19 RNA. As previously calculated, the benefit of such pooling appears to reach a maxim at N=10, especially in the limit of a low population infection rate (at 1%).

Such 10-fold pooling is shown in Table 6, wherein a single sample near the LLoD (50 copies/ml) is mixed with an equal volume of 9 samples lacking COVID-19 RNA, yielding a net viral load of about 5 copies/ml. As seen in Table 6, all 3 COVID-19 markers are detected in each of the pooled samples tested. The data show both of the important attributes of “Binary” sample Collection. The signal strength at 5 copies/ml, is about 30,000 RFU, which is identical within experimental accuracy to the 50 copies/ml sample used for pooling (Table 6) and in turn identical within experimental accuracy to identical un-pooled samples at 100 copies/ml (FIGS. 6A-6C). Both the unpooled sample (at 50 copies/ml) and the pooled sample (at 5 copies/ml) are near to the range where simple counting statistics begin to contribute to the data.

### Test Content

The Problem: The platform limit of Q-RT-PCR can be multiplexed to resolve four analytes in parallel, based on the four principal emission channels on most devices including CDC, LabCorp, Quest (N1,N2,N3, P). This limit may be exceeded as evidenced for Abbott (RdRp, N), Cepheid (E,N2,P) and Eurofins (N,P). However, the “maxed” capacity suggests that the known Q-RT-PCR assays will not be able to accommodate additional testing complexity, such as might arise if alternative COVID-19 clade variants were to emerge. A recent publication has suggested however, that a stable variant has been detected comprising a mutation in the spike protein S-D614G, which has been hypothesized to be more virulent.

TABLE 6
Pooling of Contrived nasopharyngeal Samples

| | N1 | | | N2 | | | N3 | | | RNase P | | |
|---|---|---|---|---|---|---|---|---|---|---|---|---|
| | | Original | RFU | | Original | RFU | | Original | RFU | | Original | RFU |
| Specimen | Pooled | (unpooled) | Difference | Pooled | (unpooled) | Difference | Pooled | (unpooled) | Difference | Pooled | (unpooled) | Difference |
| No. | Copies/PCR | | | | | | | | | | | |
| 1 | 28372 | 37941 | 9569 | 52162 | 37535 | 14627 | 54474 | 34875 | 19599 | 63708 | 59477 | 4231 |
| 2 | 1901 | 35502 | 33601 | 7051 | 34064 | 27013 | 43202 | 35692 | 7510 | 63822 | 58669 | 5154 |
| 3 | 7096 | 36504 | 29408 | 7772 | 30066 | 22294 | 491 | 34769 | 34278 | 63590 | 56065 | 7525 |
| 4 | 54097 | 35026 | 19071 | 52847 | 24796 | 28050 | 55732 | 37250 | 18481 | 63369 | 58476 | 4893 |
| 5 | 53035 | 34549 | 18486 | 55302 | 32452 | 22850 | 55422 | 34985 | 20437 | 63288 | 59814 | 3474 |
| 6 | 42780 | 29158 | 13622 | 53682 | 27965 | 25718 | 56635 | 35545 | 21090 | 63535 | 57633 | 5902 |
| 7 | 61250 | 38769 | 22481 | 58258 | 41392 | 16866 | 56104 | 37459 | 18645 | 63464 | 59929 | 3535 |
| 8 | 57968 | 440 | 57528 | 56086 | 26561 | 29525 | 54116 | 33214 | 20901 | 63670 | 60348 | 3322 |
| 9 | 45702 | 31467 | 14236 | 56951 | 24634 | 32316 | 55258 | 34304 | 20954 | 63565 | 57664 | 5901 |
| 10 | 51537 | 32639 | 18898 | 55067 | 29572 | 25495 | 52673 | 33719 | 18953 | 63656 | 58067 | 5589 |

Based on test content capacity alone, detection of both SARS-CoV2 and the S-D614G spike protein mutant will prove difficult to detect on the same q-RT-PCR test. Thus Q-RT-PCR might not be useful as a tool to screen for both variants.

The Solution. The process by which new coronavirus content can be added to DETECTX-RV is very efficient. It is based on the robust probe capacity of the arrays (144) and on the highly standardized methods of PCR primer design and microarray probe design (at one base pair hybridization specificity). As an example, the presumed importance of the S-D614G mutant was only recently published (Apr. 29, 2020). The variant comprises a SNP G-A transition converting Asp to Gly. New probes specific for the wild type and new variant along with a set of mismatched control probes were designed within a day, and submitted for fabrication, and were completed May 11, 2020. Microarray fabrication with these new probes was added to an otherwise identical DETECTX-RV microarray and were completed on May 15, 2020. In parallel, a pair of test amplicons were designed and produced by SGI methods possessing the wild type and new COVID-19 SNP. In parallel, 4 candidate PCR primer pairs have been designed. Probe selectivity was confirmed with the SGI template, and in parallel, inclusivity and exclusivity confirmed experimentally with the full panel of coronavirus research standards in-house from ATCC-BEI.

### Specificity

The Problem. While Q-RT-PCR can deliver adequate test specificity it is well-known that the TaqMan probe-template interaction does not adequately resolve SNPs in many cases (6,7) due to the fact that in a TaqMan assay, primer binding, probe binding and the Taq exonuclease activity must all occur at the same time and thus cannot be optimized independently. The problem is exacerbated in the present case (S-D614G) because the SNP generates a “run” of 3G's, which are difficult to accommodate.

The Solution. In this respect, the microarray technology of the present invention is beneficial as it has the capability of routinely generating “all or none” SNP discrimination due to uncoupling of probe binding from PCR. Further, a separate washing step is included for improved specificity. Here, a first set of hybridization tests are shown on a set of probe candidates to detect and resolve the SNP variants which define SARS-CoV2 Clade variation at the Spike protein (D-614G). Methods of probe design were used. Array manufacture was performed in the standard 12-well format, but all other aspects of probe formulation and deposition were identical to those deployed in the 96 and 384 well Plate formats. Six PCR primer pairs were designed and optimized for the standard Tandem PCR (RT-PCR+Labeling PCR) amplification process. Since both “sense” and “antisense” probes were tested, different asymmetric Labelling PCR reactions were deployed, which differed in which of the 2 PCR primers had the 5′-CY3 label in the second PCR reaction (labeling PCR).

### Hybridization in 12-Well Slide Format

To evaluate hybridization feasibility in 12-well slides, 50 probes candidates were printed on the slides to detect and resolve the 2 SNP variants which define SARS-CoV2 Clade variation at the Spike protein (D-614G). Proprietary methods of probe design developed at PDx (PathogenDx, Scottsdale, Ariz.) were used in the design. All aspects of probe formulation and deposition were identical to those used for 96-well and 384-well plates.

A PCR primer pair was designed and optimized for standard (tandem) 2-Step RT-PCR and labeling PCR. Since both “sense” and “antisense” probes were tested, different asymmetric labelling PCR reactions were deployed, which differed in which of the 2 PCR primers had the 5′-CY3 label. The template for this study was a pair of synthetic templates. Each template contained the defining SNP (A or G) embedded in the Wuhan reference sequence for the Spike protein.

Subsequent to standard hybridization and washing in the slide format (similar to 96-well and 384-well plate format), the two SNP variants were resolved with signal (relative fluorescence units, RFU) strength differences in the range from 15-40 (see Table 7) which approaches the theoretical limit for resolving SNPs by hybridization. For expediency, two “Sense Strand” and Two “Antisense Strand” candidates from the probe set were chosen for inclusion in the 384-well Plate “Mini-RV” print content. All four of these probes displayed very good sensitivity and SNP specificity. This Example conforms that the present tandem PCR (RT-PCR+labeling PCR) reaction coupled to microarray hybridization can cleanly resolve two SARS-Cov2 variants which differ by a single RNA base change the Spike protein.

### Scalability

The Problem. Scalability of test capacity is a huge challenge particularly during a pandemic. Assuming 1000 COVID-19 test sites throughput the US, this would require the ability for each site to support at least 10,000 tests/shift/site. At present Roche and Abbott lead the pack with Q-RT-PCR capacity, amounting to 250-900 tests/shift and 150 tests/shift respectively. This microarray technology supports population scale nucleic acid screening.

The Solution. In the present deployment of DETECTX-RV, the core array format (12×12) is deployed as 12 tests per slide. Eight such slides are routinely processed in parallel with ordinary fluid handling, thus allowing multiples of 96 tests in parallel.

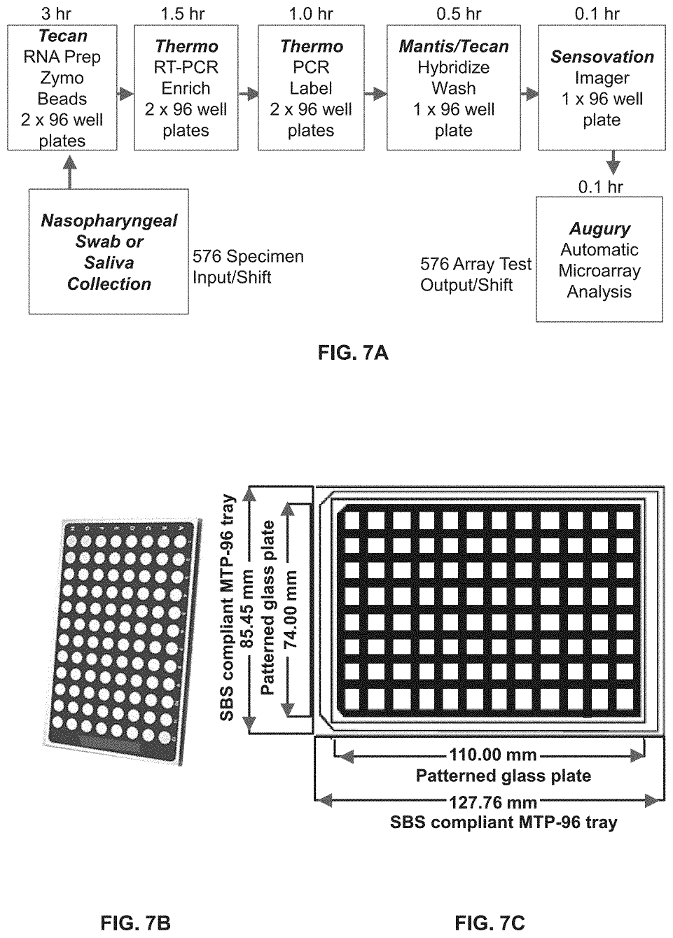

At present, coupled to ordinary 96-well magnetic bead RNA purification, the hybridization steps are in all cases faster than the RNA preparation. Automation and system integration are deployed with industry leading partners. Technologies, which have been integrated for DETECTX-RV (FIG. 7A) are each already approved for invitro diagnostic (IVD) use for workflow required for DETECTX-RV testing viz, RNA preparation via magnetic beads (“Tecan”, Tecan Trading AG) RT-PCR and PCR (Thermo) Open Architecture, Ambient Temperature Binding and washing (Tecan) and microarray imaging (Sensovation). The AUGURY software discussed in Example 1 was developed at PathogenDx and has all functionalities in place to support DETECTX-RV data acquisition and analysis and has been modified to process both 96-well and 384-well plates. Its capacity to manage and upload such data into a secure Cloud Network is also complete and fully validated for RUO use. AUGURY is in place among 100 Regulated Testing Labs. Additionally, AUGURY may be operated on a customer's slide imager or computer. This is an advantage as it obviates the requirement for uploading large size images to the cloud which may be time consuming. Smaller size dot score files and output reports may continue to be uploaded to the data repository in the cloud.

TABLE 7
Preliminary Spike Hybridization on Multiple Probes Reveals SNP Resolution

| | 614 “D” Gene fragment used as | | | | | | 614 “G” Gene fragment used as | | | | | | | | |
|---|---|---|---|---|---|---|---|---|---|---|---|---|---|---|---|
| | template for PCR | | | | | | template for PCR | | | | | | Summary | | |
| | PCR primer | | | | | | PCR primer | | | | | | Average | Average | Specifi- |
| | Set 1 | Set 2 | Set 3 | Set 4 | Set 5 | Set 6 | Set 1 | Set 2 | Set 3 | Set 4 | Set 5 | Set 6 | “on” signal | “off” signal | city ratio |
| Negative control probe | 818 | 1378 | 1306 | 933 | 1079 | 1414 | 1353 | 1667 | 1754 | 681 | 1182 | 1506 | N/A | 1256 | N/A |
| Universal sense | 62680 | 62844 | 62585 | 62792 | 62846 | 62966 | 62692 | 62739 | 59582 | 62912 | 62980 | 62382 | 62500 | not | N/A |
| probe (1.1) | | | | | | | | | | | | | | shown | |
| 614D sense probe (1.1)* | 44032 | 43713 | 27743 | 40724 | 43321 | 40221 | 937 | 992 | 785 | 1070 | 788 | 1117 | 39959 | 948 | 42:1 |
| 614G sense probe (1.1) | 2910 | 2293 | 1344 | 4040 | 2936 | 2742 | 57171 | 54694 | 41786 | 51479 | 52386 | 47918 | 50906 | 2711 | 19:1 |
| 614D sense probe (1.2) | 32106 | 26908 | 15862 | 26590 | 29157 | 29425 | 782 | 464 | 244 | 464 | 493 | 436 | 26675 | 480 | 32:1 |
| 614G sense probe (1.2)* | 1684 | 1238 | 284 | 743 | 1130 | 897 | 38030 | 40558 | 30402 | 38493 | 38089 | 38909 | 37413 | 996 | 38:1 |
| 614D sense probe (1.3) | 23244 | 26480 | 9676 | 27537 | 26787 | 19197 | 734 | 691 | 782 | 704 | 842 | 668 | 22153 | 737 | 30:1 |
| 614G sense probe (1.3) | 1663 | 1335 | 1372 | 1592 | 1778 | 1584 | 12536 | 13520 | 7302 | 13813 | 12849 | 11782 | 11967 | 1554 | 8:1 |
| 614D sense probe (1.4) | 62650 | 62275 | 51469 | 62764 | 62758 | 60077 | 12087 | 14478 | 7047 | 12071 | 11956 | 9954 | 60332 | 11266 | 5:1 |
| 614G sense probe (1.4) | 4570 | 4528 | 2530 | 3930 | 4856 | 4415 | 62112 | 61074 | 46297 | 55602 | 53042 | 55918 | 55674 | 4138 | 13:1 |
| Negative control probe | 1084 | 1319 | 1276 | 996 | 1565 | 1356 | 1018 | 1417 | 1767 | 675 | 1124 | 1918 | N/A | 1293 | N/A |
| Universal sense | 62556 | 62369 | 62425 | 62448 | 62356 | 62579 | 62441 | 62311 | 62164 | 62398 | 62360 | 62466 | 62406 | not | N/A |
| probe (1.1) | | | | | | | | | | | | | | shown | |
| 614D sense probe (1.1)* | 62556 | 62369 | 62390 | 62448 | 59860 | 60257 | 11481 | 14199 | 15072 | 11668 | 12673 | 14795 | 61646 | 13314 | 5:1 |
| 614G sense probe (1.1)* | 2025 | 2770 | 2440 | 2719 | 2387 | 3645 | 62441 | 62311 | 62164 | 62398 | 62360 | 62466 | 62356 | 2664 | 23:1 |
| 614D sense probe (1.2) | 44098 | 44683 | 43784 | 47299 | 43836 | 43526 | 5681 | 2628 | 3668 | 1717 | 2241 | 2750 | 44538 | 3114 | 14:1 |
| 614G sense probe (1.2) | 1319 | 1444 | 1352 | 1516 | 1393 | 1071 | 50541 | 52272 | 46864 | 52110 | 46551 | 49087 | 49571 | 1349 | 37:1 |
| 614D sense probe (1.3) | 25571 | 28872 | 21946 | 29427 | 25272 | 26117 | 1474 | 1745 | 2490 | 1749 | 1821 | 2893 | 26200 | 2029 | 13:1 |
| 614G sense probe (1.3) | 1208 | 1513 | 1042 | 1183 | 1297 | 1300 | 25524 | 26758 | 20659 | 27328 | 28548 | 34719 | 27256 | 1257 | 22:1 |
| 614D sense probe (1.4) | 62556 | 62369 | 62425 | 62448 | 62356 | 62579 | 37925 | 37274 | 30141 | 36901 | 34103 | 37302 | 62455 | 35607 | 2:1 |
| 614G sense probe (1.4) | 4597 | 7848 | 4909 | 5042 | 5793 | 6225 | 62441 | 62311 | 62164 | 62398 | 62360 | 62466 | 62356 | 5736 | 11:1 |

*Currently on 12-probe 384-well array

TABLE 8
Streamlined COVID-19 Analysis, DETECTX-RV-V2

| | | Target | Micro- | |
|---|---|---|---|---|
| Row | | Sites/ | array | PCR |
| # | Viral Target | Virus | Probes | Primers |
| 1 | SARS-CoV2 (hCoV-19) | N2 | 2 | 1 set |
| 2 | SARS-CoV | N2 | 1 | |
| 3 | hCoV-19/pangolin (groups 1 | N2 | 1 | |
| | and 2) | | | |
| 4 | hCoV-19/bat/Yunnan/RaTG13 | N2 | 1 | |
| | (2013) | | | |
| 5 | Bat_SARS-like_CoVZC45 and | N2 | 1 | |
| | XC21 | | | |
| 6 | hCoV-19/bat/Yunnan/RmYN02 | N2 | 1 | |
| 7 | SARS-CoV2 (hCoV-19) | N1 | 2 | 1 set |
| 8 | SARS-CoV2 (Mutation) | S-D614G | 2 | 1 set |
| 9 | Internal Control | RNAse P | 1 | 1 set |

### Example 3

### DETECTX-RV-V2

The full content of the original DETECTX-RV assay is described in Example 2 and Table 4. Table 8 shows a variant (DETECTX-RV-V2) of that Pan Coronavirus format. It is based on SARS-CoV2 analysis at (N1,N2) as in the original assay and differs in the inclusion of 2 new microarray probes and an additional RT-PCR primer pair to interrogate the recently described novel S-D614G mutant (5) in the same assay.

The streamlined DETECTX-RV-V2 assay deploys 12 microarray probes, which when printed in N=12 multiplicity, become a highly redundant 144 probe array suitable for printing in the present 12-well slide format, and in the more automation-friendly 96-well Society for Biomolecular Screening (SBS) format (FIGS. 7B-7C). DETECTX-RV-v2 additionally contains a set of 4 other coronavirus (rows 3-6, Table 8), which have been previously identified by cluster analysis (GISAID—Initiative) as being the closest SARS-CoV2 homologues. These targets provide functionally relevant “species specificity” controls that help confirm that the signals obtained from SARS-CoV2 (COVID-19) or its S-D614G mutant are specific. It must be noted that although the DETECTX-RV-V2 test variant is simpler in design and execution than the original DETECTX-RV prototype (Table 4) its test content is at least 3× greater than any Q-RT-PCR assay.

### Structure of the 96-Well Format for DETECTX-RV-V2

The 96-well late format (FIGS. 7B-7C) for COVID-19 testing, developed by Schott glass (NEXTERION) uses epoxy-silane coated, Teflon masked slides. They serve as an excellent substrate for microarrays. The 96-well plate SBS format is better suited for large scale, COVID-19 testing. Although the plate format is slightly more expensive than the slide format at small scale, the COGS for arrays in plates are less than on slides, at production>714,240 arrays/month.

The 96-well DETECTX-RV-V2 workflow has been integrated into off-the-shelf Tecan automation (Freedom Evo-2 100 Base) beginning with magnetic bead-based RNA extraction (Zymo) and ending with automated microarray hybridization and washing. The intervening PCR reactions are mediated by Tandem Thermo-ABI cyclers and imaging is performed on a Sensovation CCD based imager. Data generated is fed into AUGURY software discussed in Example 2 for autonomous plate reading, microarray data compilation and analysis.

The major strength of the DETECTX-RV-V2 technology is its large-scale public health application in any setting including at-home, at-work, healthcare institutions and transportation hub sample collection for diagnosis and detection of active and asymptomatic individuals. Current use of nasopharyngeal swabs is not suitable for such collection, due in most cases to the difficulty of sample collection and the instability of RNA on such swabs, using the currently used transport media of the day.

### High Throughput Automation

The Tecan robot or other commercial equivalents can process multiple 96-well plates in parallel, thus sample throughput of (6) 96-well microarray plates/shift is possible (FIGS. 7A-7C). Upon transition to a 384 well format, the Tecan and related commercial robots can be reprogrammed for the higher-throughput 384-well format.

### DETECTX-RV-V3

Deployment of DETECTX-RV-V2 enables 12,000 arrays/day in a 96-well array plate format providing a 360,000 arrays/month capacity. While DETECTX-RV-V2 will retain its 12×12=144 element structure (in 7 mm wells), the 384-well structure (3.5 mm wells) will accommodate a 6×6=36 probe array. The core probe content for SARS-CoV2 (N1, N2)S-D614G variant and Human RNase-P (P) internal control can all be included along with SARS CoV and MERS CoV as species specificity controls as 12 probes, printed in triplicate. The DETECTX-RV-V2 format may be modified to include pan Influenza A and Influence B probes to generate a targeted pan-respiratory virus test (DETECTX-RV-V3). The DETECTX-RV-V3 format has substantial benefits since it readily adapts to increase system testing throughput to more than 2,104 tests/shift, which exceeds existing commercial testing technologies and at the same time achieves a 3× reduction in test cost, from manufacturing & reagent economies of scale.

### Manufacturing

DETECTX-RV-V2 & DETECTX-RV-V3 are each manufacturable in 24 hours with a single printer in batches of 62 plates, comprising 6,000 arrays/day (96-well plate) 24,000 arrays/batch/day (384-well plate). Each printer completes two batches per 24-hour day.

### Example 4

### Development of a Fully Featured Pan-Coronavirus-Influenza Test

### DETECTX-RV System Architecture.

The entire suite of Pan-Coronavirus content (Table 4) has been designed, developed and manufactured and is resident in the DETECTX-RV version of the assay. The full Pan-Corona Respiratory Virus content suite is validated using standardized viral reagents from ATCC-BEI, which are spiked into the same matrices (nasal and saliva). The test format employed for such expanded validation uses the LLoD and N=30 repeats testing protocols.

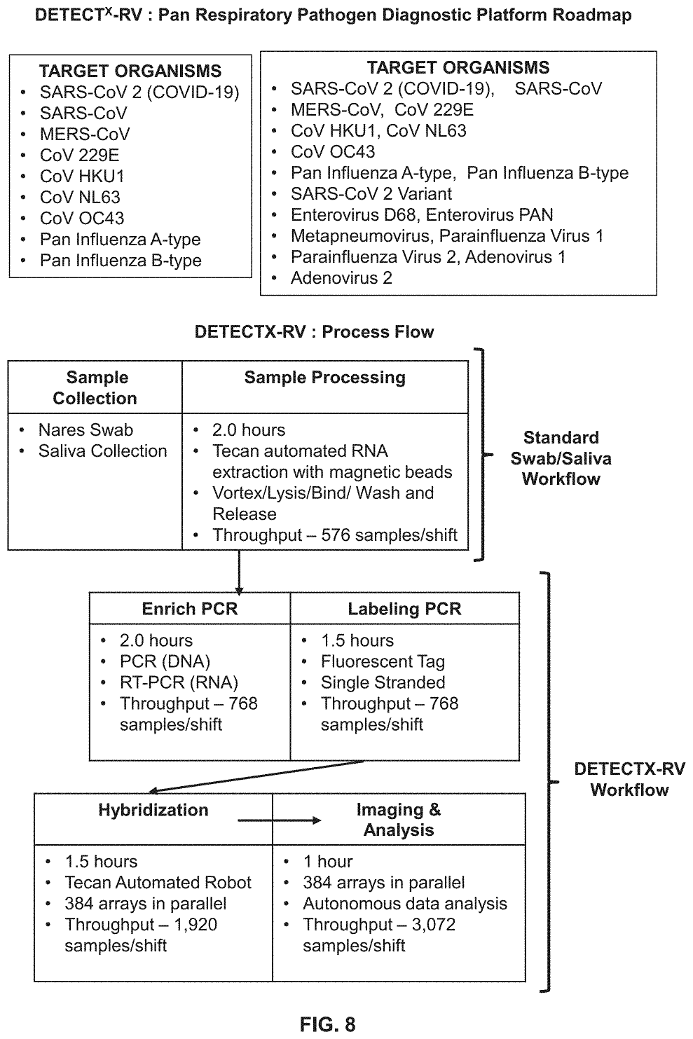

Early stages of COVID-19 clade development are in progress, which could be selected for stable changes in environmental durability, virulence or acute symptomology. PathogenDx monitors such data on a daily basis. At such time that solid evidence emerges for development of stable COVID-19 clade variants, new content was immediately added to DETECTX-RV (FIG. 8).

The process by which new coronavirus content can be added to DETECTX-RV is very efficient due to the robust probe capacity of the arrays (144), and the highly standardized methods of PCR primer design and microarray probe design (at one base pair hybridization specificity).

If a new SARS-CoV2 subtype were identified in the literature, based on one or more regions with local sequence change in one of the domains already Interrogated in DETECTX-RV (N1, N2 or N3), PCR primer design would not change. The only modification is design of one or more new probes specific for the new variant added to the existing DETECTX-RV microarray. In parallel, a test amplicon would be produced by ordinary SGI methods possessing the new COVID-19 sequence markers. Probe selectivity would be confirmed with the SGI template, and in parallel, inclusivity and exclusivity confirmed experimentally with the full panel of coronavirus research standards in-house from ATCC-BEI.

On the other hand, if the new content were in regions not yet being interrogated, the process remains the same, with the added task of designing and fabricating a primer pair to amplify the COVID-19 region of interest. The primer design process occurs in parallel to probe design with a 2-week turnaround for the desired DETECTX-RV test modification.

### DETECTX-RV Enhanced Content (DETECTX-RV-V2)

The DETECTX-RV assay coupled to nasopharyngeal swab collection is presently being launched into CLIA certified labs for human diagnostics screening. Its oligonucleotide probe content (Table 4) comprises a 12×12 array, at present, with RNA targets comprising sites within a set of 10 respiratory viruses and a human RNA control (RNase P). Of these, SARS CoV2, SARS-CoV and SARS COV2 (mutation) support pandemic testing. The remainder of the test content (other coronaviruses and Influenza) are present as probes within the present 12×12 array and used as specificity controls.

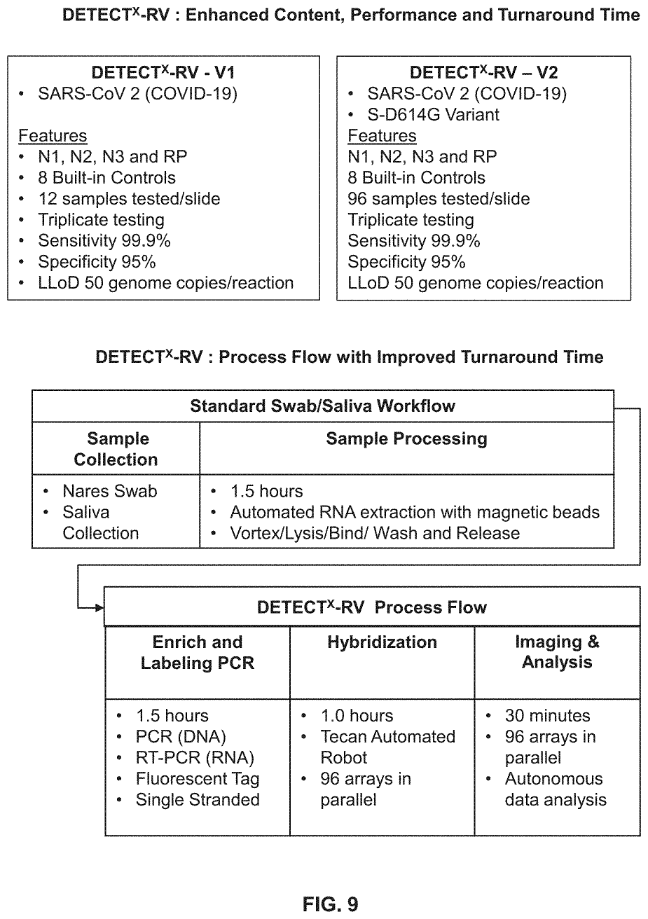

In the Tandem, Asymmetric, Two-Step implementation of the present invention, DETECTX-RV workflow begins with viral RNA that had been extracted from a nasopharyngeal Swab Sample followed by two Endpoint PCR reactions in tandem. The first PCR, an “Enrich” PCR (FIG. 9) performs (N=4 multiplex) endpoint RT-PCR reactions on COVID-19 RNA to generate a set of primary DNA amplicons, each directed to one of several important regions of the COVID-19 genome N1, N2, N3 (Table 4). The primary DNA amplicon product serves as the template for a second PCR reaction The second PCR reaction is set-up using CY-3 fluorescent labeled primers (“Labelling” PCR) in 4-fold or 8-fold excess over unlabeled reverse primers which are not dye labelled. The second PCR is set-up as asymmetric PCR—a specialized version of endpoint PCR and produces a large excess of the CY-3 dye tagged strand of interest. The second PCR product is single stranded and therefore can be used directly for microarray hybridization without clean-up or thermal denaturation. This technology is robust for large scale respiratory virus screening of clinical samples in at-home, at-work and healthcare institutional settings.

The DETECTX-RV workflow shown in FIG. 9 can generate 576 samples-worth of microarray data/shift; which can be doubled with doubling up-front automation of RNA extraction. The data is analyzed autonomously via AUGURY software.

### Example 5

### Sample Collection

The COVID-19 pandemic has confirmed what many had known from field study of zoonotic disease: namely that the “Viral Transport Media” (VTM) used to collect virus on swabs, are poor stabilizers of viral RNA. To address this, a novel chemical stabilizer from GENTEGRA LLC (GTR) as well as inexpensive polymer stabilizers (PVS) along with well-known lab-based RNAse inhibitor (RNA-Shield) are used to allow for stable field collection of respiratory virus samples on swabs without refrigeration. Stabilized swab collection (COVID-19, Coronavirus and Influenza stability over one week at 30° C.) enables better clinical collection of nares swabs and saliva fluid also enables at-home nasal swab collection for population scale screening in centralized labs. Emphasis is to support very large-scale clinical collection (nares) plus at-home (lower nasal) collection.

### Modified Swab Design

A modified swab design that includes chemical stabilizers of viral RNA initiated in collaboration with GENTEGRA LLC enables samples to be transported at ambient temperature. This improved collection design may be employed with the DETECTX-RV-V2 platform to support very large-scale clinical collection and at-home collection.

### Modified Sample Processing Hardware and Software for System Integration

The technologies for integration into DETECTX-RV are approved for in vitro diagnostics use for the type of workflow required for DETECTX-RV testing—RNA preparation via magnetic beads (Tecan) RT-PCR and PCR (Thermo Fisher Scientific), open architecture, ambient temperature binding and washing (Tecan) and microarray imaging (Sensovation AG). The AUGURY software has all functionalities in place to support DETECTX-RV data acquisition and analysis. Its capacity to manage and upload such data into a secure cloud network is also complete and fully validated for RUO use.

### Modified Saliva Collection by Chemical Stabilization of Viral RNA.

A “mouthwash” based saliva collection technology (QUIKSAL) is employed for collecting saliva samples. In a separate set of studies, 200 nasopharyngeal swabs are collected per the standard RevolutionDx and Lucid Lab protocols along with matched QuiKSal mouthwash collection from the same individual (400 matched swabs and Saliva). The swab and half of the mouthwash is analyzed in accordance with standard Q-RT-PCR workflow, while the remainder of the mouthwash was split and shipped at ambient temperature and −20 C in transport medium for analysis at PathogenDx on the DETECTX-RV-V2 microarray. The samples analyzed at PathogenDx have no associated personal identifiers or medical information other than the Cq values obtained from Q-RT-PCR testing at RevolutionDx.

Feasibility of Sample Pooling from Swabs and Saliva for Population Scale Screening

Pooling of swab and saliva samples among pre-symptomatic individuals is a powerful tool to enable contact tracing. This is established by the findings that demonstrated pooling of specimens with the highest COVID-19 load from at least 64 nasopharyngeal swab samples via Q-RT-PCR is free of false negatives when the input (positive) sample used for pooling is a clear, “strong positive” and characterized by a Cq value<30 (FIGS. 3A-3C, 4A and 4B). Specifically, the threshold for determination of “COVID-19 Positive” is Cq<35 for most Q-RT-PCR assays. At this threshold, the intrinsic “False Negative” rate is about 20% to about 40%.

Sample pooling is a powerful public health screening tool. However, for the most useful pooling levels (N≥10) for many COVID-19 positive samples (those with Cq>30) Q-RT-PCR generates an unacceptably high “Pooled False Negative Rate”. If that occurs, sample pooling in combination with Q-RT-PCR would not be adopted as a routine public health or industrial hygiene tool.

As shown in Table 6, data with contrived nasopharyngeal samples near the LLoD (at 50 genome copies/ml) suggests that DETECTX-RV may have the sensitivity needed to enable expanded pooling (N=10) with a reduced risk of false (pooled) negatives. Therefore, contrived samples are used to refine the sensitivity and specificity of N=10 pooling similar to that shown in Table 6, with technical emphasis on increasing cycle number from 30-35 in the Enrichment RT-PCR reaction. Pooling is then performed prior to RNA extraction, on the same swab and saliva samples freshly obtained. Raw samples (unstabilized swabs or stabilized swabs or stabilized saliva) are measured immediately by both DETECTX-RV-V2 and by Q-RT-PCR. Immediately upon identification of “true “positives”, the set is divided into quartiles, based on the semi-quantitative Q-RT-PCR data (that is, Very High, High, Medium, Low) for viral load based on the Cq value associated with each. 20 uL of each such positive sample are immediately mixed with 20 uL of 9 of the many “negatives” to yield 200 μL of pooled sample and transferred directly into Zymo RNA lysis buffer for freezing prior to RNA extraction. This approach permits the nasopharyngeal swab or saliva studies to yield up 40-80 unique N=10 pooled samples, where data for each pooled sample (Table 8) is directly compared to the “positive” from which it originated.

### Example 6

### Analysis of Clinical Samples (Nasopharyngeal Swabs)

### Clinical Sample Evaluation:

PathogenDx received 50 blinded nasopharyngeal swab samples in flash frozen Abbott Transport Media from Testing Matters Laboratory (TM Labs—Sunrise, Fla., CLIA certified) to evaluate the performance of the PathogenDx DETECTX-RV assay in comparison to the FDA-EUA approved Abbott Real-Time SARS-CoV2 qPCR assay.

Each of the 50 samples were collected on the same day/same time, one sample was collected from the right nostril and one from the left nostril. The two separate samples (each separately labelled and stored identically in transport medium) were taken back to TM Labs where one sample was flash frozen and shipped to PathogenDx and the second sample was processed and screened according to the Abbott Real-Time SARS-CoV2 qPCR assay FDA-EUA protocol. The results from the Abbott testing at TM Labs were shared after PathogenDx had screened the 50 samples using the DETECTX-RV assay.

The 50 matched samples that were sent to PathogenDx, arrived frozen on dry ice and were stored at −80° C. until use. The samples were thawed on ice and 400 μL of the 2 mL sample was used as the input for the Zymo Quick-DNA/RNA Viral MagBead purification. The purified RNA was then used to screen for SARS-CoV2 in these patient samples according to the PathogenDx product insert using the Promega AccessQuick RT-PCR system coupled to the PathogenDx PCR and the corresponding microarray test.

There were 50 total samples tested as well as the PathogenDx external positive and negative controls. Table 9 shows the results of the analysis.

TABLE 9
Comparison of Q-RT-PCR and DETECTX-RV analysis

| | Abbott | | | | | | | | | |
|---|---|---|---|---|---|---|---|---|---|---|
| | Q-RT-PCR | | PathogenDx DETECTX-RV | | | | PathogenDx DETECTX-RV | | | |
| | COVID-19 | | SARS-COV-2 (Run 1) | | | | SARS-COV-2 (Run 2) | | | |
| Sample | Ct | POS/ | RFU Value | | | POS/ | RFU Value | | | POS/ |
| ID | Value | NEG | N1 | N2 | N3 | NEG | N1 | N2 | N3 | NEG |
| 18997 | — | NEG | — | — | — | NEG | | | | |
| 18977 | — | NEG | — | — | — | NEG | | | | |
| 18955 | 15.4 | POS | 43951 | 42054 | 45570 | POS | 45156 | 41096 | 52115 | POS |
| 18902 | — | NEG | — | — | — | NEG | | | | |
| 18907 | 24.21 | POS | 16988 | 18392 | 40621 | POS | 11354 | — | 41910 | POS |
| 18943 | — | NEG | — | — | — | NEG | | | | |
| 18974 | — | NEG | — | 11452 | 40725 | POS | — | — | — | NEG |
| 18913 | 25.67 | POS | 37474 | 37443 | 47522 | POS | 31044 | 34238 | 51157 | POS |
| 19032 | — | NEG | — | — | — | NEG | | | | |
| 18962 | — | NEG | — | — | — | NEG | | | | |
| 18969 | — | NEG | — | — | — | NEG | | | | |
| 18994 | — | NEG | — | — | — | NEG | | | | |
| 18983 | — | NEG | — | — | — | NEG | | | | |
| 19029 | — | NEG | — | — | — | NEG | | | | |
| 18989 | — | NEG | — | — | — | NEG | | | | |
| 18935 | 11.56 | POS | 42479 | 40111 | 42063 | POS | 46701 | 40965 | 52325 | POS |
| 19026 | — | NEG | — | — | — | NEG | | | | |
| 18906 | 26.13 | POS | 8858 | 15329 | 45827 | POS | 11021 | 8272 | 46969 | POS |
| 18958 | — | NEG | — | — | — | NEG | | | | |
| 18963 | — | NEG | — | — | — | NEG | | | | |
| 19005 | — | NEG | — | — | — | NEG | | | | |
| 19016 | — | NEG | — | — | — | NEG | | | | |
| 18993 | — | NEG | — | — | — | NEG | | | | |
| 18986 | — | NEG | — | — | — | NEG | | | | |
| 19014 | — | NEG | — | — | — | NEG | | | | |
| 19027 | — | NEG | — | — | — | NEG | | | | |
| 18928 | — | NEG | — | — | — | NEG | | | | |
| 18867 | — | NEG | 30246 | 34971 | 51303 | POS | 18297 | 21713 | 48564 | POS |
| 19020 | — | NEG | — | — | — | NEG | | | | |
| 18871 | — | NEG | — | — | — | NEG | | | | |
| 19030 | 26.25 | POS | 16515 | 18861 | 46116 | POS | — | — | 39022 | RERUN |
| 18953 | — | NEG | — | — | — | NEG | | | | |
| 19022 | — | NEG | — | — | — | NEG | | | | |
| 18927 | — | NEG | — | — | — | NEG | | | | |
| 18972 | — | NEG | — | — | — | NEG | | | | |
| 19003 | — | NEG | — | — | — | NEG | | | | |
| 18870 | — | NEG | — | — | — | NEG | | | | |
| 18978 | — | NEG | — | — | — | NEG | | | | |
| 19024 | — | NEG | — | — | — | NEG | | | | |
| 18910 | — | NEG | — | — | — | NEG | | | | |
| 18981 | — | NEG | — | — | — | NEG | | | | |
| 19017 | — | NEG | — | — | — | NEG | | | | |
| 18990 | — | NEG | — | — | — | NEG | | | | |
| 19000 | — | NEG | — | — | — | NEG | | | | |
| 19007 | — | NEG | — | — | — | NEG | | | | |
| 19025 | — | NEG | — | — | — | NEG | | | | |
| 19019 | — | NEG | — | — | — | NEG | | | | |
| 18937 | 25.3 | POS | 27209 | 26359 | 31789 | POS | 28012 | 31267 | 50736 | POS |
| 18967 | — | NEG | — | — | — | NEG | | | | |
| 19009 | — | NEG | — | — | — | NEG | | | | |

Run 1—The DETECTX-RV assay demonstrated 100% concordance with the samples called positive (N=7) using the Abbott system and 93% concordance with the samples called negative (N=43) using the Abbott system. The PathogenDx, DETECTX-RV assay identified 2 additional samples as positive, that were identified as negative by Abbott testing and one additional sample as needing to be rerun. Measurements were repeated for all 9 samples of the samples that were identified as positive using the DETECTX-RV assay.

Run 2—The DETECTX-RV assay demonstrated 86% concordance (6 DETECTX-RV/7 Abbott) with the samples called positive (N=7) using the Abbott system. The one sample that was discordant was identified as a rerun on the second run. The rerun confirmed the positive signal from Run 1 for the two samples that were identified as negative by Abbott testing. The one previous sample that was identified as a rerun came back as negative on the second run. Table 10 summarizes the results from Run 1 and Run 2.

TABLE 10
Summary of results from Run 1 and Run 2

| | POS (N = 7) | NEG (N = 43) |
|---|---|---|
| Run 1 | 100% POS | 95% NEG + 2 POS |
| Run 2 | 86% POS + 1 RERUN | 97% NEG + 1 POS |

### Example 7

### Analysis of Environmental Samples (Surface Swabs and Air)

One of the greatest challenges in performing environmental monitoring of air and surfaces for viral contamination is the collection and stabilization of the viral RNA prior to analysis. To overcome this challenge, the strategy of utilizing a dilute RNA stabilization solution (from GENTEGRA LLC) called “ATA” here was evaluated at 1:40 dilution in 1×PBS and/or DNase/RNase Free Water for stabilization of COVID-19 RNA collected from surfaces on swabs or from the air into a fluid collection solution, using a device from Bertin Corporation, as an example.

### Environmental Monitoring of Air

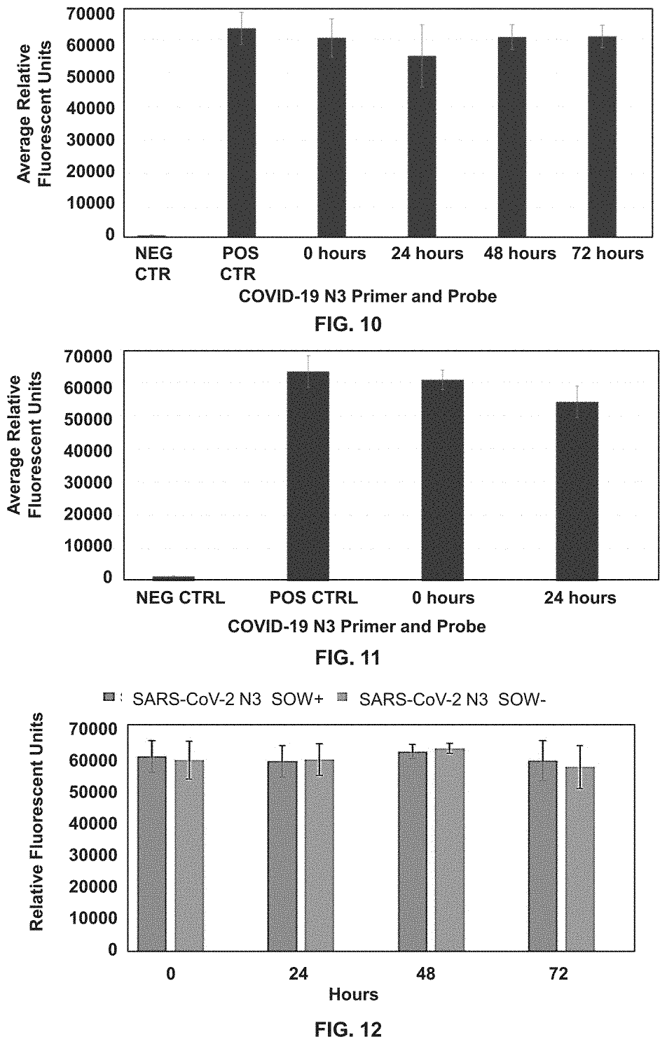

To determine if the air is contaminated with bacteria, fungi, and/or virus air was collected using the Coriolis Micro Air Sampler from Bertin. In this utilization, the stability of viral RNA was evaluated during and up to 72 hours post collection. In this demonstration the GENTEGRA RNA stabilizer (“ATA”) was diluted at 1:40 dilution in 5 mL of 1×PBS, pH 7.2 or molecular grade water. Purified 5 μL of SARS-CoV2 RNA was spiked at 200,000 copies/μL directly into the collection cone and ran the instrument to dryness, which took ˜30 min, during which the spiked sample was exposed to the particulate contamination resulting from @2m3 of collected air input. Post air collection the dry viral RNA plus dried stabilizer and accumulated airborne contaminants were resuspended in 1 mL of molecular grade water and stored the samples at room temperature (0, 24, 48, and 72 hours) until RNA purification was performed. The RNA was extracted and purified using the Zymo Quick DNA/RNA Viral MagBead collection kit and the samples were ran on the DETECTX-RV assay by PathogenDx. RNA collection and stability for the entire 72 hour period as demonstrated in the FIG. 10, which presents signals obtained from the N3 region of COVID-19 as measured on the DETECTX-RV assay produced via the present invention. Data are presented as raw microarray hybridization signals obtained from probes for the N3 region, as a function of post-collection storage time at RT (in hours). The positive control constitutes an identical matched, unprocessed spiked COVID-19 sample that had not gone through air collection, air drying or storage. The data show that the 30 minutes of air collection (0 hours) did not give rise to measurable RNA loss, nor did up to 72 hours of RT storage of the dried air-collection sample prior to analysis.

### Environmental Monitoring of Surfaces by Swabbing

To determine if the surface is contaminated with a microbe (COVID-19 virus in the present example) surface swab samples were collected using nylon flocculated and rayon swabs. In this utilization, the stability of viral RNA during and up to 24 hours post collection was evaluated. In this demonstration, the “ATA” RNA stabilizer was diluted 1:40 in 5 mL of 1×PBS, pH 7.2 or molecular grade water. Purified 5 μL of SARS-CoV2 RNA was spiked at 200,000 copies/μL then applied it directly onto a stainless-steel surface. The swab was removed from its sterile case and three drops of the dilute “ATA” stabilizer were placed onto the swab to moisten it. The surface was swabbed to collect the viral RNA. The swab was placed directly back into the sterile container and allowed to sit at room temperature for 24 hours. Post surface collection and either (0 hrs) or (24 hrs) of ambient temperature swab storage, 1 mL of 1×PBS, pH 7.2 was added to the swab in the container and vortexed for 10 seconds. 400 μL of the resuspended viral RNA was removed for viral RNA preparation. The RNA was extracted and purified using the Zymo Quick DNA/RNA Viral MagBead collection kit and the samples were run on the DETECTX-RV assay, monitoring the fluorescence signal from the COVID-19 (N3) region. The positive control constitutes an identical matched, unprocessed spiked COVID-19 samples that had not been applied to the surface or gone through swabbing or storage. The data demonstrate RNA recovery and stability from a surface swab, subsequent to ordinary ambient storage of the swab for 24 hrs, as assessed by analysis via the present invention, as demonstrated in FIG. 11.

### Example 8

### Analysis of Mouthwash Samples

The purpose of this study was to demonstrate that the present invention could be used to detect COVID-19 RNA in a novel oral rinse solution (QuiKSal from CLC Corporation) which had been spiked into it at clinically meaningful levels, then analyzed subsequent to several days of unrefrigerated ambient temperature storage, to emulate overnight shipping from point of collection to a central lab for COVID-19 analysis by the present invention. Two versions of QuiKSal were tested. One possessed a tracking Dye (SOW+) and one without the dye (SOW−).

The QuiKSal procedure asks the patient to swish 1 mL of the QuiKSal and spit the QuiKSal into the sterile storage container. The collection procedure was mimicked by spiking in a high and a low SARS-CoV2 RNA into 1 mL of QuiKSal. Eight 1 mL aliquots of Oral Rinse Solution were created, with and without SARS-CoV2 RNA spike. Two of the spiked sampled aliquots had 200,000 copies/mL (high) of a SARS-CoV2 standard (Integrated DNA Technologies) while the other six aliquots were spiked to 20,000 copies/mL (low). Following the addition of the RNA to the samples the samples were stored from 0 to 72 hours at room temperature to evaluate the stability of the RNA in the QuiKSal mouthwash. Following incubation, the RNA was isolated using the Zymo Quick-DNA/RNA Viral MagBead kit by removing 400 μL of the QuiKSal for sample preparation per the manufacturer's instructions. Following sample preparation, the samples were analyzed using the PathogenDx DETECTX-RV test, based on the teaching of the present invention.

Array data (FIG. 12) showing detection of SARS-CoV2 N3 target gene relative fluorescent units (RFU) at various time points after spike into SOW+ (with dye) and SOW− (minus dye). No signal was obtained from the no template control oral rinse (not shown). Signals above 10,000 RFU are considered positive.

The present invention was capable of detecting COVID-19 RNA from the QuiKSal oral rinse with or without dye. COVID-19 RNA in that stabilized mouthwash was detectable via the present invention for up to 72 hours at room temperature.

### Example 9

### Printing and Quality Control

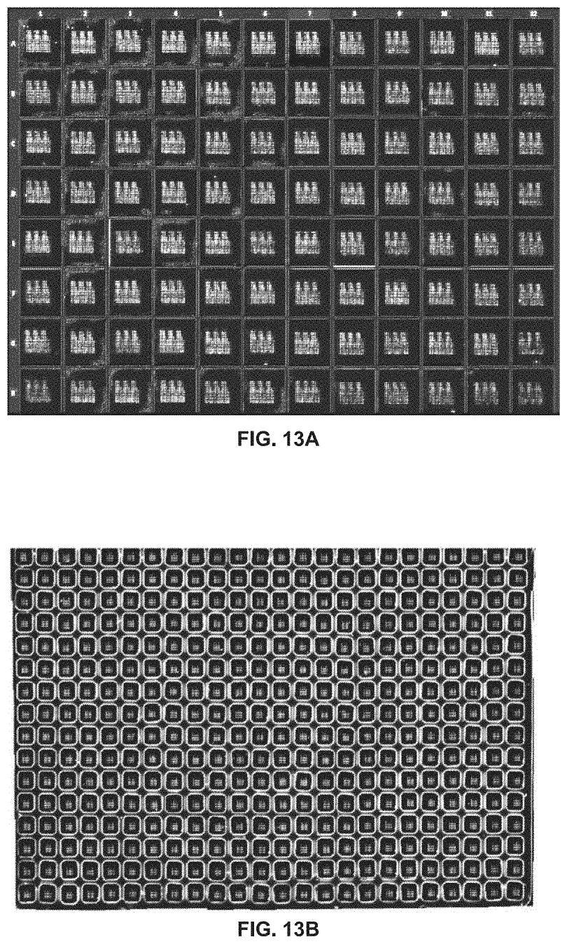

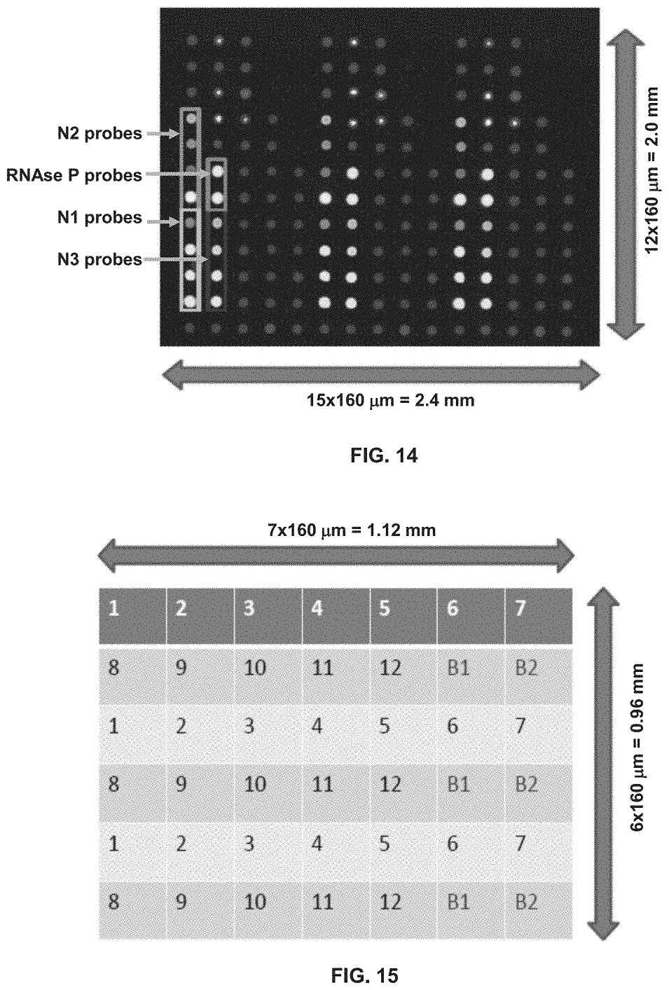

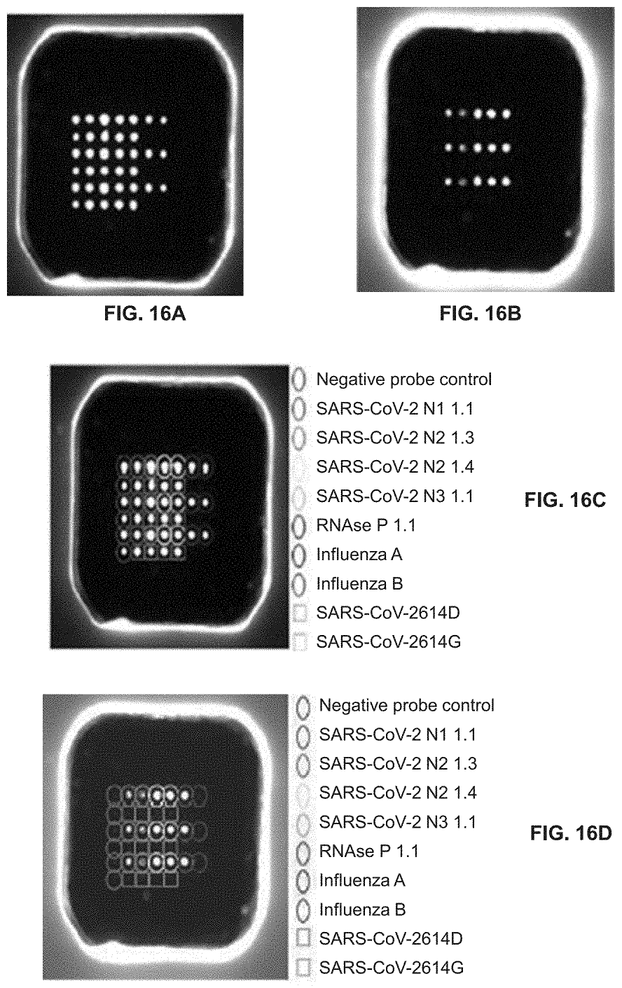

96-well plates were printed with the hybridization probes under conditions optimized to eliminate dust and fiber contamination in the wells. Optical Inspection suggested that there are no measurable failures in printing (FIG. 13A, pate #9901005001). Similarly, 384-well plates were printed with the hybridization probes. The plates were inspected using Sensation Imaging and reveal no measurable failures (FIG. 13B, plate #1 9980001001). Therefore, no further changes to printing parameters and slide processing (UV and well mounting) are required. The array structure and probe layout for the 96 well plate (FIG. 13A) are shown in FIGS. 8 and 14. The probes and probe layout for the 384-well printing (FIG. 13B) are exactly as displayed in FIGS. 16A-16D and as described in Table 12.

### Hybridization Analysis

A small number of validated clinical nasopharyngeal swab samples obtained from Boca Biolistics having 11 positives and 1 validated nasopharyngeal negative control were subjected to standard 2-step tandem RT-PCR (RT-PCR+PCR). Standard hybridization and washing were performed with an increase in hybridization (136 μL) and wash (200 μL) volumes, followed by imaging from the bottom of the fully assembled 96-well plate (plate #9901005001). FIG. 14 shows one of the positive samples from one well of the 96-well plate. A gradient of probe affinity was used for each of the locus analyzed using N1, N2, N3 and RNAse P probes. Four of the loci (N1, N2, N3, RNAse P) are for COVID, while the rest are species controls including other coronavirus, Influenza A and Influenza B. As seen, the array structure has well-characterized sample signals for all targets (N1, N2, N3, RNAse P probes). Negligible cross hybridization is observed among the various controls.

### Pilot Study on Clinical Nasopharyngeal Isolates on Identical DETECTX-RV Arrays in 96-Well Vs 12-Well Slide Format

A small number of validated clinical nasopharyngeal samples obtained from Boca Biolistics having 11 positives and 1 validated nasopharyngeal negative control were subjected to standard 2-step tandem RT-PCR (RT-PCR+PCR). Samples were analyzed on the 96-well slide format with direct comparison to match samples on the 12-well slide format and are shown in Table 11. Signal intensity was optimized by increasing labelling PCR reaction volume and sample volume.

### Probe Design and Probe Material Assembly of Mini-RV 384-Well Microarray

The probe content for the smaller version of DETECTX-RV (Mini-RV V1) was designed and is shown in Table 12. FIG. 15 shows a 6×7 probe layout for the Mini-RV 384-well microarray where the contents are printed in triplicate.

TABLE 11
Comparison of DETECTX-RV data on 96-well plates and 12-well slides.

| 384-well plate Sample # → | 17 | 18 | 19 | 20 | 21 | 22 | 25 | 26 | 27 | 28 | 29 | 30 |
|---|---|---|---|---|---|---|---|---|---|---|---|---|
| H-RNAse P positive control | D | D | D | D | D | D | D | D | D | D | D | D |
| Negative RT-PCR control | ND | ND | ND | ND | ND | ND | ND | ND | ND | ND | ND | ND |
| SARS-CoV2 N3 | D | D | D | D | ND | D | D | D | D | D | D | D |
| SARS-CoV2 N1 | D | D | D | D | ND | D | D | D | D | D | D | D |
| SARS-CoV2 N2 | D | D | D | D | ND | ND | D | D | D | D | D | D |
| 12-well slide Sample # → | 1 | 2 | 3 | 4 | 5 | 6 | 7 | 8 | 9 | 10 | 11 | 12 |
| H-RNAse P positive control | D | D | D | D | D | D | D | D | D | D | D | D |
| Negative RT-PCR control | ND | ND | ND | ND | ND | ND | ND | ND | ND | ND | ND | ND |
| SARS-CoV2 N3 | D | D | D | D | ND | D | D | D | D | D | D | D |
| SARS-CoV2 N1 | D | D | D | D | ND | D | D | D | D | ND | D | D |
| SARS-CoV2 N2 | D | D | D | D | ND | D | D | D | D | D | D | D |

D = detected, signal above threshold; ND = not detected, signal below threshold

### Example 10

### Mini-RV Hybridization in 384-Well Format

SARS-CoV2 probe specificity and characteristics of probe prints were evaluated for clinical nasopharyngeal swab samples.

### Materials:

384-well test print—9980001001

### DETECTX-RV kit

SARS-CoV2 standard at 200 copies/reaction (Exact Diagnostics LLC)

Probes were printed in triplicate and amplified at 200 copies/reaction. A pooled PCR sample was created from 24 individual PCR reactions amplifying SARS-CoV2. FIGS. 16A-16D shows data for a representative well. A clear replicate fluorescent signal was obtained between wells for RNAse P, SARS-CoV2 N1, N2 and N3 probes (FIGS. 16A, 16B). FIGS. 16C and 16D show the results of imaging analysis for the CY5 (Probe label) and CY3 (amplicon) label. These data demonstrate feasibility of the 2-step labeling protocol and functionality of the 384-well plate.

### Example 11

### Optimization of Microarray Manufacture

By increasing the amount of UV cross linking from 300 mJ to 500 mJ, the signal strength obtained for COVID-19 microarray analysis in the 96-well and 384-well plate format was comparable (Tables 13 and 14) to that obtained with 12-well slide hybridization as assessed by LLoD and clinical specimen analysis.

TABLE 12
Probe content in Mini-RV, 384-well plate format

| | |
|---|---|
| 1 | Negative hybridization control probe |
| 2 | SARS-CoV2 N1 probe |
| 3 | SARS-CoV2 N2 probe |
| 4 | SARS-CoV2 N2 probe alternate |
| 5 | SARS-CoV2 N3 probe |
| 6 | RNAse P probe |
| 7 | Influenza A probe segment (M) |
| 8 | Influenza B probe segment (NS) |
| 9 | SARS-CoV2 (S) 614D probe antisense |
| 10 | SARS-CoV2 (S) 614G probe antisense |
| 11 | SARS-CoV2 (S) 614D probe sense |
| 12 | SARS-CoV2 (S) 614G probe sense |
| B1 | Blank (for make-up/manufacturer error correction) |
| B2 | Blank (for make-up/manufacturer error correction) |

TABLE 13
Comparison of SARS-CoV2 Hybridization Signals on 12-well, 96-well
and 384-well plates for 30 Contrived LLoD Samples. (62.5 copies/ml
in Boca nasopharyngeal negatives using the 2-Step method)

| | |
|---|---|
| Average | Standard Deviation |

12-Well

| | | |
|---|---|---|
| SARS.COV2-N1-RE1.1 | 48543 | 9553 |
| SARS.COV2-N2-RE1.3 | 51253 | 11844 |
| SARS.COV2-N3-RE1.1 | 57398 | 12004 |
| RNAse.P.Probe-pub1.1 | 60697 | 11038 |

96-Well

| | | |
|---|---|---|
| 62-Negcont-B | 537 | 508 |
| SARS.COV2-N1-RE1.1 | 38377 | 25385 |
| SARS.COV2-N2-RE1.3 | 48524 | 13774 |
| SARS.COV2-N3-RE1.1 | 56905 | 11754 |
| RNAse.P.Probe-pub1.1 | 60312 | 11108 |

384-Well

| | | |
|---|---|---|
| 62-Negcont-B | 2328 | 872 |
| SARS.COV2-N1-RE1.1 | 48919 | 10759 |
| SARS.COV2-N2-RE1.3 | 37186 | 7833 |
| SARS.COV2-N2-RE1.4 | 54071 | 10080 |
| SARS.COV2-N3-RE1.1 | 54087 | 9993 |
| RNAse.P.Probe-pub1.1 | 55129 | 4339 |

TABLE 14
Comparison of SARS-CoV2 Hybridization on 12-well, 96-well
and 384-well plates for 30 positive and 30 negative clinical
samples (Boca/NP/VTM using the 2-Step method)

| | | | |
|---|---|---|---|
| | Positive | | Negative |
| Average | Standard | Average | Standard |
| Positive | Deviation | Negatives | Deviation |

12-Well

| | | | | |
|---|---|---|---|---|
| 62-Negcont-B | 1640 | 312 | 1711 | 209 |
| SARS.COV2-N1-RE1.1 | 32850 | 19701 | 1322 | 2343 |
| SARS.COV2-N2-RE1.3 | 38570 | 15524 | 2980 | 6620 |
| SARS.COV2-N2-RE1.4 | 43670 | 16656 | 3779 | 6748 |
| SARS.COV2-N3-RE1.1 | 59723 | 5485 | 5182 | 10348 |
| RNAse.P.Probe-pub1.1 | 62532 | 319 | 63073 | 165 |

96-Well

| | | | | |
|---|---|---|---|---|
| 62-Negcont-B | 122 | 351 | 347 | 336 |
| SARS.COV2-N1-RE1.1 | 35577 | 20782 | 251 | 1474 |
| SARS.COV2-N2-RE1.3 | 26098 | 15252 | 1383 | 1220 |
| SARS.COV2-N2-RE1.4 | 54771 | 13048 | 1852 | 8090 |
| RNAse.P.Probe-pub1.1 | 62475 | 298 | 62611 | 788 |

384-Well

| | | | | |
|---|---|---|---|---|
| 62-Negcont-B | 2233 | 761 | 2512 | 510 |
| SARS.COV2-N1-RE1.1 | 30764 | 16572 | 1354 | 1698 |
| SARS.COV2-N2-RE1.3 | 28451 | 15541 | 2781 | 2658 |
| SARS.COV2-N2-RE1.4 | 51946 | 11150 | 5203 | 10023 |
| SARS.COV2-N3-RE1.1 | 54192 | 6684 | 5224 | 8237 |
| RNAse.P.Probe-pub1.1 | 57343 | 1681 | 57494 | 1496 |

### Example 12

### Performance Optimization

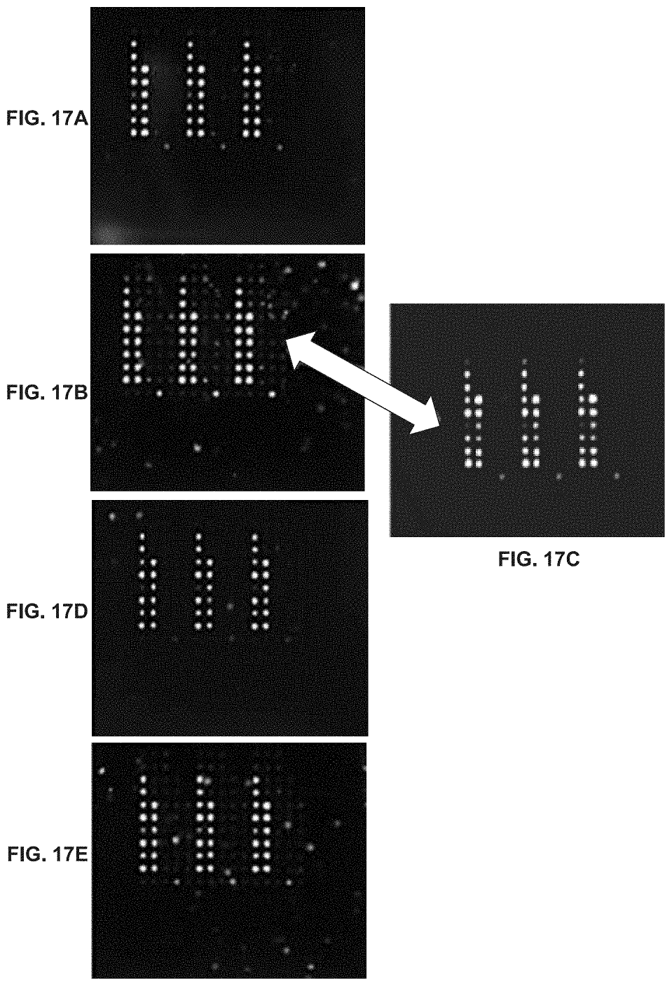

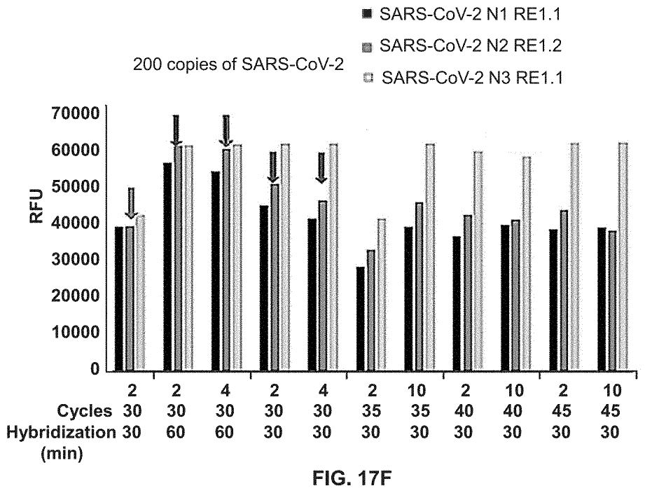

Methods to improve signal strength and overall performance were analyzed for 96-well plates (FIGS. 17A-17D) and led to the following basic principles for 96-well plates, which were similarly deployed in the analysis of 384-well plates.
a) Plates must be cross-linked prior to mounting of the 96-well (or 384-well) polycarbonate top.
b) A modest increase in signal strength is obtained by mixing and/or an extension of hybridization time from 30 min to 60 min (FIG. 17E). Mixing alone improves signal strength and may be facilitated with a plate shaker. Optimization data for the hybridization in 96-well format are summarized in FIG. 17F.
c) Image quality is improved by introducing a 1 min plate centrifugation. This step is performed prior to loading the plates onto the Sensovation Imager.

### Asymmetric One-Step RT-PCR Optimization

Validation of Asymmetric One-Step RT-PCR using purified SARS-CoV2 RNA

### Materials:

1. 12-well glass slides—99030002 print series.

2. DETECTX-RV kit.

3. Purified SARS-CoV2 RNA (ATCC, NR-52285)

4. Labeling primers

### Optimization 1

To determine if the Tandem 2-step (RT-PCR+Labelling PCR) reaction can be combined to a single step (Asymmetric One-Step RT-PCR) to reduce assay times, first, different primer ratios (labeled:unlabeled) were used in the PCR reaction to establish optimal cycle number to achieve sensitivities similar to the 2-step reaction (LoD˜2 copies/reaction, 125 copies/mL)

Four different primer concentrations and ratios (labeled:unlabeled 4:1, 4:1, double concentration, 8:1, 2:1) were used. Three different cycling conditions were used over a dilution (500 copies/reaction=62,500 copies/mL to 2 copies/reaction=125 copies/mL) of purified SARS-CoV2 RNA. The purified SARS-CoV2 RNA was diluted in sterile water from 500, 250, 100, 50, 25, 10, 5 and 2 copies/reaction. NTC (No Template Control) and External extraction controls were also used. The PCR parameters were as follows;

Cycling conditions: 35, 40, 45

RT-PCR Program: 45° C., 45 min

PCR Program:

| | |
|---|---|
| Initial denature | 95° C., 2 min |
| Cycling | 95° C.-30 sec; 55° C.-30 sec; 68° C.-30 sec |
| Final extension | 68° C.-5 min |

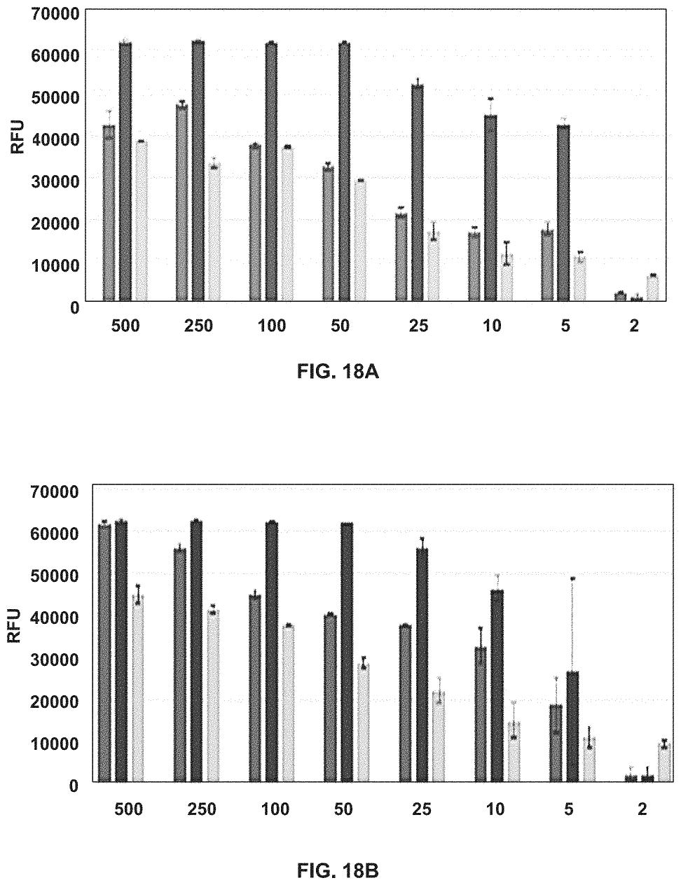

FIGS. 18A and 18B show the results of this optimization for the Asymmetric One-Step RT-PCR reaction applied to SARS-CoV-2 in 12-well microarrays for 40 PCR cycles and primer ratios of 4:1 and 8:1 respectively. Both ratios displayed a dropout at 35 and 45 cycles but performed consistently and robustly at 40 cycles. Based on these results it is concluded that an 4:1 primer ratio at 40 cycles provides the strongest signal over the range of concentrations tested. The LLoD is between 5 copies/reaction and 10 copies/reaction.

In conclusion, Asymmetric One-Step RT-PCR provides a slightly higher LLoD compared with the 2-step tandem RT-PCR (Asymmetric One-Step RT-PCR 5-10 copies/reaction=1250-625 copies/mL versus tandem RT-PCR 2 copies/reaction=125 copies/mL).

### Optimization 2

### Materials:

1. LLoD samples: Negative nasopharyngeal swab/VTM (Boca Biolistics) spiked with 25 copies/reaction (62.5 copies/ml) of purified SARS-CoV2 RNA (ATCC, NR-52285).
2. Clinical samples Positive and negative nasopharyngeal swab samples (Boca Biolistics).
3. DETECTX-RV kit.
4. 12-well glass slides—99030002 print series.
5. Labeling primer

Using the same sample as used for the analysis shown in Example 11 and Table 13, a formal LLoD was obtained for the Asymmetric One-Step RT-PCR (Tables 15 and 16), which was determined to be relatively superior to that discussed in the previous section (Example 12, ‘Optimization 1’). The data in Tables 15 and 16 are identical within experimental accuracy to that observed using the 2-Step (RT-PCR+PCR) reaction.

Furthermore, data obtained for clinical sensitivity and specificity analysis using 30 Positive and 30 negative nasopharyngeal swab samples (Table 17) showed unaltered specificity and negative predictive value (NPV), but a preliminary reduction in sensitivity from 100% to 79% due to a general reduction in hybridization signal strength.

The reduction in signal strength was remedied by the following modifications:
i) increasing concentration of input RNA template;
ii) increasing primer concentration;
iii) employing RNA samples analyzed within 48 hours of extraction

### Optimization 3

### Materials:

1. Clinical samples—Positive nasopharyngeal swab samples (Boca Biolistics).
2. DETECTX-RV kit.
3. 384-well test print—9980001001
4. Labeling primers

Performance of the 384-well DETECTX-RV microarray was performed against a set of 30 positive nasopharyngeal swab samples. This analysis differed from the previous example (Example 12, ‘Optimization 2’) in that, RNA was freshly extracted and used immediately without freeze/thawing or storage, the primer concentration is increased 2-fold to 400 nM. Samples were evaluated based on average and standard deviation signal intensity, sensitivity, specificity, positive predictive value (PPV) and negative predictive value (NPV).

TABLE 15
Lowest limit of detection analysis for Asymmetric One-Step RT-PCR

| | | Standard | (a) True | (b) False | (c) False | (d) True | | LoD (copies/ | Sensi- | Specifi- | | |
|---|---|---|---|---|---|---|---|---|---|---|---|---|
| Probe Description | Average | Deviation | Positives | Positives | Negatives | Negatives | LoB | reaction) | tivity | city | PPV | NPV |
| 62-Negcont-B | 2155 | 370 | 30 | N/A | 0 | N/A | N/A | 25 | 100 | N/A | N/A | N/A |
| SARS.C0V2-N1-pub | 32556 | 9721 | 30 | N/A | 0 | N/A | N/A | 25 | 100 | N/A | N/A | N/A |
| SARS.COV2-N2-pub | 48152 | 11944 | 30 | N/A | 1 | N/A | N/A | 25 | 97 | N/A | N/A | N/A |
| SARS.COV2-N3-pub | 30106 | 8777 | 30 | N/A | 0 | N/A | N/A | 25 | 100 | N/A | N/A | N/A |
| SARS.COV2-N1-RE1.1 | 14887 | 7673 | 30 | N/A | 0 | N/A | N/A | 25 | 100 | N/A | N/A | N/A |
| SARS.COV2-N2-RE1.3 | 38222 | 12691 | 30 | N/A | 1 | N/A | N/A | 25 | 97 | N/A | N/A | N/A |
| SARS.COV2-N2-RE1.4 | 52709 | 11996 | 30 | N/A | 0 | N/A | N/A | 25 | 100 | N/A | N/A | N/A |
| SARS.COV2-N1-RE1.1 | 14290 | 7419 | 30 | N/A | 1 | N/A | N/A | 25 | 97 | N/A | N/A | N/A |
| RNAse.P.Probe-pub1.1 | 6224 | 6480 | 30 | N/A | 0 | N/A | N/A | 25 | 100 | N/A | N/A | N/A |

TABLE 16
Lowest limit of detection analysis for Asymmetric One-Step RT-PCR

| | | Standard | (a) True | (b) False | (c) False | (d) True | | LoD (copies/ | Sensi- | Specifi- | | |
|---|---|---|---|---|---|---|---|---|---|---|---|---|
| Probe Description | Average | Deviation | Positives | Positives | Negatives | Negatives | LoB | reaction) | tivity | city | PPV | NPV |
| 62-Negcont-B | 2040 | 243 | 30 | N/A | 0 | N/A | N/A | 25 | 100 | N/A | N/A | N/A |
| SARS.COV2-N1-pub | 42768 | 8284 | 30 | N/A | 0 | N/A | N/A | 25 | 100 | N/A | N/A | N/A |
| SARS.COV2-N2-pub | 38869 | 10563 | 30 | N/A | 1 | N/A | N/A | 25 | 97 | N/A | N/A | N/A |
| SARS.COV2-N3-pub | 38788 | 6433 | 30 | N/A | 0 | N/A | N/A | 25 | 100 | N/A | N/A | N/A |
| SARS.COV2-N1-RE1.1 | 27733 | 10172 | 30 | N/A | 1 | N/A | N/A | 25 | 97 | N/A | N/A | N/A |
| SARS.COV2-N2-RE1.3 | 30135 | 12727 | 30 | N/A | 1 | N/A | N/A | 25 | 97 | N/A | N/A | N/A |
| SARS.COV2-N2-RE1.4 | 46156 | 11074 | 30 | N/A | 1 | N/A | N/A | 25 | 97 | N/A | N/A | N/A |
| SARS.COV2-N1-RE1.1 | 14349 | 4334 | 30 | N/A | 1 | N/A | N/A | 25 | 97 | N/A | N/A | N/A |
| RNAse.P.Probe-pub1.1 | 3684 | 3395 | 30 | N/A | 0 | N/A | N/A | 25 | 100 | N/A | N/A | N/A |

TABLE 17
Clinical sensitivity and specificity analysis for
Asymmetric One-Step RT-PCR

| | | Limit of | | | | |
|---|---|---|---|---|---|---|
| | Limit of blank | detection | | | | |
| | (LoB ) | (LoD) | Sensitivity | Specificity | PPV | NPV |
| 62-Negcont-B | 2455 | N/A | 100 | 100 | 100 | 100 |
| SARS.COV2-N1-pub | 2372 | N/A | 79 | 100 | 100 | 79 |
| SARS.COV2-N2-pub | 2835 | N/A | 79 | 100 | 100 | 79 |
| SARS.COV2-N3-pub | 2293 | N/A | 79 | 100 | 100 | 79 |
| SARS.COV2-N1-RE1.1 | 2184 | N/A | 79 | 100 | 100 | 79 |
| SARS.COV2-N2-RE1.3 | 2102 | N/A | 79 | 97 | 97 | 79 |
| SARS.COV2-N2-RE1.4 | 4941 | N/A | 79 | 100 | 100 | 79 |
| SARS.COV2-N3-RE1.1 | 605 | N/A | 79 | 100 | 100 | 79 |
| RNAse.P.Probe-pub1.1 | 40038 | N/A | 100 | 100 | 100 | 100 |

### Results:

An improved performance was observed with clinical isolates (Table 18) over the previous optimization described above (‘Optimization 2’). An improvement in clinical sensitivity was observed for all probes (range of clinical sensitivity, 88%-100%). The overall AUGURY readouts however report a specificity of 100% since AUGURY aggregates hybridization data from all three independent loci tests.

### Optimization 4

### Materials:

1. LLoD samples: Freshly collected positive and negative nasopharyngeal swab samples (TriCore) spiked with 25 copies/400 μl reaction (62.5 copies/ml) of purified SARS-CoV2 RNA.
2. Clinical samples: Freshly collected positive and negative nasopharyngeal swab samples (TriCore).
3. DETECTX-RV kit.
4. 96-well glass microarray print series—9903003 plates
5. Labeling primer

Thirty positive and 30 negative samples were used for LLoD analysis. Freshly prepared and negative NP-VTM samples from TriCore (New Mexico) samples doped with purified. SARS-CoV-2 RNA standard were used. All 60 NP-VTM samples were analyzed using the 4:1 asymmetric PCR primer ratio (at 2× higher concentration). in the Promega AccessQuick RT-PCR system. Each hybridization probe was analyzed individually to yield average and standard deviation of signal intensity (RFU), sensitivity, specificity, PPV and NPV. Tables 18 and 19 summarizes the results of the LLoD and the Clinical Sensitivity/Specificity analysis respectively.

The LLoD analysis (Tables 19 and 20) was found to be identical within experimental accuracy to the Asymmetric One-Step RT-PCR optimization obtain earlier ((‘Optimization 3’) as well as the 2-step RT-PCR reaction discussed above. Thus, these data confirm what was previously seen in 12 well slides, namely that the LLoD obtained with the Asymmetric One-Step RT-PCR protocol is identical, within experimental accuracy to that obtained via the Asymmetric, Tandem 2-Step RT-PCR reaction profile and is not affected by transition from 12 well to 96-well processing.

Analysis of the full set of 30+ and 30− TriCore samples, each previously analyzed via an industry standard Roche predicate q-rt-PCR assay (Tables 21 and 22) yielded clinical sensitivity of 100% for both the Asymmetric One-Step RT-PCR and Asymmetric Two Step RT-PCR methods, for the full set of 30 positive and 30 negative samples.

TABLE 18
Analysis of clinical nasopharyngeal swab samples on a DETECTX-RV microarray using Asymmetric One-Step RT-PCR

| | | | | | | | | | | 7 | 8 | | | |
|---|---|---|---|---|---|---|---|---|---|---|---|---|---|---|
| | Position | * 384 | * 384 | 1 | 2 | 3 | 4 | 5 | 6 | influenza | influenza | 9 | 10 | No |
| | on R&D | array | array | CoV2 IDT | SARS IDT | MERS IDT | CoV2 gRNA | (D)614gene | 614(G)gene | A gene | B gene | influenza | influenza | template |
| Probe name | array | (current) | (next) | plasmid | plasmid | plasmid | (D) | fragment | fragment | fragment | fragment | A gRNA | B gRNA | control |
| Negative Control probe | 1 | * | * | 1421 | 1254 | 921 | 886 | 1726 | 2312 | 1259 | 1009 | 1325 | 1206 | 2360 |
| SARS.COV2-N1-RE1.1 | 2 | * | * | 36815 | 1231 | 95 | 54332 | 225 | 644 | 169 | 60 | 295 | 413 | 758 |
| SARS.COV2-N2-RE1.3 | 3 | * | * | 38270 | 1198 | 1448 | 47713 | 939 | 1517 | 907 | 955 | 1139 | 1410 | 1729 |
| SARS.COV2-N2-RE1.4 | 4 | * | * | 62149 | 2410 | 2017 | 61697 | 1875 | 2013 | 2580 | 2423 | 2276 | 1904 | 3571 |
| SARS.COV2-N3-RE1.1 | 5 | * | * | 60538 | 48906 | 3721 | 61693 | 3523 | 3867 | 3254 | 3661 | 4126 | 3819 | 1814 |
| RNAse.P.Probe | 6 | * | * | 2080 | 3282 | 2407 | 2139 | 1452 | 2023 | 2185 | 2803 | 1409 | 2434 | 1432 |
| InfA.7.univ-pubRev | 7 | * | * | −62 | −5 | 138 | 5 | −70 | 670 | 47983 | 362 | 36700 | 772 | −170 |
| InfB.8.univ-pub | 8 | * | * | −286 | 5 | −11 | −333 | −191 | 25 | 543 | 62289 | 745 | 57508 | −194 |
| Universal D + G sense probe (1.1) | 13 | | * | 779 | 647 | 377 | 41182 | 61839 | 62125 | 575 | 629 | 561 | 456 | 1288 |
| 614D sense probe (1.4) | 16 | | * | 1184 | 636 | 853 | 36175 | 61651 | 10531 | 680 | 622 | 430 | 728 | 1188 |
| 614D sense probe (1.1) | 11 | * | | 552 | 176 | 195 | 13969 | 45654 | 1378 | 613 | 518 | 1155 | 2019 | 849 |
| 614D sense probe (1.2) | 14 | | | 1298 | 342 | 285 | 6149 | 37398 | 2280 | 245 | −2 | −296 | −36 | 695 |
| 614D sense probe (1.3) | 15 | | | 840 | 630 | 712 | 4435 | 32101 | 1380 | 804 | 679 | 1264 | 516 | 1186 |
| 614G sense probe (1.4) | 19 | | * | 789 | 736 | 822 | 1346 | 11304 | 59019 | 916 | 1048 | 587 | 1466 | 1317 |
| 614G sense probe (1.1) | 17 | | | 1135 | 903 | 955 | 1736 | 7397 | 47967 | 968 | 1257 | 1447 | 984 | 1171 |
| 614G sense probe (1.2) | 12 | * | | 1002 | 117 | 139 | 240 | 3048 | 36606 | 661 | 538 | 2008 | 480 | 393 |
| 614G sense probe (1.3) | 18 | | | 1631 | 1500 | 1487 | 2268 | 4681 | 17963 | 1745 | 1856 | 1142 | 1350 | 2036 |
| Universal D + G antisense probe (1.1) | 36 | | | 246 | 1538 | 1338 | 2529 | 1886 | 2072 | 323 | 302 | 1237 | 869 | 186 |
| 614D antisense probe (1.4) | 20 | | | 506 | 241 | 477 | 1367 | 274 | 585 | 355 | 682 | 479 | 2132 | 977 |
| 614D antisense probe (1.1) | 9 | * | | 752 | 541 | 496 | 696 | 280 | 278 | 743 | 529 | 746 | 1828 | 913 |
| 614D antisense probe (1.2) | 37 | | | 706 | 726 | 964 | 633 | 932 | 1156 | 916 | 299 | 221 | 326 | 1254 |
| 614D antisense probe (1.3) | 38 | | | 1370 | 1257 | 1177 | 1241 | 1367 | 2066 | 1274 | 1053 | 659 | 750 | 2263 |
| 614G antisense probe (1.4) | 21 | | | 1169 | 1131 | 821 | 691 | 635 | 976 | 1051 | 606 | 1230 | 345 | 1235 |
| 614G antisense probe (1.1) | 10 | * | | 1264 | 1514 | 1472 | 1256 | 911 | 1359 | 1288 | 954 | 1429 | 1025 | 1527 |
| 614G antisense probe (1.2) | 39 | | | 1330 | 1162 | 1387 | 1425 | 1231 | 1738 | 1353 | 1043 | 889 | 826 | 2180 |
| 614G antisense probe (1.3) | 40 | | | 2309 | 2843 | 3013 | 2680 | 2615 | 2804 | 3015 | 2427 | 9517 | 4717 | 5021 |
| SARS.CoV2-N2-RE1.5 | | | | 39160 | 548 | 1996 | 48636 | 89 | 432 | 1006 | 865 | 2369 | 2645 | 1039 |
| SARS.CoV2-N2-RE1.6 | | | | 62149 | 2156 | 4407 | 61697 | 2610 | 3024 | 2530 | 2862 | 2456 | 2631 | 2057 |
| SARS.CoV1-N2-RE1.5 | | | | 433 | 45995 | 161 | 782 | −56 | 121 | 386 | 372 | 2592 | 706 | 116 |
| SARS.CoV1-N2-RE1.6 | | | | 114 | 48560 | 95 | 79 | 805 | 2108 | 479 | −170 | 338 | 266 | 751 |
| hCoV19/PANG1-N2-RE1.2 | | | | 9921 | 379 | 628 | 13736 | 360 | 872 | 402 | −162 | 5 | 164 | 1236 |
| hCoV19/PANG1-N2-RE1.4 | | | | 689 | 795 | 687 | 1268 | 1104 | 479 | 677 | 450 | 771 | 391 | 1242 |
| hCoV19/PANG2-N2-RE1.2 | | | | 2729 | 869 | 934 | 3829 | 918 | 1525 | 985 | 890 | 467 | 685 | 1928 |
| hCoV19/PANG2-N2-RE1.4 | | | | 2167 | 16061 | 2824 | 4880 | 2485 | 4792 | 2232 | 9856 | 2885 | 2613 | 3893 |
| BAT2.CoV-N2-RE1.2 | | | | 4368 | 1955 | 2137 | 7769 | 1697 | 2115 | 2164 | 1938 | 2779 | 2343 | 2614 |
| BAT2.CoV-N2-RE1.4 | | | | 704 | 825 | 570 | 994 | 238 | 730 | 129 | 323 | 274 | 803 | 864 |
| SARS-rel.CoV-N2-RE1.2 | | | | 695 | 463 | 703 | 414 | 644 | 762 | 818 | 504 | 604 | 1121 | 1011 |
| SARS-rel.CoV-N2-RE1.4 | | | | 1271 | 386 | 640 | 2313 | 406 | 538 | 794 | 589 | 878 | 1588 | 623 |
| hCoV19/BATYUN-N2-RE1.2 | | | | 2243 | 1161 | 1729 | 3706 | 1131 | 1202 | 1353 | 1294 | 1973 | 1564 | 1447 |
| hCoV19/BATYUN-N2-RE1.5 | | | | 1697 | 946 | 1407 | 1540 | 718 | 1069 | 847 | 669 | 1643 | 1255 | 1131 |
| RNAse.P.Probe-pub1.3 | | | | 62149 | 61822 | 61921 | 61697 | 61839 | 62125 | 62099 | 62388 | 61165 | 61732 | 62625 |
| RNAse.P.Probe-RE1.4 | | | | 62070 | 61822 | 61921 | 61578 | 61837 | 62125 | 62011 | 62361 | 61120 | 61602 | 62656 |
| RNAse.P.Probe-pub2.1 | | | | 1204 | 2380 | 3812 | 1626 | 1394 | 1551 | 1304 | 1726 | 5618 | 6899 | 903 |
| RNAse.P.Probe-RE2.2 | | | | −261 | −620 | −614 | −676 | 337 | −164 | −703 | −161 | −1094 | −530 | −41 |
| RdRP_Ber_P2_CoV2 | | | | −123 | −287 | −131 | −68 | −48 | −106 | −212 | −227 | −811 | −101 | −42 |
| RdRP_P2_CoV2_RE 1.1 | | | | −1134 | −1223 | −1059 | −1130 | −1183 | −1077 | −1126 | −964 | −1842 | −1067 | −1008 |
| RdRP_P2_PAN_RE 1.1 | | | | 1151 | 729 | 1259 | 1334 | 876 | 1145 | 1259 | 971 | 686 | 1446 | 862 |
| E_Sarb_Pan_RE1.2 | | | | 2817 | 5010 | 3941 | 3161 | 3546 | 3840 | 3165 | 2743 | 7209 | 3221 | 4507 |
| B2M_RE1.1 | | | | 746 | 758 | 1017 | 946 | 714 | 1707 | 1080 | 1442 | 776 | 1159 | 974 |
| B2M_RE2.1 | | | | 302 | −144 | −27 | −5 | −84 | −13 | 112 | 480 | −135 | 175 | 162 |

TABLE 19
Analysis of clinical nasopharyngeal swab samples on a DETECTX-RV microarray using Asymmetric One-Step RT-PCR

| | | | | | | | | | | | | | Average |
|---|---|---|---|---|---|---|---|---|---|---|---|---|---|
| | | | | | | | | | | | | | signals |
| | | | | | | | | | | | | | from |
| | | | CoV2 | | | | PS2 | PS5 | CoV2gRNA | Neg1 | Neg4 | NTC | clinical |
| Clinical Samples | PS2 | PS5 | gRNA | Neg1 | Neg4 | NTC | (infA, B) | (infA, B) | (infA, B) | (infA, B) | (infA, B) | (infA, B) | sample |
| 62-Negcont-B | 1601 | 962 | 1028 | 2101 | 1603 | 1110 | 1825 | 1853 | 938 | 1458 | 1058 | 1245 | 1640 |
| SARS.COV2-N1- | 31578 | 29980 | 61449 | −5 | −259 | 1407 | 12105 | 14927 | 61362 | −11 | −50 | 170 | 32850 |
| RE1.1 | | | | | | | | | | | | | |
| SARS.COV2-N2- | 17229 | 16374 | 49596 | 960 | 661 | 2050 | 12280 | 11392 | 50163 | 628 | 871 | 1994 | 38570 |
| RE1.3 | | | | | | | | | | | | | |
| SARS.COV2-N2- | 47605 | 46614 | 61603 | 1922 | 2346 | 2893 | 38540 | 39854 | 61479 | 1574 | 2286 | 3895 | 43670 |
| RE1.4 | | | | | | | | | | | | | |
| SARS.COV2-N3- | 60036 | 49602 | 61602 | 4327 | 4941 | 2141 | 43957 | 42055 | 61498 | 3921 | 3351 | 4090 | 59723 |
| RE1.1 | | | | | | | | | | | | | |
| RNAse.P.Probe- | 3307 | 3890 | 1702 | 4561 | 4585 | 2454 | 2565 | 2643 | 2451 | 2985 | 3082 | 3162 | |
| pub1.1 | | | | | | | | | | | | | |
| InfA.7.univ- | 422 | 1257 | 369 | 443 | 2033 | 552 | 39579 | 40285 | 20903 | 38128 | 22691 | 42846 | |
| pubRev | | | | | | | | | | | | | |
| InfB.8.univ-pub | 102 | 953 | 112 | 34 | 1437 | 414 | 45076 | 39136 | 38437 | 39559 | 35394 | 40884 | |
| 614U-SE-S1- | 14908 | 13172 | 41545 | 335 | 425 | 461 | 6546 | 6309 | 38526 | 316 | 229 | 321 | |
| RE1.1 * | | | | | | | | | | | | | |
| 614D-SE-S1- | 833 | 785 | 37215 | 336 | 489 | 410 | 833 | 547 | 34632 | 361 | 922 | 1103 | |
| RE1.4 ¶ | | | | | | | | | | | | | |
| 614G-SE-S1- | 5728 | 4792 | 1762 | 920 | 803 | 559 | 2815 | 3218 | 1584 | 493 | 1104 | 835 | |
| RE1.4 § | | | | | | | | | | | | | |
| SARS.CoV2-N2- | 20132 | 17893 | 54453 | 370 | 797 | 637 | 13826 | 14895 | 53076 | 429 | 2129 | 1494 | |
| RE1.5 | | | | | | | | | | | | | |
| SARS.CoV2-N2- | 44125 | 44266 | 61603 | 1466 | 2298 | 3423 | 38153 | 39052 | 61498 | 1748 | 2830 | 2944 | |
| RE1.6 | | | | | | | | | | | | | |
| SARS.CoV1-N2- | 656 | 888 | 820 | 1026 | 1634 | 564 | 1224 | 7027 | 936 | 1730 | 2996 | 1261 | |
| RE1.5 | | | | | | | | | | | | | |
| SARS.CoV1-N2- | 821 | 1239 | 35 | 948 | 1678 | 655 | 1106 | 1400 | −151 | 1120 | 1936 | 469 | |
| RE1.6 | | | | | | | | | | | | | |

* Spike = D + G, ¶ D Variant = Wuhan like, § G Variant = European like

TABLE 20
Limit of detection analysis for Asymmetric One-Step RT-PCR using 12-well array format and a 4:1 primer mix.

| | Aver- | Standard | (a) True | (b) False | (c) False | (d) True | | LoD copies/ | Sensi- | Specifi- | | |
|---|---|---|---|---|---|---|---|---|---|---|---|---|
| Probe Description | age | Deviation | Positives | Positives | Negatives | Negatives | LoB | reaction | tivity | city | PPV | NPV |
| 62-Negcont-B | 2155 | 370 | 30 | N/A | 0 | N/A | N/A | 25 | 100.0 | N/A | N/A | N/A |
| SARS.COV2-N1-pub | 32556 | 9721 | 30 | N/A | 0 | N/A | N/A | 25 | 100.0 | N/A | N/A | N/A |
| SARS.COV2-N2-pub | 48152 | 11944 | 30 | N/A | 1 | N/A | N/A | 25 | 96.8 | N/A | N/A | N/A |
| SARS.COV2-N3-pub | 30106 | 8777 | 30 | N/A | 0 | N/A | N/A | 25 | 100.0 | N/A | N/A | N/A |
| SARS.COV2-N1-RE1.1 | 14887 | 7673 | 30 | N/A | 0 | N/A | N/A | 25 | 100.0 | N/A | N/A | N/A |
| SARS.COV2-N2-RE1.3 | 38222 | 12691 | 30 | N/A | 1 | N/A | N/A | 25 | 96.8 | N/A | N/A | N/A |
| SARS.COV2-N2-RE1.4 | 52709 | 11996 | 30 | N/A | 0 | N/A | N/A | 25 | 100.0 | N/A | N/A | N/A |
| SARS.COV2-N1-RE1.1 | 14290 | 7419 | 30 | N/A | 1 | N/A | N/A | 25 | 96.8 | N/A | N/A | N/A |
| RNAse.P.Probe-pub1.1 | 6224 | 6480 | 30 | N/A | 0 | N/A | N/A | 25 | 100.0 | N/A | N/A | N/A |

TABLE 21
Clinical Sensitivity and Specificity Analysis for Asymmetric One-Step RT-PCR

| | | Standard | Average | Standard | | (b) | (c) False | (d) True | | | | Speci- | | |
|---|---|---|---|---|---|---|---|---|---|---|---|---|---|---|
| Probe | Average | Deviation | Nega- | Deviation | (a) True | False | Nega- | Nega- | | | Sensi- | fi- | | |
| Description | Positives | Positives | tives | Negatives | Positive | Positive | tives | tives | LoB | LoD | tivity | city | PPV | NPV |
| 62-Negcont-B | 1581 | 1477 | 1195 | 620 | 30 | 0 | 0 | 30 | N/A | N/A | 100 | 100 | 100 | 100 |
| SARS.COV2- | 49470 | 15630 | 2111 | 5626 | 30 | 2 | 1 | 30 | N/A | N/A | 97 | 94 | 94 | 97 |
| N1-RE1.1 | | | | | | | | | | | | | | |
| SARS.COV2- | 50383 | 16691 | 2306 | 4298 | 30 | 2 | 2 | 30 | N/A | N/A | 94 | 94 | 94 | 94 |
| N2-RE1.3 | | | | | | | | | | | | | | |
| SARS.COV2- | 55425 | 11305 | 18258 | 9984 | 30 | 0 | 0 | 30 | N/A | N/A | 100 | 100 | 100 | 100 |
| N2-RE1.4 | | | | | | | | | | | | | | |
| SARS.COV2- | 53144 | 14343 | 4403 | 8808 | 30 | 2 | 0 | 30 | N/A | N/A | 100 | 94 | 94 | 100 |
| N3-RE1.1 | | | | | | | | | | | | | | |
| RNAse.P.Probe- | 6009 | 7451 | 18237 | 13677 | 30 | 0 | 0 | 30 | N/A | N/A | 100 | 100 | 100 | 100 |
| pub1.1 | | | | | | | | | | | | | | |
| Overall Results | N/A | N/A | N/A | N/A | 30 | 2 | 0 | 30 | N/A | N/A | 100 | 94 | 94 | 100 |

TABLE 22
Clinical Sensitivity and Specificity Analysis for Standard DETECTX-RV (RT-PCR + Labeling PCR)

| | | Standard | Average | Standard | | (b) | (c) False | (d) True | | | | Speci- | | |
|---|---|---|---|---|---|---|---|---|---|---|---|---|---|---|
| Probe | Average | Deviation | Nega- | Deviation | (a) True | False | Nega- | Nega- | | | Sensi- | fi- | | |
| Description | Positives | Positives | tives | Negatives | Positive | Positive | tives | tives | LoB | LoD | tivity | city | PPV | NPV |
| 62-Negcont-B | 1626 | 426 | 2196 | 1928 | 30 | 0 | 0 | 30 | N/A | N/A | 100 | 100 | 100 | 100 |
| SARS.COV2- | 57318 | 5808 | 4316 | 15424 | 30 | 3 | 0 | 30 | N/A | N/A | 100 | 91 | 91 | 100 |
| N1-RE1.1 | | | | | | | | | | | | | | |
| SARS.COV2- | 58079 | 3173 | 7929 | 15774 | 30 | 4 | 0 | 30 | N/A | N/A | 100 | 88 | 88 | 100 |
| N2-RE1.3 | | | | | | | | | | | | | | |
| SARS.COV2- | 59119 | 469 | 10853 | 20248 | 30 | 5 | 0 | 30 | N/A | N/A | 100 | 86 | 86 | 100 |
| N2-RE1.4 | | | | | | | | | | | | | | |
| SARS.COV2- | 59354 | 473 | 12372 | 23104 | 30 | 6 | 0 | 30 | N/A | N/A | 100 | 83 | 83 | 100 |
| N3-RE1.1 | | | | | | | | | | | | | | |
| RNAse.P.Probe- | 59103 | 461 | 60174 | 698 | 30 | 0 | 0 | 30 | N/A | N/A | 100 | 100 | 100 | 100 |
| pub1.1 | | | | | | | | | | | | | | |
| Overall Results | N/A | N/A | N/A | N/A | 30 | 4 | 0 | 30 | N/A | N/A | 100 | 88 | 88 | 100 |

Clinical specificity at the local probe level on the other hand were not 100%, being about 94% for the Asymmetric One-Step RT-PCR method and 100% for the 2-Step RT-PCR method. Thus, the 2-Step method detected 3 false positives and the Asymmetric One-Step RT-PCR detected 2 false positives in the same set of 30 TriCore Negatives. This discordance was resolved by third party sequencing and it was shown that the positives detected by microarray analysis did in fact contain SARS-CoV-2, which had not been detected by the predicate Q-RTPCR assay.

A detailed analysis of individual probe Clinical Sensitivity revealed that 2 probes ([N1, 1.1], [N2, 1.3]) have generated 1 and 2 false negatives respectively. These 3 rare events did not affect the Overall AUGURY sensitivity, which remained at 100% because of its use of multiple probes to make a call.

### Conclusions

Asymmetric One-Step RT-PCR performance with Clinical Isolates in the 96-well format has improved significantly over prior optimizations due to use of RNA extracted from fresh samples and 2× increase of Primer Concentration 2×. Sensitivity among all probes tested has yielded an increase in Average N1,N2 and N3 probe Clinical Sensitivity to about 97% (range=94%-100%). However, the aggregated AUGURY readouts obtained provide a 100% Sensitivity, since Augury aggregates hybridization data from all (3) independent loci tests.

### Optimization 5

### Optimized Clinical Validation of Mini-RV Panel:

### Materials and Methods:

1. The 12-well Mini-RV Array (R&D Format) was deployed.
2. Testing was performed on fresh clinical isolates (Boca Biolistics) via the Asymmetric One-Step RT-PCR reaction for the entire set of targets (S,N1,N2,N3, P, PanA, PanB) but with a more efficient set of PCR primers for RNAse P.
3. Influenza A and Influenza B was tested by use of purified Influenza A or Influenza B gRNA (ATCC reference standards) added to positive or negative clinical isolates for detection using “PanA” and “PanB” probes on the array.
4. Optimized Asymmetric, One-Step RT-PCR was deployed and standard Hybridization/Wash Conditions.
5. Results: Using a small number of clinical isolates (2 positives, 2 negatives) the full panel of probes described in Table 12 (S, N1, N2, N3, PanA, PanB) were used to analyze the product of the Asymmetric, One-Step RT-PCR reaction. To offset low RNase P signals seen in the previous optimization (Tables 18-22), an alternative, higher efficiency RNAse P primer pair was used (SEQ ID: 43 and SEQ ID: 44). Additionally, the RNase-P primer concentration was increased 2× Table 23 shows a summary of the data for the 12-well format.

TABLE 23
Asymmetric One-Step RT-PCR of Full Mini-RV Array (S, N1, N2, N3, P, PanA, PanB) for
Clinical Isolates on 12-well slides using higher efficiency RNAse P primers
(SEQ ID: 43 and SEQ ID: 44) as internal control.

| | | | PS-1 | PS-1 | CoV2 | | | Neg-1 | Neg-2 | |
|---|---|---|---|---|---|---|---|---|---|---|
| | PS-1 | PS-2 | (InfA) | (InfB) | gRNA | Neg-1 | Neg-2 | (InfA) | (InfB) | NTC |
| 62-Negcont-B | 895 | 1332 | 826 | 1241 | 777 | 1658 | 1763 | 1441 | 1231 | 1368 |
| SARS.COV2-N1-RE1.1 | 61824 | 48746 | 51658 | 61633 | 36639 | 751 | 1164 | 224 | 166 | 3336 |
| SARS.COV2-N2-RE1.3 | 50873 | 38694 | 39954 | 48652 | 17141 | 598 | 1102 | 566 | 873 | 589 |
| SARS.COV2-N2-RE1.4 | 61857 | 62211 | 62104 | 61720 | 37147 | 883 | 738 | 879 | 1068 | 1012 |
| SARS.COV2-N3-RE1.1 | 61807 | 56525 | 61989 | 61689 | 54650 | 2668 | 1979 | 2941 | 3422 | 2596 |
| RNAse.P.Probe-pub1.1 | 53845 | 39257 | 38696 | 44316 | 2148 | 52419 | 40222 | 44258 | 50423 | 1471 |
| InfA.7.univ-pubRev | 132 | −176 | 34385 | 32 | −38 | 481 | 64 | 49966 | 997 | 1 |
| InfB.8.univ-pub | −171 | −184 | 2231 | −290 | −403 | −78 | −312 | 209 | 13251 | −42 |
| 614U-SE-S1-RE1.1 | 61838 | 43200 | 48452 | 58826 | 13240 | 729 | 582 | 335 | 350 | 714 |
| 614D-SE-S1-RE1.4 | 13956 | 4858 | 7082 | 12120 | 9885 | 33 | 79 | 809 | 306 | 209 |
| 614G-SE-S1-RE1.4 | 49820 | 35581 | 39167 | 44014 | 1467 | 734 | 653 | 752 | 549 | 568 |

### Asymmetric One-Step RT-PCR Performance Optimization

Freshly collected NP/VTM samples (TriCore) matched with a complete set of Roche Cobas 6800 Q-RT-PCR Ct thresholds were used. Analysis was performed on 384-well plates using 30 positive and 30 negative clinical isolates in an RNAse P modified multiplex PCR reaction. The data obtained (Table 23) was in good agreement with that obtained for the 96-well format using previous RNAse P primers (Tables 18-22) and further provided a better RNAse P signal than previously observed (Tables 18-22).

### Gel Analysis and Sequencing

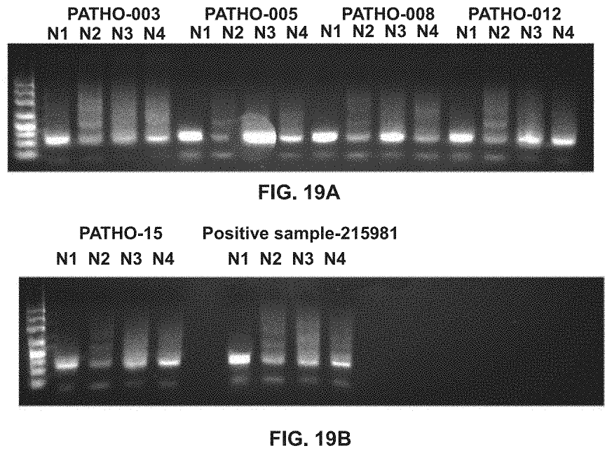

Four sequencing primers with M13 tags were created and the amplicons generated using the Asymmetric One-Step RT-PCR method (Asymmetric One-Step RT-PCR) were analyzed by gel electrophoresis. FIGS. 19A and 19B show that discordant samples each produce an amplicon fragment of the correct size associated with the expected SARS-CoV2 amplification. N1, N2, N3 refer to microarray probes specific the N1, N2, and N3 sites in SARS-CoV-2 and P refers to probe specific for human RNAse P, which are used as an internal positive control.

### Sequence Analysis of Discordant Clinical Samples.

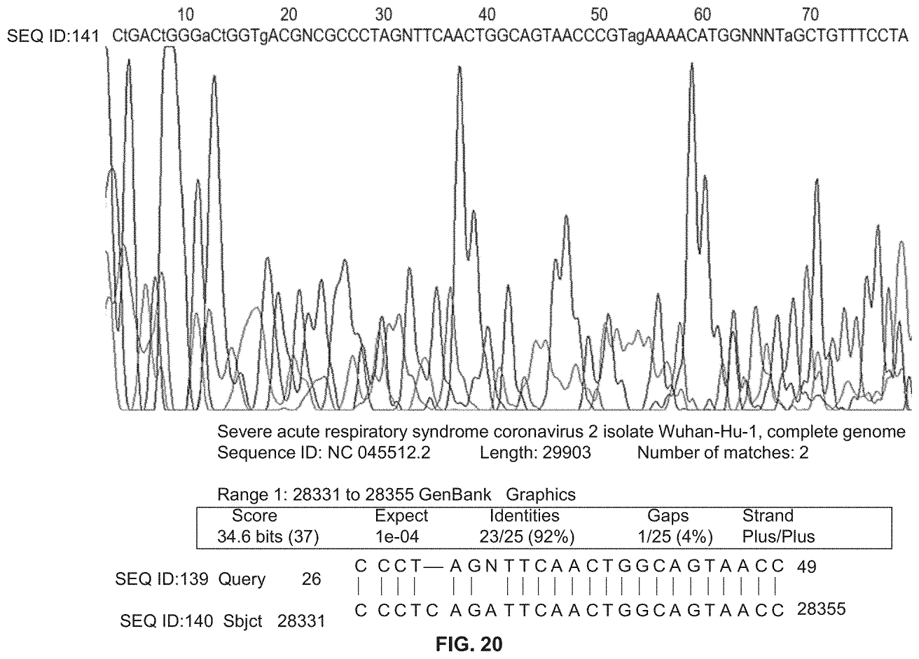

TriCore samples identified as “Negative” by Cobas but identified as “Positive” on multiple repeats of the DETECTX-RV assay were sequenced (third party sequencing, University of Arizona). The sequencing data shown in FIG. 20 for a representative PATHO-003 sample (N1-M13F) was found to agree with the gel data. This confirmed that the discordant samples each contain measurable SARs-CoV2 infection (loci N1 & N2).

### Improved Sequence Analysis of Discordant Clinical Samples.

Clinical samples (30 positive/30 negative NP/VTM, TriCore) tested using the Cobas 6800 platform were used as clinical reference samples to evaluate sensitivity and specificity of the DETECTX-RV assay using Two-Step Tandem and Asymmetric One-Step RT-PCR reaction methods. To confirm accuracy of the DETECTX-RV “Positive” readouts, Sanger sequencing was performed within the N Region, on all 6 discordant samples and one of the many “Positive” samples, which had been identified as “Positive” by both COBAS and DETECTX-RV. The results shown in Table 24 are in agreement with DETECTX-RV—all 6 discordant samples were identified by Sanger sequencing as containing measurable SARS-CoV-2 RNA. Sequence heterogeneity (N) in several of the negative samples was also observed.

TABLE 24
SARS-CoV-2 Sequencing of “Discordant” Clinical NP/VTM Samples

| | | | Discordant and | | | |
|---|---|---|---|---|---|---|
| | | | SARS-CoV-2 | | | |
| | | | Sequences | Percent | One | Two |
| Sample | SEQ ID Number | Primer Alignment | Aligned | Alignment | Step | Step |
| Discordant-1 | SEQ ID: 98 | TTCCNNCGGNAGGCCNGCCATTGGGC | Discordant-1 | 62% | + | + |
| | SEQ ID: 99 | \|\|\|* \|\|\| \|*\|*\| **\|\|\|\| \|\|\| | SARS-CoV-2 | | | |
| | | TTCTT CGGAATGTCGCGCATT GGC | | | | |
| | | -AAACNTGGACNNNNGCGTGNTTTCC | Discordant-1 | | | |
| | | \|\|\|\| \|\|\|*\| \|\| \|\| \|\|\|\|\| | SARS-CoV-2 | | | |
| | | -AAACATGGTCATA GC TG TTTCCT | | | | |
| Discordant-2 | SEQ ID: 100 | TTCTTCGGGAGGGCGNGCATTGGGCN | Discordant-2 | 84% | − | + |
| | SEQ ID: 99 | \|\|\|\|\|\|\|\|*\|*\|*\|\| \|\|\|\|\| \|\|\| | SARS-CoV-2 | | | |
| | | TTCTTCGGAATGTCGCGCATT GGCA | | | | |
| | | -AACATGGGTCATAGCTGTTTCCT | Discordant-2 | | | |
| | | \|\|\|\|\|\|\| \|\|\|\|\|\|\|\|\|\|\|\|\|\|\|\| | SARS-CoV-2 | | | |
| | | -AACATG GTCATAGCTGTTTCCT | | | | |
| Discordant-3 | SEQ ID: 101 | TTCTTCGGGAANGTCGCGGCATNGGC | Discordant-3 | 84% | − | + |
| | SEQ ID: 99 | \|\|\|\|\|\|\|\| \|\| \|\|\|\|\|\| \|\|\| \|\|\| | SARS-CoV-2 | | | |
| | | TTCTTCGG AATGTCGCG CATTGGC | | | | |
| | | -AAACATGGGGTCATAGGCTGNTTTCCT | Discordant-3 | | | |
| | | \|\|\|\|\|\|\|\| \|\|\|\|\|\|\|\| \|\|\| \|\|\|\|\|\| | SARS-CoV-2 | | | |
| | | -AAACATG GTCATAG CTG TTTCCT | | | | |
| Discordant-4 | SEQ ID: 102 | TTCTTCGGGAAGGTCGNGGCATTGGC | Discordant-4 | 76% | − | + |
| | SEQ ID: 99 | \|\|\|\|\|\|\|\| \|\| \|\|\| \| \|\|\|\|\|\|\| | SARS-CoV-2 | | | |
| | | TTCTTCGG AATGTCGCG CATTGGC | | | | |
| | | -AAACATGGGNTCATAGGCNTGATTTCCT | Discordant-4 | | | |
| | | \|\|\|\|\|\|\|\| \|\|\|\|\|\|\| \|\|\| \|\|\|\|\|\| | SARS-CoV-2 | | | |
| | | -AAACATG GTCATAG CTG TTTCCT | | | | |
| Discordant-5 | SEQ ID: 103 | TTCTTCGGGAANGTCGCGCATNGGCA | Discordant-5 | 75% | + | + |
| | SEQ ID: 99 | \|\|\|\|\|\|\|\| \|\| \|\|\|\|\|\|\|\|\| \|\|\|\| | SARS-CoV-2 | | | |
| | | TTCTTCGG AATGTCGCGCATTGGCA | | | | |
| | | -AACANGGTCATAGCTGGTTTCCT | Discordant-5 | | | |
| | | \|\|\|\|\| \|\|\|\|\|\|\|\|\|\|\| \|\|\|\|\|\| | SARS-CoV-2 | | | |
| | | -AACATGGTCATAGCTG TTTCCT | | | | |
| Discordant-6 | SEQ ID: 104 | TTCTTCGGGAAGGTCGCGGCATTGGC | Discordant-6 | 97% | − | Rerun |
| | SEQ ID: 99 | \|\|\|\|\|\|\|\| \|\| \|\|\|\|\| \|\|\|\|\|\|\|\| | SARS-CoV-2 | | | |
| | | TTCTTCGG AATGTCGC GCATTGGC | | | | |
| | | -AAACATGGATCATAGTNTGNTTTCCT | Discordant-6 | | | |
| | | \|\|\|\|\|\|\|\|\| \|\|\|\|\|\| \|\| \|\|\|\|\|\| | SARS-CoV-2 | | | |
| | | -AAACATGG TCATAG CTG TTTCCT | | | | |
| SARS-CoV-2 | SEQ ID: 105 | TTCTTCGGAATGTCGCGCATTGGCAA | Positive Control | 99% | + | + |
| Positive | SEQ ID: 99 | \|\|\|\|\|\|\|\|\|\|\|\|\|\|\|\|\|\|\|\|\|\|\|\|\|\| | SARS-CoV-2 | | | |
| Control | | TTCTTCGGAATGTCGCGCATTGGCAA | | | | |
| | | -ACATGGTCATAGCNTGTTTCCT | Positive Control | | | |
| | | \|\|\|\|\|\|\|\|\|\|\|\|\|\| \|\|\|\|\|\|\|\| | SARS-CoV-2 | | | |
| | | -ACATGGTCATAGC TGTTTCCT | | | | |

99002-M13R (N2-Reverse) Reverse Complement

### Example 13

Incorporation of Influenza Probes into the Array

Influenza A & B probes and primers were added to the 12-well array during analysis using the 2-step method. The hybridization data (Table 25) show that the influenza probes and primers may be used as such with no further refinement.

### Example 14

### Raw Sample Feasibility Testing

### Materials:

1. Mouthwash from patients diagnosed with SARS-CoV2
2. Asymmetric One-Step RT-PCR and labelling primers
3. Two-step RT-PCR
4. DETECTX-RV kit
5. Tris-HCl pH 9 with MgCl₂ at pH 3, 4, 5, 6, 8 and 10 mM

Reducing the time taken from obtaining the sample to performing the microarray analysis is expected to significantly increase the number of sample that may be processed per day, a factor that is critical during a pandemic. To establish the feasibility of bypassing the RNA isolation step in the method, experiments were performed using clinical mouthwash samples (QuikSal) from patients diagnosed with SARS-CoV2. The data in Table 26 shows the results of analysis in samples where the RNA extraction step was omitted. It was observed that raising the pH to the ordinary PCR range of pH 9.0 and adding Mg⁺² to coordinate EDTA and citrate in the QuikSal, mouthwash enabled microarray analysis in crude samples with no further RNA purification.

### Example 15

### Automation and Analysis: 96-Well and 384-Well Plates

RNA Extraction using Zymo Magnetic Beads and RNA loading onto PCR plates for RT-PCR was established using Tecan. Hybridization and Washing Automation for Asymmetric One-Step RT-PCR in 96-well format was completed for the Tecan and a first 96-well plate. It was run through with 20 positive Clinical Isolates (TriCore)+76 negative (water-only) samples. The corresponding 384 well software was also tested with clinical samples using a Tecan code modified for 384-well plate operation, capable of 384-well function with a 96-pipette head.

TABLE 25
Incorporation of Influenza Lead primers and probes in the Two-step PCR and hybridization analysis

Influenza A primer set 1
RT-PCR
Forward Primer SEQ ID: 17 TTTATGGCTAAAGACAAGACCRATCCTG
Reverse Primer SEQ ID: 18 TTTTTAAGGGCATTYTGGACAAAKCGTC
Label-PCR
Forward Primer SEQ ID: 39 TTTCAAGACCRATCCTGTCACCTCTGAC
Reverse Primer SEQ ID: 40 /5CY3/TTTAAGGGCATTYTGGACAAAKCGTCTA
Influenza A primer set 2
RT-PCR
Forward Primer SEQ ID: 17 TTTATGGCTAAAGACAAGACCRATCCTG
Reverse Primer SEQ ID: 40 TTTAAGGGCATTYTGGACAAAKCGTCTA
Label-PCR
Forward Primer SEQ ID: 39 TTTCAAGACCRATCCTGTCACCTCTGAC
Reverse Primer SEQ ID: 81 /5CY3/TTTGGGCATTYTGGACAAAKCGTCTACG
Influenza B primer set 1
RT-PCR
Forward Primer SEQ ID: 19 TTTGGATGAAGAAGATGGCCATCGGATC
Reverse Primer SEQ ID: 20 TTTTCTAATTGTCTCCCTCTTCTGGTGA
Label-PCR
Forward Primer SEQ ID: 82 TTTGGATCCTCAACTCACTCTTCGAGCG
Reverse Primer SEQ ID: 42 /5CY3/TTTTAATCGGTGCTCTTGACCAAATTGG
Influenza B primer set 2
RT-PCR
Forward Primer SEQ ID: 82 TTTGGATCCTCAACTCACTCTTCGAGCG
Reverse Primer SEQ ID: 106 TTTTCCCTCTTCTGGTGATAATCGGTGC
Label-PCR
Forward Primer SEQ ID: 41 TTTGCGTCTCAATGAAGGACATTCAAAG
Reverse Primer SEQ ID: 42 /5CY3/TTTTAATCGGTGCTCTTGACCAAATTGG

| | | Influ- | Influ- | Influ- | | | | | |
|---|---|---|---|---|---|---|---|---|---|
| | | enza | enza | enza | Influenza | Influenza | Influenza | Influenza | Influenza |
| | | A | A | A | A | B | B | B | B |
| | | primer | primer | primer | primer | primer | primer | primer | primer |
| | | set 1 | set 2 | set 1 | set 2 | set 1 | set 2 | set 1 | set 2 |
| | | infA | | No template | | infB | | No template | |
| | Probe Name | Target | | control | | Target | | control | |
| # | (specificity) | Well 1 | Well 2 | Well 5 | Well 6 | Well 9 | Well 10 | Well 11 | Well 12 |
| 1 | 62-Negcont-B | 1807 | 1714 | 1727 | 1429 | 1679 | 1359 | 1406 | 1211 |
| 2 | SARS.COV2-N1-pub | 636 | 693 | 889 | 455 | 534 | 530 | 588 | 493 |
| 3 | SARS.COV2-N1-RE1.1 | 730 | 750 | 633 | 630 | 859 | 657 | 641 | 640 |
| 4 | SARS.COV2-N1-RE1.2 | 305 | 185 | 168 | 29 | 160 | 93 | 128 | 103 |
| 5 | SARS.COV2-N1-RE1.3 | 161 | 290 | 355 | −6 | 146 | 60 | 179 | 135 |
| 6 | SARS.COV2-N2-pub | 894 | 968 | 976 | 732 | 630 | 399 | 707 | 650 |
| 7 | SARS.COV2-N2-RE1.1 | 1112 | 1300 | 1150 | 987 | 1006 | 1017 | 1264 | 1018 |
| 8 | SARS.COV2-N2-RE1.2 | 724 | 755 | 567 | 482 | 661 | 610 | 590 | 558 |
| 9 | SARS.COV2-N2-RE1.3 | 1532 | 1796 | 1449 | 1086 | 1493 | 1205 | 1245 | 1255 |
| 10 | SARS.CoV1-N2-pbVAR | 660 | 979 | 1008 | 640 | 1118 | 803 | 598 | 551 |
| 11 | SARS.CoV1-N2-RE1.1 | 580 | 521 | 1157 | 477 | 468 | 560 | 759 | 571 |
| 12 | SARS.CoV1-N2-RE1.2 | 744 | 588 | 1278 | 749 | 939 | 885 | 869 | 719 |
| 13 | SARS.CoV1-N2-RE1.3 | 1045 | 915 | 967 | 1005 | 1298 | 856 | 1157 | 1001 |
| 14 | SARS.COV2-N3-pub | 335 | 168 | 246 | 164 | 248 | 50 | −35 | 89 |
| 15 | SARS.COV2-N3-RE1.1 | 344 | 313 | 365 | 283 | 374 | 234 | 224 | 269 |
| 16 | SARS.COV2-N3-RE1.2 | −12 | 15 | −26 | −56 | −70 | −13 | −98 | −22 |
| 17 | SARS.COV2-N3-RE1.3 | 204 | 297 | 123 | 207 | 238 | 260 | 284 | 231 |
| 18 | RNAse.P.Probe-pub1.1 | 1128 | 892 | 853 | 714 | 768 | 652 | 814 | 807 |
| 19 | RNAse.P.Probe-pub1.2 | 1013 | 854 | 981 | 891 | 940 | 773 | 863 | 981 |
| 20 | InfA.7.univ-Fwd.RE1.1 | 573 | 593 | 553 | 382 | 541 | 220 | 465 | 541 |
| 21 | InfA.7.univ-pubRev | 22662 | 21290 | 289 | 284 | 355 | 157 | 292 | 247 |
| 22 | InfA.7.univ-RE1.1 | 38326 | 35727 | 331 | 305 | 178 | 378 | 292 | 160 |
| 23 | InfA.7.univ-RE1.3 | 28211 | 29917 | 40 | −9 | 82 | 184 | 55 | −34 |
| 24 | InfB.8.univ-pub | 70 | −26 | 1097 | 80 | 59985 | 60694 | −2 | 30 |
| 25 | InfB.8.univ-RE1.1 | 193 | 197 | 214 | 179 | 58811 | 53771 | 216 | 285 |
| 26 | InfB.8.univ-RE1.3 | 455 | 434 | 422 | 469 | 22626 | 16671 | 494 | 390 |
| 27 | H1N1.4.Sel-RE1.1 | −39 | −56 | −29 | 50 | 142 | −29 | 8 | 5 |
| 28 | H1N1.4.Sel-RE1.3 | −45 | −18 | −39 | 10 | −15 | −103 | 2 | −13 |
| 29 | upE.Lu-RE1.1 | 713 | 578 | 674 | 819 | 703 | 831 | 754 | 566 |
| 30 | upE.Lu-RE1.2 | 475 | 410 | 305 | 262 | 250 | 252 | 294 | 227 |
| 31 | MERS.N2.RE-1.1 | 297 | 75 | 141 | 174 | 235 | 125 | 355 | 313 |
| 32 | MERS.N2.RE-1.2 | 1092 | 1067 | 989 | 1097 | 928 | 604 | 909 | 831 |
| 33 | MERS.N3.pub-1.1 | 262 | 397 | 289 | 217 | 189 | 361 | 247 | 220 |
| 34 | MERS.N3.RE-1.1 | 1182 | 1110 | 1266 | 801 | 832 | 853 | 972 | 710 |
| 35 | 62-KEL578t-1.2-A | 1038 | 1105 | 1056 | 1064 | 729 | 788 | 820 | 791 |
| 36 | 62-Duf-67T-SE-6.1b | 870 | 1140 | 1749 | 929 | 881 | 1223 | 967 | 981 |
| 37 | SARS.CoV1-N2B-RE1.1 | 355 | 390 | 650 | 432 | 483 | 536 | 964 | 404 |
| 38 | SARS.CoV2-1ab-pub | −155 | −153 | −121 | −95 | −30 | −158 | −141 | −111 |
| 39 | SARS.CoV2-lab-RE1.1 | 598 | 615 | 536 | 362 | 657 | 580 | 538 | 480 |
| 40 | SARS.CoV2-lab-RE1.3 | 41 | 140 | 83 | 61 | 156 | 144 | 111 | 8 |
| 41 | HCoV.229E.M-RE1.1 | 809 | 806 | 896 | 786 | 972 | 832 | 997 | 751 |
| 42 | HCoV.229E.M-RE1.2 | 649 | 545 | 511 | 816 | 675 | 286 | 526 | 300 |
| 43 | HCoV.HKU1.N-RE1.1 | 1024 | 1213 | 1382 | 1008 | 977 | 792 | 1003 | 726 |
| 44 | HCoV.HKU1.N-RE1.2 | −557 | −599 | −376 | −589 | −628 | −563 | −529 | −526 |
| 45 | HCoV.NL63.N-RE1.1 | −641 | −623 | −593 | −590 | −504 | −472 | −548 | −553 |
| 46 | HCoV.NL63.N-RE1.2 | −551 | −618 | −531 | −590 | −602 | −527 | −569 | −559 |
| 47 | HCoV.0C43.M-RE1.1 | −582 | −589 | −536 | −608 | −522 | −497 | −527 | −575 |
| 48 | HCoV.0C43.M-RE1.2 | −643 | −614 | −576 | −611 | −590 | −513 | −553 | −553 |
| 49 | SARS.CoV1-N1-RE1.2 | 368 | 358 | 333 | 352 | 225 | 222 | 373 | 284 |
| 50 | SARS.CoV1-N1-RE1.3 | 371 | 370 | 411 | 528 | 515 | 415 | 473 | 552 |

TABLE 26
Asymmetric One-Step RT-PCR and Two-step
RT-PCR analysis in total RNA samples

Slide Number/Type
9903 Series
Extraction Kit/Primer Set

| | |
|---|---|
| One-Step | Two-Step |

Mouthwash Samples
Sample 6
Probe Description

| | 8 mM MgCl2 | 8 mM MgCl2 |
|---|---|---|
| 62-Negcont-B | 3932 | 1748 |
| SARS.COV2-N1-pub | 15307 | 5763 |
| SARS.COV2-N1-RE1.1 | 8982 | 1514 |
| SARS.COV2-N1-RE1.2 | 1574 | 424 |
| SARS.COV2-N1-RE1.4 | 125 | 16 |
| SARS.COV2-N2-pub | 7935 | 34384 |
| SARS.COV2-N2-RE1.1 | 3843 | 5488 |
| SARS.COV2-N2-RE1.2 | 3334 | 4791 |
| SARS.COV2-N2-RE1.3 | 6777 | 11085 |
| SARS.CoV1-N2-pbVAR | 1607 | 1225 |
| SARS.CoV1-N2-RE1.4 | 1816 | 1067 |
| SARS.CoV1-N2-RE1.2 | 1912 | 1138 |
| SARS.CoV1-N2-RE1.3 | 2956 | 1109 |
| SARS.COV2-N3-pub | 22759 | 25631 |
| SARS.COV2-N3-RE1.1 | 11702 | 20475 |
| SARS.COV2-N3-RE1.2 | 5522 | 15413 |
| SARS.COV2-N3-RE1.3 | 5365 | 8871 |
| RNAse.P.Probe-pub1.1 | 3847 | 52902 |
| RNAse.P.Probe-pub1.2 | 1506 | 63217 |
| SARS.COV2-N2-RE1.4 | 11577 | 39409 |

### Results

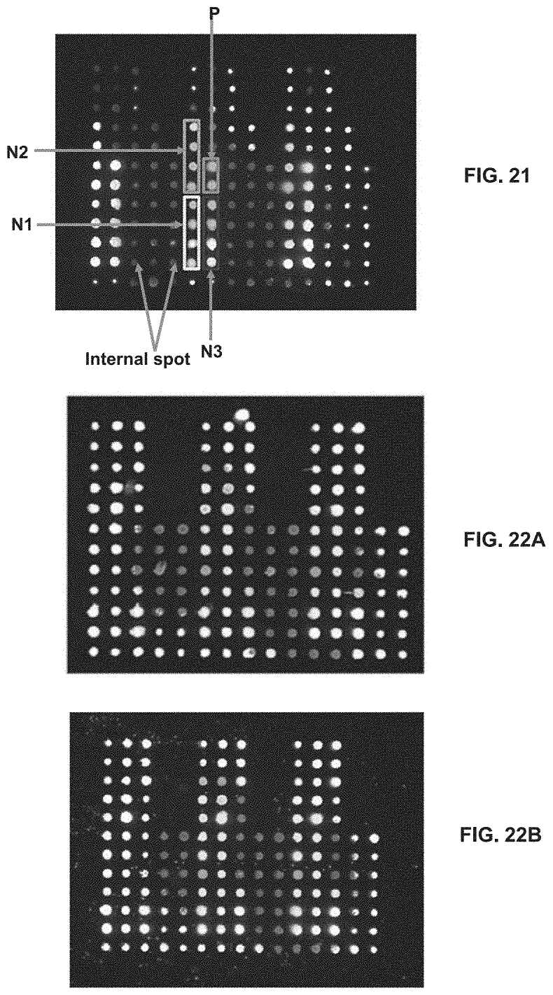

FIG. 21 shows one well (C3) from the slide (Tricor, COVID-19 Positive sample), which is statistical identical to all 20 of the COVID-19 wells. Nineteen of the twenty positives were correctly identified AUGURY as COVID positive.

### Automation End-to-End Mini-RV 96-Well Format.

### Time and Resources Required to Execute the Automation Script:

Approximate time elapsed to process 1×96 well slide
i. RNA extraction—4 h 10 min (including 90 min dry time)
ii. PCR plate preparation—12.5 min
iii. PCR amplification (Asymmetric One-Step RT-PCR)—2 h 40 min
iv. Hybridization script—1 h 45 min
v. Slide imaging—15 min

Total time˜9 h

Tip boxes required to process 1×96 well slide
i. RNA extraction—4×200 μl+6.5×1000
ii. PCR plate preparation—1×50 μl
iii. Hybridization script—0.5×1000 μl+2.5×200 μl+1×50 μl

Total tip boxes; 1 ml-7 boxes, 200 μl—6.5 boxes, 50 μI-2 boxes

Two full runs were performed with the Tecan EVO using the 96-well format;

Run 1. Comparison of automation versus manual: A series of contrived samples were created using irradiated SARS-CoV2 lysate in VTM. A checkered board pattern was created to evaluate the robotics and the potential for cross-contamination.

Results: The hybridization signals obtained (FIGS. 22A and 22B) were found to be stronger than that observed in FIG. 21. Data obtained using automation (FIG. 22A, well A1, slide 9903003012) was in excellent agreement with the manual method (FIG. 22B, well G1, slide 9903003012).

Run 2. Clinical sample evaluation: Known positive (25 samples) and negative (22 samples) COVID samples from TriCore were employed, including 49 water blanks. A checkered board pattern was created as above to evaluate the robotics and the potential for cross-contamination.

Results: The hybridization signals obtained were found to be stronger than that observed in FIG. 21. Data obtained using automation was in excellent agreement with the manual method. AUGURY correctly identified all 25 COVID positive samples and 21 of the COVID negative samples

### Example 16

### In Silico Analysis of Human Respiratory Syncytial Virus (HRSV) Feasibility

Adding a RSV test to the previously discussed content (SARS-CoV2, Clade Variant and Influenza A, B) was considered be valuable in this analysis. To test this, a fast-track analysis for implementing a HRSV test with the SARS-CoV2 content was performed.

Assay Design. An established Q-RT-PCR assays for HRSV (Table 27) was modified using PDx design principles (Table 28) into a PDx format comprising a single RT-PCR reaction and 3 probes (Pan HRSV probe, Subfamily A probe, Subfamily B Probe).

Inclusivity analysis Table 29 shows an inclusivity analysis of the primers and probes for the Hu et al and the PDx assays using the following sequences—HRSV (taxid:11250), HRSV-A (taxid:208893) and HRSV-B (taxid:208895). The analysis revealed that PDx probes have adequate Inclusivity and well suited to distinguish HRSV A subtype from HRSV B subtypes.

TABLE 27
A well-referenced⁵ RT-PCR assay to detect HRSV subtypes A and B

| | | |
|---|---|---|
| NC_038235.1.HRSV.A (SEQ ID: 107) | 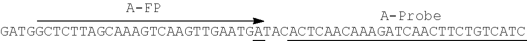 | 1198 |
| NC_001781.1.HRSV.B (SEQ ID: 108) | 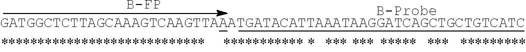 | 1198 |
| NC_038235.1.HRSV.A (SEQ ID: 109) | 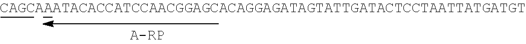 | 1258 |
| NC_001781.1.HRSV.B (SEQ ID: 110) | 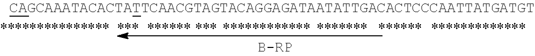 | 1258 |

*Hu, A, Cofella, M., Tam, J.S., Rappaport, R., Cheng, S., 2003. Journal of Clinical Microbiology 41,149-154.

TABLE 28
PDX redesign with common PCR primers for HRSV subtypes A and B and probes centered over the mismatches

| | | |
|---|---|---|
| NC_038235.1.HRSV.A (SEQ ID: 107) | 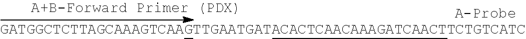 | 1198 |
| NC_001781.1.HRSV.B (SEQ ID: 108) | 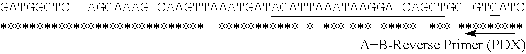 | 1198 |
| NC_038235.1.HRSV.A (SEQ ID: 109) | CAGCAAATACACCATCCAACGGAGCACAGGAGATAGTATTGATACTCCTAATTATGATGT | 1258 |
| NC_001781.1.HRSV.B (SEQ ID: 110) | 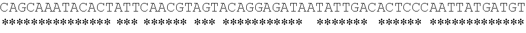 | 1258 |

TABLE 29
Inclusivity analysis of Hu et al. vs PDx Probes

| | | | | Number of sequences with | | |
|---|---|---|---|---|---|---|
| | Primer/ | | | 100% complementarity | | |
| Assay | Probe | SEQ ID NO | Sequence (5′ to 3′) | HRSV | HRSV-A | HRSV-B |
| Hu et al. (2003) | A-FP | SEQ ID: 111 | GCTCTTAGCAAAGTCAAGTTGAATGA | 1971 | 331 | 196 |
| A (N gene) | | | | (2258)¹ | (530)¹ | |
| | A-RP | SEQ ID: 112 | TGCTCCGTTGGATGGTGTATT | 536 | 262 | NSC |
| | | | | (1403)² | (543)² | |
| | A-probe | SEQ ID: 113 | ACACTCAACAAAGATCAACTTCTGTCATCCAGC | 449 | 247 | NSC |
| | | | | (1408)³ | (531)³ | |
| Hu et al. (2003) | B-FP | SEQ ID: 114 | GATGGCTCTTAGCAAAGTCAAGTTAA | 102 | 1 | 25? |
| B (N gene) | | | | (1106)⁴ | (234)⁴ | |
| | B-RP | SEQ ID: 115 | TGTCAATATTATCTCCTGTACTACGTTGAA | 1075 | NSC | 265 |
| | B-probe | SEQ ID: 116 | TGATACATTAAATAAGGATCAGCTGCTGTCATCCA | 904 | NSC | 226 |
| PathogenDx | A + B FP | SEQ ID: 117 | AAARATGGCTCTTAGCAAAGTCAAG | 2429 | 530 | 233 |
| proposed | A + B RP | SEQ ID: 118 | CGTTGRATRGTRTATTTGCTGGATG | 2439 | 536 | 266 |
| A + B (N gene) | A-probe | SEQ ID: 119 | ACACTCAACAAAGATCAACTTCT | 1406 | 536 | NSC |
| | B-probe | SEQ ID: 120 | ACATTAAATAAGGATCAGCTGCT | 910 | NSC | 229 |

NSC No significant complementarity
¹Hits increase when removing 3′TGA
²Hits increase changing 3′ TGTATT to TRTATT
³Hits increase when 1 mismatch allowed
⁴Hits increase when 3′-TTAA removed

Exclusivity. Table 30 shows that for both the Hu et al and the PDx assays using the following sequences—Homo sapiens (taxid:9606), HCoV229E (taxid:11137), HCoV-OC43 (taxid:31631), HCoV-HKU1 (taxid:290028), HCoV-NL63 (taxid:277944), MERS-CoV (taxid:1335626), Human metapneumovirus (taxid:162145), Human adenovirus sp. (taxid:1907210), HPIV-1 (taxid:12730), HPIV-2 (taxid:1979160), HPIV4 (taxid:1979161), Influenza A virus (taxid:11320), Influenza B virus (taxid:11520), Enterovirus (taxid:12059), Human parainfluenza virus 4b (taxid:11226), Streptococcus pneumoniae (taxid:1313), HRSV (taxid:11250), Rhinovirus (taxid:12059), Chlamydia pneumoniae (taxid:83558), Haemophilus virus HP2 (taxid:157239), Legionella pneumophila (taxid:446), Mycobacterium tuberculosis (taxid:1773), Streptococcus pyogenes (taxid:1314), Bordetella pertussis (taxid:520), Mycoplasma pneumoniae (taxid:2104), Pneumocystis jirovecii (taxid:42068), Candida albicans (taxid:5476), Pseudomonas aeruginosa (taxid:287), Staphylococcus epidermidis (taxid:1282), Streptococcus salivarius (taxid:1304), HPIV-3 (taxid:11216); and exclude: HCoV-SARS (taxid:694009), SARS-CoV2 (taxid:2697049).

As seen from Table 30, there is negligible experimental cross reaction with human DNA/RNA or any of the standard panel of respiratory pathogens required for analysis of Exclusivity in SARS-CoV-2 testing.

Feasibility. The calculations obtained suggests feasibility of implementing the HRSV assay capacity to the present 12 probe Mini-RV assay.

### Example 17

### Automation of 96-Well and 384-Well Plates

A hybridization script on the Tecan was upgraded to reduce reagent waste. A new hybridization script was tested and found to provide results equivalent to non-automated two-step RT-PCR (with labeling) as shown in Table 31. The script was edited for compatibility with plate processing ancillary equipment and the protocol used to run the Zymo kits.

### Example 18

### Asymmetric One-Step RT-PCR QC Test Development & Validation

A QC/QA test protocol was developed (for [S, N1, N2, N3, P, PanA, PanB). Multiple Tricore samples were pooled to generate a stock solution of purified clinically derived RNA for QC/QA. Table 32 summarizes the results from this analysis.

TABLE 30
Exclusivity analysis of Primers &Probes for Hu et al. vs PDx¹

| | Primer/ | | | Homo | |
|---|---|---|---|---|---|
| Assay | Probe | | seq 5′ to 3′ | sapiens | Non-human |
| Hu et al. (2003) | A-FP | SEQ ID: 111 | GCTCTTAGCAAAGTCAAGTTGAATGA | 69% | Streptococcus |
| A (N gene) | | | | | salivarius 62% |
| | A-RP | SEQ ID: 112 | TGCTCCGTTGGATGGTGTATT | 90% | Pseudomonas |
| | | | | | aeruginosa 67% |
| | A-probe | SEQ ID: 113 | ACACTCAACAAAGATCAACTTCTGTCATCCAGC | — | |
| Hu et al. (2003) | B-FP | SEQ ID: 114 | GATGGCTCTTAGCAAAGTCAAGTTAA | 69% | Streptococcus |
| B (N gene) | | | | | salivarius 61% |
| | B-RP | SEQ ID: 115 | TGTCAATATTATCTCCTGTACTACGTTGAA | 57% | Legionella |
| | | | | | pneumophila 60% |
| | B-probe | SEQ ID: 116 | TGATACATTAAATAAGGATCAGCTGCTGTCATCCA | — | |
| PathogenDx | A + B FP | SEQ ID: 117 | AAARATGGCTCTTAGCAAAGTCAAG | 68% | Streptococcus |
| proposed | | | | | salivarius 64% |
| A + B (N gene) | A + B RP | SEQ ID: 118 | CGTTGRATRGTRTATTTGCTGGATG | 64% | Streptococcus |
| | | | | | pneumoniae 52% |
| | A-probe | SEQ ID: 119 | ACACTCAACAAAGATCAACTTCT | N/A² | |
| | B-probe | SEQ ID: 120 | ACATTAAATAAGGATCAGCTGCT | N/A² | |

¹Exclusivity respiratory panel % complementarity (organism with closest match). Generally, <80% total complementarity requires no deeper analysis
²Surface Bound non-PCR oligos are not subjected to sequences other than amplimers generated from the PCR primers

TABLE 31
Summary of the 96-well automated hybridization test*

| | | Standard | | Standard | | (b) | (c) False | (d) True | | | | Speci- | | |
|---|---|---|---|---|---|---|---|---|---|---|---|---|---|---|
| | Average | Deviation | Average | Deviation | (a) True | False | Nega- | Nega- | | | Sensi- | fi- | | |
| | Positives | Positives | Negatives | Negatives | Positive | Positive | tives | tives | LoB | LoD | tivity | city | PPV | NPV |
| 62-Negcont-B | 2084 | 729 | 2005 | 1197 | 30 | 0 | 0 | 30 | N/A | N/A | 100 | 100 | 100 | 100 |
| RNAse.P.Probe- | 56092 | 1324 | 59532 | 1281 | 30 | 0 | 0 | 30 | N/A | N/A | 100 | 100 | 100 | 100 |
| pub1.1 | | | | | | | | | | | | | | |
| SARS.COV2- | 55102 | 3898 | 5625 | 15613 | 30 | 3 | 0 | 30 | N/A | N/A | 100 | 90.91 | 91 | 100 |
| N1-RE1.1 | | | | | | | | | | | | | | |
| SARS.COV2- | 55190 | 3091 | 6328 | 15471 | 30 | 3 | 0 | 30 | N/A | N/A | 100 | 90.91 | 91 | 100 |
| N1-RE1.1 | | | | | | | | | | | | | | |
| SARS.COV2- | 52644 | 4131 | 7791 | 15341 | 30 | 5 | 0 | 30 | N/A | N/A | 100 | 85.71 | 86 | 100 |
| N2-RE1.3 | | | | | | | | | | | | | | |
| SARS.COV2- | 53041 | 4202 | 8336 | 14677 | 30 | 5 | 0 | 30 | N/A | N/A | 100 | 85.71 | 86 | 100 |
| N2-RE1.3 | | | | | | | | | | | | | | |
| SARS.COV2- | 56120 | 1324 | 9323 | 19459 | 30 | 5 | 0 | 30 | N/A | N/A | 100 | 85.71 | 86 | 100 |
| N2-RE1.4 | | | | | | | | | | | | | | |
| SARS.COV2- | 56120 | 1324 | 12704 | 22979 | 30 | 7 | 0 | 30 | N/A | N/A | 100 | 81.08 | 81 | 100 |
| N3-RE1.1 | | | | | | | | | | | | | | |
| SARS.COV2- | 56119 | 1321 | 12635 | 23308 | 30 | 7 | 0 | 30 | N/A | N/A | 100 | 81.08 | 81 | 100 |
| N3-RE1.1 | | | | | | | | | | | | | | |
| TOTAL | N/A | N/A | N/A | N/A | 30 | 4 | 0 | 30 | N/A | N/A | 100 | 88.24 | 88 | 100 |

*Tecan Automated Hybridization Protocol - Two-Step RT-PCR and Labeling Reaction with TriCore NP Samples

TABLE 32
Optimizing complete [S.N1, N2.N3.P, PanA, PanB] using pooled positive and pooled negative samples

| | | Positive | Positive | Positive | | Negative | Negative | Negative |
|---|---|---|---|---|---|---|---|---|
| | Positive | pooled | pooled | pooled | Negative | pooled | pooled | pooled |
| Sample | pooled | (infA) | (infB) | (infA,B) | pooled | (infA) | (infB) | (infA, B) |
| 62-Negcont-B | 2502 | 1703 | 2947 | 1012 | 1950 | 2380 | 1870 | 1728 |
| SARS.COV2-N1-RE1.1 | 62053 | 60858 | 58695 | 55988 | 2330 | 2227 | 2067 | 1417 |
| SARS.COV2-N2-RE1.3 | 53469 | 50035 | 48069 | 48001 | 696 | 860 | 751 | 223 |
| SARS.COV2-N2-RE1.4 | 62125 | 61966 | 62838 | 60932 | 1445 | 1229 | 1274 | 946 |
| SARS.COV2-N3-RE1.1 | 62378 | 62148 | 63048 | 61164 | 6858 | 4820 | 5932 | 4001 |
| RNAse.P.Probe-pub1.1 | 47022 | 39703 | 39083 | 37667 | 62564 | 61029 | 62077 | 55084 |
| InfA.7.univ-pubRev | 257 | 2913 | 450 | 2852 | 329 | 37312 | −44 | 36881 |
| InfB.8.univ-pub | 24 | −134 | 8691 | 5335 | −161 | −68 | 17062 | 16098 |
| 614D-AS-S1-RE1.1 | 905 | 721 | 1573 | −52 | 596 | 643 | 584 | 13 |
| 614G-AS-S1-RE1.1X | 1466 | 1486 | 3129 | 437 | 1176 | 1121 | 1070 | 775 |
| 614D-SE-S1-RE1.1 | 2680 | 1429 | 9252 | 100 | 745 | 680 | 818 | 238 |
| 614G-SE-S1-RE1.2 | 30306 | 14561 | 14828 | 12906 | 840 | 617 | 1104 | 710 |

### Example 19

### 96-Well and 384-Well Test Optimization

Clinical sensitivity and specificity analysis performed on the Mini-RV content in 9985 array format using Asymmetric One-Step RT-PCR revealed a 100% sensitivity and 94% specificity for each of the 96-well and 384-well samples. Tables 33 and 34 shows an improvement in specificity for the SARS.COV2-N1-RE1.1 probe. Signal strength for the RNase-P control is also improved.

### Example 20

Influenza testing on Clinical Samples (NP/VTM) using Mini-RV, Asymmetric One-Step RT-PCR
Influenza Positive TriCore Clinical Samples (N/VTM) were used for clinical evaluation of Influenza A and B primer and probes in two positive Influenza A, validated on a respiratory panel (RESPAN, TriCore) and two positive Influenza B, validated on an Influenza A/B and RSV panel (FLURSV, TriCore), analyzed on Mini-RV slide format. Table 35 shows that Influenza A and B were detected in confirmed clinical samples via standard Asymmetric One-Step RT-PCR (Zymo), with a clear discrimination between Influenza A vs Influenza B.

### Example 21

### Analysis of Mouthwash Samples Using Mini-RV Asymmetric One-Step RT-PCR.

Mouthwash/saliva samples were separated and evaluated by itself (MW-1), spiked with SARS-CoV2 viral lysate from ATCC (MW-2), or with SARS-CoV2 purified viral RNA from ATCC (MW-3). The mouthwash sample was taken through Zymo's RNA purification and amplified using the Asymmetric One-Step RT-PCR method. Amplicons were analyzed on Mini-RV format (12-well slides). Table 36 shows that SARS-CoV2 was detected in contrived mouthwash samples.

TABLE 33
Clinical Sensitivity and Specificity in 96-well format

| | | Standard | | Standard | | (b) | (c) False | (d) True | | | | | | |
|---|---|---|---|---|---|---|---|---|---|---|---|---|---|---|
| | Average | Deviation | Average | Deviation | (a) True | False | Nega- | Nega- | | | Sensi- | Specifi- | | |
| | Positives | Positives | Negatives | Negatives | Positive | Positive | tives | tives | LoB | LoD | tivity | city | PPV | NPV |
| 62-Negcont-B | 1576 | 978 | 1255 | 561 | 30 | 0 | 0 | 30 | N/A | N/A | 100 | 100 | 100 | 100 |
| RNAse.P.Probe- | 53737 | 13600 | 48392 | 23841 | 30 | 0 | 0 | 30 | N/A | N/A | 100 | 100 | 100 | 100 |
| pub1.1 | | | | | | | | | | | | | | |
| SARS.COV2- | 49089 | 17417 | 7544 | 4311 | 30 | 2 | 0 | 30 | N/A | N/A | 100 | 94 | 94 | 100 |
| N1-RE1.1 | | | | | | | | | | | | | | |
| SARS.COV2- | 40485 | 21135 | 2567 | 4861 | 30 | 2 | 0 | 30 | N/A | N/A | 100 | 94 | 94 | 100 |
| N2-RE1.3 | | | | | | | | | | | | | | |
| SARS.COV2- | 50842 | 17848 | 1702 | 734 | 30 | 2 | 0 | 30 | N/A | N/A | 100 | 94 | 94 | 100 |
| N2-RE1.4 | | | | | | | | | | | | | | |
| SARS.COV2- | 51720 | 16137 | 8152 | 4697 | 30 | 2 | 0 | 30 | N/A | N/A | 100 | 94 | 94 | 100 |
| N3-RE1.1 | | | | | | | | | | | | | | |
| Overall | N/A | N/A | N/A | N/A | 30 | 2 | 0 | 30 | N/A | N/A | 100 | 94 | 94 | 100 |

TABLE 34
Clinical Sensitivity and Specificity in 384-well format

| | Ave- | Standard | Ave- | Standard | (a) | (b) | (c) | (d) | | | | | | |
|---|---|---|---|---|---|---|---|---|---|---|---|---|---|---|
| | rage | Deviation | rage | Deviation | True | False | False | True | | LoD | | | | |
| | Posi- | Posi- | Neg- | Neg- | Posi- | Posi- | Neg- | Neg- | | copies/ | Sensi- | Speci- | | |
| | tives | tives | atives | atives | tive | tive | atives | atives | LoB | ml | tivity | ficity | PPV | NPV |
| 62-Negcont-B | 2286 | 793 | 2696 | 1177 | 30 | 0 | 0 | 30 | N/A | 62.5 | 100 | 100 | 100 | 100 |
| RNAse.P.Probe- | 49278 | 15639 | 48208 | 21205 | 30 | 0 | 0 | 30 | N/A | 62.5 | 100 | 100 | 100 | 100 |
| pub1.1 | | | | | | | | | | | | | | |
| SARS.COV2- | 41364 | 18774 | 5041 | 1377 | 30 | 2 | 3 | 30 | N/A | 62.5 | 91 | 94 | 94 | 91 |
| N1-RE1.1 | | | | | | | | | | | | | | |
| SARS.COV2- | 35109 | 18663 | 1200 | 716 | 30 | 2 | 3 | 30 | N/A | 62.5 | 91 | 94 | 94 | 91 |
| N2-RE1.3 | | | | | | | | | | | | | | |
| SARS.COV2- | 47691 | 17794 | 1465 | 648 | 30 | 2 | 3 | 30 | N/A | 62.5 | 91 | 94 | 94 | 91 |
| N2-RE1.4 | | | | | | | | | | | | | | |
| SARS.COV2- | 48073 | 16722 | 5004 | 2292 | 30 | 2 | 3 | 30 | N/A | 62.5 | 91 | 94 | 94 | 91 |
| N3-RE1.1 | | | | | | | | | | | | | | |
| Overall | N/A | N/A | N/A | N/A | 30 | 0 | 3 | 30 | N/A | 62.5 | 91 | 100 | 100 | 91 |

TABLE 35
DETECTX-RV (Mini-RV) influenza evaluation

| | | Influenza | | | | |
|---|---|---|---|---|---|---|
| | | A/B | | | | |
| | | (Positive | | | | |
| | | Control) | | | | |
| | | Influenza | | | | |
| | No | A/B | | | | |
| | Template | (10,000 | Influenza A | | Influenza B | |
| 9985 | Control | copies/ | (Clinical Sample) | | (Clinical Sample) | |
| Probes | (NTC) | reaction) | #179502 | #179504 | #170231 | #170232 |
| 62-Negcont-B | 3869 | 2584 | 2392 | 2934 | 2339 | 4421 |
| SARS.COV2-N1- | 5155 | 293 | 584 | 291 | 3342 | 1125 |
| SARS.COV2-N2- | 825 | −21 | −19 | −34 | 42 | 361 |
| SARS.COV2-N2- | 991 | 921 | 1055 | 1068 | 1460 | 1434 |
| SARS.COV2-N3- | 7727 | 1415 | 4105 | 6139 | 7031 | 4604 |
| RNAse.P.Probe- | 2498 | 1465 | 54868 | 61894 | 59917 | 54383 |
| InfA.7.univ-pubRev | −185 | 11670 | 54328 | 50269 | 658 | 555 |
| InfB.8.univ-pub | 397 | 61813 | 255 | 366 | 10304 | 30340 |
| 614D-AS-S1-RE1.1 | 321 | 549 | 135 | 219 | 311 | 110 |
| 614G-AS-S1- | 1056 | 845 | 654 | 925 | 905 | 1171 |
| 614D-SE-S1-RE1.1 | 421 | 416 | 189 | 515 | 176 | 569 |
| 614G-SE-S1-RE1.2 | 228 | 454 | 208 | 422 | 327 | 666 |

TABLE 36
DETECTX-RV (Mini-RV) SARS-CoV-2 evaluation in contrived mouthwash samples

| | | | Pooled | | | |
|---|---|---|---|---|---|---|
| | | SARS-CoV-2 | Sample | | | |
| | | (Positive | (Positive | | | |
| | | Control | Control) | | | |
| | | Plasmid) | SARS-CoV-2 | | | |
| | | SARS-CoV-2 | (Clinical | Contrived | | |
| 9985 | | 1000 copies/ | Positive, | Mouthwash Samples | | |
| Probes | NTC | reaction | NP/VTM) | MW-1* | MW-2^{¶} | MW-3^{§} |
| 62-Negcont-B | 3869 | 2719 | 3196 | 3486 | 2342 | 1955 |
| SARS.COV2-N1-RE1.1 | 5155 | 37071 | 61691 | 3379 | 48808 | 35396 |
| SARS.COV2-N2-RE1.3 | 825 | 21979 | 42964 | 551 | 40579 | 11916 |
| SARS.COV2-N2-RE1.4 | 991 | 51528 | 61773 | 2273 | 61671 | 40734 |
| SARS.COV2-N3-RE1.1 | 7727 | 52631 | 61944 | 4932 | 61745 | 44499 |
| RNAse.P.Probe-pub1.1 | 2498 | 2482 | 46178 | 39598 | 13423 | 36278 |
| InfA.7.univ-pubRev | −185 | −36 | 47 | 128 | 27 | 162 |
| InfB.8.univ-pub | 397 | 485 | 50 | −115 | 112 | −17 |
| 614D-AS-S1-RE1.1 | 321 | 1031 | 716 | 833 | 1420 | 727 |
| 614G-AS-S1-RE1.1X | 1056 | 1834 | 1615 | 1201 | 1608 | 979 |
| 614D-SE-S1-RE1.1 | 421 | 1336 | 3467 | 637 | 18936 | 1133 |
| 614G-SE-S1-RE1.2 | 228 | 1207 | 36769 | 964 | 1444 | 552 |

*MW-1 - Mouthwash collected, not spiked with SARS-CoV-2
^{¶}MW-2 - Mouthwash, spiked with SARS-CoV-2 Viral Lysate, 1 × 108 PFU (BEI. Wuhan)
^{§}MW-3 - Mouthwash, spiked with SARS-CoV-2 purified RNA at 1000 copies total for input to RNA extraction (BEI. Wuhan)

### Example 22

### Design of a Pan-Cold Coronavirus Probe Assay

The assay is based on RdRp and has an inclusivity of (NL63+OC43+229E+HKU1). Primers and Probes used for the assay are shown in Tables 37 and 38. In Silico analysis demonstrates that the primers and probes are specific for their targets and do not demonstrate off target interactions—less than 80% homology to any off-target sequence. Table 39 shows the exclusivity analysis using the following sequences—Homo sapiens (taxid:9606), HCoV-229E (taxid:11137), HCoV-OC43 (taxid:31631), HCoV-HKU1 (taxid:290028), HCoV-NL63 (taxid:277944), MERS-CoV (taxid:1335626), Human metapneumovirus (taxid:162145), Human adenovirus sp. (taxid:1907210), HPIV-1 (taxid:12730), HPIV-2 (taxid:1979160), HPIV4 (taxid:1979161), Influenza A virus (taxid:11320), Influenza B virus (taxid:11520), Enterovirus (taxid:12059), Human parainfluenza virus 4b (taxid:11226), Streptococcus pneumoniae (taxid:1313), HRSV (taxid: 11250), Rhinovirus (taxid:12059), Chlamydia pneumoniae (taxid:83558), Haemophilus virus HP2 (taxid:157239), Legionella pneumophila (taxid:446), Mycobacterium tuberculosis (taxid:1773), Streptococcus pyogenes (taxid:1314), Bordetella pertussis (taxid:520), Mycoplasma pneumoniae (taxid:2104), Pneumocystis jirovecii (taxid:42068), Candida albicans (taxid:5476), Pseudomonas aeruginosa (taxid:287), Staphylococcus epidermidis (taxid:1282), Streptococcus salivarius (taxid:1304), HPIV-3 (taxid:11216); and exclude: HCoV-SARS (taxid:694009), SARS-CoV2 (taxid:2697049). No complementarity (<80%) was observed compared to the full standard exclusivity panel. Inclusivity analysis (Table 40) using the sequences HCoV-NL63 (taxid:277944), HCoV-OC43 (taxid:31631), HCoV-229E (taxid:11137), HCoV-HKU1 (taxid:290028) MERS-CoV (taxid:1335626) showed >98% compared to GenBank (NL63+OC43+229E+HKU1) plus several other animal Coronavirus.

TABLE 37
Primer sequences for Pan-cold Coronavirus probe assay

| SEQ ID NO. | Target | Gene | Primer Sequence (5′ to 3′) |
|---|---|---|---|
| SEQ ID: 23 | SARS-CoV-2 | N1 | TTTTAATGGACCCCAAAATCAGCGAAAT |
| | Nucleocapsid | | |
| SEQ ID: 24 | SARS-CoV-2 | N1 | (FL)TTTTTCTGGTTACTGCCAGTTGAATC |
| | Nucleocapsid | | TG |
| SEQ ID: 25 | CoV Nucleocapsid | N2 | TTTACTGATTACAAACATTGGCCGCAAA |
| SEQ ID: 74 | CoV Nucleocapsid | N2 | (FL)TTTTGCCAATGCGCGACATTCCGAA |
| | | | GAA |
| SEQ ID: 27 | CoV Nucleocapsid | N3 | TTTAGGGAGCCTTGAATACACCAAAAGA |
| SEQ ID: 28 | CoV Nucleocapsid | N3 | (FL)TTTAAGTTGTAGCACGATTGCAGCA |
| | | | TTG |
| SEQ ID: 75 | SARS-CoV-2 Spike | S | TTTAGTGTTATAACACCAGGAACAAATA |
| | Gene | | |
| SEQ ID: 76 | SARS-CoV-2 Spike | S | (FL)TTTTGCATGAATAGCAACAGGGACT |
| | Gene | | TCT |
| SEQ ID: 77 | Pan-CoV RdRp | RdRp | TTTTTTAATAAGTATTTTAAGCAYTGGAG |
| | | | T |
| SEQ ID: 78 | Pan-CoV RdRp | RdRp | (FL)TTTAAGAGTGTGTTAAAATTTGAACA |
| | | | ATG |
| SEQ ID: 79 | Pan-CoV RdRp | RdRp | TTTTGTTTAAGAAGTATTTTAARTATTGG |
| | | | G |
| SEQ ID: 80 | Pan-Coy RdRp | RdRp | (FL)TTTAATAGTGTATTRAAATTAGCACA |
| | | | ATG |
| SEQ ID: 39 | Influenza A | M | TTTCAAGACCRATCCTGTCACCTCTGAC |
| SEQ ID: 81 | Influenza B | M | TTTGGATCCTCAACTCACTCTTCGAGCG |
| SEQ ID: 82 | Influenza A | NS1 | (FL)TTTGGGCATTYTGGACAAAKCGTCT |
| | | | ACG |
| SEQ ID: 42 | Influenza B | NS1 | (FL)TTTTAATCGGTGCTCTTGACCAAATT |
| | | | GG |
| SEQ ID: 83 | HRSV | N | TTTAAARATGGCTCTTAGCAAAGTCAAG |
| SEQ ID: 84 | HRSV | N | (FL)TTTCGTTGRATRGTRTATTTGCTGG |
| | | | ATG |
| SEQ ID: 43 | Human RNAse P | RNAse P | TTTGTTTGCAGATTTGGACCTGCGAGC |
| | control | | G |
| SEQ ID: 44 | Human RNAse P | RNAse P | (FL)TTTAAGGTGAGCGGCTGTCTCCACA |
| | control | | AGT |

(FL) = fluorescent label.

TABLE 38
Nucleic acid probe sequences used for hybridization in Pan-cold
Coronavirus probe assay

| SEQ ID NO. | Target | Detects | Probe Sequence |
|---|---|---|---|
| SEQ ID: 45 | SARS-CoV-2 | SARS-CoV-2 614G, | TTTTTTTCCGCATTACGTTT |
| | | SARS-CoV-2 614D | GGTGTTTTTT |
| SEQ ID: 48 | SARS-CoV-2 | SARS-CoV-2 614G, | TTTTTTACAATTTGCCCCC |
| | | SARS-CoV-2 614D | AGCGTCTTTTT |
| SEQ ID: 49 | SARS | SARS | TTTTTTTTTGCTCCRAGTG |
| | | | CCTCTTTTTTT |
| SEQ ID: 85 | CoV Bat precursor | Bat_SARS-like CoV | TTTTTTGTTTGCACCTAGT |
| | | | GCTTCCTTTTT |
| SEQ ID: 86 | CoV Pangolin | Pangolin CoV S. China | TTTTTTTTTGCTCCTAGCG |
| | precursor | | CTTCTTTTTTT |
| SEQ ID: 53 | CoV Bat precursor - | Bat precursor (Yunnan | TTTTTGTTTGCACCCAGTG |
| | Yunnan 2013 | 2013) | CTTCTGCTCTTTT |
| SEQ ID: 54 | CoV Bat precursor - | New bat CoVs (Yunnan | TTTTTTACAATTCGCTCCC |
| | Yunnan 2019 | 2019) | AGCGTCTTTTT |
| SEQ ID: 55 | CoV Nucleocapsid | SARS-CoV-2 614G, | TTTTTCTGGCACCCGCAAT |
| | | SARS-CoV-2 614D, | CCTGTCTTTTT |
| | | SARS, Bat-SARS-like | |
| | | CoV, Pangolin CoV, S. | |
| | | China, Bat precursor | |
| | | (Yunnan 2013), New | |
| | | Bat CoVs (Yunnan | |
| | | 2019) | |
| SEQ ID: 87 | SARS-CoV-2 614 | SARS-CoV-2 614G, | TTTTTCTCTTTATCAGGRT |
| | All | SARS-CoV-2 614D | GTTAACTGCTTTTTT |
| SEQ ID: 88 | SARS-CoV-2 614 | SARS-CoV-2 614G | TTTTTTCCTATCAGGGTGT |
| | “G” | | TAACTTTTTTT |
| SEQ ID: 89 | SARS-CoV-2 614 | SARS-CoV-2 614D | TTTTTCTTATCAGGATGTTA |
| | “D” | | ACTTTTTTTT |
| SEQ ID: 90 | HCoV-OC43 | HCoV-OC43 | TTTTTATATCATCCTAACAC |
| | | | TGTTGATTGTTTTTT |
| SEQ ID: 91 | NHCoV-NL63 | HCoV-NL63 | TTTTTTTATCATCCTAATTG |
| | | | TAGTGACTGTTTTTT |
| SEQ ID: 92 | NHCoV-HKU1 | HCoV-HKU1 | TTTTTGTATCATCCTAATAC |
| | | | TGTGGATTGTTTTTT |
| SEQ ID: 93 | HCoV-229E | HCoV-229E | TTTTTTTATCATCCTGATTG |
| | | | TGTTGATTGCTTTTT |
| SEQ ID: 94 | MERS | MERS-CoV | TTTTTAATTGCGTTAATTGT |
| | | | ACTGATGACCTTTTT |
| SEQ ID: 67 | Influenza A | Influenza A | TTTTTTTCGTGCCCAGTGA |
| | | | GCGAGTTTTTT |
| SEQ ID: 95 | Influenza B | Influenza B | TTTTCCAATTCGAGCAGCT |
| | | | GAAACTGCGGTGTTTTT |
| SEQ ID: 96 | HRSV-A | HRSV-A | TTTTTCACACTCAACAAAG |
| | | | ATCAACTTCTTCTTCTT |
| SEQ ID: 97 | HRSV-B | HRSV-B | TTTTTCGATACATTAAATAA |
| | | | GGATCAGCTTTTTTT |
| SEQ ID: 71 | RNAse P control | Human RNAse P | TTTTTTTTCTGACCTGAAG |
| | | | GCTCTGCGCGTTTTT |
| SEQ ID: 73 | Negative Control | Human RNAse P | TTTTTTCTACTACCTATGCT |
| | | | GATTCACTCTTTTT |

TABLE 39
Pan CoV - Exclusivity respiratory panel % complementarity¹
(Organism with closest match)

| | | | |
|---|---|---|---|
| | | Homo | |
| SEQ ID NO | Sequence | sapiens | Non-human |

Forward Primer seq (5′ to 3′)

| | | | | | |
|---|---|---|---|---|---|
| PathogenDx | NL63/OC43 | SEQ ID: 121 | TTTAATAAGTATTTTAAGCAYTGGAGT | 66% | Streptococcus |
| | | | | | pyogenes 59% |
| Proposed | 229E | SEQ ID: 122 | TGTTTAAGAAGTATTTTAARTATTGGG | 70% | Staphylococcus |
| Pan-CoV | HKU1 | | | | epidermis 60% |
| (RdRP gene) | MERS | | | | |

Reverse Primer seq (5′ to 3′)

| | | | | |
|---|---|---|---|---|
| 299E | SEQ ID: 123 | AAGAGTGTGTTAAAATTTGAACAATG | 73% | Streptococcus |
| | | | | pneumoniae |
| | | | | 73% |
| NL63 | SEQ ID: 124 | AATAGTGTATTRAAATTAGCACAATG | 79% | Staphylococcus |
| OC43 | | | | epidermis |
| HKU1 | | | | 61% |
| MERS | | | | |

Probe sequence (seq 5′ to 3′)

| | | | | |
|---|---|---|---|---|
| NL63 | SEQ ID: 125 | TTATCATCCTAATTGTAGTGACTGT | N/A² | N/A² |
| 229E | SEQ ID: 126 | TTATCATCCTGATTGTGTTGATTGC | N/A² | N/A² |
| OC43 | SEQ ID: 127 | ATATCATCCTAACACTGTTGATTGT | N/A² | N/A² |
| HKU1 | SEQ ID: 128 | GTATCATCCTAATACTGTGGATTGT | N/A² | N/A² |
| MERS* | SEQ ID: 129 | AATTGCGTTAATTGTACTGATGACC | N/A² | N/A² |

*shifted to avoid palindromic seq
¹Generally, <80% total complementarity requires no deeper analysis
²Surface Bound non-PCR oligos are not subjected to sequences other than amplimers generated from the PCR primers

TABLE 40
Pan CoV - Inclusivity Analysis (Genbank). Number of sequences with 100%
complementarity (unless noted)

| | | | | | | |
|---|---|---|---|---|---|---|
| | | HCoV- | HCoV- | HCoV- | HCoV- | MERS- |
| SEQ ID NO. | Sequence used for comparison | NL63 | OC43 | 229E | HKU1 | CoV |

Forward Primer seq (5′ to 3′)

| | | | | | | | | |
|---|---|---|---|---|---|---|---|---|
| PathogenDx | NL63 | SEQ ID: 130 | TTYAATAAGTAYTTTAAGCAYTGGAGT | 89/89 | 245/247 | N/A | N/A | N/A |
| | OC43 | | | 100% | 99.2% | | | |
| Proposed | 229E | SEQ ID: 131 | TSTTTRABAAGTAYTTTAARTATTGGG | N/A | N/A | 51/51 | 47/47 | 578/581 |
| Pan-CoV | HKU1 | | | | | 100% | 100% | 99.5% |
| (RdRP gene) | MERS | | | | | | | |

Reverse Primer seq (5′ to 3′)

| | | | | | | | |
|---|---|---|---|---|---|---|---|
| 299E | SEQ ID: 132 | AAGAGTGTGTTAAAATTTGAACAATG | N/A | N/A | 51/51 | N/A | N/A |
| | | | | | 100% | | |
| NL63 | SEQ ID: 133 | AAHARTRYRTTRAAATTAGCACAATG | 89/89 | 245/247 | N/A | 53/53 | 580/581 |
| OC43 | | | 100% | 99.2% | | 100% | 99.8% |
| HKU1 | | | | | | | |
| MERS | | | | | | | |

Probe sequence (seq 5′ to 3′)

| | | | | | | | |
|---|---|---|---|---|---|---|---|
| NL63 | SEQ ID: 134 | TTATCATCCTAATTGTAGTGACTGT | 88/89 | N/A | N/A | N/A | N/A |
| | | | 99% | | | | |
| 229E | SEQ ID: 135 | TTATCATCCTGATTGTGTTGATTGC | N/A | N/A | 51/51 | N/A | N/A |
| | | | | | 100% | | |
| OC43 | SEQ ID: 136 | ATATCATCCTAACACKGTTGATTGT | N/A | 244/247 | N/A | N/A | N/A |
| | | | | 98.7% | | | |
| HKU1 | SEQ ID: 137 | GTATCATCCTAATACTGTGGATTGT | N/A | N/A | N/A | 47/47 | N/A |
| | | | | | | 100% | |
| MERS* | SEQ ID: 138 | ATTGCGTTAATTGTACTGATGACC | N/A | N/A | N/A | N/A | 577/580 |
| | | | | | | | 99.5% |

*shifted to avoid palindromic seq

### Example 23

### Analysis of Contrived Samples

Emory Test Samples. Emory contrived a sample with heat inactivated CoV2 virus (BEI standards) in VTM, covering a dilution series from 10⁶ to 0 virus/ml. The sample was then shipped to PDx in double-blinded form. PDx performed the full manual process in the 96-well format (that is, Zymo RNA purification+One-Step PCR+Hybridization/Wash+Imaging+AUGURY). The results obtained were then reported to Emory, which was tasked with reporting concordance with the number of virus particles/ml originally added.
Training Samples. PDx completed validation and shipped to Emory, a full suite of stepwise training materials, “Imaging Test”; “Hybridization/Wash+Imaging Test”; “PCR+Hybridization/Wash+Imaging Test”. Two sets of blinded contrived samples were made from both γ-inactivated and heat inactivated reference standards (BEI) in pooled nasal fluid, diluted from 10⁴ copies/ml to 10 copies/ml (Tables 41 and 42).

### Results:

Emory testing of blinded contrived samples from PDx: Emory's data for the test samples provided by PDx, which required the full processing workflow (RT-PCR+Hybridization/Wash+Imaging+AUGURY) had readouts of the blinded PDx-contrived samples that were identical within experimental accuracy to that obtained independently at PDx.
PDx testing of double-blinded contrived samples from Emory: PDx's data readout on the double blinded samples provided by Emory (Tables 43 and 44) indicated a LoD of −1000 viral copies/ml for the contrived heat inactivated CoV-2 samples. It is interesting to note that the apparent LoD obtained by PDx is identical within experimental accuracy to that obtained by Emory and PDx (1000 copies/ml, Table 45).

It should be noted that if the PDx standard were relaxed to that used in most Q-RT-PCR assays, that is, probe detected comprising a positive readout, the LoD thus obtained by PDx

analysis on the Emory samples would be in the 10-100 copies/ml range, because reruns would have been identified as “positives” using the less stringent analytical standard (Tables 43).

It is also interesting to note that for the contrived samples prepared by PDx and measured by Emory (Table 42) and the contrived samples prepared by Emory and measured by PDx (Table 40) all samples lacking CoV-2 gave 100% negative results per PDx analysis (Tables 41, 42 and 44) indicative of desired specificity even when the samples were contrived to contain significant amounts of another coronavirus (OC43) as in the Emory contrived samples (Tables 44).

TABLE 41
Emory testing of samples provided by PathogenDx*

| | Heat inactivated | | | | Irradiated cell lysate | | | | KP | KP | | | |
|---|---|---|---|---|---|---|---|---|---|---|---|---|---|
| | 10 | 1 | 0.1 | 0.01 | 10 | 1 | 0.1 | 0.01 | mouthwash | swab | Positive | Negative | 116 |
| | 1 | 2 | 3 | 4 | 5 | 6 | 7 | 8 | 9 | 10 | control | control | X |
| RNAse.P.Probe-pub1.1 | + | + | + | + | + | + | + | + | + | + | + | − | + |
| SARS.COV2-N3-RE1.1 | + | + | + | +/− | + | + | + | + | − | − | + | − | + |
| SARS.COV2-N2-RE1.4 | + | +/− | − | − | + | + | +/− | − | − | − | + | − | + |
| SARS.COV2-N2-RE1.3 | +/− | − | − | − | + | − | − | − | − | − | − | − | + |
| SARS.COV2-N1-RE1.1 | + | +/− | − | − | + | + | +/− | − | − | − | + | − | + |
| 62-Negcont-B | − | − | − | − | − | − | − | − | − | − | − | − | − |
| Overall call | POS | POS | RERUN | NEG | POS | POS | POS | RERUN | NEG | NEG | POS | NEG | POS |

*units are in copies/ml input into the RNA extraction

TABLE 42
PathogenDx run testing*

| | Heat inactivated | | | | Irradiated cell lysate | | | | KP | KP | | | |
|---|---|---|---|---|---|---|---|---|---|---|---|---|---|
| | 10 | 1 | 0.1 | 0.01 | 10 | 1 | 0.1 | 0.01 | mouthwash | swab | Positive | Negative | 116 |
| | 1 | 2 | 3 | 4 | 5 | 6 | 7 | 8 | 9 | 10 | control | control | X |
| RNAse.P.Probe-pub1.1 | + | + | + | + | + | + | + | + | + | + | + | − | + |
| SARS.COV2-N3-RE1.1 | + | + | + | +/− | + | + | + | + | − | − | + | − | + |
| SARS.COV2-N2-RE1.4 | + | +/− | − | − | + | + | +/− | − | − | − | + | − | + |
| SARS.COV2-N2-RE1.3 | +/− | − | − | − | + | − | − | − | − | − | − | − | + |
| SARS.COV2-N1-RE1.1 | + | +/− | − | − | + | + | +/− | − | − | − | + | − | + |
| 62-Negcont-B | − | − | − | − | − | − | − | − | − | − | − | − | − |
| Overall call | POS | POS | RERUN | NEG | POS | POS | POS | RERUN | NEG | NEG | POS | NEG | POS |

*units are in copies/ml input into the RNA extraction

TABLE 43
Summary of PDx Validation Data on Blinded Emory Samples*

| Cov-2 virus | PDx | |
|---|---|---|
| particles/ml VTM | Positive | |
| (prepared by Emory) | calls | Comments |
| 10⁶ | 2 of 2 | |
| 10⁵ | 2 of 2 | |
| 10⁴ | 2 of 2 | |
| 10³ | 1 of 3 | +1 “re-run” |
| 10² | 0 of 3 | +1 “re-runs” |
| 10 | 0 of 3 | +2 “re-runs” |
| 0 | 0 of 3 | No false positives detected |
| | | at 0 CoV-2 virus/ml |
| 0 (OC43) | 0 of 4 | OC43 was not detected at both |
| | | 100 and 1000 OC43 virus/ml |

*PDx Scoring Criteria:
“Positive calls” = (≥2) N probes;
“re-run” = (1) N probe;
“Negative calls” = (0) N probes

### Example 24

### Optimization of the Mini-RV Assay in the 96-Well Format: Approaching 2-Step on Slides

Improvements made in Hybridization/Washing of the Mini-RV array were implemented manually, but via pipetting that can map over directly to the Tecan.

Method—96-well plates
1. Samples were extracted using standard manual protocol (Zymo).
2. Clinical samples used had previously been tested on the Roche COBAS 8800 platform (TriCore). Positive Clinical samples (13<Cq<35) as measured by Roche Cobas, were subdivided into high, med and low Cq levels
3. Contrived samples were prepared from TriCore/Cobas Negatives at 25 gRNA copies/400 μL=62.5 copies/ml
4. RT-PCR was performed using the following conditions:

Asymmetric One-Step RT-PCR conditions

| | | |
|---|---|---|
| i. Access Quick Master mix (2x) | 25 | μl |
| ii. RT-PCR primer | 2 | μl |
| iii. AMV reverse transcriptase | 1 | μl |
| iv. Water | 17 | μl |
| v. Sample | 5 | μl |

Access Quick RT-PCR

Step i. 45° C., 45 min, 1 cycle

Step ii. 94° C., 2 min, 1 cycle

Step iii. 94° C., 30 sec, 40 cycles

Step iv. 55° C., 30 sec, 40 cycles

Step v. 68° C., 30 sec, 40 cycles

Step vi. 68° C., 7 min, 1 cycle

Step vii. 4° C., ∞
5. Manual hybridization and washing of RT-PCR product was performed on the 96-well plates.

TABLE 44
PDx Analysis of Contrived Samples Prepared by Emory

| | Blinded | | | | PDx | |
|---|---|---|---|---|---|---|
| Sample | Sample | Overall | | Cov-2 virus/ml | Positive | |
| # | ID | Call | gRNA/ml | (prepared by Emory) | calls | Comment |
| 1 | 8819 | NEG | OC43, 1000 | 10⁶ | 2 of 2 | |
| 2 | 8833 | POS | 10⁶ | 10⁵ | 2 of 2 | |
| 3 | 8814 | NEG | 0 | 10⁴ | 2 of 2 | |
| 4 | 8826 | NEG | OC43, 1000 | 10³ | 1 of 3 | 1 ″re-run″ |
| 5 | 8812 | NEG | 10 | 10² | 0 of 3 | 1 ″re-run″ |
| 6 | 8809 | POS | 10⁵ | 10 | 0 of 3 | 2 ″re-run″ |
| 7 | 8806 | RERUN | 10² | 0 | 0 of 3 | |
| 8 | 8813 | NEG | OC43, 100 | 0 (OC43) | 0 of 4 | |

| | | | |
|---|---|---|---|
| 9 | 8821 | POS | 10⁴ |
| 10 | 8832 | NEG | 10² |
| 11 | 8808 | RERUN | 10³ |
| 12 | 8804 | NEG | OC43, 100 |
| 13 | 8817 | NEG | 10² |
| 14 | 8820 | RERUN | 10 |
| 15 | 8815 | RERUN | 10 |
| 16 | 8811 | NEG | 0 |
| 17 | 8825 | POS | 10⁵ |
| 18 | 8822 | NEG | 0 |
| 19 | 8818 | POS | 10³ |
| 20 | 8823 | POS | 10⁶ |
| 21 | 8805 | POS | 10⁴ |
| 22 | 8827 | NEG | 10³ |
| 23 | PDx External | POS | |
| | Extraction Control | | |
| 24 | PDx NEG Control | NEG | |

PDx Analysis criteria:
“Positive” = >2 positive N probe signals
“Re-run” = 1 positive N probe signals
“Negative” = 0 positive N probe signals

TABLE 45
Summary of Emory Training Data on Blinded PDx Samples

| | LoD obtained on PDx | |
|---|---|---|
| Analysts | samples (copies/ml) | Contrived sample type |
| Both Emory and PDx | 100 < to > 1000 | γ-Irradiated CoV-2 |
| Both Emory and PDx | 1000 | Heat Inactivated CoV-2 |

### Results—96-Well Plates:

Contrived negative samples TriCore Negative NP at 62.5 copies/ml.

Clinical LoD for Asymmetric One-Step RT-PCR in 96-Well Mini-RV approached matched 2-step PCR with slides (<62.5 copies/mL) (Tables 46 and 47). The LoB appeared to approach slides with manual operation.

Clinical Isolates. TriCore NP Positive and Negative. Analysis of clinical samples showed 91% specificity and 100% selectivity (N1, N2, N3, P) (Tables 48 and 49). Three false Negatives were found to coincide with clinical samples with Cq>30.

TABLE 46
Negative Clinical Samples for LoB (Cq>35)

| DetectX RV Call | — | — | — | — | — | — | — | — | — | — |
|---|---|---|---|---|---|---|---|---|---|---|
| Roche Cq | 35-38 | 35-38 | 35-38 | 35-38 | 35-38 | 35-38 | 35-38 | 35-38 | 35-38 | 35-38 |
| Well description | PATHO- | PATHO- | PATHO- | PATHO- | PATHO- | PATHO- | PATHO- | PATHO- | PATHO- | PATHO- |
| | 001 | 002 | 003 | 004 | 005 | 006 | 007 | 008 | 009 | 010 |
| Threshold: 6K; 4K; 10K | Well 33 | Well 34 | Well 35 | Well 36 | Well 37 | Well 38 | Well 39 | Well 40 | Well 41 | Well 42 |
| 614D-SE-S1-RE1.4 | 1569 | 1739 | 2443 | 941 | 2494 | 796 | 667 | 1391 | 948 | 605 |
| 614G-SE-S1-RE1.4 | 1612 | 2030 | 2198 | 1715 | 2098 | 1355 | 882 | 1639 | 1060 | 743 |
| 614U-SE-S1-RE1.1 | 1041 | 1780 | 2285 | 1432 | 2725 | 1060 | 839 | 1533 | 1448 | 1545 |
| 62-Negcont-B | 190 | 205 | 2040 | 1600 | 2624 | 963 | 1742 | 2710 | 220 | 203 |
| InfA | 261 | 398 | 226 | −7 | −70 | 26 | 477 | 1518 | −65 | −7 |
| InfB | 40 | 41 | 477 | 200 | 1264 | 164 | −80 | −32 | 82 | 236 |
| RNAse.P.Probe-pub1.1 | 64285 | 64356 | 63611 | 62691 | 63406 | 62341 | 61277 | 62185 | 64297 | 64283 |
| SARS.COV2-N1-pub | 5141 | 5626 | 8989 | 6180 | 10508 | 6720 | 6390 | 8833 | 5856 | 8904 |
| SARS.COV2-N1-RE1.1 | 1001 | 1421 | 4129 | 2343 | 2972 | 1623 | 2177 | 3855 | 1408 | 2965 |
| SARS.COV2-N2-RE1.3 | 623 | 773 | 1945 | 979 | 2509 | 1321 | 1536 | 1205 | 866 | 434 |
| SARS.COV2-N2-RE1.4 | 667 | 826 | 2133 | 1538 | 2572 | 2536 | 1126 | 2320 | 533 | 617 |
| SARS.COV2-N3-RE1.1 | 3914 | 4892 | 7773 | 7221 | 3844 | 2518 | 7985 | 5725 | 2888 | 2671 |
| DetectX RV Call | — | RERUN | — | — | — | — | — | — | — | — |
| Roche Cq | 35-38 | 35-38 | 35-38 | 35-38 | 35-38 | 35-38 | 35-38 | 35-38 | 35-38 | 35-38 |
| Well description | PATHO- | PATHO- | PATHO- | PATHO- | PATHO- | PATHO- | PATHO- | PATHO- | PATHO- | PATHO- |
| | 011 | 012 | 013 | 014 | 015 | 016 | 017 | 018 | 019 | 020 |
| Threshold: 6K; 4K; 10K | Well 43 | Well 44 | Well 45 | Well 46 | Well 47 | Well 48 | Well 49 | Well 50 | Well 51 | Well 52 |
| 614D-SE-S1-RE1.4 | 1832 | 1227 | 2043 | 829 | 1640 | 1093 | 1317 | 1577 | 2254 | 1666 |
| 614G-SE-S1-RE1.4 | 2022 | 1462 | 1352 | 644 | 1926 | 2411 | 1804 | 1816 | 2135 | 1623 |
| 614U-SE-S1-RE1.1 | 1629 | 1272 | 1911 | 1069 | 2048 | 2077 | 1449 | 1101 | 2227 | 1296 |
| 62-Negcont-B | 1216 | 877 | 2236 | 1336 | 3313 | 776 | −33 | 94 | 1845 | 1139 |
| InfA | 1509 | 71 | 285 | 733 | −83 | 56 | 295 | 71 | 243 | 473 |
| InfB | 10 | 189 | 551 | −3 | 487 | 24 | 92 | 4 | 374 | −14 |
| RNAse.P.Probe-pub1.1 | 64134 | 62543 | 63437 | 62572 | 62492 | 62954 | 64236 | 64248 | 36197 | 62468 |
| SARS.COV2-N1-pub | 5137 | 5810 | 6049 | 6931 | 10119 | 10668 | 3815 | 5094 | 2547 | 4992 |
| SARS.COV2-N1-RE1.1 | 2550 | 2272 | 2627 | 2283 | 3367 | 2808 | 833 | 616 | 1754 | 1357 |
| SARS.COV2-N2-RE1.3 | 2887 | 1427 | 1618 | 1343 | 1896 | 3665 | 339 | 210 | 1119 | 1316 |
| SARS.COV2-N2-RE1.4 | 3226 | 1625 | 2066 | 832 | 2308 | 2195 | 636 | 291 | 1453 | 1201 |
| SARS.COV2-N3-RE1.1 | 7911 | 10842 | 4860 | 5189 | 8967 | 8296 | 2476 | 5302 | 2325 | 5931 |
| DetectX RV Call | — | — | — | RERUN | — | — | RERUN | — | RERUN | — |
| Roche Cq | 35-38 | 35-38 | 35-38 | 35-38 | 35-38 | 35-38 | 35-38 | 35-38 | 35-38 | 35-38 |
| Well description | PATHO- | PATHO- | PATHO- | PATHO- | PATHO- | PATHO- | PATHO- | PATHO- | PATHO- | PATHO- |
| | 021 | 022 | 023 | 024 | 025 | 026 | 027 | 028 | 029 | 030 |
| Threshold: 6K; 4K; 10K | Well 53 | Well 54 | Well 55 | Well 56 | Well 57 | Well 58 | Well 59 | Well 60 | Well 61 | Well 62 |
| 614D-SE-S1-RE1.4 | 2209 | 1557 | 1528 | 1917 | 1496 | 1546 | 1512 | 1343 | 2429 | 1993 |
| 614G-SE-S1-RE1.4 | 1908 | 1751 | 2100 | 6149 | 1872 | 1574 | 1127 | 1448 | 2875 | 1367 |
| 614U-SE-S1-RE1.1 | 2539 | 1673 | 2186 | 1831 | 981 | 1300 | 1167 | 1500 | 2672 | 2048 |
| 62-Negcont-B | 3050 | 1662 | 1365 | 2221 | 39 | 39 | 1331 | 909 | 1299 | 1813 |
| InfA | −36 | −15 | 426 | 1138 | 240 | 85 | 121 | 132 | 750 | −16 |
| InfB | 1320 | 642 | 530 | 113 | 87 | 215 | 20 | 178 | −58 | 1192 |
| RNAse.P.Probe-pub1.1 | 62897 | 62942 | 61442 | 62268 | 64177 | 63884 | 62980 | 62404 | 63589 | 63575 |
| SARS.COV2-N1-pub | 7549 | 5376 | 6517 | 8961 | 10142 | 6738 | 7125 | 8100 | 9074 | 8946 |
| SARS.COV2-N1-RE1.1 | 3326 | 2497 | 1688 | 2222 | 1925 | 1840 | 3237 | 2777 | 2373 | 3262 |
| SARS.COV2-N2-RE1.3 | 3254 | 1772 | 1884 | 1791 | 887 | 1148 | 1984 | 2587 | 2259 | 3146 |
| SARS.COV2-N2-RE1.4 | 3584 | 1620 | 2119 | 2114 | 1194 | 1183 | 2023 | 986 | 2432 | 2223 |
| SARS.COV2-N3-RE1.1 | 7887 | 6507 | 5352 | 11168 | 1388 | 1935 | 11533 | 9546 | 11583 | 8293 |

TABLE 47
Contrived Samples for LOD (at 62.5 copies/ml)

| DETECTX RV Call | + | + | + | + | + | _ + | + | + | + | + | + | + |
|---|---|---|---|---|---|---|---|---|---|---|---|---|
| Roche Cq | na | na | na | na | na | na | na | na | na | na | na | na |
| Well description | LoD | LoD | LoD | LoD | LoD | LoD | LoD | LoD | LoD | LoD | LoD | LoD |
| Threshold: 6K; 4K; 10K | Well 65 | Well 66 | Well 67 | Well 68 | Well 69 | Well 70 | Well 71 | Well 72 | Well 73 | Well 74 | Well 75 | Well 76 |
| 614D-SE-S1-RE1.4 | 3484 | 3245 | 3556 | 3327 | 2871 | 2955 | 3215 | 4688 | 2376 | 3106 | 3127 | 3608 |
| 614G-SE-S1-RE1.4 | 1642 | 1542 | 1745 | 1886 | 2449 | 1759 | 1776 | 2333 | 1610 | 1892 | 1186 | 2330 |
| 614U-SE-S1-RE1.1 | 6504 | 8639 | 5886 | 6293 | 7253 | 5724 | 6247 | 9771 | 5196 | 4319 | 5278 | 5000 |
| 62-Negcont-B | 174 | 236 | 1229 | 1935 | 1646 | 1417 | 1720 | 1462 | −11 | 10 | 457 | 1441 |
| InfA.7.univ-pubRev | 304 | 756 | 516 | −250 | 397 | 578 | −58 | 1252 | 796 | 447 | 698 | −8 |
| InfB.8.univ-pub | −19 | 8 | −35 | 285 | 137 | −128 | 489 | −186 | 86 | 72 | −61 | 106 |
| RNAse.P.Probe-pub1.1 | 64140 | 63980 | 63208 | 62801 | 62778 | 62654 | 62379 | 62257 | 64023 | 64035 | 62275 | 62997 |
| SARS.COV2-N1-pub | 47158 | 50266 | 47720 | 49275 | 48853 | 46530 | 47431 | 50286 | 51818 | 50304 | 46915 | 47317 |
| SARS.COV2-N1-RE1.1 | 30375 | 38749 | 38833 | 39965 | 40642 | 38697 | 39535 | 38895 | 39039 | 35832 | 37634 | 37858 |
| SARS.COV2-N2-RE1.3 | 12315 | 11772 | 16989 | 18934 | 22002 | 18178 | 20475 | 29839 | 17666 | 10185 | 21361 | 15242 |
| SARS.COV2-N2-RE1.4 | 41784 | 43045 | 45998 | 48386 | 50500 | 45570 | 48290 | 52478 | 41701 | 40695 | 49169 | 46306 |
| SARS.COV2-N3-RE1.1 | 42343 | 44361 | 47653 | 47233 | 50989 | 45577 | 51288 | 55057 | 44002 | 43018 | 47688 | 48393 |
| DetectX RV Call | + | + | + | + | + | + | + | + | + | + | + | + |
| Roche Cq | na | na | na | na | na | na | na | na | na | na | na | na |
| Well description | LoD | LoD | LoD | LoD | LoD | LoD | LoD | LoD | LoD | LoD | LoD | LoD |
| Threshold: 6K; 4K; 10K | Well 77 | Well 78 | Well 79 | Well 80 | Well 81 | Well 82 | Well 83 | Well 84 | Well 85 | Well 86 | Well 87 | Well 88 |
| 614D-SE-S1-RE1.4 | 2706 | 2104 | 2870 | 3725 | 4289 | 4027 | 3342 | 3156 | 3265 | 2343 | 3175 | 4059 |
| 614G-SE-S1-RE1.4 | 1711 | 1502 | 1344 | 2229 | 1739 | 1900 | 1758 | 1791 | 1595 | 1154 | 2043 | 2621 |
| 614U-SE-S1-RE1.1 | 4927 | 4992 | 3914 | 5663 | 7510 | 7646 | 5182 | 4995 | 5502 | 4124 | 6109 | 8547 |
| 62-Negcont-B | 869 | 1174 | 3551 | 2318 | 3 | 298 | 1737 | 1479 | 1561 | 1242 | 1181 | 3322 |
| InfA.7.univ-pubRev | −97 | −55 | 1234 | 827 | 675 | 600 | 728 | 447 | 399 | 61 | 1440 | 342 |
| InfB.8.univ-pub | 171 | 74 | 258 | 573 | 90 | 59 | 196 | −290 | 94 | 184 | 31 | 472 |
| RNAse.P.Probe-pub1.1 | 62233 | 62560 | 61052 | 62383 | 64039 | 63991 | 62844 | 62609 | 62597 | 63192 | 61990 | 62505 |
| SARS.COV2-N1-pub | 44165 | 47728 | 50017 | 48117 | 59064 | 55916 | 49944 | 50132 | 47374 | 48650 | 46566 | 53293 |
| SARS.COV2-N1-RE1.1 | 34365 | 39082 | 35650 | 39708 | 41211 | 39946 | 40245 | 39444 | 38059 | 40858 | 38973 | 40578 |
| SARS.COV2-N2-RE1.3 | 14193 | 17099 | 17656 | 17698 | 20779 | 20034 | 22299 | 20606 | 20893 | 20264 | 23322 | 29091 |
| SARS.COV2-N2-RE1.4 | 45517 | 45484 | 47099 | 48945 | 46318 | 46005 | 47927 | 49480 | 47143 | 47961 | 49109 | 52795 |
| SARS.COV2-N3-RE1.1 | 49069 | 46998 | 49050 | 50124 | 50115 | 47555 | 47408 | 46946 | 46820 | 46577 | 49863 | 54869 |

TABLE 48
96-Well Mini-RV analysis of positive clinical isolates for optimized manual hybridization-wash

| DetectX | Cycle | Well | Threshold: | 614D-SE- | 614G-SE- | 614U-SE- |
|---|---|---|---|---|---|---|
| RV Call | number | description | 6K; 4K; 10K | S1-RE1.4 | SI-RE1.4 | S1-RE1.1 |
| + | 14.46 | 217215 | Well 1 | 10035 | 61638 | 62910 |
| + | 14.06 | 217372 | Well 2 | 8625 | 61438 | 63436 |
| + | 13.75 | 214495 | Well 3 | 8788 | 56914 | 61021 |
| + | 14.43 | 217142 | Well 4 | 11462 | 60770 | 62023 |
| + | 14.93 | 215025 | Well 5 | 9763 | 56460 | 61720 |
| + | 15.97 | 217179 | Well 6 | 6412 | 51400 | 61943 |
| + | 16 | 216036 | Well 7 | 7039 | 47960 | 60133 |
| + | 15.72 | 216106 | Well 8 | 7966 | 54076 | 61563 |
| + | 15.85 | 216019 | Well 9 | 6044 | 43578 | 63518 |
| + | 15.07 | 216052 | Well 10 | 9204 | 59996 | 63589 |
| + | 27.5 | 217370 | Well 11 | 1137 | 11291 | 38534 |
| + | 27.03 | 217358 | Well 12 | 1798 | 4990 | 19265 |
| + | 27.04 | 217213 | Well 13 | 760 | 1314 | 1750 |
| + | 24.09 | 217235 | Well 14 | 3098 | 24720 | 49041 |
| + | 20.27 | 217347 | Well 15 | 3358 | 39157 | 61303 |
| + | 22.07 | 217348 | Well 16 | 3648 | 37331 | 61276 |
| + | 20.48 | 217354 | Well 17 | 8164 | 23487 | 40566 |
| + | 19.25 | 217353 | Well 18 | 5120 | 14394 | 35556 |
| + | 15.63 | 217355 | Well 19 | 18839 | 47553 | 62866 |
| + | 28.48 | 217351 | Well 20 | 1242 | 1800 | 1873 |
| − | 31.03 | 217345 | Well 21 | 795 | 693 | 1101 |
| + | 32.34 | 217217 | Well 22 | 1515 | 1739 | 1601 |
| + | 30.53 | 217212 | Well 23 | 1974 | 2027 | 2018 |
| + | 31.74 | 217210 | Well 24 | 1570 | 2371 | 1764 |
| + | 30.48 | 217344 | Well 25 | 1161 | 1962 | 2068 |
| − | 32.6 | 217357 | Well 26 | 1314 | 1597 | 1148 |
| RERUN | 31.92 | 217360 | Well 27 | 1343 | 1577 | 1594 |
| − | 34.35 | 217356 | Well 28 | 1202 | 1693 | 1303 |
| + | 30.68 | 216048 | Well 29 | 1813 | 2761 | 5045 |
| + | 32.13 | 216133 | Well 30 | 1303 | 1478 | 1206 |
| RERUN | | NTC | Well 31 | 2631 | 2882 | 2009 |
| RERUN | | NTC | Well 32 | 2434 | 2264 | 2378 |
| − | 35-38 | PATHO-001 | Well 33 | 1569 | 1612 | 1041 |
| − | 35-38 | PATHO-002 | Well 34 | 1739 | 2030 | 1780 |
| − | 35-38 | PATHO-003 | Well 35 | 2443 | 2198 | 2285 |
| − | 35-38 | PATHO-004 | Well 36 | 941 | 1715 | 1432 |
| − | 35-38 | PATHO-005 | Well 37 | 2494 | 2098 | 2725 |
| − | 35-38 | PATHO-006 | Well 38 | 796 | 1355 | 1060 |
| − | 35-38 | PATHO-007 | Well 39 | 667 | 882 | 839 |
| − | 35-38 | PATHO-008 | Well 40 | 1391 | 1639 | 1533 |
| − | 35-38 | PATHO-009 | Well 41 | 948 | 1060 | 1448 |
| − | 35-38 | PATHO-010 | Well 42 | 605 | 743 | 1545 |
| − | 35-38 | PATHO-011 | Well 43 | 1832 | 2022 | 1629 |
| RERUN | 35-38 | PATHO-012 | Well 44 | 1227 | 1462 | 1272 |
| − | 35-38 | PATHO-013 | Well 45 | 2043 | 1352 | 1911 |
| − | 35-38 | PATHO-014 | Well 46 | 829 | 644 | 1069 |
| − | 35-38 | PATHO-015 | Well 47 | 1640 | 1926 | 2048 |
| − | 35-38 | PATHO-016 | Well 48 | 1093 | 2411 | 2077 |
| − | 35-38 | PATHO-017 | Well 49 | 1317 | 1804 | 1449 |
| − | 35-38 | PATHO-018 | Well 50 | 1577 | 1816 | 1101 |
| − | 35-38 | PATHO-019 | Well 51 | 2254 | 2135 | 2227 |
| − | 35-38 | PATHO-020 | Well 52 | 1666 | 1623 | 1296 |
| − | 35-38 | PATHO-021 | Well 53 | 2209 | 1908 | 2539 |
| − | 35-38 | PATHO-022 | Well 54 | 1557 | 1751 | 1673 |
| − | 35-38 | PATHO-023 | Well 55 | 1528 | 2100 | 2186 |
| RERUN | 35-38 | PATHO-024 | Well 56 | 1917 | 6149 | 1831 |
| − | 35-38 | PATHO-025 | Well 57 | 1496 | 1872 | 981 |
| − | 35-38 | PATHO-026 | Well 58 | 1546 | 1574 | 1300 |
| RERUN | 35-38 | PATHO-027 | Well 59 | 1512 | 1127 | 1167 |
| − | 35-38 | PATHO-028 | Well 60 | 1343 | 1448 | 1500 |
| RERUN | 35-38 | PATHO-029 | Well 61 | 2429 | 2875 | 2672 |
| − | 35-38 | PATHO-030 | Well 62 | 1993 | 1367 | 2048 |
| RERUN | | NTC | Well 63 | 1742 | 1938 | 1771 |
| RERUN | | NTC | Well 64 | 2740 | 2525 | 2529 |
| + | | LoD | Well 65 | 3484 | 1642 | 6504 |
| + | | LoD | Well 66 | 3245 | 1542 | 8639 |
| + | | LoD | Well 67 | 3556 | 1745 | 5886 |
| + | | LoD | Well 68 | 3327 | 1886 | 6293 |
| + | | LoD | Well 69 | 2871 | 2449 | 7253 |
| + | | LoD | Well 70 | 2955 | 1759 | 5724 |
| + | | LoD | Well 71 | 3215 | 1776 | 6247 |
| + | | LoD | Well 72 | 4688 | 2333 | 9771 |
| + | | LoD | Well 73 | 2376 | 1610 | 5196 |
| + | | LoD | Well 74 | 3106 | 1892 | 4319 |
| + | | LoD | Well 75 | 3127 | 1186 | 5278 |
| + | | LoD | Well 76 | 3608 | 2330 | 5000 |
| + | | LoD | Well 77 | 2706 | 1711 | 4927 |
| + | | LoD | Well 78 | 2104 | 1502 | 4992 |
| + | | LoD | Well 79 | 2870 | 1344 | 3914 |
| + | | LoD | Well 80 | 3725 | 2229 | 5663 |
| + | | LoD | Well 81 | 4289 | 1739 | 7510 |
| + | | LoD | Well 82 | 4027 | 1900 | 7646 |
| + | | LoD | Well 83 | 3342 | 1758 | 5182 |
| + | | LoD | Well 84 | 3156 | 1791 | 4995 |
| + | | LoD | Well 85 | 3265 | 1595 | 5502 |
| + | | LoD | Well 86 | 2343 | 1154 | 4124 |
| + | | LoD | Well 87 | 3175 | 2043 | 6109 |
| + | | LoD | Well 88 | 4059 | 2621 | 8547 |
| − | | Empty | Well 89 | 1039 | 549 | 1173 |
| − | | | Well 90 | 1768 | 2246 | 2237 |
| RERUN | | | Well 91 | 1499 | 696 | 1323 |
| − | | | Well 92 | 1206 | 1493 | 1182 |
| − | | | Well 93 | 2039 | 1451 | 2308 |
| − | | | Well 94 | 1394 | 1811 | 1402 |
| + | | | Well 95 | 1397 | 1315 | 1064 |
| − | | | Well 96 | 877 | 1388 | 1448 |
| | DetectX | 62- | InfA.7. | InfB.8. | RNAse.P. | SARS. |
| | RV Call | Negcont-B | univ-pubRev | univ-pub | Probe-pub1.1 | COV2-N1-pub |
| | + | 66 | 274 | 478 | 63613 | 63199 |
| | + | 1 | 112 | 538 | 64162 | 63751 |
| | + | 862 | 78 | 6 | 61649 | 61309 |
| | + | 1203 | −140 | 215 | 62686 | 62416 |
| | + | 1261 | 67 | 576 | 62411 | 62047 |
| | + | 780 | 581 | 75 | 62684 | 62266 |
| | + | 1325 | 864 | −388 | 60847 | 60465 |
| | + | 2049 | 725 | −220 | 62314 | 61917 |
| | + | 273 | 73 | 610 | 52569 | 63846 |
| | + | −2 | 458 | 761 | 46378 | 63902 |
| | + | 1553 | 595 | −15 | 62525 | 61794 |
| | + | 1521 | 1130 | −56 | 62947 | 50482 |
| | + | 1460 | 1636 | −4 | 62057 | 35143 |
| | + | 1445 | 1605 | −29 | 63607 | 63324 |
| | + | 1795 | 1593 | −137 | 62010 | 61660 |
| | + | 2420 | 947 | −126 | 50836 | 61634 |
| | + | 1113 | 909 | −26 | 63983 | 63320 |
| | + | 597 | 970 | −22 | 63914 | 56749 |
| | + | 1317 | 637 | −60 | 63679 | 63303 |
| | + | 4748 | 253 | −73 | 63108 | 10421 |
| | − | 1171 | 360 | −2 | 62688 | 3861 |
| | + | 5128 | 737 | −99 | 63040 | 5924 |
| | + | 5598 | 1254 | −118 | 62350 | 4926 |
| | + | 5923 | 596 | −72 | 62685 | 7121 |
| | + | −2 | 391 | 39 | 61127 | 14979 |
| | − | 269 | 513 | −34 | 64223 | 6064 |
| | RERUN | 1022 | 1649 | −63 | 62233 | 3443 |
| | − | 1150 | 777 | −22 | 62705 | 4624 |
| | + | 2341 | −28 | 1070 | 62700 | 40330 |
| | + | 1034 | 179 | 148 | 62487 | 13099 |
| | RERUN | 2602 | 2150 | −186 | 9239 | 8657 |
| | RERUN | 3401 | 173 | 1250 | 10046 | 12014 |
| | − | 190 | 261 | 40 | 64285 | 5141 |
| | − | 205 | 398 | 41 | 64356 | 5626 |
| | − | 2040 | 226 | 477 | 63611 | 8989 |
| | − | 1600 | −7 | 200 | 62691 | 6180 |
| | − | 2624 | −70 | 1264 | 63406 | 10508 |
| | − | 963 | 26 | 164 | 62341 | 6720 |
| | − | 1742 | 477 | −80 | 61277 | 6390 |
| | − | 2710 | 1518 | −32 | 62185 | 8833 |
| | − | 220 | −65 | 82 | 64297 | 5856 |
| | − | 203 | −7 | 236 | 64283 | 8904 |
| | − | 1216 | 1509 | 10 | 64134 | 5137 |
| | RERUN | 877 | 71 | 189 | 62543 | 5810 |
| | − | 2236 | 285 | 551 | 63437 | 6049 |
| | − | 1336 | 733 | −3 | 62572 | 6931 |
| | − | 3313 | −83 | 487 | 62492 | 10119 |
| | − | 776 | 56 | 24 | 62954 | 10668 |
| | − | −33 | 295 | 92 | 64236 | 3815 |
| | − | 94 | 71 | 4 | 64248 | 5094 |
| | − | 1845 | 243 | 374 | 36197 | 2547 |
| | − | 1139 | 473 | −14 | 62468 | 4992 |
| | − | 3050 | −36 | 1320 | 62897 | 7549 |
| | − | 1662 | −15 | 642 | 62942 | 5376 |
| | − | 1365 | 426 | 530 | 61442 | 6517 |
| | RERUN | 2221 | 1138 | 113 | 62268 | 8961 |
| | − | 39 | 240 | 87 | 64177 | 10142 |
| | − | 39 | 85 | 215 | 63884 | 6738 |
| | RERUN | 1331 | 121 | 20 | 62980 | 7125 |
| | − | 909 | 132 | 178 | 62404 | 8100 |
| | RERUN | 1299 | 750 | −58 | 63589 | 9074 |
| | − | 1813 | −16 | 1192 | 63575 | 8946 |
| | RERUN | 917 | 1021 | 468 | 6875 | 12850 |
| | RERUN | 1907 | 1542 | −41 | 7237 | 15457 |
| | + | 174 | 304 | −19 | 64140 | 47158 |
| | + | 236 | 756 | 8 | 63980 | 50266 |
| | + | 1229 | 516 | −35 | 63208 | 47720 |
| | + | 1935 | −250 | 285 | 62801 | 49275 |
| | + | 1646 | 397 | 137 | 62778 | 48853 |
| | + | 1417 | 578 | −128 | 62654 | 46530 |
| | + | 1720 | −58 | 489 | 62379 | 47431 |
| | + | 1462 | 1252 | −186 | 62257 | 50286 |
| | + | −11 | 796 | 86 | 64023 | 51818 |
| | + | 10 | 447 | 72 | 64035 | 50304 |
| | + | 457 | 698 | −61 | 62275 | 46915 |
| | + | 1441 | −8 | 106 | 62997 | 47317 |
| | + | 869 | −97 | 171 | 62233 | 44165 |
| | + | 1174 | −55 | 74 | 62560 | 47728 |
| | + | 3551 | 1234 | 258 | 61052 | 50017 |
| | + | 2318 | 827 | 573 | 62383 | 48117 |
| | + | 3 | 675 | 90 | 64039 | 59064 |
| | + | 298 | 600 | 59 | 63991 | 55916 |
| | + | 1737 | 728 | 196 | 62844 | 49944 |
| | + | 1479 | 447 | −290 | 62609 | 50132 |
| | + | 1561 | 399 | 94 | 62597 | 47374 |
| | + | 1242 | 61 | 184 | 63192 | 48650 |
| | + | 1181 | 1440 | 31 | 61990 | 46566 |
| | + | 3322 | 342 | 472 | 62505 | 53293 |
| | − | 461 | −6 | 679 | 5670 | 9646 |
| | − | −11 | 709 | 233 | 8598 | 16006 |
| | RERUN | 1346 | −113 | 275 | 11659 | 15042 |
| | − | 837 | 84 | 5 | 4996 | 10852 |
| | − | 2237 | 45 | 961 | 6829 | 11735 |
| | − | 1262 | 202 | 228 | 6505 | 9046 |
| | + | 1532 | 128 | 339 | 6512 | 12167 |
| | − | 1569 | 948 | −143 | 3207 | 5496 |
| | | DetectX | SARS. | SARS. | SARS. | SARS. |
| | | RV Call | COV2-N1-RE1.1 | COV2-N2-RE1.3 | COV2-N2-RE1.4 | COV2-N3-RE1.1 |
| | | + | 63343 | 62085 | 63263 | 63477 |
| | | + | 63894 | 55180 | 63719 | 63939 |
| | | + | 61533 | 61277 | 61269 | 61502 |
| | | + | 62557 | 62247 | 62278 | 62474 |
| | | + | 62251 | 62019 | 62101 | 62303 |
| | | + | 62478 | 62202 | 62244 | 62471 |
| | | + | 60691 | 60479 | 60617 | 60719 |
| | | + | 62146 | 61836 | 61828 | 62130 |
| | | + | 63859 | 60277 | 63700 | 63833 |
| | | + | 63996 | 51748 | 63771 | 63995 |
| | | + | 46992 | 36999 | 62044 | 62190 |
| | | + | 40029 | 16939 | 54703 | 47822 |
| | | + | 18255 | 4099 | 20504 | 22385 |
| | | + | 61686 | 40548 | 63209 | 63376 |
| | | + | 61858 | 54570 | 61591 | 61736 |
| | | + | 61836 | 54169 | 61566 | 61766 |
| | | + | 41200 | 23301 | 53807 | 54707 |
| | | + | 41288 | 18293 | 47393 | 53551 |
| | | + | 63441 | 44179 | 63217 | 63376 |
| | | + | 7902 | 4135 | 7594 | 12818 |
| | | − | 1476 | 1809 | 2671 | 6874 |
| | | + | 8463 | 6573 | 3962 | 9896 |
| | | + | 15918 | 8517 | 5099 | 14687 |
| | | + | 9032 | 4278 | 2747 | 10886 |
| | | + | 3692 | 4607 | 10158 | 15234 |
| | | − | 1614 | 739 | 681 | 4211 |
| | | RERUN | 543 | 1984 | 2478 | 12966 |
| | | − | 1553 | 1222 | 1475 | 7812 |
| | | + | 27405 | 9906 | 38716 | 41757 |
| | | + | 5329 | 2873 | 4488 | 14454 |
| | | RERUN | 3174 | 4261 | 3911 | 4639 |
| | | RERUN | 5307 | 1872 | 2373 | 14001 |
| | | − | 1001 | 623 | 667 | 3914 |
| | | − | 1421 | 773 | 826 | 4892 |
| | | − | 4129 | 1945 | 2133 | 7773 |
| | | − | 2343 | 979 | 1538 | 7221 |
| | | − | 2972 | 2509 | 2572 | 3844 |
| | | − | 1623 | 1321 | 2536 | 2518 |
| | | − | 2177 | 1536 | 1126 | 7985 |
| | | − | 3855 | 1205 | 2320 | 5725 |
| | | − | 1408 | 866 | 533 | 2888 |
| | | − | 2965 | 434 | 617 | 2671 |
| | | − | 2550 | 2887 | 3226 | 7911 |
| | | RERUN | 2272 | 1427 | 1625 | 10842 |
| | | − | 2627 | 1618 | 2066 | 4860 |
| | | − | 2283 | 1343 | 832 | 5189 |
| | | − | 3367 | 1896 | 2308 | 8967 |
| | | − | 2808 | 3665 | 2195 | 8296 |
| | | − | 833 | 339 | 636 | 2476 |
| | | − | 616 | 210 | 291 | 5302 |
| | | − | 1754 | 1119 | 1453 | 2325 |
| | | − | 1357 | 1316 | 1201 | 5931 |
| | | − | 3326 | 3254 | 3584 | 7887 |
| | | − | 2497 | 1772 | 1620 | 6507 |
| | | − | 1688 | 1884 | 2119 | 5352 |
| | | RERUN | 2222 | 1791 | 2114 | 11168 |
| | | − | 1925 | 887 | 1194 | 1388 |
| | | − | 1840 | 1148 | 1183 | 1935 |
| | | RERUN | 3237 | 1984 | 2023 | 11533 |
| | | − | 2777 | 2587 | 986 | 9546 |
| | | RERUN | 2373 | 2259 | 2432 | 11583 |
| | | − | 3262 | 3146 | 2223 | 8293 |
| | | RERUN | 5624 | 1504 | 1747 | 11872 |
| | | RERUN | 4113 | 2688 | 3144 | 19038 |
| | | + | 30375 | 12315 | 41784 | 42343 |
| | | + | 38749 | 11772 | 43045 | 44361 |
| | | + | 38833 | 16989 | 45998 | 47653 |
| | | + | 39965 | 18934 | 48386 | 47233 |
| | | + | 40642 | 22002 | 50500 | 50989 |
| | | + | 38697 | 18178 | 45570 | 45577 |
| | | + | 39535 | 20475 | 48290 | 51288 |
| | | + | 38895 | 29839 | 52478 | 55057 |
| | | + | 39039 | 17666 | 41701 | 44002 |
| | | + | 35832 | 10185 | 40695 | 43018 |
| | | + | 37634 | 21361 | 49169 | 47688 |
| | | + | 37858 | 15242 | 46306 | 48393 |
| | | + | 34365 | 14193 | 45517 | 49069 |
| | | + | 39082 | 17099 | 45484 | 46998 |
| | | + | 35650 | 17656 | 47099 | 49050 |
| | | + | 39708 | 17698 | 48945 | 50124 |
| | | + | 41211 | 20779 | 46318 | 50115 |
| | | + | 39946 | 20034 | 46005 | 47555 |
| | | + | 40245 | 22299 | 47927 | 47408 |
| | | + | 39444 | 20606 | 49480 | 46946 |
| | | + | 38059 | 20893 | 47143 | 46820 |
| | | + | 40858 | 20264 | 47961 | 46577 |
| | | + | 38973 | 23322 | 49109 | 49863 |
| | | + | 40578 | 29091 | 52795 | 54869 |
| | | − | 1322 | 864 | 1030 | 873 |
| | | − | 4014 | 973 | 1306 | 9239 |
| | | RERUN | 3282 | 3908 | 2298 | 11617 |
| | | − | 4432 | 1429 | 1082 | 3273 |
| | | − | 3323 | 2362 | 1734 | 2942 |
| | | − | 2507 | 1511 | 1643 | 3843 |
| | | + | 4989 | 6348 | 2609 | 19776 |
| | | − | 1436 | 2221 | 4107 | 3768 |

TABLE 49
Analysis of clinical isolates

| | | Standard | | Standard | | | | | | | | | | |
|---|---|---|---|---|---|---|---|---|---|---|---|---|---|---|
| Probe | Average | deviation | Average | deviation | (a) True | (b) False | (c) False | (d) True | | | Sensi- | Speci- | | |
| name | positives | positives | negatives | negatives | positive | positive | negative | negative | LoB | LoD | tivity | ficity | PPV | NPV |
| RNase P | 61474 | 4084 | 62272 | 5002 | 30 | 0 | 0 | 30 | 70501 | 14045 | 100 | 100 | 100 | 100 |
| N1 | 38209 | 26123 | 2317 | 871 | 30 | 0 | 3 | 30 | 3749 | 46351 | 91 | 100 | 100 | 91 |
| N21.3 | 31303 | 25588 | 1624 | 882 | 30 | 0 | 3 | 30 | 3075 | 44666 | 91 | 100 | 100 | 91 |
| N21.4 | 39763 | 27126 | 1672 | 827 | 30 | 0 | 3 | 30 | 3033 | 46831 | 91 | 100 | 100 | 91 |
| N3 | 42311 | 24102 | 6224 | 3041 | 30 | 0 | 3 | 30 | 11226 | 47045 | 91 | 100 | 100 | 91 |
| Overall Call | N/A | N/A | N/A | N/A | 30 | 0 | 3 | 30 | N/A | N/A | 91 | 100 | 100 | 91 |

### Example 25

### Well-to-Well Cross Contamination in Mini-RV Workflow

To test the potential of cross contamination of the negative sample wells with the positive samples various checkerboard patterns were tested in the Min-RV 96-well workflow. The goals of these experiments were:
1. Measure the rate of well-to-well transfer of high copy number positive samples into negatives.
2. Quantitate the baseline cross contamination rate due to well-well transfer during processing.
3. Perform a full workflow (Zymo→Asymmetric One-Step RT-PCR→Hybridization/Wash→Sensovation 96-well imaging) in 96-well format.
4. Testing of positive and negative samples were performed in a checkerboard pattern.
5. Measure rate of well-to-well transfer during manual and automated (Tecan) workflows.

Four different checkerboard patterns were tested in duplicate (8×96 data points) for pooled positive Boca NP swab samples (in VTM) extracted on the Tecan robot. Table 50 shows the four checkerboard patterns used with the positive wells shown in bold numerals. Tables 51 and 52 show the full representative data sets for Checkerboard patterns 2 and 3. These data revealed no well-to-well cross contamination across all 8 sets of experiments (602 negatives) and are summarized in Table 53.

### Increase Efficiency of Asymmetric One-Step RT-PCR Obtained at 39 PCR Cycles/Reaction

To reduce time needed to perform the assay, temperature and time parameters in the Asymmetric One-Step RT-PCR were varied. The general change in the method steps were as follows:
1. Hold the total number of PCR cycles to <40, to minimize the perceived risk of false positives, which might occur during >40 cycles of endpoint PCR.
2. Test the increase of Taq Polymerase 2× and 3× in the current Mini-RV Asymmetric One-Step RT-PCR master mix, to reduce product mediated polymerase inhibition at high cycle number.
3. Test the effect of a 30% reduction of heat denaturation time in the PCR cycle (30 sec->20 sec) to reduce the thermal footprint accumulated by Taq over <40 cycles.
4. Determine using the present Mini-RV Asymmetric One-Step RT-PCR Reaction, the effect of a 2× and 3× increase in [Taq] on analytical LoD and clinical sensitivity, as assessed by hybridization in the 96-Well format
5. Determine using the present Mini-RV Asymmetric One-Step RT-PCR Reaction, the effect of a reduction of heat denaturation time from 30 sec to 20 sec on analytical LoD and clinical sensitivity, as assessed by hybridization in the 96-Well format.

TABLE 50
Checkerboard pattern for testing well-to-well cross contamination in Mini-RV workflow

| Checkerboard 1 | | | | | | | | | | | | | 24 Positive Samples | | | | |
|---|---|---|---|---|---|---|---|---|---|---|---|---|---|---|---|---|---|
| | 1 | 2 | 3 | 4 | 5 | 6 | 7 | 8 | 9 | 10 | 11 | 12 | | | | | |
| A | 1 | 9 | 17 | 25 | 33 | 41 | 49 | 57 | 65 | 73 | 81 | 89 | | | | | |
| B | 2 | 10 | 18 | 26 | 34 | 42 | 50 | 58 | 66 | 74 | 82 | 90 | One Step PCR | | | | |
| C | 3 | 11 | 19 | 27 | 35 | 43 | 51 | 59 | 67 | 75 | 83 | 91 | Reagent | 1 | X | 100 | X |
| D | 4 | 12 | 20 | 28 | 36 | 44 | 52 | 60 | 68 | 76 | 84 | 92 | Master Mix | 25 | μL | 2500 | μL |
| E | 5 | 13 | 21 | 29 | 37 | 45 | 53 | 61 | 69 | 77 | 85 | 93 | Primer | 2 | μL | 200 | μL |
| F | 6 | 14 | 22 | 30 | 38 | 46 | 54 | 62 | 70 | 78 | 86 | 94 | AMV | 1 | μL | 100 | μL |
| G | 7 | 15 | 23 | 31 | 39 | 47 | 55 | 63 | 71 | 79 | 87 | 95 | H2O | 17 | μL | 1700 | μL |
| H | 8 | 16 | 24 | 32 | 40 | 48 | 56 | 64 | 72 | 80 | 88 | 96 | | | | | |
| Checkerboard 2 | | | | | | | | | | | | | 16 Positive Samples | | | | |
| | 1 | 2 | 3 | 4 | 5 | 6 | 7 | 8 | 9 | 10 | 11 | 12 | | | | | |
| A | 1 | 9 | 17 | 25 | 33 | 41 | 49 | 57 | 65 | 73 | 81 | 89 | | | | | |
| B | 2 | 10 | 18 | 26 | 34 | 42 | 50 | 58 | 66 | 74 | 82 | 90 | One Step PCR | | | | |
| C | 3 | 11 | 19 | 27 | 35 | 43 | 51 | 59 | 67 | 75 | 83 | 91 | Reagent | 1 | X | 100 | X |
| D | 4 | 12 | 20 | 28 | 36 | 44 | 52 | 60 | 68 | 76 | 84 | 92 | Master Mix | 25 | μL | 2500 | μL |
| E | 5 | 13 | 21 | 29 | 37 | 45 | 53 | 61 | 69 | 77 | 85 | 93 | Primer | 2 | μL | 200 | μL |
| F | 6 | 14 | 22 | 30 | 38 | 46 | 54 | 62 | 70 | 78 | 86 | 94 | AMV | 1 | μL | 100 | μL |
| G | 7 | 15 | 23 | 31 | 39 | 47 | 55 | 63 | 71 | 79 | 87 | 95 | H2O | 17 | μL | 1700 | μL |
| H | 8 | 16 | 24 | 32 | 40 | 48 | 56 | 64 | 72 | 80 | 88 | 96 | | | | | |
| Checkerboard 3 | | | | | | | | | | | | | 25 Positive Samples | | | | |
| | 1 | 2 | 3 | 4 | 5 | 6 | 7 | 8 | 9 | 10 | 11 | 12 | | | | | |
| A | 1 | 9 | 17 | 25 | 33 | 41 | 49 | 57 | 65 | 73 | 81 | 89 | | | | | |
| B | 2 | 10 | 18 | 26 | 34 | 42 | 50 | 58 | 66 | 74 | 82 | 90 | One Step PCR | | | | |
| C | 3 | 11 | 19 | 27 | 35 | 43 | 51 | 59 | 67 | 75 | 83 | 91 | Reagent | 1 | X | 100 | X |
| D | 4 | 12 | 20 | 28 | 36 | 44 | 52 | 60 | 68 | 76 | 84 | 92 | Master Mix | 25 | μL | 2500 | μL |
| E | 5 | 13 | 21 | 29 | 37 | 45 | 53 | 61 | 69 | 77 | 85 | 93 | Primer | 2 | μL | 200 | μL |
| F | 6 | 14 | 22 | 30 | 38 | 46 | 54 | 62 | 70 | 78 | 86 | 94 | AMV | 1 | μL | 100 | μL |
| G | 7 | 15 | 23 | 31 | 39 | 47 | 55 | 63 | 71 | 79 | 87 | 95 | H2O | 17 | μL | 1700 | μL |
| H | 8 | 16 | 24 | 32 | 40 | 48 | 56 | 64 | 72 | 80 | 88 | 96 | | | | | |
| Checkerboard 4 | | | | | | | | | | | | | 18 Positive Samples | | | | |
| | 1 | 2 | 3 | 4 | 5 | 6 | 7 | 8 | 9 | 10 | 11 | 12 | | | | | |
| A | 1 | 9 | 17 | 25 | 33 | 41 | 49 | 57 | 65 | 73 | 81 | 89 | | | | | |
| B | 2 | 10 | 18 | 26 | 34 | 42 | 50 | 58 | 66 | 74 | 82 | 90 | One Step PCR | | | | |
| C | 3 | 11 | 19 | 27 | 35 | 43 | 51 | 59 | 67 | 75 | 83 | 91 | Reagent | 1 | X | 100 | X |
| D | 4 | 12 | 20 | 28 | 36 | 44 | 52 | 60 | 68 | 76 | 84 | 92 | Master Mix | 25 | μL | 2500 | μL |
| E | 5 | 13 | 21 | 29 | 37 | 45 | 53 | 61 | 69 | 77 | 85 | 93 | Primer | 2 | μL | 200 | μL |
| F | 6 | 14 | 22 | 30 | 38 | 46 | 54 | 62 | 70 | 78 | 86 | 94 | AMV | 1 | μL | 100 | μL |
| G | 7 | 15 | 23 | 31 | 39 | 47 | 55 | 63 | 71 | 79 | 87 | 95 | H2O | 17 | μL | 1700 | μL |

| | | | | | | | | | | | | |
|---|---|---|---|---|---|---|---|---|---|---|---|---|
| H | 8 | 16 | 24 | 32 | 40 | 48 | 56 | 64 | 72 | 80 | 88 | 96 |

TABLE 51
Representative data set for Checkerboard pattern# 2

| | Well | InfA.7. | No | RNAse.P. | No | SARS.COV2- |
|---|---|---|---|---|---|---|
| Sample | # | univ-pubRev | Print | Probe-pub1.1 | Print | N3-RE1.1 |
| NEG | 1 | 118 | −1907 | 4432 | −1859 | 12122 |
| NEG | 2 | 23 | −1350 | 5391 | −1408 | 7426 |
| NEG | 3 | 571 | −1824 | 4933 | −1917 | 2562 |
| NEG | 4 | −3 | −1362 | 3733 | −1439 | 3123 |
| NEG | 5 | 366 | −4043 | 2532 | −4034 | 9973 |
| NEG | 6 | 1573 | −2715 | 4594 | −2703 | 10370 |
| NEG | 7 | −86 | −2370 | 4989 | −2334 | 9584 |
| NEG | 8 | −43 | −2237 | 4110 | −2678 | 1691 |
| NEG | 9 | −22 | −1295 | 3486 | −1315 | 10057 |
| + | 10 | 5 | −1458 | 36946 | −1510 | 20840 |
| NEG | 11 | −15 | −1951 | 3076 | −1888 | 3325 |
| + | 12 | 287 | −2069 | 29118 | −2017 | 15066 |
| NEG | 13 | 7 | −3019 | 5728 | −3004 | 3425 |
| + | 14 | −260 | −2710 | 26602 | −2687 | 19669 |
| NEG | 15 | 204 | −3033 | 5242 | −3027 | 141 |
| + | 16 | 26 | −2918 | 17040 | −2969 | 20502 |
| NEG | 17 | −15 | −1188 | 3356 | −1184 | 2912 |
| NEG | 18 | −39 | −1541 | 2954 | −1494 | 575 |
| NEG | 19 | 94 | −1541 | 4230 | −1524 | 3397 |
| NEG | 20 | −94 | −1351 | 3758 | −1393 | 5250 |
| NEG | 21 | 914 | −2729 | 4694 | −2723 | 3664 |
| NEG | 22 | −125 | −2539 | 3382 | −2440 | 835 |
| NEG | 23 | −349 | −2850 | 2377 | −2791 | 7492 |
| NEG | 24 | −49 | −2710 | 5214 | −2712 | 8411 |
| NEG | 25 | 166 | −2030 | 1783 | −2001 | 5722 |
| NEG | 26 | −93 | −1227 | 2955 | −1226 | 545 |
| NEG | 27 | −11 | −1759 | 2986 | −1780 | 9535 |
| NEG | 28 | −11 | −1450 | 4041 | −1408 | 7221 |
| NEG | 29 | 319 | −3344 | 4139 | −3389 | −8 |
| NEG | 30 | 67 | −3059 | 4797 | −3050 | 315 |
| NEG | 31 | 60 | −3720 | 5904 | −3643 | 6836 |
| NEG | 32 | −18 | −2875 | 2786 | −2788 | 106 |
| + | 33 | −3 | −1123 | 22244 | −1072 | 13071 |
| NEG | 34 | −25 | −1253 | 4395 | −1299 | 6322 |
| + | 35 | 22 | −1336 | 21314 | −1359 | 14531 |
| NEG | 36 | 449 | −1624 | 3231 | −1676 | 1318 |
| + | 37 | −251 | −1984 | 22828 | −1941 | 14254 |
| NEG | 38 | −39 | −2819 | 4467 | −2798 | 171 |
| + | 39 | −178 | −2456 | 37117 | −2498 | 24316 |
| NEG | 40 | −51 | −2019 | 3588 | −2086 | 4616 |
| NEG | 41 | −59 | −1251 | 2833 | −1255 | 757 |
| NEG | 42 | −21 | −1314 | 3870 | −1355 | 5794 |
| NEG | 43 | −12 | −1438 | 4139 | −1378 | 2315 |
| NEG | 44 | −34 | −1433 | 3208 | −1362 | 5800 |
| NEG | 45 | 62 | −2446 | 3407 | −2448 | 4137 |
| NEG | 46 | −174 | −2355 | 9551 | −2386 | 5179 |
| NEG | 47 | 675 | −2771 | 2271 | −2834 | 6312 |
| NEG | 48 | 85 | −2636 | 2591 | −2679 | −36 |
| NEG | 49 | −154 | −1511 | 3073 | −1370 | 3192 |
| NEG | 50 | −7 | −1119 | 3379 | −1209 | 2876 |
| NEG | 51 | 156 | −2002 | 3021 | −2001 | 2784 |
| NEG | 52 | 2 | −1603 | 3891 | −1578 | 5283 |
| NEG | 53 | 1212 | −3493 | 2878 | −3543 | 12708 |
| NEG | 54 | 90 | −2658 | 2792 | −2738 | 4064 |
| NEG | 55 | −91 | −3323 | 9352 | −3391 | 1560 |
| NEG | 56 | −30 | −2234 | 3919 | −2976 | 12009 |
| NEG | 57 | −16 | −1545 | 3351 | −1453 | 12146 |
| + | 58 | −4 | −1596 | 21193 | −1547 | 16615 |
| NEG | 59 | 123 | −1337 | 3298 | −1375 | 10052 |
| + | 60 | −13 | −1451 | 18003 | −1185 | 8505 |
| NEG | 61 | 279 | −2906 | 5274 | −2948 | 4098 |
| + | 62 | 215 | −3052 | 19537 | −3092 | 11803 |
| NEG | 63 | −68 | −2820 | 7514 | −2866 | 5668 |
| + | 64 | 157 | −2904 | 14972 | −2926 | 23291 |
| NEG | 65 | 54 | −1218 | 2857 | −1184 | 448 |
| NEG | 66 | −16 | −1285 | 3554 | −1298 | 829 |
| NEG | 67 | −105 | −1581 | 3410 | −1600 | 8279 |
| NEG | 68 | −39 | −1137 | 2967 | −461 | 760 |
| NEG | 69 | 892 | −1735 | 4661 | −1721 | 3944 |
| NEG | 70 | −172 | −1878 | 4205 | −1893 | 12119 |
| NEG | 71 | −192 | −2011 | 3670 | −2041 | 6645 |
| NEG | 72 | 87 | −2408 | 2435 | −2521 | 1437 |
| NEG | 73 | 319 | −1746 | 3599 | −1751 | 7396 |
| NEG | 74 | 167 | −1339 | 2771 | −1316 | 1807 |
| NEG | 75 | 397 | −1919 | 3632 | −1942 | 7432 |
| NEG | 76 | −65 | −1086 | 4935 | −1071 | 3671 |
| NEG | 77 | 316 | −3781 | 5274 | −3797 | 2848 |
| NEG | 78 | 84 | −2608 | 5373 | −2713 | 4091 |
| NEG | 79 | 1001 | −2924 | 6070 | −3082 | 2303 |
| NEG | 80 | −98 | −2394 | 2761 | −2261 | 3315 |
| + | 81 | 545 | −1241 | 24720 | −1241 | 17022 |
| NEG | 82 | 19 | −1278 | 3589 | −1348 | 2778 |
| + | 83 | −62 | −1242 | 29074 | −1262 | 20212 |
| NEG | 84 | −20 | −1119 | 4985 | −1131 | 5181 |
| + | 85 | 560 | −2495 | 36659 | −2403 | 28652 |
| NEG | 86 | 81 | −3062 | 5006 | −3089 | 3827 |
| + | 87 | −326 | −2297 | 33766 | −2324 | 27195 |
| NEG | 88 | 64 | −2818 | 5515 | −2627 | 3917 |
| NEG | 89 | 355 | −1388 | 5759 | −1362 | 649 |
| NEG | 90 | −54 | −1246 | 3419 | −1287 | 4018 |
| NEG | 91 | 21 | −934 | 3586 | −943 | 4550 |
| NEG | 92 | 195 | −1361 | 4460 | −1260 | 4451 |
| NEG | 93 | −39 | −2069 | 3075 | −1989 | 12896 |
| NEG | 94 | −189 | −2084 | 4572 | −2169 | 1273 |
| NEG | 95 | −43 | −1962 | 2353 | −1971 | 6754 |
| NEG | 96 | 211 | −2411 | 3321 | −2449 | 633 |
| | | 614G-SE- | SARS.COV2- | 614D-SE- | SARS.COV2- | 614U-SE- |
| | Sample | S1-RE1.4 | N2-RE1.4 | S1-RE1.4 | N2-RE1.3 | S1-RE1.1 |
| | NEG | 1340 | 913 | 1859 | 1134 | 1494 |
| | NEG | 2019 | 1119 | 2185 | 1397 | 1896 |
| | NEG | 1124 | 1081 | 910 | 1049 | 1209 |
| | NEG | 1721 | 1887 | 1830 | 3201 | 1523 |
| | NEG | 382 | 68 | 626 | 1503 | 201 |
| | NEG | 1677 | 436 | 1924 | 921 | 1401 |
| | NEG | 2481 | 1855 | 2236 | 2724 | 1781 |
| | NEG | 2455 | 895 | 2188 | 1482 | 1906 |
| | NEG | 2141 | 1130 | 4581 | 1609 | 2337 |
| | + | 1621 | 18838 | 1565 | 7516 | 1389 |
| | NEG | 1711 | 1667 | 1729 | 1688 | 1309 |
| | + | 1903 | 14995 | 1650 | 4842 | 1464 |
| | NEG | 1044 | 3326 | 1628 | 2192 | 984 |
| | + | 1493 | 18300 | 1964 | 6869 | 1767 |
| | NEG | 1668 | 548 | 1650 | 1500 | 1154 |
| | + | 1801 | 16373 | 1488 | 5982 | 1319 |
| | NEG | 1587 | 704 | 1746 | 1117 | 1677 |
| | NEG | 1200 | 567 | 1039 | 792 | 963 |
| | NEG | 2298 | 1087 | 2451 | 1292 | 2099 |
| | NEG | 1549 | 917 | 1413 | 1127 | 1319 |
| | NEG | 1398 | 2122 | 1471 | 2993 | 1058 |
| | NEG | 1291 | 2838 | 1572 | 4581 | 1023 |
| | NEG | 1064 | 743 | 1094 | 3007 | 779 |
| | NEG | 806 | 690 | 970 | 3096 | 698 |
| | NEG | 656 | 1793 | 2145 | 1732 | 1196 |
| | NEG | 1705 | 500 | 1693 | 533 | 1386 |
| | NEG | 1390 | 921 | 1589 | 2893 | 1289 |
| | NEG | 1651 | 396 | 1746 | 1028 | 1307 |
| | NEG | 1394 | 218 | 1981 | 1852 | 948 |
| | NEG | 1224 | 303 | 1696 | 784 | 1045 |
| | NEG | 1203 | 1238 | 1456 | 3556 | 702 |
| | NEG | 1219 | 2148 | 1199 | 5673 | 754 |
| | + | 1586 | 12327 | 1455 | 5153 | 1472 |
| | NEG | 1350 | 2043 | 1327 | 4391 | 1286 |
| | + | 1633 | 16323 | 1647 | 5435 | 1151 |
| | NEG | 2056 | 1780 | 2170 | 4173 | 1784 |
| | + | 2536 | 11441 | 2600 | 4341 | 2318 |
| | NEG | 962 | 2205 | 1516 | 1519 | 1061 |
| | + | 2106 | 13960 | 1802 | 4836 | 1589 |
| | NEG | 2362 | 1295 | 2547 | 1929 | 1783 |
| | NEG | 1522 | 586 | 1499 | 2320 | 1458 |
| | NEG | 1478 | 2373 | 1313 | 2868 | 1329 |
| | NEG | 1253 | 373 | 1270 | 630 | 943 |
| | NEG | 1440 | 4640 | 1458 | 934 | 1436 |
| | NEG | 1401 | 3987 | 1350 | 5072 | 990 |
| | NEG | 1872 | 1875 | 2308 | 2945 | 1471 |
| | NEG | 719 | 274 | 595 | 1203 | 784 |
| | NEG | 1164 | 1009 | 1048 | 1374 | 904 |
| | NEG | 1384 | 896 | 1288 | 1821 | 1371 |
| | NEG | 1720 | 1435 | 1560 | 1417 | 1496 |
| | NEG | 1209 | 6834 | 1208 | 5231 | 1037 |
| | NEG | 1285 | 288 | 910 | 473 | 1345 |
| | NEG | 1065 | 1314 | 169 | 1678 | 491 |
| | NEG | 1372 | 203 | 1654 | 823 | 841 |
| | NEG | 1926 | 1638 | 1901 | 1983 | 1071 |
| | NEG | 1393 | 225 | 1136 | 769 | 896 |
| | NEG | 1646 | 1159 | 1191 | 859 | 1361 |
| | + | 1258 | 10103 | 1161 | 3517 | 1449 |
| | NEG | 1315 | 636 | 1349 | 865 | 1580 |
| | + | 1591 | 8500 | 1550 | 3338 | 1212 |
| | NEG | 1100 | 592 | 1335 | 1356 | 1292 |
| | + | 1487 | 7647 | 1524 | 3250 | 902 |
| | NEG | 2126 | 4590 | 2470 | 1868 | 1188 |
| | + | 1518 | 11144 | 1757 | 5401 | 1117 |
| | NEG | 1168 | 174 | 1197 | 406 | 1091 |
| | NEG | 931 | 1802 | 835 | 1395 | 1134 |
| | NEG | 1723 | 897 | 1592 | 750 | 1463 |
| | NEG | 1467 | 1125 | 1587 | 1507 | 1319 |
| | NEG | 2460 | 1529 | 2356 | 2076 | 1941 |
| | NEG | 2309 | 1757 | 2110 | 2314 | 1801 |
| | NEG | 2042 | 2569 | 2222 | 2255 | 1762 |
| | NEG | 812 | 2047 | 975 | 4517 | 537 |
| | NEG | 777 | 1262 | 1029 | 1638 | 961 |
| | NEG | 1660 | 400 | 1424 | 537 | 1282 |
| | NEG | 1319 | 546 | 1343 | 632 | 1062 |
| | NEG | 2555 | 1382 | 2699 | 1995 | 2184 |
| | NEG | 388 | 1823 | 772 | 3108 | 963 |
| | NEG | 1963 | 2572 | 2032 | 2917 | 1213 |
| | NEG | 1790 | 2039 | 1422 | 2987 | 1073 |
| | NEG | 1918 | 665 | 2058 | 1220 | 1146 |
| | NEG | 1472 | 11744 | 1459 | 4644 | 1219 |
| | NEG | 1612 | 275 | 1188 | 578 | 1246 |
| | NEG | 2033 | 14068 | 2166 | 5615 | 2468 |
| | NEG | 1569 | 460 | 1786 | 1228 | 1605 |
| | NEG | 1583 | 26129 | 2239 | 8269 | 1398 |
| | NEG | 1265 | 66 | 1616 | 759 | 1117 |
| | + | 1945 | 22834 | 1847 | 11494 | 1412 |
| | NEG | 1707 | 907 | 1556 | 2269 | 1284 |
| | NEG | 1433 | 402 | 1298 | 879 | 1282 |
| | NEG | 1343 | 1634 | 1297 | 2191 | 991 |
| | NEG | 1743 | 1288 | 1551 | 2834 | 1586 |
| | NEG | 706 | 842 | 909 | 1186 | 1381 |
| | NEG | 1105 | 1856 | 1832 | 4240 | 1700 |
| | NEG | 1184 | 886 | 1290 | 1293 | 942 |
| | NEG | 1755 | 1027 | 1807 | 1802 | 1490 |
| | NEG | 1257 | 1940 | 1411 | 4380 | 1362 |
| | | | SARS.COV2- | SARS.COV2- | 62- | InfB.8. |
| | | Sample | N1-RE1.1 | N1-pub | Negcont-B | univ-pub |
| | | NEG | 3369 | 9755 | 214 | −60.5 |
| | | NEG | 3678 | 10620 | 1090 | 58.75 |
| | | NEG | 1361 | 11603 | 511 | −53.25 |
| | | NEG | 4720 | 13574 | 1016 | 14.25 |
| | | NEG | 2485 | 6281 | 346 | 453.75 |
| | | NEG | 4064 | 12580 | 534 | 214 |
| | | NEG | 4468 | 12617 | 2261 | 793 |
| | | NEG | 4293 | 12503 | 2121 | 358 |
| | | NEG | 5131 | 15599 | 921 | 394 |
| | | + | 20043 | 34141 | 753 | 262 |
| | | NEG | 1797 | 7597 | 680 | 85 |
| | | + | 15588 | 31436 | 493 | −70 |
| | | NEG | 2308 | 12208 | 199 | 100 |
| | | + | 10794 | 24673 | 2078 | 840 |
| | | NEG | 2847 | 12344 | 291 | 149 |
| | | + | 7165 | 19803 | 1945 | 127 |
| | | NEG | 2913 | 12075 | 695 | 252 |
| | | NEG | 2978 | 4303 | 481 | 113 |
| | | NEG | 4463 | 13335 | 1007 | 212 |
| | | NEG | 2996 | 11174 | 518 | 127 |
| | | NEG | 3944 | 14906 | 626 | 9 |
| | | NEG | 2253 | 8777 | 748 | 173 |
| | | NEG | 3002 | 13335 | 345 | 200 |
| | | NEG | 2242 | 10423 | 461 | 335 |
| | | NEG | 4159 | 12296 | 490 | 27 |
| | | NEG | 2010 | 8115 | 369 | 599 |
| | | NEG | 4595 | 10959 | 221 | 151 |
| | | NEG | 2177 | 8574 | 241 | 213 |
| | | NEG | 3068 | 12062 | 1042 | 537 |
| | | NEG | 3080 | 12023 | 392 | 651 |
| | | NEG | 2760 | 14284 | 771 | 73 |
| | | NEG | 3668 | 16084 | 483 | 338 |
| | | + | 10072 | 22841 | 683 | 173 |
| | | NEG | 3653 | 10387 | 654 | 72 |
| | | + | 9588 | 20000 | 575 | 54 |
| | | NEG | 2999 | 8572 | 478 | 9 |
| | | + | 7794 | 18638 | 1215 | 857 |
| | | NEG | 2693 | 10641 | 2270 | 235 |
| | | + | 12824 | 32203 | 849 | 692 |
| | | NEG | 3219 | 10379 | 2937 | 1208 |
| | | NEG | 2348 | 9343 | 518 | 119 |
| | | NEG | 4271 | 8439 | 728 | 277 |
| | | NEG | 3449 | 10383 | 244 | 293 |
| | | NEG | 1976 | 7530 | 527 | 652 |
| | | NEG | 2032 | 8129 | 567 | 133 |
| | | NEG | 3349 | 9783 | 1453 | 827 |
| | | NEG | 3382 | 9207 | 534 | 220 |
| | | NEG | 2275 | 9122 | 789 | 225 |
| | | NEG | 3681 | 9941 | 258 | 313 |
| | | NEG | 2272 | 10181 | 653 | 318 |
| | | NEG | 3602 | 10629 | 262 | −36 |
| | | NEG | 2754 | 8469 | 170 | 166 |
| | | NEG | 526 | 6014 | 1980 | 1366 |
| | | NEG | 4521 | 11224 | 141 | 707 |
| | | NEG | 3306 | 14560 | 1585 | 149 |
| | | NEG | 3732 | 10164 | 1164 | 138 |
| | | NEG | 3959 | 12784 | 357 | 23 |
| | | + | 12028 | 26975 | 363 | 52 |
| | | NEG | 4346 | 10190 | 558 | −71 |
| | | + | 7833 | 20392 | 471 | 140 |
| | | NEG | 1826 | 9222 | 429 | 159 |
| | | + | 4874 | 15940 | 1263 | 422 |
| | | NEG | 3032 | 9546 | 949 | 220 |
| | | + | 11756 | 29368 | 2146 | −67 |
| | | NEG | 3082 | 9216 | 458 | 8 |
| | | NEG | 5479 | 12094 | 516 | 327 |
| | | NEG | 3181 | 9593 | 639 | 365 |
| | | NEG | 1325 | 3297 | 709 | 657 |
| | | NEG | 2461 | 11216 | 1083 | −20 |
| | | NEG | 3204 | 6008 | 1748 | 943 |
| | | NEG | 4453 | 11224 | 1461 | 648 |
| | | NEG | 3967 | 10082 | 638 | 51 |
| | | NEG | 2657 | 7523 | 74 | 53 |
| | | NEG | 3325 | 10597 | 160 | 23 |
| | | NEG | 2824 | 9477 | −39 | 84 |
| | | NEG | 3287 | 9538 | 997 | 643 |
| | | NEG | 2140 | 9579 | 714 | 818 |
| | | NEG | 2804 | 11688 | 647 | 279 |
| | | NEG | 2297 | 12577 | 911 | −87 |
| | | NEG | 2066 | 6489 | 1212 | 354 |
| | | + | 19936 | 36119 | 960 | 367 |
| | | NEG | 3106 | 15116 | 2282 | −15 |
| | | NEG | 2622 | 11517 | 500 | −51 |
| | | NEG | 3350 | 11644 | 385 | 492 |
| | | NEG | 2604 | 6778 | 554 | 27 |
| | | NEG | 3600 | 8615 | 493 | 154 |
| | | NEG | 4397 | 10498 | 788 | 623 |
| | | NEG | 4445 | 12232 | 746 | 297 |
| | | NEG | 2793 | 10460 | 1168 | 305 |
| | | NEG | 4077 | 11616 | 584 | 45 |

TABLE 52
Representative data set for Checkerboard pattern# 3

| | Well | InfA.7. | No | RNAse.P. | No | SARS.COV2- |
|---|---|---|---|---|---|---|
| Sample | # | univ-pubRev | Print | Probe-pub1.1 | Print | N3-RE1.1 |
| + | 1 | −145 | −1347 | 13513 | −1273 | 11552 |
| NEG | 2 | 2766 | −882 | 3165 | −852 | 3560 |
| NEG | 3 | 9 | −1774 | 4377 | −1733 | 667 |
| + | 4 | 11 | −1288 | 18587 | −1368 | 6454 |
| NEG | 5 | 41 | −2969 | 3941 | −2868 | 45 |
| NEG | 6 | −6 | −2250 | 3571 | −2271 | 12648 |
| NEG | 7 | −203 | −2909 | 3938 | −2789 | 846 |
| + | 8 | −96 | −2252 | 25352 | −2267 | 11733 |
| NEG | 9 | 16 | −1396 | 3533 | −1387 | 3274 |
| + | 10 | 21 | −1003 | 25979 | −1049 | 14030 |
| NEG | 11 | 16 | −1525 | 2710 | −1536 | 6160 |
| NEG | 12 | −35 | −1715 | 2541 | −1667 | 1076 |
| NEG | 13 | 407 | −2261 | 3216 | −2258 | 1248 |
| NEG | 14 | 25 | −2492 | 4125 | −2524 | 2217 |
| + | 15 | 126 | −2361 | 38860 | −2304 | 32693 |
| NEG | 16 | 238 | −2019 | 3917 | −2001 | 1111 |
| NEG | 17 | 5 | −1278 | 3845 | −1308 | 1361 |
| NEG | 18 | 32 | −1393 | 3169 | −1409 | 2383 |
| + | 19 | 32 | −1492 | 25184 | −1402 | 13261 |
| NEG | 20 | −22 | −1314 | 3346 | −1336 | 1862 |
| NEG | 21 | −245 | −1707 | 2937 | −1700 | 1224 |
| + | 22 | −128 | −2271 | 25983 | −2171 | 16445 |
| NEG | 23 | 11 | −2297 | 4221 | −2277 | 2769 |
| NEG | 24 | 80 | −2388 | 4253 | −2353 | 3295 |
| NEG | 25 | −15 | −1961 | 3215 | −1960 | 248 |
| NEG | 26 | 167 | −1332 | 3245 | −1480 | 705 |
| NEG | 27 | 45 | −1738 | 3201 | −1716 | 2231 |
| + | 28 | −9 | −1339 | 13102 | −1379 | 5357 |
| NEG | 29 | −7 | −3055 | 5616 | −3061 | 8413 |
| NEG | 30 | 44 | −2729 | 4512 | −2673 | 4608 |
| NEG | 31 | 172 | −3557 | 4239 | −3131 | 3112 |
| + | 32 | 28 | −2707 | 31840 | −2635 | 13702 |
| NEG | 33 | −38 | −1223 | 3240 | −1268 | 4222 |
| NEG | 34 | 33 | −1412 | 3563 | −1490 | 1066 |
| NEG | 35 | −26 | −1441 | 3076 | −1485 | 2518 |
| NEG | 36 | −26 | −1489 | 2841 | −1474 | 2981 |
| + | 37 | −42 | −2611 | 14747 | −2496 | 18927 |
| NEG | 38 | 10 | −2615 | 4039 | −2734 | 1142 |
| NEG | 39 | 84 | −2543 | 2745 | −2541 | 272 |
| NEG | 40 | −32 | −2639 | 3798 | −2675 | 718 |
| NEG | 41 | 10 | −1177 | 2797 | −1151 | 2445 |
| NEG | 42 | 42 | −1096 | 2637 | −1143 | 751 |
| + | 43 | 747 | −1520 | 19273 | −1469 | 9631 |
| NEG | 44 | −117 | −1717 | 2698 | −1623 | 1254 |
| NEG | 45 | 495 | −1727 | 2935 | −1766 | 2195 |
| + | 46 | −348 | −1791 | 24816 | −1770 | 14867 |
| NEG | 47 | −113 | −2044 | 4076 | −1789 | 3977 |
| NEG | 48 | 65 | −2745 | 2638 | −2994 | 8603 |
| NEG | 49 | 9 | −1374 | 2610 | −1434 | 3297 |
| + | 50 | 87 | −1488 | 21434 | −1588 | 11066 |
| NEG | 51 | 133 | −1734 | 2250 | −1809 | 1037 |
| NEG | 52 | −124 | −1525 | 2035 | −1531 | 1662 |
| NEG | 53 | −65 | −3628 | 2709 | −3661 | 2327 |
| NEG | 54 | −170 | −2115 | 3168 | −2085 | 1475 |
| + | 55 | −55 | −2428 | 18535 | −2501 | 5147 |
| NEG | 56 | 834 | −2289 | 2946 | −2293 | 2633 |
| + | 57 | 165 | −1263 | 12569 | −1301 | 13662 |
| NEG | 58 | 205 | −1246 | 2901 | −1251 | 2153 |
| NEG | 59 | 10 | −1354 | 3013 | −1366 | 1729 |
| + | 60 | −101 | −1068 | 8987 | −1165 | 4430 |
| NEG | 61 | −126 | −1901 | 2632 | −1946 | 517 |
| NEG | 62 | 664 | −2451 | 2219 | −2472 | 1783 |
| NEG | 63 | −19 | −2174 | 3240 | −2137 | 700 |
| + | 64 | 14 | −2512 | 16859 | −2438 | 4932 |
| NEG | 65 | 50 | −1145 | 3136 | −1126 | 1829 |
| NEG | 66 | −8 | −1238 | 2665 | −1210 | 1673 |
| + | 67 | 321 | −1214 | 14727 | −1217 | 5624 |
| NEG | 68 | 55 | −1161 | 2521 | −1194 | 2944 |
| NEG | 69 | 374 | −1941 | 2697 | −1992 | 1095 |
| NEG | 70 | 37 | −2400 | 3026 | −2448 | 1389 |
| + | 71 | −138 | −2553 | 20514 | −2593 | 6508 |
| NEG | 72 | 216 | −2252 | 3174 | −2168 | −66 |
| NEG | 73 | 190 | −1550 | 2717 | −1677 | 3094 |
| + | 74 | 153 | −884 | 14993 | −872 | 8301 |
| NEG | 75 | 534 | −1736 | 2617 | −1710 | 1856 |
| NEG | 76 | 22 | −1268 | 2237 | −1298 | 2637 |
| NEG | 77 | −41 | −2503 | 2987 | −2625 | 1892 |
| + | 78 | −224 | −1808 | 31837 | −1772 | 15165 |
| NEG | 79 | −61 | −1767 | 2893 | −1892 | 2702 |
| NEG | 80 | −111 | −1849 | 2025 | −1891 | 830 |
| NEG | 81 | −83 | −1320 | 3112 | −1397 | 3628 |
| NEG | 82 | 107 | −1386 | 3660 | −1418 | 4359 |
| NEG | 83 | 617 | −1309 | 3033 | −1263 | 1347 |
| NEG | 84 | −184 | −1158 | 2819 | −1157 | 1921 |
| + | 85 | 1034 | −2012 | 27788 | −1967 | 17653 |
| NEG | 86 | 1218 | −2373 | 4368 | −2280 | 510 |
| NEG | 87 | 302 | −1949 | 2976 | −1868 | 834 |
| + | 88 | −383 | −1983 | 36246 | −1988 | 14961 |
| NEG | 89 | 14 | −1011 | 2460 | −778 | 3793 |
| NEG | 90 | 164 | −782 | 3460 | −813 | 2387 |
| NEG | 91 | 113 | −1221 | 2598 | −1134 | 2683 |
| + | 92 | 72 | −858 | 18886 | −869 | 8671 |
| NEG | 93 | −91 | −1607 | 2637 | −1504 | 542 |
| NEG | 94 | 27 | −1525 | 3170 | −1571 | 1585 |
| + | 95 | 13 | −1313 | 14643 | −1451 | 3177 |
| NEG | 96 | 79 | −1573 | 3658 | −1590 | 2961 |
| | | 614G-SE- | SARS.COV2- | 614D-SE- | SARS.COV2- | 614U-SE- |
| | Sample | S1-RE1.4 | N2-RE1.4 | S1-RE1.4 | N2-RE1.3 | S1-RE1.1 |
| | + | 1560 | 12396 | 2134 | 4859 | 1561 |
| | NEG | 1628 | 751 | 1594 | 902 | 1235 |
| | NEG | 1913 | 548 | 2041 | 1175 | 1460 |
| | + | 1372 | 9753 | 1613 | 4169 | 1613 |
| | NEG | 1835 | 632 | 1572 | 2223 | 1508 |
| | NEG | 1369 | 1189 | 1698 | 3266 | 1121 |
| | NEG | 2835 | 1939 | 2641 | 3441 | 2066 |
| | + | 1449 | 12305 | 1947 | 7464 | 1758 |
| | NEG | 1820 | 1681 | 1689 | 3315 | 1305 |
| | + | 1823 | 12938 | 2368 | 4919 | 2312 |
| | NEG | 1330 | 773 | 1540 | 1438 | 1269 |
| | NEG | 1911 | 847 | 1831 | 1242 | 1748 |
| | NEG | 2013 | 1088 | 1817 | 2047 | 1260 |
| | NEG | 1151 | 173 | 1701 | 1368 | 1414 |
| | + | 1758 | 34169 | 2216 | 13289 | 1722 |
| | NEG | 1408 | −83 | 2009 | 2624 | 1924 |
| | NEG | 1195 | 337 | 1401 | 1437 | 1445 |
| | NEG | 1167 | 1352 | 1315 | 2067 | 1240 |
| | + | 1198 | 13348 | 1397 | 4601 | 1440 |
| | NEG | 1341 | 989 | 1098 | 1944 | 1255 |
| | NEG | 1820 | 2415 | 1639 | 3156 | 1780 |
| | + | 1741 | 18779 | 1628 | 7542 | 1925 |
| | NEG | 1321 | 142 | 1212 | 1656 | 1109 |
| | NEG | 1292 | 87 | 797 | 1726 | 1237 |
| | NEG | 761 | 1357 | 1127 | 4518 | 669 |
| | NEG | 1375 | 194 | 1535 | 270 | 1132 |
| | NEG | 1240 | 493 | 1414 | 2531 | 1320 |
| | + | 1416 | 5263 | 1515 | 2952 | 1349 |
| | NEG | 2409 | 1801 | 2010 | 3168 | 1808 |
| | NEG | 1632 | 200 | 1678 | 1982 | 1174 |
| | NEG | 2352 | −5 | 2259 | 1827 | 1529 |
| | + | 1874 | 19138 | 1450 | 8440 | 898 |
| | NEG | 1057 | 455 | 1182 | 432 | 987 |
| | NEG | 1108 | 96 | 1067 | 373 | 907 |
| | NEG | 1598 | 503 | 1432 | 935 | 1112 |
| | NEG | 1452 | 817 | 1392 | 1396 | 1591 |
| | + | 1470 | 7935 | 1371 | 3939 | 1343 |
| | NEG | 783 | 73 | 1218 | 1810 | 984 |
| | NEG | 1939 | 227 | 2267 | 2080 | 1103 |
| | NEG | 1332 | 873 | 2099 | 4887 | 1305 |
| | NEG | 1438 | 754 | 1224 | 904 | 1196 |
| | NEG | 1385 | 560 | 1192 | 767 | 1307 |
| | + | 1544 | 10717 | 1549 | 4542 | 1739 |
| | NEG | 1044 | 367 | 1277 | 1017 | 1086 |
| | NEG | 1999 | 1773 | 1752 | 3412 | 1607 |
| | + | 2509 | 15026 | 2130 | 5967 | 2176 |
| | NEG | 3511 | 2553 | 3199 | 3015 | 2668 |
| | NEG | 818 | 689 | 840 | 4999 | 459 |
| | NEG | 747 | 371 | 1250 | 1475 | 1154 |
| | + | 1175 | 10325 | 565 | 5222 | 1412 |
| | NEG | 1034 | 22 | 1119 | 546 | 853 |
| | NEG | 1560 | 652 | 1666 | 3295 | 1304 |
| | NEG | 902 | 730 | 1034 | 1161 | 798 |
| | NEG | 2465 | 2174 | 3223 | 2197 | 2875 |
| | + | 2273 | 7791 | 2450 | 4658 | 1888 |
| | NEG | 2110 | −454 | 2466 | 1452 | 1207 |
| | + | 1140 | 9272 | 1002 | 4419 | 1257 |
| | NEG | 1191 | 505 | 1076 | 3265 | 984 |
| | NEG | 1192 | 3107 | 1498 | 1368 | 1073 |
| | + | 1932 | 6573 | 1612 | 3908 | 1895 |
| | NEG | 1323 | 159 | 1475 | 1657 | 1310 |
| | NEG | 1614 | 334 | 1549 | 3134 | 1730 |
| | NEG | 2672 | 543 | 1789 | 2708 | 1550 |
| | + | 2028 | 10263 | 1962 | 6662 | 998 |
| | NEG | 1120 | 376 | 1209 | 1077 | 1192 |
| | NEG | 1161 | 404 | 929 | 642 | 1118 |
| | + | 1390 | 7049 | 1639 | 3302 | 1567 |
| | NEG | 1630 | 569 | 1227 | 678 | 1111 |
| | NEG | 1366 | −67 | 1472 | 1244 | 1185 |
| | NEG | 1287 | 867 | 852 | 3758 | 1430 |
| | + | 1335 | 12211 | 1052 | 5686 | 883 |
| | NEG | 1971 | 564 | 1303 | 2897 | 1497 |
| | NEG | 1033 | 49 | 857 | 580 | 775 |
| | + | 1246 | 6437 | 1320 | 3428 | 1174 |
| | NEG | 1032 | 100 | 1172 | 802 | 812 |
| | NEG | 1398 | 261 | 1224 | 2744 | 1449 |
| | NEG | 1632 | 472 | 1711 | 2117 | 1139 |
| | + | 2148 | 15265 | 2648 | 5991 | 3746 |
| | NEG | 2355 | 413 | 2411 | 2861 | 1465 |
| | NEG | 1391 | 979 | 1395 | 1883 | 1285 |
| | NEG | 1132 | 517 | 1219 | 675 | 990 |
| | NEG | 1407 | 1674 | 1466 | 1386 | 1173 |
| | NEG | 1322 | 189 | 1301 | 673 | 1096 |
| | NEG | 1103 | 953 | 1274 | 2668 | 1508 |
| | + | 1170 | 17719 | 1586 | 7198 | 2980 |
| | NEG | 1083 | 2564 | 1558 | 5268 | 869 |
| | NEG | 3039 | 183 | 3533 | 2648 | 2412 |
| | + | 2030 | 19467 | 2355 | 10461 | 2474 |
| | NEG | 1095 | 342 | 1089 | 1237 | 1477 |
| | NEG | 1568 | 714 | 1138 | 1251 | 1010 |
| | NEG | 1365 | 982 | 1267 | 3620 | 1479 |
| | + | 1180 | 9678 | 885 | 5339 | 1339 |
| | NEG | 1063 | 1226 | 1584 | 3335 | 1207 |
| | NEG | 837 | 244 | 1388 | 1048 | 1085 |
| | + | 1266 | 6639 | 1455 | 5249 | 1489 |
| | NEG | 1076 | 306 | 1467 | 1412 | 1374 |
| | | | SARS.COV2- | SARS.COV2- | 62- | InfB.8. |
| | | Sample | N1-RE1.1 | N1-pub | Negcont-B | univ-pub |
| | | + | 9479 | 22501 | 1008 | 309 |
| | | NEG | 2750 | 12791 | 157 | −12 |
| | | NEG | 4903 | 12923 | 715 | 81 |
| | | + | 9425 | 18071 | 311 | 113 |
| | | NEG | 3099 | 13406 | 1425 | 85 |
| | | NEG | 5306 | 15088 | 832 | 46 |
| | | NEG | 4737 | 14011 | 2974 | 625 |
| | | + | 13782 | 25693 | 1596 | 546 |
| | | NEG | 3511 | 12867 | 822 | 181 |
| | | + | 10684 | 22896 | 731 | 71 |
| | | NEG | 2732 | 9330 | 843 | 215 |
| | | NEG | 3936 | 9460 | 1797 | 63 |
| | | NEG | 5101 | 15333 | 634 | 51 |
| | | NEG | 3699 | 9857 | 1890 | 286 |
| | | + | 28227 | 40198 | 647 | 85 |
| | | NEG | 3857 | 11013 | 2076 | 640 |
| | | NEG | 4135 | 11629 | 680 | 47 |
| | | NEG | 4458 | 11356 | 463 | 153 |
| | | + | 9350 | 23518 | 677 | 14 |
| | | NEG | 3932 | 10956 | 737 | 441 |
| | | NEG | 2874 | 10312 | 1119 | 335 |
| | | + | 11633 | 26389 | 1945 | 296 |
| | | NEG | 3992 | 9252 | 944 | 747 |
| | | NEG | 4851 | 12835 | 1302 | 603 |
| | | NEG | 4749 | 11845 | 95 | 489 |
| | | NEG | 1854 | 9174 | 56 | −6 |
| | | NEG | 4036 | 15147 | 250 | 10 |
| | | + | 5422 | 14555 | 420 | 205 |
| | | NEG | 3138 | 11740 | 2868 | 782 |
| | | NEG | 4201 | 11540 | 499 | 39 |
| | | NEG | 3834 | 11775 | 2611 | 250 |
| | | + | 16093 | 32257 | 604 | 155 |
| | | NEG | 2577 | 7125 | 420 | 528 |
| | | NEG | 3877 | 9215 | 203 | 129 |
| | | NEG | 2903 | 10539 | 666 | 86 |
| | | NEG | 2977 | 9879 | 555 | 310 |
| | | + | 8192 | 18537 | 274 | 155 |
| | | NEG | 2956 | 8002 | 1112 | 160 |
| | | NEG | 2592 | 14261 | 665 | 434 |
| | | NEG | 3930 | 11612 | 1690 | 533 |
| | | NEG | 2186 | 6180 | 544 | −54 |
| | | NEG | 2987 | 7288 | 633 | 436 |
| | | + | 8797 | 17595 | 959 | 56 |
| | | NEG | 1519 | 5525 | 628 | 416 |
| | | NEG | 3647 | 8520 | 806 | 169 |
| | | + | 9030 | 19347 | 1584 | 614 |
| | | NEG | 3722 | 8044 | 2173 | 913 |
| | | NEG | 3683 | 8515 | 688 | 72 |
| | | NEG | 2079 | 6407 | 543 | 198 |
| | | + | 12167 | 26093 | 85 | 56 |
| | | NEG | 2677 | 7378 | 345 | 28 |
| | | NEG | 2536 | 6944 | 488 | 238 |
| | | NEG | 1696 | 6693 | 173 | 185 |
| | | NEG | 3130 | 9902 | 1847 | 1018 |
| | | + | 5871 | 13814 | 1827 | 973 |
| | | NEG | 4380 | 11465 | 1176 | 785 |
| | | + | 9722 | 18937 | 376 | −61 |
| | | NEG | 1804 | 6497 | 394 | 45 |
| | | NEG | 2443 | 5677 | 325 | 125 |
| | | + | 7573 | 14010 | 851 | 885 |
| | | NEG | 2365 | 7134 | 477 | 333 |
| | | NEG | 3060 | 8111 | 1173 | 386 |
| | | NEG | 3599 | 8336 | 824 | 1155 |
| | | + | 15475 | 29930 | 1799 | 323 |
| | | NEG | 3484 | 7877 | 414 | 40 |
| | | NEG | 2097 | 5971 | 247 | 289 |
| | | + | 8403 | 18913 | 658 | −31 |
| | | NEG | 2998 | 6709 | 234 | 156 |
| | | NEG | 2579 | 13838 | 576 | 82 |
| | | NEG | 2911 | 9277 | 561 | 291 |
| | | + | 15055 | 31208 | 397 | 161 |
| | | NEG | 5934 | 9809 | 1740 | 710 |
| | | NEG | 1522 | 8566 | 330 | 5 |
| | | + | 8230 | 18240 | 328 | −35 |
| | | NEG | 6401 | 12795 | 199 | 19 |
| | | NEG | 2199 | 5421 | 202 | 553 |
| | | NEG | 2312 | 5170 | 1441 | 270 |
| | | + | 14104 | 28376 | 970 | 545 |
| | | NEG | 3160 | 7311 | 1575 | 691 |
| | | NEG | 3178 | 6344 | 889 | 528 |
| | | NEG | 2943 | 7512 | 485 | 94 |
| | | NEG | 2486 | 8965 | 361 | 139 |
| | | NEG | 2929 | 6760 | 427 | 15 |
| | | NEG | 2107 | 5202 | 561 | 219 |
| | | + | 14960 | 29760 | 157 | 66 |
| | | NEG | 2567 | 8793 | 1312 | 8 |
| | | NEG | 4472 | 8238 | 932 | 694 |
| | | + | 23545 | 36200 | 2543 | 626 |
| | | NEG | 2768 | 7072 | 490 | 67 |
| | | NEG | 2520 | 5202 | 426 | −84 |
| | | NEG | 3474 | 7996 | 486 | 17 |
| | | + | 11890 | 26105 | 422 | 25 |
| | | NEG | 3071 | 7377 | 929 | 179 |
| | | NEG | 2830 | 6893 | 671 | 466 |
| | | + | 5288 | 8768 | 1138 | 168 |
| | | NEG | 3785 | 6930 | 1911 | 156 |

TABLE 53
Summary of checkerboard analysis for
well-to-well cross contamination

| | # | # | # | # | |
|---|---|---|---|---|---|
| | Positive | Negative | Positive | Negative | Overall |
| Checker- | Samples | Samples | Samples | Samples | Call |
| board | Added | Added | Detected | Detected | (POS/NEG) |
| 1.1 | 24 | 72 | 24 | 72 | 100%/100% |
| 1.2 | 24 | 72 | 24 | 72 | 100%/100% |
| 2.1 | 16 | 16 | 16 | 16 | 100%/100% |
| 2.2 | 16 | 16 | 16 | 16 | 100%/100% |
| 3.1 | 25 | 25 | 25 | 25 | 100%/100% |
| 3.2 | 25 | 25 | 25 | 25 | 100%/100% |
| 4.1 | 18 | 18 | 18 | 18 | 100%/100% |
| 4.2 | 18 | 18 | 18 | 18 | 100%/100% |

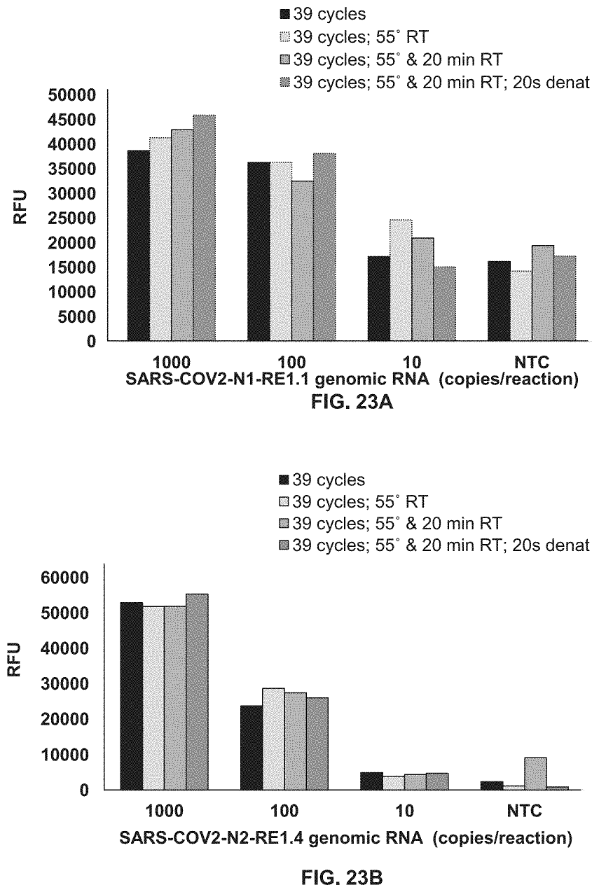

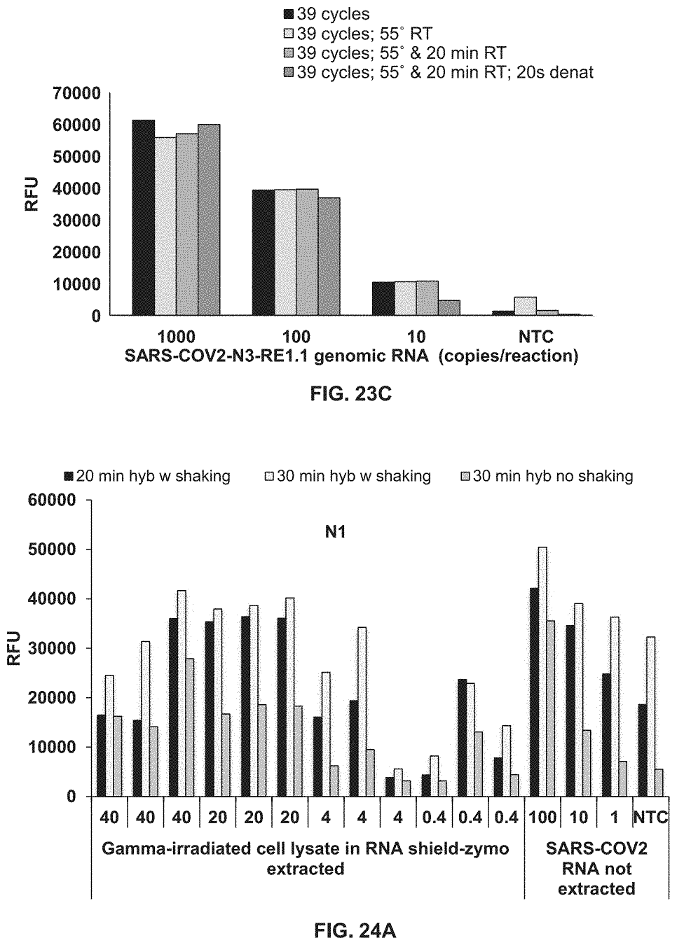

A summary of 4 different RT-PCR reaction protocols is shown in Table 54. Results from these studies summarized in FIGS. 23A-23C show that the Asymmetric One-Step PCR reaction can accommodate an increase in temperature from 37° C. to 55° C. in the reverse transcription phase of the reaction, without significantly altering efficiency of the Asymmetric One-Step PCR, as assessed by hybridization analysis in the 96 well Mini-RV format. It was also established that at 55° C., reverse transcription time could be reduced from 45 min to 20 min and additionally, heat denaturation time (protocol D, step 3 Table 54) could be reduced from 30 sec to 20 sec with no loss of RT-PCR efficiency. Importantly, by deploying protocol D (Table 54) the total duration for completing of the Asymmetric One-Step RT-PCR is reduced to 2 hours (Reverse transcription time at 30 min+PCR time at 1.5 hours).

TABLE 54
RT-PCR reaction protocols used to test potential reduction in assay time

| Protocol A | | | | | Protocol B | | | | |
|---|---|---|---|---|---|---|---|---|---|
| AccessQuick RT-PCR | | | | | AccessQuick RT-PCR | | | | |
| Steps | Temperature | Time | | Cycles | Steps | Temperature | Time | | Cycles |
| 1 | 45° C. | 45 | min | 1 | 1 | 55° C. | 45 | min | 1 |
| 2 | 94° C. | 2 | min | 1 | 2 | 94° C. | 2 | min | 1 |
| 3 | 94° C. | 30 | sec | 39 | 3 | 94° C. | 30 | sec | 39 |
| 4 | 55° C. | 30 | sec | | 4 | 55° C. | 30 | sec | |
| 5 | 68° C. | 30 | sec | | 5 | 68° C. | 30 | sec | |
| 6 | 68° C. | 7 | min | 1 | 6 | 68° C. | 7 | min | 1 |
| 7 | 4° C. | ∞ | | | 7 | 4° C. | ∞ | | |
| Protocol C | | | | | Protocol D | | | | |
| AccessQuick RT-PCR | | | | | AccessQuick RT-PCR | | | | |
| Steps | Temperature | Time | | Cycles | Steps | Temperature | Time | | Cycles |
| 1 | 55° C. | 20 | min | 1 | 1 | 55° C. | 20 | min | 1 |
| 2 | 94° C. | 2 | min | 1 | 2 | 94° C. | 2 | min | 1 |
| 3 | 94° C. | 30 | sec | 39 | 3 | 94° C. | 20 | sec | 39 |
| 4 | 55° C. | 30 | sec | | 4 | 55° C. | 30 | sec | |
| 5 | 68° C. | 30 | sec | | 5 | 68° C. | 30 | sec | |
| 6 | 68° C. | 7 | min | 1 | 6 | 68° C. | 7 | min | 1 |

| | | | | | |
|---|---|---|---|---|---|
| 7 | 4° C. | ∞ | 7 | 4° C. | ∞ |

This modification to the protocol also has additional advantages. Increasing the reverse transcription temperature to 55° C. makes the protocol compatible with a concurrent Uracil-DNA Glycosylase enzyme (UNG, Cod UNG from ArticZymes Technologies) reaction (see below). Further, reducing the time for heat denaturation from 30 sec to 20 sec reduces the Taq Thermal footprint during the RT-PCR reaction.

Mitigate Potential Assay Contamination Due to Low Copy Number RNA Sample Contamination by Ambient High Copy Number Amplicon Products from Previous Assays

Amplicon contamination has the undesired consequence of generating false positive results in the assay. This problem may be offset by the introduction of Uracil-DNA Glycosylase into the reverse transcription phase of the Asymmetric One-Step RT-PCR reaction. One of the requirements for using UNG is a reaction temperature of 55° C. As discussed above increasing the temperature from 37° C. to 55° C. during reverse transcription does not alter efficiency of the Asymmetric One-Step PCR (Table 54, FIGS. 23A-23C) thereby supporting a modified protocol where UNG and dUTP are introduced into the master mix. Cod UNG from ArticZymes Technologies is used for this purpose. The utility of UNG is established by testing the effect of 50% substitution of dTTP with dUTP and verifying no not significant alteration in analytical LoD occurs in the present Mini-RV workflow (Zymo)→Asymmetric One-Step RT-PCR→Hybridization/Wash→Sensovation (96-well imaging)

### Validation of Higher Temperature Reverse Transcription for UNG Deployment

Further validation for employing a higher temperature for the reverse transcription was obtained using multiple clinical isolates (NP-VTM from TriCore) and contrived samples (in nasal fluid) titrated with gamma irradiated CoV-2 virion (BEI, 5,000 virion/ml to 500/ml).

### Protocol:

Sample 1—Eight (8) Positive clinical samples.
NP/VTM (TriCore)→Ceres→RT-PCR→Hybridization (96-well)

Sample 2—Eight (8) Negative clinical samples.
NP/VTM (TriCore)→Ceres→RT-PCR→Hybridization (96-well)

Sample 3—Four (4) gamma irradiated virus (BEI). 5000, 1000, 500, 0 copies/mL
VTM+10% Nasal Fluid (Lee Bio)→Ceres→SRT-PCR→Hybridization (96-well)

Three different RT-PCR conditions were tested with each of the above sample sets as shown in Table 55.

TABLE 55
RT-PCR conditions for testing UNG deployment

| Condition 1 | | | | | Condition 2 | | | | | Condition 3 | | | | |
|---|---|---|---|---|---|---|---|---|---|---|---|---|---|---|
| Standard reverse | | | | | High temperature reverse | | | | | High temperature reverse | | | | |
| transcription 45° C., 45 min | | | | | transcription 55° C., 45 min | | | | | transcription 55° C., 20 min | | | | |
| AccessQuick RT-PCR | | | | | AccessQuick RT-PCR | | | | | AccessQuick RT-PCR | | | | |
| parameters | | | | | parameters | | | | | parameters | | | | |
| Steps | T (° C.) | Time | | Cycles | Steps | T (° C.) | Time | | Cycles | Steps | T (° C.) | Time | | Cycles |
| 1 | 45 | 45 | min | 1 | 1 | 45 | 45 | min | 1 | 1 | 45 | 45 | min | 1 |
| 2 | 94 | 2 | min | 1 | 2 | 94 | 2 | min | 1 | 2 | 94 | 2 | min | 1 |
| 3 | 94 | 30 | sec | 40 | 3 | 94 | 30 | sec | 40 | 3 | 94 | 30 | sec | 40 |
| 4 | 55 | 30 | sec | | 4 | 55 | 30 | sec | | 4 | 55 | 30 | sec | |
| 5 | 68 | 30 | sec | | 5 | 68 | 30 | sec | | 5 | 68 | 30 | sec | |
| 6 | 68 | 7 | min | 1 | 6 | 68 | 7 | min | 1 | 6 | 68 | 7 | min | 1 |

| | | | | | | | | |
|---|---|---|---|---|---|---|---|---|
| 7 | 4 | ∞ | 7 | 4 | ∞ | 7 | 4 | ∞ |

The data shown in Tables 56 and 57 confirms no change in N1 and N2 signals for these samples. Interestingly, the combination of 55° C. and a reduced, 20 min reverse transcription incubation step was statistically identical to 45° C. and 45 min, confirming that that the combined RT-PCR reaction can be performed about 25 min faster than the standard protocol.

TABLE 56
Validation of higher temperature reverse transcription in TriCore samples

| | | | Condition 1 | | Condition 2 | | Condition 3 | | One-way |
|---|---|---|---|---|---|---|---|---|---|
| SARS-COV2 | | | Average | Standard | Average | Standard | Average | Standard | ANOVA |
| probe | Sample | Cq | RFU | deviation | RFU | deviation | RFU | deviation | p-value |
| N1 | TriCore | 13.75- | 31475 | 20498 | 30956 | 19613 | 29372 | 20289 | 0.97 |
| | Clinical | 31.65 | | | | | | | |
| | Positive | | | | | | | | |
| | TriCore | >35 | 2312 | 2027 | 1469 | 795 | 698 | 659 | 0.07 |
| | Clinical | | | | | | | | |
| | Positive | | | | | | | | |
| N2 | TriCore | 13.75- | 41015 | 21583 | 41172 | 20199 | 39517 | 22069 | 0.99 |
| | Clinical | 31.65 | | | | | | | |
| | Positive | | | | | | | | |
| | TriCore | >35 | 1638 | 559 | 7333 | 14980 | 790 | 373 | 0.28 |
| | Clinical | | | | | | | | |
| | Positive | | | | | | | | |

TABLE 57
Validation of higher temperature reverse transcription in γ-irradiated virion samples

| | Gamma- | | | | | | | |
|---|---|---|---|---|---|---|---|---|
| | Irradiated | Condition 1 | | Condition 2 | | Condition 3 | | One-way |
| SARS-COV2 | virions | Average | Standard | Average | Standard | Average | Standard | ANOVA |
| probe | copies/mL | RFU | deviation | RFU | deviation | RFU | deviation | p-value |
| N1 | 5000 | 15776 | 7935 | 15411 | 9934 | 16783 | 9466 | 0.97 |
| | 1000 | 5642 | 2357 | 2821 | 1215 | 4255 | 1341 | 0.12 |
| | 500 | 3193 | 1132 | 3507 | 1633 | 2012 | 1728 | 0.38 |
| | 0 | 2969 | 682 | 2466 | 1093 | 1043 | 444 | O.QI |
| N2 | 5000 | 29720 | 11369 | 26923 | 13706 | 29065 | 11935 | 0.95 |
| | 1000 | 7411 | 3740 | 3739 | 783 | 5349 | 1946 | 0.17 |
| | 500 | 3603 | 966 | 3461 | 2418 | 2713 | 2961 | 0.84 |
| | 0 | 1053 | 452 | 1483 | 474 | 594 | 596 | 0.1 |

### Fine Tuning of Hybridization/Wash in 96-Well Format Using a Vibratory Plate Shaker

Using an on-board plate shaker permits fluid phase mixing and laminar flow over the array surface during ambient temperature hybridization and washing, which helps reduce by at least 30%, the number of hybridization/wash steps in 96-well format. Two experiments were performed to test this.

Experiment 1: To test the effect of shaking on signals, 24 pooled positive samples were prepared (Boca) and tested under 3 separate hybridization conditions as follows:

X1—Plate remains static for 30 min hybridization incubation period.
X2—Plate is shaken at 1000 RPM for 30 min hybridization incubation period.
X3—Hybridization cocktail is mixed by pipetting up and down during hybridization period.
Results: Table 58 showed that condition X2 gave the highest average RFU across 8 wells on the appropriate probes, along with a lower standard deviation and lower background. These data reveal that shaking during hybridization improves signal strength when compared with the static hybridization method (X1) and the pipetting method (X).

TABLE 58
Comparison of static, shaking and pipetting hybridization method

| | X1 | | X2 | | X3 | |
|---|---|---|---|---|---|---|
| | Static Hybridization | | Shake at 1000 RPM | | Pipette to mix | |
| | Average | | Average | | Average | |
| | across | Std | across | Std | across | Std. |
| | 8 wells | Dev | 8 wells | Dev | 8 wells | Dev |
| SARS.COV2-N2-RE1.3 | 45967 | 2155 | 58539 | 2053 | 34560 | 1229 |
| SARS.COV2-N2-RE1.3 | 52055 | 3942 | 60242 | 337 | 36302 | 829 |
| SARS.COV2-N2-RE1.2 | 40439 | 1415 | 45335 | 4543 | 21923 | 2807 |
| SARS.COV2-N2-RE1.1 | 42262 | 3507 | 53268 | 6185 | 20897 | 7331 |
| RNAse. P. Probe-pub 1.2 | 61403 | 467 | 60418 | 357 | 59383 | 431 |
| RNAse. P. Probe-pub 1.1 | 61421 | 452 | 60426 | 364 | 58997 | 514 |
| SARS.COV2-N3-RE1.3 | 57433 | 5118 | 59976 | 581 | 44605 | 1535 |
| SARS.COV2-N1-RE1.2 | 33539 | 6896 | 46886 | 10031 | 27384 | 9464 |
| SARS.COV2-N3-RE1.2 | 55612 | 5070 | 60166 | 431 | 40722 | 6945 |
| SARS.COV2-N1-RE1.2 | 33665 | 5370 | 45564 | 7351 | 13312 | 11187 |
| SARS.COV2-N1-RE1.1 | 61432 | 424 | 60437 | 360 | 58662 | 1715 |
| SARS.COV2-N3-RE1.1 | 61293 | 423 | 60435 | 362 | 55161 | 3482 |
| SARS.COV2-N1-RE1.1 | 61277 | 382 | 60297 | 407 | 58487 | 1519 |
| 62-Negcont-B | 2266 | 818 | 4031 | 3155 | 2126 | 601 |
| SARS.COV2-N3-RE1.1 | 60725 | 1117 | 60428 | 361 | 55329 | 4479 |

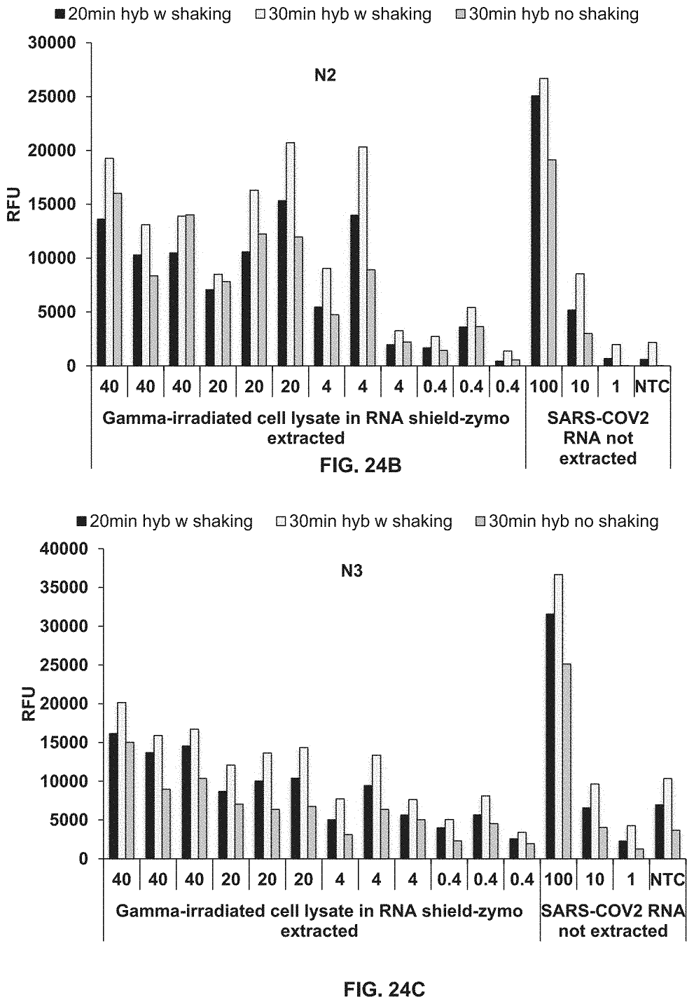

Experiment 2: RNA extracted (Zymo kit) from contrived samples (gamma irradiated cell lysates+nasal fluid in RNA Shield™ reagent (Zymo research) was used as the first sample at 0.4-40 copies per reaction. SARS-COV2 RNA was used as a second sample at 1-100 copies per reaction. RT-PCR parameters described in Protocol C (Table 54) was used. Results: The data in FIGS. 24A-24C clearly show that mixing during the 30 min hybridization increases hybridization signal strength about 2-fold among all probes tested.

Evaluate Simplified Alternatives to Standard Magnetic Bead CoV-2 Purification from NP/VTM and Mouthwash

A CERES NANOTRAP (Ceres Nanosciences, Inc.) technology for RNA extraction was evaluated for reducing time and costs of raw sample processing over the Zymo Quick-DNA/RNA Viral technology.

Alternate methods for reducing assay time and costs during raw sample processing were tested including the CERES NANOTRAP (Ceres Nanosciences Inc.) and Chitosan Coated Magnetic Beads (Creative Diagnostics Inc). Specifically, compared to Zymo's Quick-DNA/RNA Viral method the CERES NANOTRAP method is 1.5 hours faster requiring ⅓^{rd} of total manipulations, consumes 75% less consumables and may be automated for 96-well format.

### Contrived NP/VTM Samples

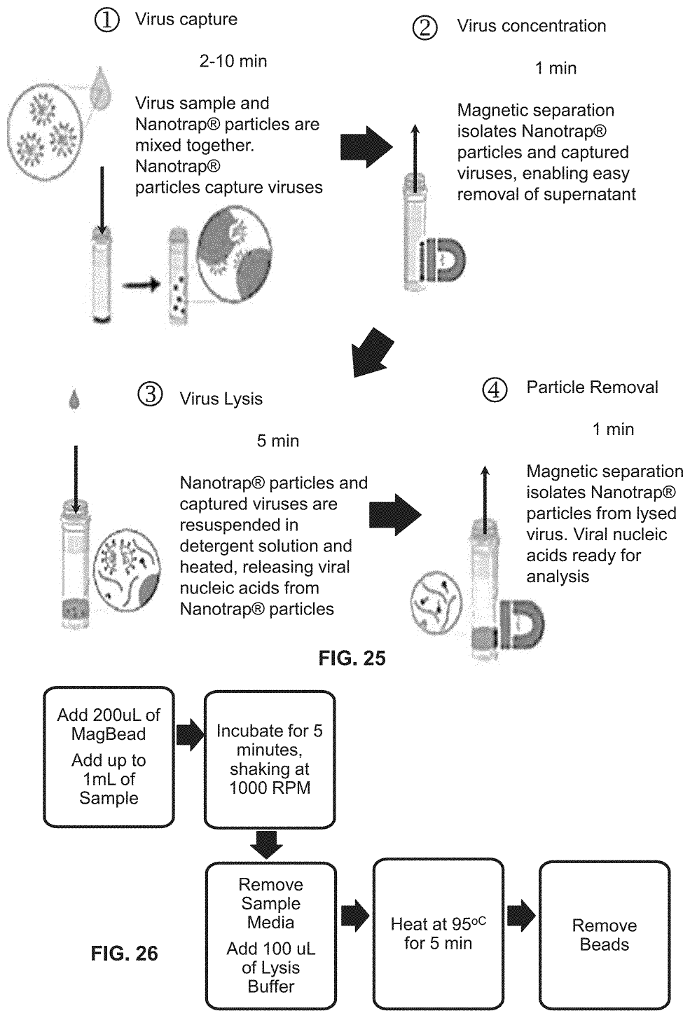

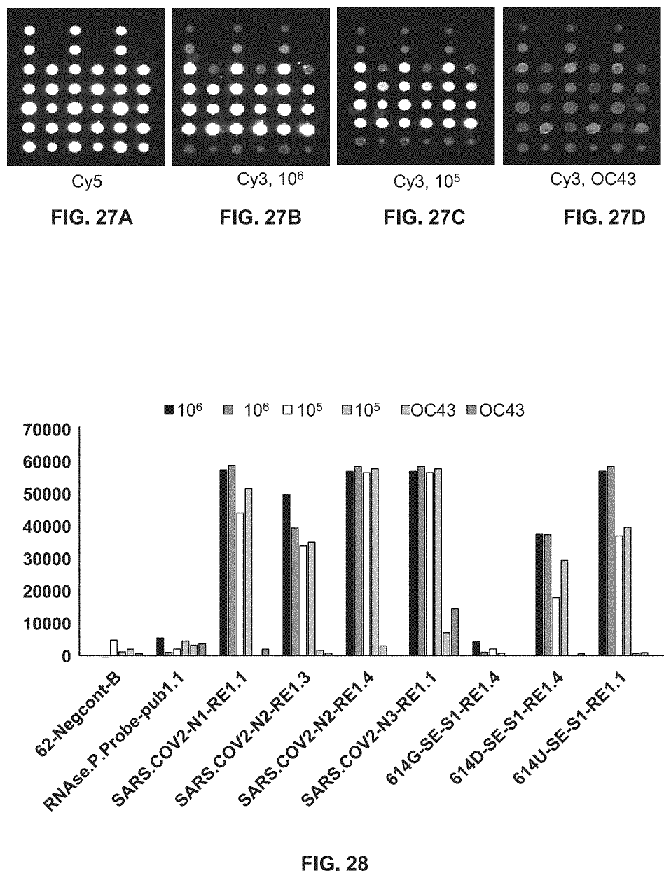

A comparison between the Zymo method described earlier with the CERES NANOTRAP method was performed for contrived NP/VTM samples prepared by Emory (heat-killed Cov-2 virus from BEI, in VTM). The CERES NANOTRAP method (FIGS. 25-26) was deployed on the raw samples to yield a pellet that was heat lysed in 1% Triton-X-100 in Molecular Grade Water before direct use in Asymmetric One-Pot RT-PCR, followed by Mini-RV analysis in the 96-well format. The results of these experiments are shown in FIGS. 27A-27D, 28 and 29 and Tables 59-61.

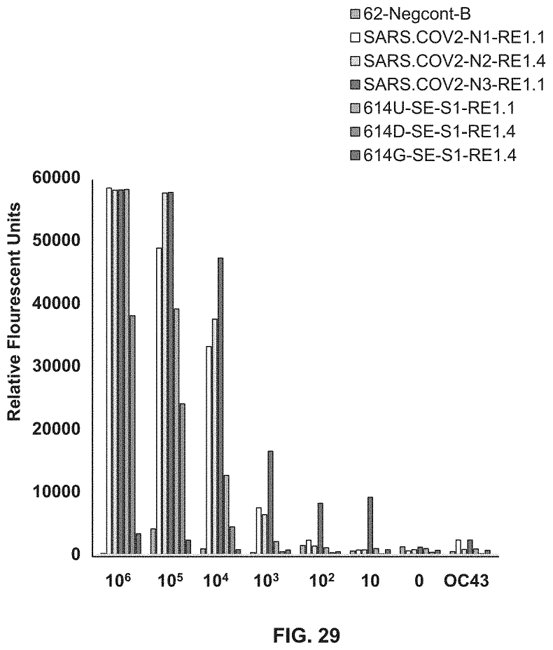

TABLE 59
Average RFU from data shown in FIG. 29

| | | SARS.COV2- | SARS.COV2- | SARS.COV2- | 614U-SE- | 614D-SE- | 614G-SE- |
|---|---|---|---|---|---|---|---|
| | 62-Negcont-B | N1-RE1.1 | N2-RE1.4 | N3-RE1.1 | S1-RE1.1 | S1-RE1.4 | S1-RE1.4 |
| 10⁶ | 131 | 58313 | 57988 | 57999 | 58088 | 37984 | 3296 |
| 10⁵ | 4071 | 48742 | 57533 | 57611 | 39089 | 23960 | 2242 |
| 10⁴ | 887 | 33078 | 37461 | 47154 | 12553 | 4366 | 734 |
| 10³ | 272 | 7420 | 6335 | 16416 | 2026 | 398 | 694 |
| 10² | 1410 | 2253 | 1335 | 8144 | 1066 | 263 | 408 |
| 10 | 525 | 684 | 727 | 9090 | 923 | 73 | 743 |
| 0 | 1179 | 551 | 739 | 1140 | 918 | 335 | 643 |
| OC43 1000 | 437 | 2266 | 773 | 2266 | 869 | 105 | 641 |
| OC43 100 | 1413 | 1289 | 1863 | 1026 | 1026 | 489 | 246 |

Among all 3 SARS-CoV-2 probes tested, the sensitivity of the Mini-RV assay subsequent to CERES NANOTRAP is identical or superior to that obtained using Quick-DNA/RNA Viral method for sample processing.

TABLE 60
Comparison of the Zymo and Ceres sample preparation methods

| | | | | | | |
|---|---|---|---|---|---|---|
| | SARS.COV2- | SARS.COV2- | SARS.COV2- | 614U-SE- | 614D-SE- | 614G-SE- |
| 62-Negcont-B | N1-RE1.1 | N2-RE1.4 | N3-RE1.1 | S1-RE1.1 | S1-RE1.4 | S1-RE1.4 |

Zymo Quick-DNA/RNA Viral method

| | | | | | | | |
|---|---|---|---|---|---|---|---|
| 10⁶ | 896 | 47139 | 55897 | 62143 | 29221 | 13325 | 2665 |
| 10⁶ | 1808 | 55909 | 53708 | 62344 | 37797 | 25071 | 3751 |
| 10⁵ | 1718 | 38649 | 40395 | 54269 | 10208 | 4424 | 1842 |
| 10⁵ | 933 | 37272 | 31301 | 48548 | 10566 | 4974 | 2484 |
| 10⁴ | 422 | 18915 | 17861 | 35288 | 3085 | 2238 | 1859 |
| 10⁴ | 2252 | 16966 | 27864 | 37627 | 3640 | 3046 | 2473 |
| 10³ | 1368 | 8840 | 11798 | 18995 | 2271 | 2212 | 2342 |
| 10³ | 2011 | 8059 | 7444 | 18287 | 2686 | 2669 | 2196 |
| 10³ | 1200 | 3244 | 5002 | 31634 | 3081 | 3001 | 2956 |
| 10² | 1103 | 3987 | 2694 | 3050 | 3334 | 1906 | 2574 |
| 10² | 17 | 3771 | 1605 | 4689 | 2369 | 3048 | 2785 |
| 10² | 3107 | 2520 | 682 | 15590 | 2718 | 1956 | 2204 |
| 10 | 740 | 1264 | 2803 | 3325 | 2386 | 2560 | 2530 |
| 10 | 4141 | 4491 | 2980 | 15011 | 4070 | 2541 | 3374 |
| 10 | 3089 | 4057 | 2924 | 12836 | 3333 | 3167 | 3355 |
| 0 | 2456 | 3339 | 1624 | 4413 | 2635 | 2940 | 3001 |
| 0 | 914 | 3445 | 1187 | 1317 | 1918 | 2325 | 2137 |
| 0 | 1920 | 1901 | 2111 | 6152 | 2761 | 2728 | 2670 |
| OC43 | 744 | 6579 | 2470 | 1897 | 2473 | 2319 | 2376 |
| OC43 | 2584 | 1558 | 2255 | 2164 | 2635 | 2484 | 2735 |
| OC43 | 2295 | 2754 | 1833 | 3665 | 2947 | 2237 | 2721 |
| OC43 | 2547 | 3974 | 1783 | 1925 | 3200 | 2980 | 3131 |

CERES NANOTRAP method

| | | | | | | | |
|---|---|---|---|---|---|---|---|
| 10⁶ | 250 | 57479 | 57171 | 57220 | 57289 | 38216 | 4936 |
| 10⁶ | 11 | 59147 | 58805 | 58778 | 58886 | 37751 | 1656 |
| 10⁵ | 6569 | 44814 | 57138 | 57290 | 37991 | 18235 | 2982 |
| 10⁵ | 1573 | 52670 | 57929 | 57933 | 40187 | 29685 | 1501 |
| 10⁴ | −394 | 34067 | 37511 | 47610 | 12552 | 4869 | 1357 |
| 10⁴ | 2167 | 32089 | 37411 | 46697 | 12553 | 3863 | 111 |
| 10³ | 271 | 8264 | 5231 | 17420 | 2401 | 834 | 609 |
| 10³ | 454 | 2721 | 3397 | 11240 | 1035 | −169 | 839 |
| 10³ | 91 | 11275 | 10378 | 20588 | 2643 | 529 | 633 |
| 10² | 1724 | 251 | 411 | 7294 | 739 | 236 | 189 |
| 10² | 1375 | 4602 | 852 | 7084 | 1664 | 513 | 747 |
| 10² | 1133 | 1907 | 2743 | 10052 | 794 | 39 | 289 |
| 10 | 591 | 1047 | 1535 | 7319 | 939 | 5 | 785 |
| 10 | 243 | 855 | 164 | 7037 | 788 | 66 | 716 |
| 10 | 741 | 148 | 480 | 12913 | 1042 | 149 | 729 |
| 0 | 588 | 260 | 296 | 2093 | 577 | 160 | 801 |
| 0 | 1248 | 982 | 679 | 241 | 1013 | 363 | 487 |
| 0 | 1702 | 410 | 1243 | 1087 | 1165 | 482 | 643 |
| OC43 1000 | 176 | 3011 | 888 | 3011 | 830 | 140 | 707 |
| OC43 1000 | 698 | 1522 | 658 | 1522 | 909 | 70 | 575 |
| OC43 100 | 2307 | 399 | 3585 | 967 | 967 | 44 | 86 |
| OC43 100 | 520 | 2179 | 141 | 1086 | 1086 | 935 | 405 |

TABLE 61
Comparison of the CERES NANOTRAP method for various sample inputs

| | | | | | | | |
|---|---|---|---|---|---|---|---|
| Well | | SARS.COV2- | SARS.COV2- | SARS.COV2- | 614U-SE- | 614D-SE- | 614G-SE- |
| number | 62-Negcont-B | N1-RE1.1 | N2-RE1.4 | N3-RE1.1 | S1-RE1.1 | S1-RE1.4 | S1-RE1.4 |

5 μL Input

| | | | | | | | | |
|---|---|---|---|---|---|---|---|---|
| 10⁴ | Well 1 | −394 | 34067 | 37511 | 47610 | 12552 | 4869 | 1357 |
| 10⁴ | Well 2 | 2167 | 32089 | 37411 | 46697 | 12553 | 3863 | 111 |
| 10³ | Well 3 | 271 | 8264 | 5231 | 17420 | 2401 | 834 | 609 |
| 10³ | Well 4 | 454 | 2721 | 3397 | 11240 | 1035 | −169 | 839 |
| 10² | Well 5 | 91 | 11275 | 10378 | 20588 | 2643 | 529 | 633 |
| 10² | Well 6 | 1724 | 251 | 411 | 7294 | 739 | 236 | 189 |
| 10² | Well 7 | 1375 | 4602 | 852 | 7084 | 1664 | 513 | 747 |
| 10³ | Well 8 | 1133 | 1907 | 2743 | 10052 | 794 | 39 | 289 |
| 10 | Well 9 | 591 | 1047 | 1535 | 7319 | 939 | 5 | 785 |
| 10 | Well 10 | 243 | 855 | 164 | 7037 | 788 | 66 | 716 |
| 10 | Well 11 | 741 | 148 | 480 | 12913 | 1042 | 149 | 729 |
| 0 | Well 12 | 588 | 260 | 296 | 2093 | 577 | 160 | 801 |
| 0 | Well 13 | 1248 | 982 | 679 | 241 | 1013 | 363 | 487 |
| 0 | Well 14 | 1702 | 410 | 1243 | 1087 | 1165 | 482 | 643 |
| OC43 | Well 15 | 176 | 3011 | 888 | 3011 | 830 | 140 | 707 |
| OC43 | Well 16 | 698 | 1522 | 658 | 1522 | 909 | 70 | 575 |

10 μL Input

| | | | | | | | | |
|---|---|---|---|---|---|---|---|---|
| 10⁴ | Well 25 | 508 | 31130 | 35936 | 36363 | 9159 | 1571 | 1 |
| 10⁴ | Well 26 | 96 | 28367 | 37529 | 37733 | 9360 | 2261 | 400 |
| 10³ | Well 27 | 801 | 11549 | 16029 | 23418 | 2364 | 234 | 579 |
| 10³ | Well 28 | 2517 | 6724 | 5685 | 11865 | 2251 | 751 | 915 |
| 10² | Well 29 | 1399 | 9409 | 14780 | 27387 | 2622 | 1280 | 235 |
| 10² | Well 30 | 91 | 660 | 1407 | 3042 | 472 | 279 | 491 |
| 10² | Well 31 | 751 | 2155 | 1513 | 6435 | 696 | 346 | 221 |
| 10³ | Well 32 | 189 | 1490 | 1170 | 3681 | 1322 | 45 | 980 |
| 10 | Well 33 | 1218 | 54 | 1215 | 4366 | 287 | 220 | 487 |
| 10 | Well 34 | 1536 | 668 | 716 | 1044 | 904 | 30 | 509 |
| 10 | Well 35 | 1325 | 1563 | 2139 | 7748 | 671 | 134 | 277 |
| 0 | Well 36 | 1054 | 284 | 636 | 5169 | 624 | 207 | 339 |
| 0 | Well 37 | 36 | 1423 | 1026 | 9562 | 1188 | 936 | 689 |
| 0 | Well 38 | 1135 | 747 | 1148 | 1345 | 994 | 303 | 407 |
| OC43 | Well 39 | 1053 | 1498 | 2731 | 932 | 692 | 342 | 373 |
| OC43 | Well 40 | 949 | 990 | 640 | 4995 | 712 | 303 | 200 |

15 μL Input

| | | | | | | | | |
|---|---|---|---|---|---|---|---|---|
| 10⁴ | Well 41 | 713 | 31449 | 39029 | 44974 | 18520 | 4425 | 180 |
| 10⁴ | Well 42 | 624 | 33111 | 37242 | 40909 | 14729 | 3098 | −22 |
| 10³ | Well 43 | 1110 | 14349 | 18064 | 28520 | 3414 | 106 | −86 |
| 10³ | Well 44 | 1917 | 4070 | 7575 | 11626 | 2144 | −53 | 668 |
| 10³ | Well 45 | 849 | 15887 | 18735 | 32519 | 3206 | 283 | −50 |
| 10² | Well 46 | 322 | 2366 | 1268 | 8336 | 952 | 75 | 514 |
| 10² | Well 47 | 968 | 2696 | 3649 | 3395 | 1270 | 64 | 466 |
| 10² | Well 48 | 1454 | 3282 | 3230 | 3102 | 1311 | 134 | 507 |
| 10 | Well 49 | 1523 | −93 | 2046 | 2658 | 725 | 745 | 1167 |
| 10 | Well 50 | 1245 | 863 | 86 | 3717 | 1027 | 181 | 880 |
| 10 | Well 51 | 805 | 1726 | 1070 | 10586 | 1637 | 459 | 265 |
| 0 | Well 52 | 981 | 189 | 1559 | 12322 | 622 | 856 | 781 |
| 0 | Well 53 | 561 | 1033 | 1240 | 2424 | 577 | 713 | 74 |
| 0 | Well 54 | 1942 | 81 | 29 | 2846 | 1613 | 1201 | 593 |
| OC43 | Well 55 | 217 | 1466 | 1025 | 3771 | 815 | 3 | 241 |
| OC43 | Well 56 | 918 | −111 | 972 | 2819 | 1692 | 705 | 576 |

### Clinical NP/VTM Samples

Clinical samples (positive and negative NP/VTM samples) previously characterized at TriCore via the Roche, Cobas 6800 SARS-CoV-2 platform, were analyzed to generate a Q-RT-PCR based Cq values for each clinical isolate. All samples were subjected to viral capture and enrichment using CERES NANOTRAP, followed by direct heat lysis of the resulting viral pellet in 1% Triton-X-100 as described above. The lysate (5 μL) from each of the 61 samples was used as input without additional purification, in the Asymmetric One-Step RT-PCR, followed by Mini-RV hybridization analysis. Two types of analysis were performed on the hybridization data.

Analysis 1. Hybridization signals (RFU) from all Mini-RV probes in the positive and negative TriCore samples was used to generate mean and standard deviation for the LoB, which was then used to determine the RFU threshold to be deployed in analysis of the samples. The Clinical and Laboratory Standards Institute (CLSI) standard was applied in threshold determination. To account for user differences, LoB was modified using the equation;

LoB=(3*Standard Deviation)+Average

Using this threshold value (Table 62), clinical sensitivity, and specificity, PPV and NPV were calculated for each probe in the Mini-RV test, and in turn for the overall call generated by AUGURY from those multiplex probe data (Table 62).

Analysis 2. To facilitate analysis of the relationship between Q-RT-PCR signal strength (Cq) and the Ceres+Mini-RV signal strength (RFU), the data from TriCore, NP/VTM clinical positives was rank-ordered based on their Cobas Cq value—lowest Cq (highest viral load) at the top and highest Cq (lowest viral load) at the bottom (Table 63). Cq values from Roche Cobas 6800 for the negative samples is shown in Table 64.

### Results

The data show 100% clinical sensitivity and clinical specificity (Table 62, bottom row). Highest affinity Mini-RV probes (SARS.COV2-N2-RE1.3 and SARS.COV2-N3-RE1.1) remained positive even in the highest Cq (lowest viral load) positive samples, producing clearly defined calls, throughout (Table 62, columns 7,9). Additionally, the relationship between Q-RT-PCR (Cq) values and RFU signals (Table 63) manifest in the comparison of high affinity versus medium affinity Mini-RV probes enables microarray-based quantitation of Cov-2 RNA load.

TABLE 62
One-Pot Bias Labeling RT-PCR-CERES NANOTRAP

| | Threshold |
|---|---|
| 62-Negcont-B | 2309 |
| RNAse.P.Probe-pub1.1 | N/A |
| SARS.COV2-N1-RE1.1 | 2263 |
| SARS.COV2-N2-RE1.3 | 2145 |
| SARS.COV2-N2-RE1.4 | 2128 |
| SARS.COV2-N3-RE1.1 | 5662 |
| 614U-SE-S1-RE1.1 | 1466 |
| 614D-SE-S1-RE1.4 | N/A |
| 614G-SE-S1-RE1.4 | N/A |

| | | | | | (a) | (b) | (c) | (d) | | | | | |
|---|---|---|---|---|---|---|---|---|---|---|---|---|---|
| | Average | Standard | Average | Standard | True | False | False | True | | | | | |
| | Posi- | Devi- | Nega- | Devi- | Posi- | Posi- | Nega- | Nega- | | Sensi- | Speci- | | |
| | tives | ation | tives | ation | tive | tive | tives | tives | LoB | tivity | ficity | PPV | NPV |
| 62-Negcont-B | 543 | 361 | 853 | 485 | 30 | 0 | 0 | 31 | 1652 | 100 | 100 | 100 | 100 |
| RNAse.P.Probe-pub1.1 | 54843 | 12349 | 35575 | 13431 | 30 | 0 | 0 | 31 | N/A | 100 | 100 | 100 | 100 |
| SARS.COV2-N1-RE1.1 | 22384 | 23833 | 973 | 430 | 30 | 0 | 11 | 31 | 1680 | 74 | 100 | 100 | 74 |
| SARS.COV2-N2-RE1.3 | 16605 | 18344 | 1268 | 292 | 30 | 0 | 1 | 31 | 1749 | 97 | 100 | 100 | 97 |
| SARS.COV2-N2-RE1.4 | 25298 | 25435 | 615 | 504 | 30 | 0 | 11 | 31 | 1445 | 74 | 100 | 100 | 74 |
| SARS.COV2-N3-RE1.1 | 36436 | 16105 | 2271 | 1131 | 30 | 0 | 0 | 31 | 4130 | 100 | 100 | 100 | 100 |
| 614U-SE-S1-RE1.1 | 16317 | 22839 | 569 | 299 | 30 | 0 | 15 | 31 | 1061 | 68 | 100 | 100 | 67 |
| 614D-SE-S1-RE1.4 | 877 | 701 | 428 | 344 | 30 | 0 | N/A | 31 | 993 | N/A | 100 | 100 | N/A |
| 614G-SE-S1-RE1.4 | 8695 | 14376 | 474 | 324 | 30 | 0 | N/A | 31 | 1007 | N/A | 100 | 100 | N/A |
| Overall Call | N/A | N/A | N/A | N/A | 30 | 0 | 0 | 31 | N/A | 100 | 100 | 100 | 100 |

TABLE 63
Analysis of clinical positive samples using CERES NANOTRAP + Mini-RV

| | | PDx | | | | | | | | | |
|---|---|---|---|---|---|---|---|---|---|---|---|
| | | Over- | 62- | | | | | | 614U- | 614D- | 614G- |
| Patient | Ct | all | Negcont- | RNAse.P.Probe- | SARS.COV2- | SARS.COV2- | SARS.COV2- | SARS.COV2- | SE-S1- | SE-S1- | SE-S1- |
| ID | Value | Call | B | pub1.1 | N1-RE1.1 | N2-RE1.3 | N2-RE1.4 | N3-RE1.1 | RE1.1 | RE1.4 | RE1.4 |
| 216708 | 16.3 | + | 1025 | 60184 | 60031 | 42974 | 59873 | 59842 | 57442 | 1564 | 34799 |
| 216005 | 17 | + | 532 | 61079 | 39962 | 14688 | 40212 | 39328 | 33600 | 412 | 7758 |
| 216002 | 17.6 | + | 382 | 60937 | 15357 | 2951 | 9051 | 15378 | 740 | 66 | 302 |
| 215989 | 18.8 | + | 1214 | 60636 | 60733 | 59869 | 60499 | 60580 | 60441 | 2790 | 43307 |
| 216565 | 19.4 | + | 791 | 61053 | 42644 | 35231 | 60325 | 47596 | 16915 | 466 | 4334 |
| 215997 | 19.5 | + | 791 | 60641 | 61080 | 57518 | 60766 | 60899 | 60772 | 2658 | 42760 |
| 215988 | 19.6 | + | 171 | 61208 | 13544 | 3714 | 15728 | 15301 | 1001 | 786 | 1069 |
| 215999 | 19.8 | + | 671 | 61032 | 60921 | 41919 | 60692 | 60753 | 55691 | 1458 | 35558 |
| 215992 | 20.5 | + | 335 | 60833 | 55607 | 38292 | 60412 | 60202 | 34336 | 1168 | 8008 |
| 215982 | 21.4 | + | 320 | 60990 | 34856 | 22652 | 42393 | 37004 | 7092 | 406 | 1875 |
| 215993 | 21.4 | + | 399 | 60757 | 26397 | 9905 | 36512 | 33408 | 3419 | 555 | 1149 |
| 215995 | 23.2 | + | 435 | 61025 | 9130 | 4462 | 8029 | 32541 | 314 | 563 | 837 |
| 216001 | 23.9 | + | 232 | 61511 | 469 | 1842 | 20 | 14758 | 98 | 361 | 512 |
| 215983 | 24.1 | + | 271 | 61561 | 40190 | 26371 | 50555 | 47049 | 39053 | 750 | 12218 |
| 216003 | 24.2 | + | 1372 | 52363 | 60745 | 49426 | 60579 | 60625 | 60468 | 2503 | 39610 |
| 215996 | 24.8 | + | 262 | 58165 | 11307 | 7098 | 22354 | 36237 | 4127 | 34 | 1227 |
| 215998 | 25.2 | + | 861 | 61126 | 60506 | 46522 | 60843 | 60913 | 60715 | 1435 | 37778 |
| 216701 | 25.9 | + | −116 | 60209 | 41064 | 27185 | 49944 | 54044 | 37956 | 856 | 12876 |
| 216564 | 26.1 | + | 137 | 39263 | 21521 | 12341 | 36847 | 38661 | 7944 | 257 | 1981 |
| 216566 | 26.3 | + | 52 | 44491 | 29418 | 16484 | 36751 | 39826 | 11800 | 252 | 3070 |
| 216700 | 26.5 | + | 142 | 60946 | 31126 | 19117 | 41213 | 35461 | 10845 | 35 | 2391 |
| 215987 | 27 | + | 801 | 61899 | −14 | 3017 | 584 | 15086 | 526 | 1259 | 1253 |
| 215994 | 29.2 | + | 741 | 39522 | 363 | 2949 | 862 | 35620 | 487 | 1253 | 970 |
| 216007 | 29.9 | + | 234 | 61208 | 138 | 2243 | 1357 | 16169 | 189 | 789 | 395 |
| 215991 | 30.7 | + | 828 | 43373 | 396 | 2761 | 638 | 34696 | 275 | 754 | 381 |
| 215986 | 30.9 | + | 759 | 60999 | 360 | 3427 | 1772 | 29400 | 567 | 953 | 2165 |
| 215984 | 32.1 | + | 560 | 16179 | 533 | 2968 | 752 | 37023 | 738 | 441 | 631 |
| 215990 | 33.4 | + | 662 | 38405 | −36 | 5394 | 911 | 27314 | 711 | 1061 | 822 |
| 215981 | 34.3 | + | 1188 | 62578 | −72 | 4987 | 1020 | 18036 | 972 | 1622 | 729 |
| 215985 | 34.5 | + | 552 | 14973 | 450 | 2849 | 34 | 41135 | 596 | 812 | 1066 |

TABLE 64
Analysis of clinical negative samples using CERES NANOTRAP + Mini-RV

| | Ct Value from | PDx Overall | | RNAse.P.Probe- | SARS.COV2- |
|---|---|---|---|---|---|
| Patient ID | Roche Cobas 6800 | Call | 62-Negcont-B | pub1.1 | N1-RE1.1 |
| PATHO-001 | T1 > 35; T2 > 38 | − | 1443 | 25554 | 1071 |
| PATHO-002 | T1 > 35; T2 > 38 | − | 528 | 23470 | 847 |
| PATHO-003 | T1 > 35; T2 > 38 | − | 728 | 41852 | 817 |
| PATHO-004 | T1 > 35; T2 > 38 | − | 303 | 43625 | 462 |
| PATHO-005 | T1 > 35; T2 > 38 | − | 725 | 54472 | 1205 |
| PATHO-006 | T1 > 35; T2 > 38 | − | 251 | 36887 | 683 |
| PATHO-007 | T1 > 35; T2 > 38 | − | 1307 | 60550 | 1313 |
| PATHO-009 | T1 > 35; T2 > 38 | − | 1291 | 24409 | 966 |
| PATHO-010 | T1 > 35; T2 > 38 | − | 718 | 36868 | 554 |
| PATHO-011 | T1 > 35; T2 > 38 | − | 862 | 37259 | 506 |
| PATHO-012 | T1 > 35; T2 > 38 | − | 152 | 14057 | 62 |
| PATHO-013 | T1 > 35; T2 > 38 | − | 1207 | 54393 | 884 |
| PATHO-014 | T1 > 35; T2 > 38 | − | 428 | 18317 | 852 |
| PATHO-015 | T1 > 35; T2 > 38 | − | 856 | 16642 | 983 |
| PATHO-016 | T1 > 35; T2 > 38 | − | 292 | 51315 | 962 |
| PATHO-017 | T1 > 35; T2 > 38 | − | 694 | 42375 | 453 |
| PATHO-018 | T1 > 35; T2 > 38 | − | 241 | 14001 | 265 |
| PATHO-019 | T1 > 35; T2 > 38 | − | 602 | 10226 | 905 |
| PATHO-020 | T1 > 35; T2 > 38 | − | 791 | 37377 | 817 |
| PATHO-021 | T1 > 35; T2 > 38 | − | 1352 | 55209 | 684 |
| PATHO-022 | T1 > 35; T2 > 38 | − | 1760 | 47587 | 1313 |
| PATHO-023 | T1 > 35; T2 > 38 | − | 1506 | 35160 | 1517 |
| PATHO-024 | T1 > 35; T2 > 38 | − | 529 | 21418 | 1064 |
| PATHO-025 | T1 > 35; T2 > 38 | − | 751 | 41096 | 2052 |
| PATHO-026 | T1 > 35; T2 > 38 | − | 647 | 35665 | 886 |
| PATHO-027 | T1 > 35; T2 > 38 | − | 718 | 40323 | 1532 |
| PATHO-028 | T1 > 35; T2 > 38 | − | 181 | 32818 | 849 |
| PATHO-029 | T1 > 35; T2 > 38 | − | 2028 | 39589 | 1361 |
| PATHO-030 | T1 > 35; T2 > 38 | − | 1135 | 25638 | 1223 |
| LoB Threshold | N/A | N/A | 2309 | N/A | 2263 |

| | SARS.COV2- | SARS.COV2- | SARS.COV2- | 614U-SE- | 614D-SE- | 614G-SE- |
|---|---|---|---|---|---|---|
| Patient ID | N2-RE1.3 | N2-RE1.4 | N3-RE1.1 | S1-RE1.1 | S1-RE1.4 | S1-RE1.4 |
| PATHO-001 | 1250 | 405 | 953 | 255 | 131 | 148 |
| PATHO-002 | 759 | −32 | 2755 | 450 | 215 | 191 |
| PATHO-003 | 1111 | 417 | 3177 | 466 | 175 | 626 |
| PATHO-004 | 1239 | 181 | 2498 | 181 | 197 | 338 |
| PATHO-005 | 1220 | 1468 | 2247 | 503 | 603 | 479 |
| PATHO-006 | 1167 | 457 | 1302 | 322 | 254 | 302 |
| PATHO-007 | 546 | 668 | 3975 | 527 | 314 | −10 |
| PATHO-009 | 1323 | 24 | 1402 | 631 | 426 | 178 |
| PATHO-010 | 1347 | 447 | 1961 | 282 | 409 | 348 |
| PATHO-011 | 1640 | 661 | 2102 | 389 | −13 | 163 |
| PATHO-012 | 1036 | 167 | 857 | 512 | 221 | 165 |
| PATHO-013 | 1303 | 379 | 2087 | 593 | 242 | 98 |
| PATHO-014 | 1106 | 846 | 2416 | 425 | 475 | 65 |
| PATHO-015 | 961 | 153 | 1857 | 257 | 38 | 220 |
| PATHO-016 | 1639 | 963 | 4367 | 356 | −24 | 473 |
| PATHO-017 | 1182 | 8 | 1705 | 359 | 40 | 213 |
| PATHO-018 | 938 | 197 | 125 | 436 | 23 | 267 |
| PATHO-019 | 985 | −4 | 614 | 707 | 116 | 275 |
| PATHO-020 | 1113 | 291 | 2603 | 249 | 223 | 557 |
| PATHO-021 | 1705 | 920 | 2657 | 1194 | 797 | 1051 |
| PATHO-022 | 1772 | 2134 | 3656 | 1235 | 1011 | 863 |
| PATHO-023 | 1726 | 742 | 3535 | 1095 | 782 | 780 |
| PATHO-024 | 1268 | 1017 | 1666 | 447 | 338 | 459 |
| PATHO-025 | 1171 | 408 | 819 | 575 | 889 | 1057 |
| PATHO-026 | 1543 | 677 | 4850 | 573 | 839 | 742 |
| PATHO-027 | 1440 | 941 | 2718 | 703 | 521 | 503 |
| PATHO-028 | 1178 | 543 | 2412 | 403 | 436 | 579 |
| PATHO-029 | 1248 | 1401 | 3490 | 524 | 1017 | 1135 |
| PATHO-030 | 1634 | 781 | 1714 | 1206 | 1253 | 946 |
| LoB Threshold | 2145 | 2128 | 5662 | 1466 | N/A | N/A |

### Example 26

### Clinical Sensitivity and Specificity Using the CERES NANOTRAP Mini-RV Technology

The protocol used 200 μL of beads and elution in 100 μL of extraction buffer (0.5% TritonX-100 in water) and additionally, a wash step after the first pelleting step. Clinical sensitivity and specificity analysis of the CERES NANOTRAP Mini-RV technology using 30 Tricore (Cobas-Pos)

and 30 (Cobas-Neg) NP-VTM samples were 100% relative to the Cobas predicate. Probe threshold was calculated from LoB data obtained from the matched clinical negative samples using the formula;

Threshold=3 ×(STV)+Mean

where Mean is the Mean value of RFU signal and STV is one standard deviation about that mean.

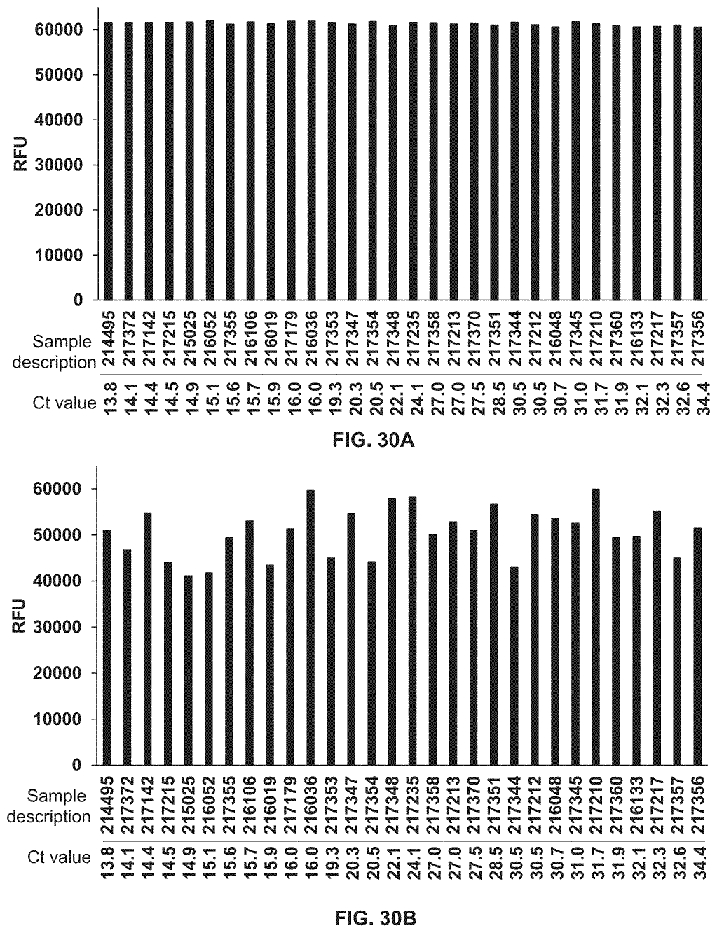

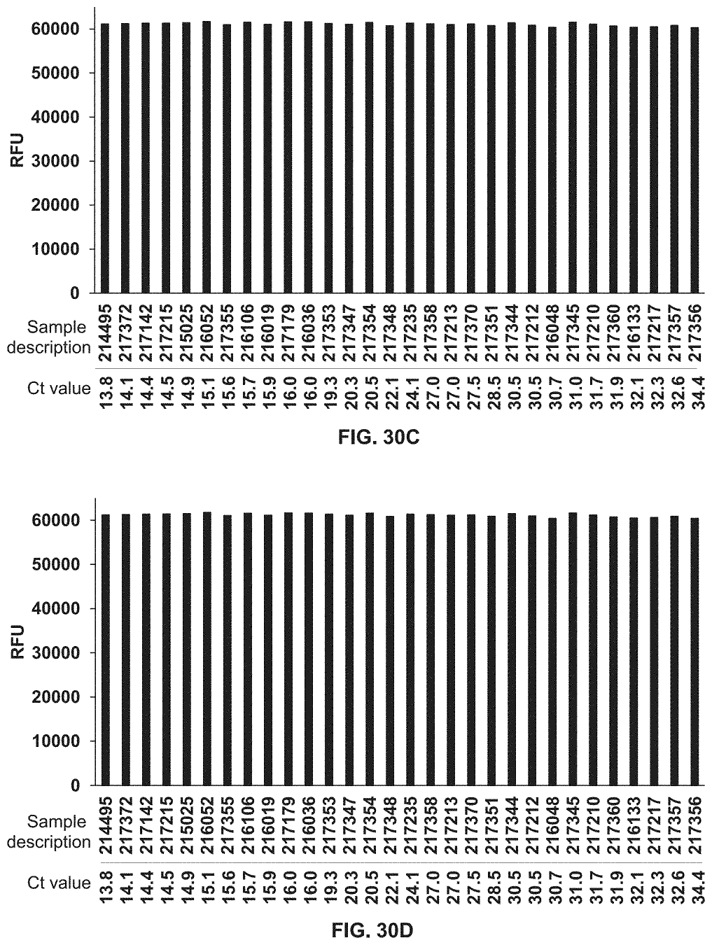

FIGS. 30A-30D show the 30 “Cobas-Positive” TriCore samples arranged such that the apparent viral load decreases from left to right (lowest Cq value→highest Cq value). Thus, using the modified Ceres bead protocol, a signal/threshold ratio greater than 10 was obtained for all COVID-19 probes (N1, N2 and N3) in all of the 30 samples even at the Limit of Detection for the Cobas Assay (Cq values˜35). The RFU signals obtained in these experiments provide support for using the CERES NANOTRAP Mini-RV technology even at the Cobas Limit of Detection (˜35) when pooled testing is desired.

### LoD Analysis Using the CERES NANOTRAP Mini-RV Technology

In addition to clinical sensitivity and specificity analysis, determination of LoD in units of virions/ml, were performed using virus that were subjected to heat, radiative or chemical denaturation. On contrived samples distributed as PT standards (FDA's SARS-CoV-2 Reference Panel Comparative Data) the Roche Cobas Q-RT PCR assay delivered a LoD of 1,800 copies/mL. Thus, all 30 positive clinical samples studied here (TriCore, with Cobas Predicate) are expected to contain >1,800 copies/mL

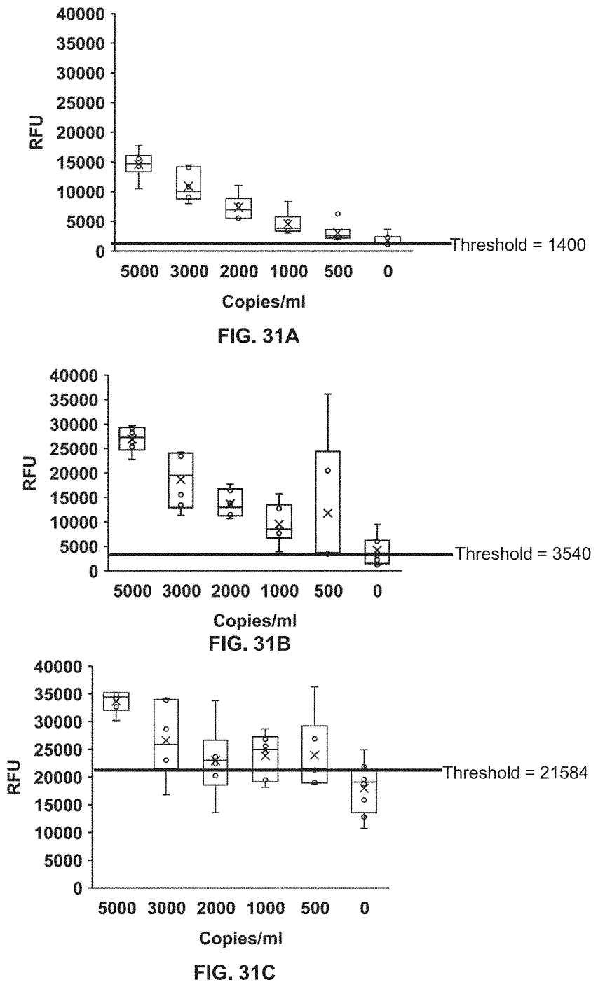

As shown in FIGS. 31A-31C, using the same procedural improvements deployed with the TriCore clinical samples the Signal/Threshold values and the resulting LoD values obtained in the contrived samples were somewhat lower than would be expected from the clinical isolates. The improvements made to the CERES NANOTRAP Mini-RV protocol suggest a LoD in the 500 copies/mL range. To further refine the LoD to harmonize with the clinical results (Roche Cobas LoD 1800 copies/mL), modifications were done to the protocol as follows:

### Experiment 1:

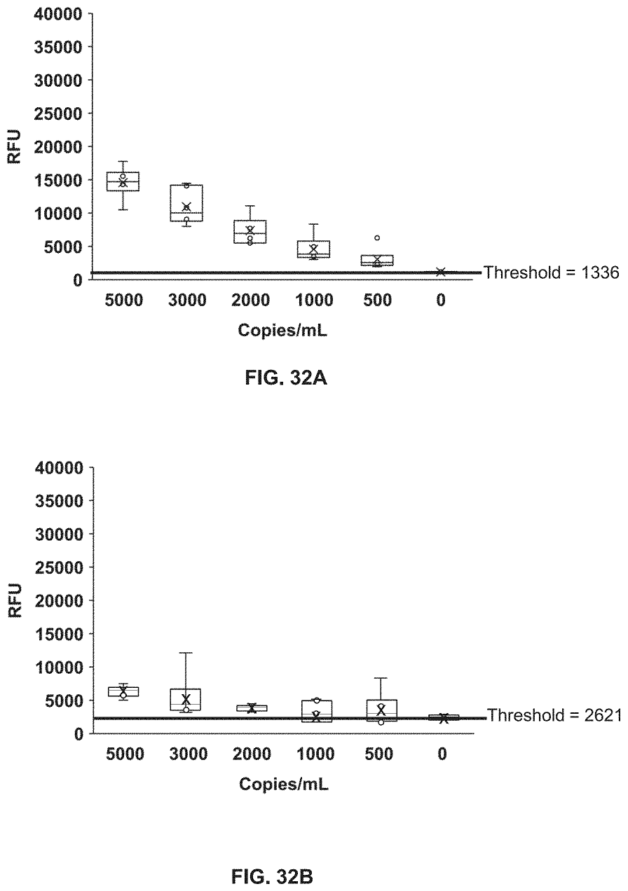

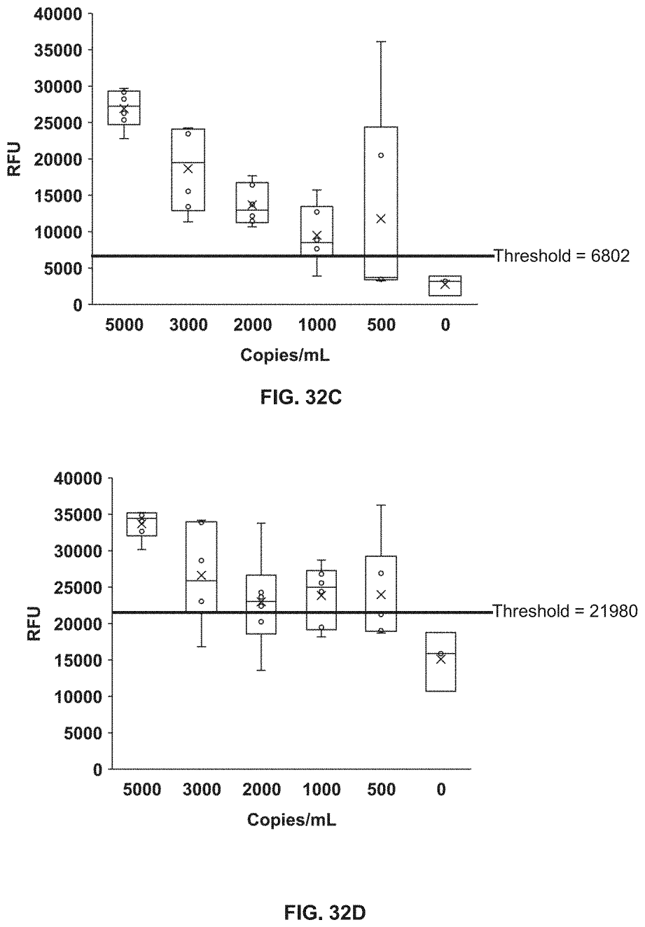

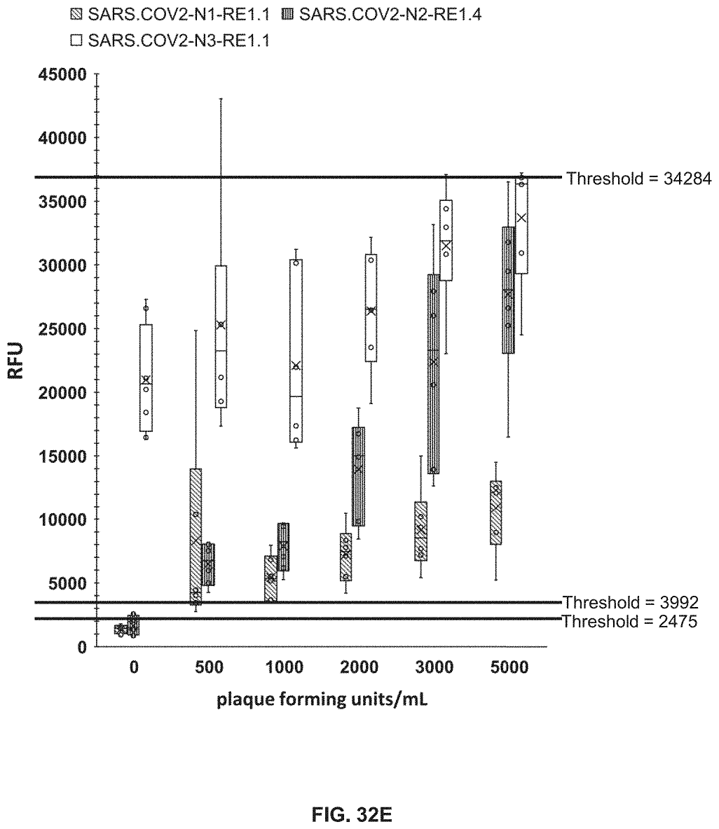

A finer dilution of the heat inactivated SARS-CoV-2 in VTM was tested at 5000, 3000, 2000, 1000 and 500 copies/mL (N=6 for each concentration). The samples were prepared in 500 μL of VTM and processed using the Ceres protocol with a final elution/lysis volume of 100 μL. The results showed 100% detection capability down to 500 copies/mL for N1 and N2, whereas a high background and variability for the N3 probe (FIGS. 32A-32E).

### Experiment 2:

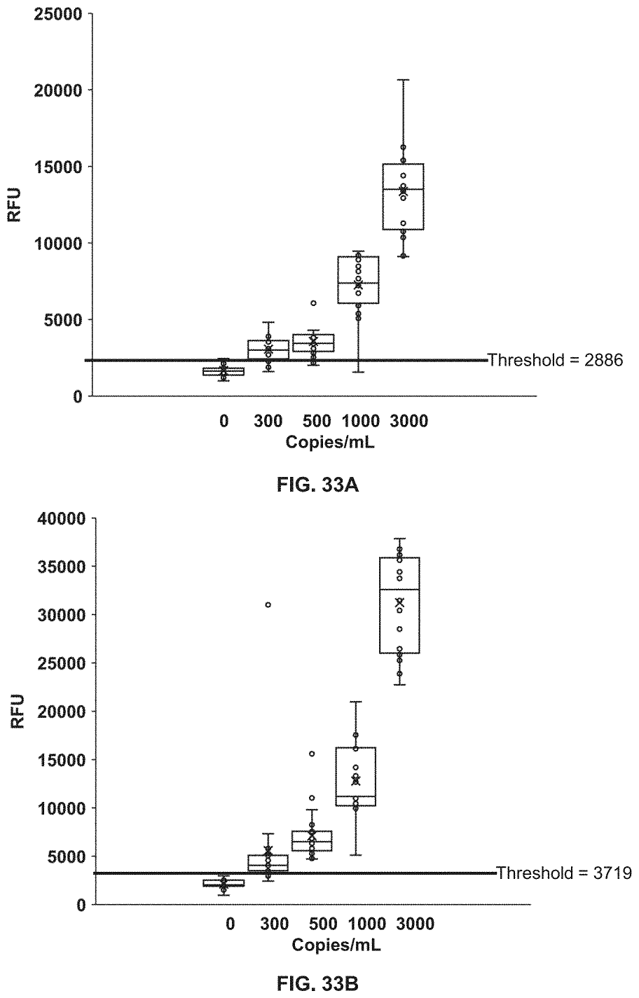

Based on the result from Experiment 1 above, additional LoD experiments were performed with increased sample number towards obtaining 95% positive results at 500 copies/mL. For this purpose, three sets of LoD samples were created at 3000, 1000, 500, 300, and 0 cp/mL (N=20 each) in VTM. Additionally, fresh vial of SARS-CoV-2 heat inactivated virus was used and prepared in VTM containing 10% glycerol prior to diluting to the concentrations tested. The results in FIGS. 33A-33B, demonstrate the ability of this method to yield an LoD at or just below 500 cp/mL. At 3000 cp/mL, the RFU values are closer to that observed with clinical samples. It is clear from this experiment that improper storage and/or degradation of the virus through multiple freeze thaws is a key contributor to LoD values obtained.

### Experiment 3:

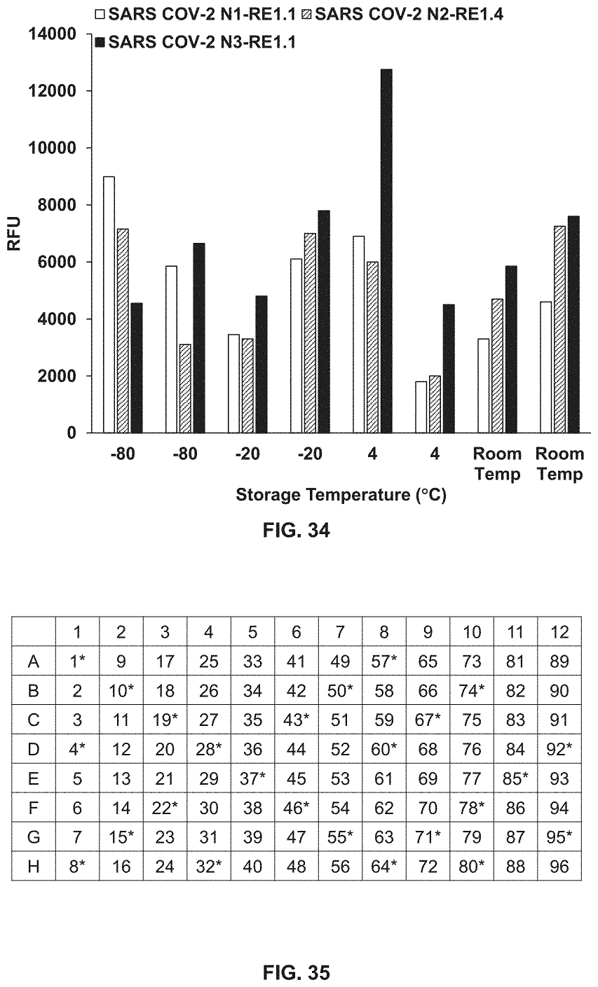

In the last experiment the hypothesis that freeze thaws might impact the background signal was tested. A large, pooled sample was created in which heat inactivated SARS-CoV-2 virus was diluted into VTM at 5000 cp/mL. The samples were aliquoted and stored at −80, −20, 4, and room temperature for 72 hours. The samples were then thawed or removed from the refrigerator and prepared using the CERES NANOTRAP beads followed by the DETECTX-RV protocol. No differences in background or signal strength were observed due to the different storage conditions or freeze thaws (FIG. 34).

### Example 27

LoD Analysis of the CERES NANOTRAP Mini-RV Technology Pairing with Heat-Denatured CoV-2 (BEI) in VTM.

The clinical and LoD results presented in the previous example demonstrated excellent sensitivity and specificity and an LoD at or below 500 copies/mL. In addition, it was noted that there was variability in the baseline signal for the N3 probe. To further refine the protocol, pooling studies were undertaken and additionally, the platform was evaluated for multiplexed detection of Influenza A and B.

Experiment 1: A fully automated Ceres run was performed on the Tecan EVO150. In order to evaluate the run a checkerboard pattern (FIG. 35) was created and in the asterisked wells was added, clinical negative sample spiked with 25000 or 5000 copies/mL of irradiated SARS-CoV-2. This analysis revealed that 97% of the wells were called correctly, with two negative samples called as positive, and one positive sample called as negative.

Experiment 2: Three different lots of SARS-CoV-2 viral material (heat inactivated and gamma irradiated) were tested under different storage conditions (Table 65). A dilution from 30,000 to 1,000 copies/mL was used for each source material. The results shown in Table 65 demonstrate that absence of 10% glycerol displayed the lowest overall RFU and poor LoD followed by the heat inactivated virus stored in 10% glycerol. The best performing material was gamma irradiated lysates, which exhibited strong RFU signals down to 1000 copies/mL.

Experiment 3: To evaluate the ability to pool using the CERES NANOTRAP beads a series of positive samples with Ct values ranging from ˜15 to ˜35 by 5 Ct values was evaluated in relation to pooling 4 and/or 8 samples. To create the pooled samples, 100 μL of each sample was combined into a single tube such that, for example, a pool of 4 samples has a final volume of 400 μL. To each of the pooled samples was added 200 μL of CERES NANOTRAP beads and the pooled sample eluted into 100 μL of lysis buffer.

The results from this analysis demonstrate that with a pooling size of 4 or 8 samples with a Ct value of ˜30 is detectable (Table 66). The RFU values for that sample demonstrate linearity from the sample alone (10,000 RFU), 4:1 (-5,000 RFU), and 8:1 (-2500 RFU) starting at a Ct value of ˜25.

Experiment 4: To evaluate the ability of including Influenza A and B in the multiplexed array, the PCR conditions were modified to accommodate the incorporation of UNG. A comparison of the current room temperature (RT) condition at 45° to the 55°—conditions needed for UNG denaturation is shown in Table 67. The data shows that the change from 45° to 55° increases the RFU signals at the lower concentration without any adversely impacting signal strength. An LoD of 100 copies/mL was obtained using gRNA on the Zymo platform.

Experiment 5: Next, to test specificity of the platform for Influenza A and B, a series of samples were extracted using both Zymo and Ceres protocols. The samples were acquired through Tricore and were tested using the Respiratory Virus Panel by Real Time PCR (BioFire Diagnostics) with an LoD˜300 copies/mL for Influenza A (RESPAN). The results of this analysis shown in Table 68 demonstrate specificity within the assay. Table 69 shows that the CERES NANOTRAP beads capture/lysis/analysis protocol described above for CoV-2 (0.5 ml VTM+0.2 ml Ceres, magnetic bead isolation, elution & lysis in 0.1 ml) may also be adopted for detecting InfB signals on clinical positives that were greater than 8× the threshold obtained from matched clinical negatives.

TABLE 65
Comparison of cell lysates under different storage conditions

| Heat inactivated cell lysate | | | | Heat inactivated cell lysate 10% glycerol | | | | Gamma-irradiated cell lysate | | | |
|---|---|---|---|---|---|---|---|---|---|---|---|
| | RNAase | SARS | SARS | | RNAase | SARS | SARS | | RNAase | SARS | SARS |
| copies/ | P Probe | COV-2 | COV-2 | copies/ | P Probe | COV-2 | COV-2 | copies/ | P Probe | COV-2 | COV-2 |
| mL | pub1.1 | N1 RE1.1* | N2 RE1.4^{¶} | mL | pub1.1 | N1 RE1.1* | N2 RE1.4^{¶} | mL | pub1.1 | N1 RE1.1* | N2 RE1.4^{¶} |
| | 47318 | 12381 | 27959 | | 57261 | 26168 | 39634 | | 54384 | 33810 | 46348 |
| 30K | 48826 | 8368 | 41434 | 30K | 56497 | 25473 | 41102 | 30K | 54294 | 29690 | 48684 |
| | 55016 | 11608 | 26506 | | 55182 | 23042 | 50369 | | 57171 | 33843 | 47384 |
| | 56838 | 1374 | 1548 | | 59248 | 7759 | 12921 | | 58174 | 11137 | 25297 |
| 3K | 51094 | 3531 | 2543 | 3K | 53062 | 6307 | 8648 | 3K | 58564 | 15688 | 28588 |
| | 58134 | 3110 | 5744 | | 50181 | 6059 | 7683 | | 57006 | 12938 | 26804 |
| | 56519 | 1058 | 7254 | | 58584 | 2940 | 3386 | | 48008 | 11141 | 20910 |
| 1K | 52534 | 2503 | 764 | 1K | 53593 | 7365 | 3611 | 1K | 57634 | 9339 | 13513 |
| | 56423 | 1449 | 1229 | | 53792 | 4577 | 4903 | | 51369 | 10479 | 13458 |

*Threshold = 2190;
^{¶}threshold = 3292;
RFU > LoD

TABLE 66
Pooling studies using CERES NANOTRAP Mini-RV technology

| Roche Cobas | Positive | | | |
|---|---|---|---|---|
| reported Ct | sample ID | | SARS | SARS |
| value | and pooling | | COV-2 | COV-2 |
| (Tg1/Tg2) | dilution | RNAse P | N1-RE1.1* | N2-RE1.4^{¶} |
| 34.32/34.17 | 432-Alone | 60203 | 3836 | 890 |
| | 432-4:1 | 59900 | 2873 | 673 |
| | 432-8:1 | 60406 | 2162 | 782 |
| 28.71/29.6 | 415-alone | 56995 | 9245 | 10540 |
| | 415-4:1 | 60307 | 4530 | 5339 |
| | 415-8:1 | 60175 | 3349 | 2238 |
| 25.76/26.7 | 412-alone | 54171 | 14150 | 34450 |
| | 412-4:1 | 59797 | 20210 | 30751 |
| | 412-8:1 | 60698 | 8566 | 9658 |
| 19.19/19.8 | 418-Alone | 59683 | 50496 | 59140 |
| | 418-4:1 | 59425 | 51793 | 58919 |
| | 418-8:1 | 60371 | 46331 | 59888 |
| 17.27/17.42 | 425-Alone | 42238 | 52803 | 58829 |
| | 425-4:1 | 44192 | 40680 | 59106 |
| | 425-8:1 | 52430 | 38046 | 59696 |

*Threshold = 2190;
^{¶}Threshold = 3292

TABLE 67
Effect of reverse transcription temperature on sensitivity and specificity

| | Influenza A, B | Influenza A, B | Influenza A, B | Clinical | Clinical | CoV gRNA^{¶} | |
|---|---|---|---|---|---|---|---|
| Slide 9985 | 1000 copies/reaction | 500 copies/reaction | 100 copies/reaction | infA-3b* | infA-4b* | 500 | NTC^{§} |
| | 45° C., 45 min reverse transcription | | | | | | |
| 62-Negcont-B | 613 | 533 | 978 | 2637 | 1617 | 1882 | 894 |
| 614D-SE-S1-RE1.4 | 554 | 454 | 245 | 1891 | 1345 | 22838 | 1033 |
| 614G-SE-S1-RE1.4 | −10 | 694 | 265 | 1653 | 1285 | 1755 | 1043 |
| 614U-SE-S1-RE1.4 | 509 | 462 | 885 | 1482 | 1309 | 36490 | 1092 |
| InfA 7 univ-pubRev | 44122 | 24795 | 7297 | 62387 | 1104 | 64 | 225 |
| InfB 8 univ-pub | 40626 | 39582 | 33881 | −110 | −55 | 341 | 48 |
| RNAase P Probe pub1.1 | 3321 | 4166 | 4439 | 62073 | 61697 | 5298 | 5635 |
| SARS COV-2 N1 pub | 6505 | 9633 | 557 | 6352 | 6829 | 61851 | 13421 |
| SARS COV-2 N1 RE1.1 | 1481 | 4342 | 6254 | 4504 | 3610 | 53173 | 5376 |
| SARS COV-2 N2 RE1.3 | 2704 | 1561 | 1553 | 2671 | 2975 | 35840 | 1697 |
| SARS COV-2 N2 RE1.4 | 2577 | 724 | 201 | 2180 | 2353 | 61274 | 1053 |
| SARS COV-2 N3 RE1.1 | 4765 | 4570 | 4981 | 8193 | 7288 | 61356 | 15052 |
| | 55° C., 45 min reverse transcription | | | | | | |
| 62-Negcont-B | 579 | 1776 | 724 | 2108 | 1395 | 3633 | 1514 |
| 614D-SE-S1-RE1.4 | 220 | 211 | 292 | 1851 | 845 | 19490 | 631 |
| 614G-SE-S1-RE1.4 | 15 | 303 | 329 | 1542 | 458 | 2052 | 865 |
| 614U-SE-S1-RE1.4 | 658 | 708 | 985 | 1832 | 945 | 31619 | 1267 |
| InfA 7 univ-pubRev | 43972 | 41491 | 20854 | 59646 | 9286 | 9 | 135 |
| InfB 8 univ-pub | 44453 | 41012 | 36009 | −180 | 40 | 633 | 323 |
| RNAase P Probe pub1.1 | 3410 | 3336 | 5745 | 62270 | 60936 | 5770 | 2321 |
| SARS COV-2 N1 pub | 21951 | 12594 | 16458 | 6847 | 5066 | 62714 | 10895 |
| SARS COV-2 N1 RE1.1 | 5450 | 8669 | 7638 | 5278 | 3115 | 58984 | 7296 |
| SARS COV-2 N2 RE1.3 | 1184 | 2624 | 1899 | 3336 | 2358 | 37323 | 5356 |
| SARS COV-2 N2 RE1.4 | 854 | 1955 | 1028 | 2480 | 2591 | 61442 | 2524 |
| SARS COV-2 N3 RE1.1 | 5053 | 5609 | 11129 | 4300 | 9510 | 62109 | 13624 |

*confirmed clinical samples extracted using Zymo;
^{¶}SARS CoV-2 RNA;
^{§}no template control

TABLE 68
Specificity of the platform for Influenza A for samples extracted by Zymo and Ceres methods

| Slide- 9982 | Extraction - Zymo InfA | | | | | | | | |
|---|---|---|---|---|---|---|---|---|---|
| Sample | infA-1 | infA-2 | infA-3 | infA-4 | infA-5 | infA-6 | infA-7 | infA-8 | NTC^{§} |
| 62-Negcont-B | 4178 | 3748 | 2392 | 2296 | 2934 | 1393 | 2108 | 1395 | 3$69 |
| RNAase P Probe pub1.1 | 62228 | 61848 | 54868 | 3069 | 61894 | 60′7S6 | 622′70 | 61i936 | 2498 |
| SAPS COV-2 N1 RE1.1 | 3843 | 2145 | 584 | 6524 | 291 | 1685 | 527$ | 3115 | 5155 |
| SARS COV-2 N2 RE1.4 | 1791 | 1734 | 1055 | 957 | 1068 | 313 | 2480 | 2591 | 991 |
| SARS COV-2 N3 RE1.1 | 6005 | $635 | ~IOS | 2633 | 6139 | 3593 | 4300 | 9510 | 7727 |
| InfA 7 univ-pubRev | 4389 | 2106 | 54328 | 236 | 50269 | 61072 | 59646 | 92$6 | −185 |
| InfB 8 univ-pub | −15 | 398 | 255 | 118 | 366 | 787 | −180 | 40 | 397 |

| Slide 9987 | Extraction -Ceres InfA | | | | | | | |
|---|---|---|---|---|---|---|---|---|
| Sample | Cr-infA-1 | Cr-infA-2 | Cr-infA-3 | Cr-infA-4 | Cr-infA-5 | Cr-infA-6 | NTC^{§} | NTC^{§} |
| 62-Negcont-B | 514 | 536 | 623 | 605 | 622 | 459 | 592 | 630 |
| RNAase P Probe pub1.1 | 40667 | 30593 | 42960 | 41415 | 42819 | 43331 | 280 | 464 |
| SARS COV-2 N1 RE1.1 | 1435 | 742 | 1020 | 677 | 887 | 1003 | 1609 | 1516 |
| SARS COV-2 N2 RE1.4 | 1919 | 1993 | 1207 | 1614 | 1936 | 1520 | 1056 | 1326 |
| SARS COV-2 N3 RE1.1 | 2852 | 2671 | 4168 | 3650 | 3114 | 3232 | 2122 | 3885 |
| InfA 7 univ-pubRev | 21474 | 4.3408 | 13709 | −73 | 85 | 38326 | −132 | −308 |
| InfA 7 univ-RE1.1 | 17678 | 43457 | 20880 | 604 | 519 | 39903 | −85 | 158 |
| InfA SE-PR99524 | 17321 | 45435 | 19193 | 592 | 1267 | 41711 | 778 | 295 |
| InfA SE-PR99525 | 11668 | 38574 | 10048 | 141 | 1141 | 35424 | 551 | 252 |
| InfB 8 univ-pub | −188 | −212 | −71 | −346 | −5 | −290 | 60 | −292 |

^{§}no template control

TABLE 69
Specificity of the platform for Influenza B for samples extracted by Zymo and Ceres methods

| | |
|---|---|
| Slide- 9982 | Extraction - Zymo InfA |

| Sample | infB-1 | infB-2 | infB-3 | infB-4 | infB-5 | infB-6 | infB-7 | infB-8 | NTC^{§} |
|---|---|---|---|---|---|---|---|---|---|
| 62-Negcont-B | 4268 | 3341 | 2339 | 4421 | 3565 | 5347 | 2409 | 1589 | 3869 |
| RNAase P Probe pub1.1 | 1273 | 8010 | 59917 | 54383 | 61210 | 41002 | 60997 | 39722 | 2498 |
| SARS COV-2 N1 RE1.1 | 1972 | 6733 | 3342 | 1125 | 469 | 8875 | 6118 | 10978 | 5155 |
| SARS COV-2 N2 RE1.4 | 1147 | 1727 | 1460 | 1434 | 2191 | 5331 | 1452 | 3346 | 991 |
| SARS COV-2 N3 RE1.1 | 1686 | 2440 | 7031 | 4604 | 6266 | 10289 | 6596 | 8030 | 7727 |
| InfA 7 univ-pubRev | −16 | −94 | 658 | 555 | 340 | 32 | 8100 | 11431 | −185 |
| InfB 8 univ-pub | 37775 | 763 | 10304 | 30340 | 36467 | 12787 | 13859 | 10563 | 397 |

| Slide 9987 | Extraction -Ceres InfB | | | | | | | |
|---|---|---|---|---|---|---|---|---|
| Sample | Cr-infB-1 | Cr-infB-2 | Cr-infB-3 | Cr-infB-4 | Cr-infB-5 | Cr-infB-6 | NTC^{§} | NTC^{§} |
| 62-Negcont-B | 213 | 234 | 344 | 723 | 545 | 410 | 592 | 630 |
| RNAase P Probe pub1.1 | 681 | 35042 | 46145 | 40655 | 22259 | 40078 | 280 | 464 |
| SARS COV-2 N1 RE1.1 | 1125 | 864 | 942 | 624 | 866 | 827 | 1609 | 1516 |
| SARS COV-2 N2 RE1.4 | 1474 | 1039 | 1268 | 455 | 1504 | 1421 | 1056 | 1326 |
| SARS COV-2 N3 RE1.1 | 2275 | 1895 | 3001 | 2639 | 3131 | 2982 | 2722 | 3885 |
| InfA 7 univ-pubRev | −263 | −117 | −165 | −88 | 37 | −19 | −132 | −308 |
| Infb 8 univ-PUB | 209 | 12948 | 28137 | 23356 | 18372 | 12004 | 60 | −292 |
| InfB SE-PR99519 | 1532 | 14837 | 28237 | 25538 | 16526 | 16402 | 875 | 401 |
| InfB SE-PR99520 | 1447 | 11975 | 17636 | 15775 | 14112 | 12413 | 916 | 485 |

^{§}no template control

### Example 28

### Threshold Determination for the Ceres-DETECTX-RV Combination.

Repeat measurements (n=72) from a single pooled clinical negative sample (50 mls, TriCore NP-VTM) using the Ceres processing protocol and DETECTX-RV analysis were performed as 72 independent 0.5 ml Ceres extractions. The experiments were performed on multiple days over 2 weeks, followed by Ceres processing, RT-PCR and analysis in a 96-well format for N1 and N2 CoV-2 markers.

Clinical Matrix Samples for Threshold Analysis protocol:
1. Clinical matrix=TriCore negative clinical samples (NP-VTM, Cobas 6800)
2. 200 μL Ceres beads were added to 5004 of Contrived Clinical Matrix (N=10)
3. Samples were shaken for 10 mins.
4. Quick spin was performed before adding samples to a magnetic plate and removing supernatant.
5. 100 μL of 0.5% TritonX-100 was added to the samples.
6. Samples were shaken for 2 mins followed by heating at 95° C. for 10 mins.
7. Samples were centrifuged briefly before adding to magnetic plate.
8. Eluate was transferred to a PCR plate for storage.
8. Samples were run using standard RT-PCR cycling parameters.
9. Hybridization and Washing steps were performed in 96-well format.
10. Steps 1-9 were repeated for multiple days.
11. The threshold was calculated using the formula; Threshold=3×STD+RFU(blank)

### Results:

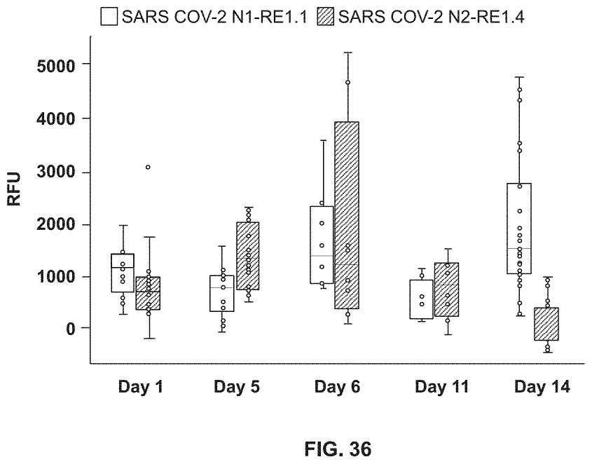

The data in Table 70 reveals a defined average with no drift in threshold values over the 5 repeat measurements. The analysis also revealed the presence of occasional outliers—e.g. day 19 for N2 and day 22 for N1 (FIG. 36), which shift the local average for these days. These outliers can be readily eliminated by adding a bead washing step in the above protocol to remove residual binding buffer.

### Example 29

### Optimization for Respiratory Syncytial Virus (RSV)

In continuation of the in silico analysis of RSV described in Example 16, primer and microarray testing was performed. Table 71 summarizes the results from an analytical sensitivity dilution series—purified human RSV-B gRNA (BEI) diluted to 1000, 100 and 10 genome copies/PCR reaction. The data demonstrates excellent specificity with no measurable signal detected above background for either of the two RSV-A probes tested in the array (HSV-A, RE1.1, 1.2). The data also demonstrate excellent sensitivity for detection of N1 and N2 above a threshold defined by the (0) genome copy control down to 10 copies/reaction.

TABLE 70
Summary of threshold analysis for clinical matrix samples

| Probe | | Day 1 | Day 5 | Day 6 | Day 11 | Day 14 | ALL |
|---|---|---|---|---|---|---|---|
| N1 | Threshold | 2258 | 2190 | 4783 | 1845 | 6021 | 4721 |
| | Average | 958 | 813 | 1739 | 674 | 2062 | 1434 |
| | Standard | 406 | 430 | 951 | 366 | 1237 | 1027 |
| | Deviation | | | | | | |
| | 95% CI | 759-1157 | 625-1002 | 1079-2398 | 420-927 | 1633-2490 | 1215-1654 |
| N2 | Threshold | 2469 | 3292 | 8081 | 2662 | 1496 | 3950 |
| | Average | 846 | 1457 | 1949 | 880 | 227 | 870 |
| | Standard | 507 | 573 | 1916 | 557 | 397 | 962 |
| | Deviation | | | | | | |
| | 95% CI | 597-1094 | 1206-1709 | 622-3277 | 494-1266 | 89-364 | 664-1076 |

### Example 30

### Concurrent Microarray Analysis of Virus, Bacteria and Fungus

The method of detecting RNA virus comprises the following steps:

1) Recovery of viral RNA by capture of the virus from a fluid sample (analyte) by pipetting or centrifugation or binding of the pathogen to a solid phase such as an appropriate magnetic bead or column, followed by lysis of the captured pathogen and then in some cases additional purification of RNA from the virus by silica-based boom chemistry as routinely deployed in magnetic beads or columns.

2) RT-PCR of the viral RNA recovered, to generate PCR amplified cDNA amplicons that are further amplified using a suitable set of fluorescently labeled primers specific for the cDNA amplicons to obtain fluorescently labeled amplicons suitable for microarray hybridization.

3) Microarray hybridization of the resulting RT-PCR amplified DNA amplicons.

4) Analysis of the microarray hybridization patterns to detect the presence of viral analytes of interest.

The above method is extended to include DNA-containing pathogens including, DNA viruses, bacteria and fungus known to cause respiratory disease by accommodating capture and analysis of both RNA-containing and DNA-containing pathogens. Such a method comprises the following steps:

TABLE 71
RSV specificity and sensitivity analysis

| | PCR machine | | | | | | | |
|---|---|---|---|---|---|---|---|---|
| | 2720 (Applied Biosystem) | | | | Veriti (Applied Biosystem) | | | |
| | HRSV (copies/reaction) | | | | HRSV (copies/reaction) | | | |
| | 1000 | 100 | 10 | NTC | 1000 | 100 | 10 | NTC |
| Sample | Well 34 | Weil 35 | Well 36 | Well 40 | Well 66 | Well 67 | Well 68 | Well 72 |
| 614D-SE-S1-RE1.4 | 40 | 78 | 47 | 148 | 4 | 109 | 15 | 130 |
| 614D-SE-S1-RE1.5 | 3102 | 2391 | 3710 | 2908 | 3466 | 2215 | 2018 | 3123 |
| 614D-SE-S1-RE1.7 | 2974 | 2466 | 2705 | 3233 | 3279 | 2417 | 2226 | 3176 |
| 614G-SE-S1-RE1.4 | 230 | −114 | 22 | 1 | −43 | −73 | 48 | 176 |
| 614G-SE-S1-RE1.5 | 2451 | 1890 | 2060 | 2328 | 3030 | 1605 | 1933 | 2419 |
| 614G-SE-S1-RE1.7 | 3380 | 2288 | 2185 | 2464 | 2595 | 2284 | 2215 | 2900 |
| 614U-SE-S1-RE1.1 | −59 | 12 | −50 | 176 | −45 | −45 | −47 | 89 |
| 614U-SE-S1-RE1.8 | 2625 | 1858 | 2225 | 2817 | 2672 | 1996 | 1911 | 2572 |
| 614U-SE-S1-RE1.9 | 2399 | 1254 | 1684 | 1325 | 1360 | 896 | 854 | 1152 |
| 62-Negcont-B | 76 | 113 | −41 | 229 | 180 | 97 | 168 | 227 |
| HRSV.A_RE 1.1 | 1889 | 519 | 1633 | 733 | 1673 | 8259 | 522 | 599 |
| HRSV.A_RE 1.2 | 4043 | 3541 | 1073 | 3375 | 1995 | 1032 | 522 | 830 |
| HRSV.B_RE 1.1 | 63637 | 63891 | 20038 | 1903 | 63641 | 49478 | 6709 | 858 |
| HRSV.B_RE 1.2 | 63582 | 56338 | 13683 | 1880 | 63564 | 42073 | 5470 | 1294 |
| HRSV.B_RE 1.3 | 63497 | 63732 | 38712 | 693 | 63404 | 54081 | 7533 | 582 |
| HRSV.B_RE 1.4 | 63512 | 63045 | 35071 | 836 | 63430 | 50738 | 4357 | 622 |
| InfA.7.univ-pubFwd | 1306 | 1313 | 1301 | 1586 | 683 | 1115 | 681 | 1241 |
| InfA.7.univ-pubRev | −154 | −133 | −235 | −25 | −199 | −76 | −81 | −30 |
| InfA.7.univ-RE1.1 | −230 | 44 | −32 | −138 | 687 | −56 | −52 | 127 |
| infA-AS-PR99526 | 1128 | 874 | 990 | 924 | 566 | 618 | 546 | 759 |
| infA-SE-PR99524 | 785 | 852 | 180 | 678 | 305 | 429 | 415 | 315 |
| infA-SE-PR99525 | 856 | 252 | 297 | 189 | 215 | 340 | 78 | 14 |
| InfB.8.univ-pub | −162 | −111 | 200 | 454 | −248 | 73 | −37 | 121 |
| infB-SE-PR99519 | 1123 | 338 | 1273 | 305 | 739 | 287 | 384 | 89 |
| infB-SE-PR99520 | 999 | 660 | 1048 | 626 | 776 | 491 | 586 | 442 |
| RNAse.P.Probe-pub1.1 | −209 | −210 | −197 | −100 | −125 | −159 | −122 | 794 |
| SARS.CoV1-N2-RE1.3 | 1497 | 1525 | 1392 | 1599 | 1534 | 921 | 993 | 1427 |
| SARS.CoV1-N2-RE1.6 | 2986 | 1649 | 1643 | 1930 | 2896 | 1253 | 1335 | 1461 |
| SARS.COV2-N1-pub | 19 | 85 | 42 | 215 | −142 | 117 | −3 | 153 |
| SARS.COV2-N1-RE1.1 | 979 | 579 | 810 | 1479 | 911 | 1155 | 546 | 1656 |
| SARS.CoV2-N2-RE1.12 | 2285 | 1645 | 1214 | 1146 | 2165 | 1031 | 2004 | 1067 |
| SARS.COV2-N2-RE1.4 | 1863 | 617 | 799 | 1000 | 947 | 504 | 385 | 98 |
| SARS.COV2-N3-RE1.1 | 1004 | 804 | 1046 | 1002 | 1008 | 573 | 556 | 488 |

1) Use methods such as pipetting and centrifugation among others to capture concurrently, RNA viruses, DNA viruses, bacteria and fungus resident in the same clinical or environmental sample. Subsequent methods of lysis are then employed to enable concurrent lysis of all of the captured pathogens. Additional purification steps such as, silica-based boom chemistry as routinely deployed in magnetic beads or columns enables concurrent capture and purification of RNA and DNA from these pathogens.

2) Use of an appropriate panel of PCR primers enables reverse transcription of viral RNA to obtain cDNA followed by PCR amplification to concurrently amplify in the same reaction (single assay), cDNA and DNA from DNA viruses, bacteria and fungus to yield a set of amplicons that are further amplified using a suitable set of fluorescently labeled primers specific for each pathogen being queried to obtain fluorescently labeled amplicons suitable for microarray hybridization.

3) Concurrent microarray hybridization of the resulting fluorescent amplicons on the same microarray enables their analysis.

4). Analysis of the microarray hybridization patterns obtained is then used to concurrently detect presence of any or all of pathogens in a sample.

### Conclusion

Rapid detection of respiratory disease-causing viruses including COVID-19 virus, other coronaviruses, Influenza A virus, Influenza B virus, RSV-A and RSV-B are crucial to controlling the COVID-19 pandemic. However, it is well known that there are other DNA containing respiratory disease-causing pathogens including DNA viruses like adenovirus and bacterial pathogens such as Mycobacterium tuberculosis and Streptococcus pneumoniae. The microarray-based detection methods described here are readily adaptable and extendable to detection of these DNA containing respiratory disease pathogens as well in a single assay. This is beneficial since it enables streamlined concurrent detection of COVID-19 virus concurrently with other respiratory disease pathogens in a single assay.

### Example 31

### Clinical Validation of Influenza A/B Analysis

Experiment 1: LoD studies in contrived clinical negative samples (NP-VTM, TriCore) were performed using inactivated flu virus (ATCC, InFA (H1NI), and InFB (Hong Kong)). Particle density in the ATCC samples was measured in infectious units via PFU assay (i.e. CEID₅₀/ml) which is approximately equal to particles/mL. In all cases, viral capture with Ceres beads was performed on the flu virus, followed by lysis and amplification of the lysate with the complete One Step RT-PCR master mix comprising the full N1,N2,N3,P,InFA,InFB multiplex described in previous reports. Hybridization was obtained in the 96-well format.

### Protocol:

1. Dilutions of Inf A (H1N1) and Inf B (Hong Kong) were made in clinical matrix (VTM+negative clinical sample) as shown in Table 72.
2. Add 200 μL Ceres beads to 500 μL sample. Shake for 10 mins.
3. Place sample on magnetic stand to collect beads and remove supernatant.
4. All 200 μL PBS to sample and shake for 2 mins. Remove supernatant.
5. Add 100 μL lysis buffer to the sample. Shake for 2 mins.
6. Heat samples at 95° C. for 10 mins.
7. Place sample on magnetic stand to collect beads.
8. Transfer RNA from tube to PCR plate for storage until use.
9. PCR parameters: 55° C., 20 min (1 cycle); 94° C., 2 min (1 cycle); 94° C., 30 sec, 55° C., 30 sec, 68° C., 30 sec (40 cycles); 68° C., 7 min, (1 cycle); 4° C.,

TABLE 72
Protocol for clinical validation of Influenza A and Influenza B

| | | | | | |
|---|---|---|---|---|---|
| | | | Stock | | Total |
| Dilution | [Stock] | [Final] | volume | Diluent | volume |
| Factor | (CEID₅₀/mL) | (CEID₅₀/mL) | (μL) | (μL) | (μL) |

Influenza A- HIN1 NR-2555 Lot 4771527 (1.6 × 10⁸ CEID₅₀/mL)

| | | | | | |
|---|---|---|---|---|---|
| 100.00 | 1.60 × 10⁸ | 1.60 × 10⁶ | 5 | 495 | 500 |
| 32.00 | 1.60 × 10⁶ | 5.00 × 10⁴ | 16 | 484 | 500 |
| 50.00 | 5.00 × 10⁴ | 1000 | 116 | 5684 | 5800 |
| 1.33 | 1000 | 750 | 3375 | 1125 | 4500 |
| 1.50 | 750 | 500 | 2000 | 1000 | 3000 |
| 5.00 | 500 | 100 | 500 | 2000 | 2500 |

Influenza B Hong Kong NR-41802 Lot 70020821 (1.8 × 10⁷ CEID₅₀/mL)

| | | | | | |
|---|---|---|---|---|---|
| 100.00 | 1.80 × 10⁷ | 1.80 × 10⁵ | 5 | 495 | 500 |
| 36.00 | 1.80 × 10⁵ | 5000 | 14 | 486 | 500 |
| 50.00 | 5000 | 100 | 60 | 2940 | 3000 |
| 10.00 | 100 | 10 | 400 | 3600 | 4000 |
| 2.00 | 10 | 5 | 1500 | 1500 | 3000 |
| 5.00 | 5 | 1 | 500 | 2000 | 2500 |

### Sample Analysis:

First, a clinical threshold was obtained using thirty-two (32) clinical negatives for the InF A and InF B probe content, using the formula;

Threshold (RFU)=3 ×(STV)+Mean

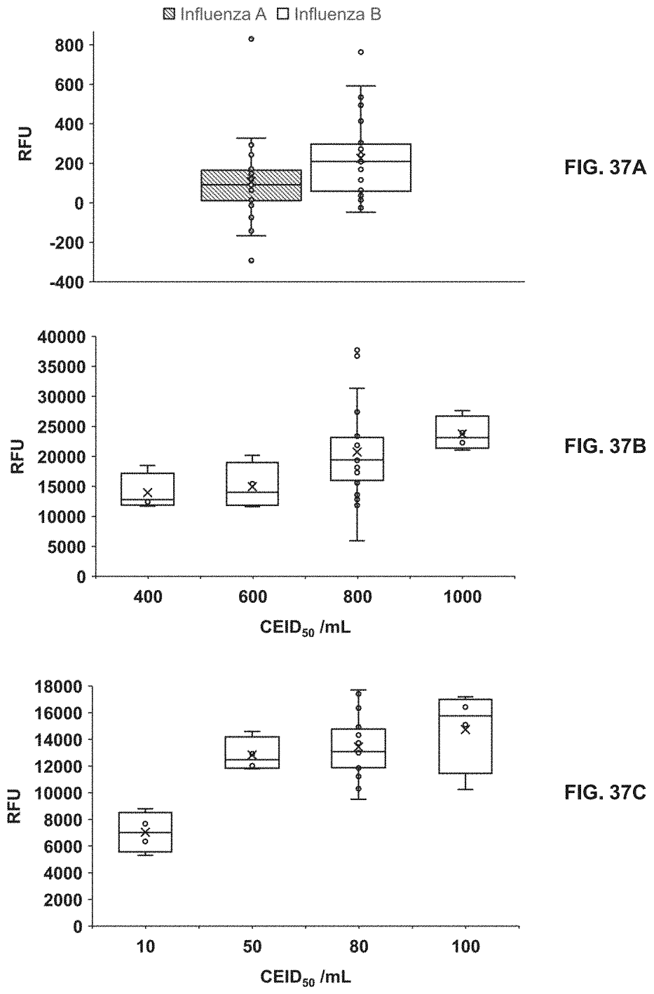

These data revealed low background values and as a result low thresholds (721 RFU for Inf A and 896 RFU for Inf B FIG. 37A).

Next, a preliminary range-seeking study was performed on contrived InF A and InF B samples prepared on a single batch of pooled clinical negatives that revealed that the LoD would be in the approximate range of 1,000 CEID₅₀/ml for InF A and 100 CEID₅₀/ml for InF B (Inf A, LoD=100-1000 CEID₅₀; Inf B, LoD=1-10 CEID₅₀). Based on this preliminary analysis, a more detailed LoD determination was performed that showed that the LoD for InF A is less than 400 CEID₅₀/ml (FIG. 37B) and LoD for InFB is less than 10 CEID₅₀/ml (FIG. 37C).

Experiment 2: An extension of the above studies was performed at multiple data points closer to the LoD. Dilutions of Inf A (H1N1) and Inf B (Hong Kong) were made in clinical matrix (45 mL VTM+5 mL pooled negative clinical sample) as shown in Table 73 and the method performed as described above for Experiment 1.

TABLE 73
Protocol for clinical validation of Influenza A and Influenza B

| | | | | | |
|---|---|---|---|---|---|
| | | | Stock | | Total |
| Dilution | [Stock] | [Final] | volume | Diluent | volume |
| Factor | (CEID₅₀/mL) | (CEID₅₀/mL) | (μL) | (μL) | (μL) |

Influenza A- HIN1 NR-2555 Lot 4771527 (1.6 × 10⁸ CEID₅₀/mL)

| | | | | | |
|---|---|---|---|---|---|
| 100.00 | 1.60 × 10⁸ | 1.60 × 10⁶ | 5 | 495 | 500 |
| 100.00 | 1.60 × 10⁶ | 16000 | 5 | 495 | 500 |
| 16.00 | 5.00 × 10⁴ | 1000 | 438 | 6563 | 7000 |
| 2.50 | 1000 | 400 | 4000 | 6000 | 10000 |
| 2.00 | 400 | 200 | 6500 | 6500 | 13000 |
| 2.00 | 200 | 100 | 1500 | 1500 | 3000 |

Influenza B Hong Kong NR-41802 Lot 70020821 (1.8 × 10⁷ CEID₅₀/mL)

| | | | | | |
|---|---|---|---|---|---|
| 100.00 | 1.80 × 10⁷ | 1.80 × 10⁵ | 5 | 495 | 500 |
| 100.00 | 1.80 × 10⁵ | 1800 | 5 | 495 | 500 |
| 18.00 | 1800 | 100 | 222 | 3778 | 4000 |
| 10.00 | 100 | 10 | 900 | 8100 | 9000 |
| 2.00 | 10 | 5 | 6000 | 6000 | 12000 |
| 5.00 | 5 | 1 | 600 | 2400 | 3000 |

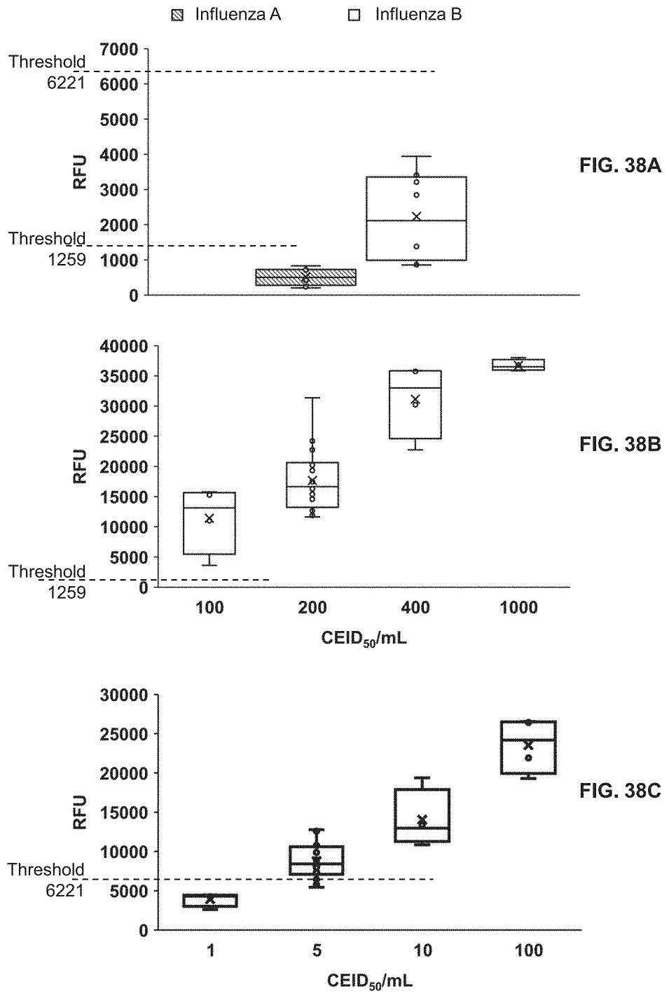

An extended clinical threshold was obtained by processing additional clinical negatives for the Inf A and Inf B probe content, using the formula used above. The extended data set revealed a statistically strong background mean and STD that is reproducible with a threshold value of 1259 RFU for Inf A and 6221 RFU for Inf B (FIG. 38A). Expanded range-seeking optimization on contrived influenza samples prepared on a single batch of pooled clinical negatives revealed that LoD for Inf A is less than 100 CEID₅₀/ml (FIG. 38B) and that the LoD for Inf B is less than 10 CEID₅₀/mL (FIG. 38C). A comparison of LoD obtained using influenza from various sources is shown in Table 74.

TABLE 74
Comparison of LoD values

| | | LOD | | |
|---|---|---|---|---|
| | Strain | (CEID₅₀) | Company | Catalog # |
| BioFire RP2.1 | Inf. A H1N1 | 1000 | Zeptometrix | 0810036CF |
| Analyte | Inf A H1-2009 | 50 | Zeptometrix | 0810249CF |
| | Inf A H3 | 10 | ATCC | VR-810 |
| | Inf B | 5 | Zeptometrix | 0810255CF |

### Example 32

### Mouthwash LoD on Clinical Samples

LoD studies were undertaken on QuikSal mouthwash negative clinical isolates. Briefly, three (3) oral rinse samples from healthy lab volunteers were pooled to generate a single 15 mL Cov-2 negative clinical sample. The pooled sample was then doped with gamma irradiated CoV-2 (BEI) ranging from 10,000 to 625 virus particles/mL (Table 75) Protocol:
1. Add 100 μL beads to 500 μL sample. Shake for 10 mins at 1000 rpm.
2. Place sample on magnetic stand for 5 min to collect beads and remove supernatant.
3. All 2004 PBS to sample and shake for 2 mins at 1000 rpm. Remove supernatant.
4. Add 100 μL lysis buffer to the sample. Shake for 2 mins at 1000 rpm.
5. Heat samples at 95° C. for 10 mins.
6. Place sample on magnetic stand to collect beads.
7. Transfer RNA from tube to PCR plate for storage at −20° C. until use.
8. PCR parameters: 55° C., 20 min (1 cycle); 94° C., 2 min (1 cycle); 94° C., 30 sec, 55° C., 30 sec, 68° C., 30 sec (40 cycles); 68° C., 7 min, (1 cycle); 4° C., 00

TABLE 75
Protocol for clinical validation of Influenza A and Influenza B
Gamma irradiated cell lysate NR-52287 (1.75 × 10⁹ copies/mL)

| | | | Stock | | Total |
|---|---|---|---|---|---|
| Dilution | [Stock] | [Final] | volume | Diluent | volume |
| Factor | (copies/mL) | (copies/mL) | (μL) | (μL) | (μL) |
| 100.00 | 1.75 × 10⁹ | 1.75 × 10⁷ | 5 | 495 | 500 |
| 100.00 | 1.75 × 10⁷ | 1.75 × 10⁵ | 5 | 495 | 500 |
| 17.50 | 1.75 × 10⁵ | 10000 | 400 | 6600 | 7000 |
| 2.00 | 10000 | 5000 | 4000 | 4000 | 8000 |
| 5.00 | 5000 | 1000 | 5400 | 21600 | 27000 |
| 1.25 | 1000 | 800 | 15200 | 3800 | 19000 |
| 1.60 | 800 | 500 | 7500 | 4500 | 12000 |

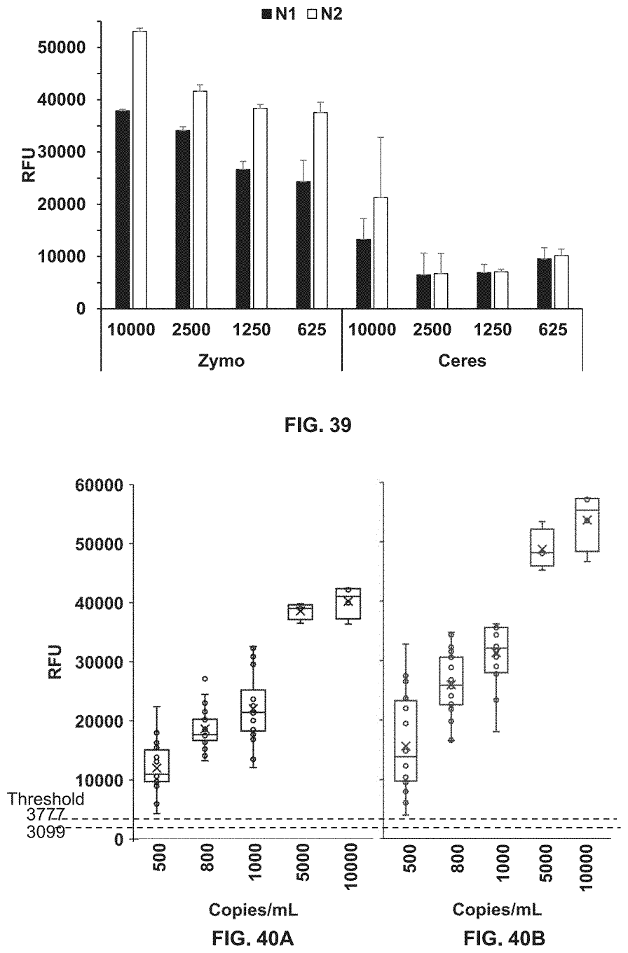

The summary of the range analysis (Zymo vs Ceres processing) is presented in FIG. 39. For Zymo magnetic bead-based RNA isolation from virally doped QuikSal negatives (FIG. 39, left panel) it can be seen that signal strength for both the N1 and N2 Cov-2 markers is >7 fold over the threshold across the entire viral density range tested. Based on that dose response, it appears that the LoD for the Zymo/One Step RT-PCR combination will be significantly lower than 625 virus copies/mL. In contrast, the preliminary range finding for Ceres magnetic bead-based viral capture on the same samples is at about 10-fold higher than the Zymo magnetic bead method (FIG. 39, right panel).

### CoV-2 LoD Analysis

As discussed above, the LoD for both N1 and N2 SARS-CoV-2 probes was <1000 virus copies/mL. Using an expanded titration to include 500, 800, 1,000 copies/mL it is observed (FIGS. 40A and 40B) that LoD values for both N1 and N2 are less than a factor of 2 below 500 copies/mL. Thus, DETECTX-RV analysis for SARS CoV-2 may be expanded to additionally include concurrent detection/measurement of both influenza A and influenza B, without compromising LoD.

The following references are cited herein:
1. Li et al., (2020) J Med Virol.; 10.1002/jmv.25786. doi:10.1002/jmv.25786
2. Feng et al., (2020) Jpn J Radiol.; 1-2. doi:10.1007/s11604-020-00967-9
3. Hu et al, (2003) J Clin Microbiol 41: 149-154. doi: 101128/jcm.41.1.149-154.2003

1. A method for detecting a Coronavirus disease 2019 (COVID-19) virus in a sample, comprising:
obtaining the sample;
isolating total RNA from the sample;
performing a combined reverse transcription and an asymmetric PCR amplification reaction on the isolated total RNA using at least one fluorescent labeled primer pair comprising an unlabeled primer and a fluorescently labeled primer, selective for a target nucleotide sequence in the COVID-19 virus to generate fluorescent labeled COVID-19 virus amplicons;
hybridizing the fluorescent labeled COVID-19 virus amplicons to a plurality of nucleic acid probes each having a sequence corresponding to a sequence determinant in the COVID-19 virus, each of said plurality of nucleic acid probes attached at a specific position on a solid microarray support;
washing the microarray at least once; and
imaging the microarray to detect at least one fluorescent signal from the hybridized fluorescent labeled COVID-19 virus amplicons, thereby detecting the COVID-19 virus in the sample.

2. The method of claim 1, further comprising detecting in the sample at least one non-COVID-19 virus comprising a Respiratory Syncytial Virus, a Middle East Respiratory Syndrome coronavirus (MERS-CoV), a Severe Acute Respiratory Syndrome Coronavirus (SARS-CoV), a 229E Coronavirus, a OC43 Coronavirus, a NL63 Coronavirus, a HKU1 Coronavirus, an Influenza A virus, or an Influenza B virus,
wherein the step of performing the combined reverse transcription and the asymmetric PCR amplification reaction on the isolated total RNA comprises using at least two fluorescent labeled primer pairs selective for the target nucleotide sequence in said COVID-19 virus and in said at least one non-COVID-19 virus to generate the fluorescent labeled virus amplicons; and
wherein the step of hybridizing comprises hybridizing the fluorescent labeled virus amplicons to the plurality of nucleic acid probes each having a sequence corresponding to a sequence determinant in said COVID-19 virus and the at least one non-COVID-19 virus.

3. The method of claim 1, further comprising calculating an intensity of the fluorescent signal, said intensity correlating with the number of virus genomes in the sample.

4. The method of claim 1, wherein the fluorescently labeled primer is in an excess of about 4-fold to about 8-fold over the unlabeled primer in the fluorescent labeled primer pair.

5. The method of claim 1, wherein the fluorescent labeled primer pair comprises the nucleotide sequences SEQ ID: 23 and SEQ ID: 24, SEQ ID: 25 and SEQ ID: 26, SEQ ID: 27 and SEQ ID: 28, SEQ ID: 29 and SEQ ID: 30, SEQ ID: 31 and SEQ ID: 32, SEQ ID: 33 and SEQ ID: 34, SEQ ID: 35 and SEQ ID: 36, SEQ ID: 37 and SEQ ID: 38, SEQ ID: 39 and SEQ ID: 40, SEQ ID: 41 and SEQ ID: 42, SEQ ID: 25 and SEQ ID: 74, SEQ ID: 75 and SEQ ID: 76, SEQ ID: 77 and SEQ ID: 78, or SEQ ID: 79 and SEQ ID: 80, or a combination thereof.

6. The method of claim 1, wherein the plurality of nucleic acid probes comprise at least one probe nucleotide sequence selected from the group consisting of SEQ ID NOS: 45-70, 85-97, 111-120 and 125-129.

7. The method of claim 1, wherein the sample is an individual sample or a pooled sample from a nasopharyngeal swab, a nasal swab, a mouth swab, a mouth wash, an aerosol, or a swab from a hard surface or a combination thereof.

8. A method for detecting at least one respiratory disease-causing virus in a sample, comprising:
obtaining a sample;
isolating total nucleic acids from the sample;
performing a combined reverse transcription and an asymmetric PCR amplification reaction on the isolated total nucleic acids using at least one fluorescent labeled primer pair, each comprising an unlabeled primer and a fluorescently labeled primer, selective for a target nucleotide sequence in the at least one respiratory disease-causing virus, to generate fluorescent labeled virus specific amplicons;
hybridizing the fluorescent labeled virus specific amplicons to a plurality of nucleic acid probes each having a sequence corresponding to a sequence determinant in the at least one respiratory disease-causing virus, each of said nucleic acid probes attached at a specific position on a solid microarray support;
washing the microarray at least once; and
imaging the microarray to detect at least one fluorescent signal from the hybridized fluorescent labeled virus specific amplicons, thereby detecting the at least one respiratory disease-causing virus in the sample.

9. The method of claim 8, further comprising calculating an intensity of the fluorescent signal, said intensity correlating with the number of virus specific genomes in the sample.

10. The method of claim 8, wherein the fluorescently labeled primer is in an excess of about 4-fold to about 8-fold over the unlabeled primer in the fluorescent labeled primer pair.

11. (canceled)

12. The method of claim 8, wherein the respiratory disease-causing virus is a Severe Acute Respiratory Syndrome Coronavirus 2 (COVID-19 virus), a Respiratory Syncytial Virus, a Middle East Respiratory Syndrome coronavirus (MERS-CoV), or a Severe Acute Respiratory Syndrome Coronavirus (SARS-CoV), or a 229E Coronavirus, or a OC43 Coronavirus, or a NL63 Coronavirus, or a HKU1 Coronavirus or an Influenza A virus or an Influenza B virus, an adenovirus, a bocavirus, a metapneumovirus, a parainfluenza, or a rhinovirus, or a combination thereof.

13. The method of claim 8, wherein the fluorescent labeled primer pair comprises nucleotide sequences SEQ ID: 23 and SEQ ID: 24, SEQ ID: 25 and SEQ ID: 26, SEQ ID: 27 and SEQ ID: 28, SEQ ID: 29 and SEQ ID: 30, SEQ ID: 31 and SEQ ID: 32, SEQ ID: 33 and SEQ ID: 34, SEQ ID: 35 and SEQ ID: 36, SEQ ID: 37 and SEQ ID: 38, SEQ ID: 39 and SEQ ID: 40, SEQ ID: 41 and SEQ ID: 42, SEQ ID: 25 and SEQ ID: 74, SEQ ID: 75 and SEQ ID: 76, SEQ ID: 77 and SEQ ID: 78, or SEQ ID: 79 and SEQ ID: 80, or a combination thereof.

14. The method of claim 8, wherein the plurality of nucleic acid probes comprise at least one probe nucleotide sequence selected from the group consisting of SEQ ID NOS: 45-70, 85-97, 111-120, and 125-129.

15-16. (canceled)

17. The method of claim 8, wherein the sample is an individual sample or a pooled sample from a nasopharyngeal swab, a nasal swab, a mouth swab, a mouth wash, an aerosol, or a swab from a hard surface or a combination thereof.

18. A method for detecting a Coronavirus disease 2019 (COVID-19) virus in a sample, comprising:
obtaining the sample;
isolating total nucleic acids from the sample;
performing a combined reverse transcription and an asymmetric PCR amplification reaction on the total nucleic acids using at least one fluorescent labeled primer pair selective for a target nucleotide sequence in the COVID-19 virus RNA to generate fluorescent labeled COVID-19 virus amplicons, each of said fluorescent labeled primer pairs comprising an unlabeled primer and a fluorescently labeled primer in an excess over the unlabeled primer;
hybridizing the fluorescent labeled COVID-19 virus amplicons to a plurality of nucleic acid probes each having a sequence corresponding to a sequence determinant in the COVID-19 virus, each of said plurality of nucleic acid probes attached at a specific position on a microarray support;
washing the microarray at least once; and
imaging the microarray to detect at least one fluorescent signal from the hybridized fluorescent labeled COVID-19 virus amplicons, thereby detecting the COVID-19 virus in the sample.

19. The method of claim 18, further comprising detecting at least one non-COVID-19 virus in the sample,
wherein the step of performing the combined reverse transcription and the asymmetric PCR amplification reaction on the isolated total nucleic acid comprises using at least two fluorescent labeled primer pairs, each comprising the unlabeled primer and the fluorescently labeled primer, selective for a target nucleotide sequence in the COVID-19 virus and the at least one non-COVID 19 virus to generate fluorescent labeled COVID-19 virus specific amplicons and fluorescent labeled non-COVID-19 virus specific amplicons; and
wherein the step of hybridizing comprises hybridizing the fluorescent labeled COVID-19 virus specific amplicons and the fluorescent labeled non-COVID-19 virus specific amplicons to the plurality of nucleic acid probes each having a sequence corresponding to the sequence determinant in the COVID-19 virus and the at least one non-COVID-19 virus.

20. The method of claim 19, wherein the non-COVID-19 virus is a Respiratory Syncytial Virus, a Middle East Respiratory Syndrome coronavirus (MERS-CoV), a Severe Acute Respiratory Syndrome Coronavirus (SARS-CoV), a 229E Coronavirus, a OC43 Coronavirus, a NL63 Coronavirus, a HKU1 Coronavirus, an Influenza A virus, an Influenza B virus, an adenovirus, a bocavirus, a metapneumovirus, a parainfluenza, or a rhinovirus, or a combination thereof.

21-24. (canceled)

25. The method of claim 18, wherein the imaging step further comprises calculating an intensity of the fluorescent signal, said intensity correlating with the number of virus genomes in the sample.

26. The method of claim 18, wherein the reverse transcription is performed at a temperature of about 45° C. to about 55° C. and for an interval of about 20 min to about 45 min.

27. (canceled)

28. The method of claim 18, wherein the fluorescent labeled primer pair comprises nucleotide sequences SEQ ID: 23 and SEQ ID: 24, SEQ ID: 25 and SEQ ID: 26, SEQ ID: 27 and SEQ ID: 28, SEQ ID: 29 and SEQ ID: 30, SEQ ID: 31 and SEQ ID: 32, SEQ ID: 33 and SEQ ID: 34, SEQ ID: 35 and SEQ ID: 36, SEQ ID: 37 and SEQ ID: 38, SEQ ID: 39 and SEQ ID: 40, SEQ ID: 41 and SEQ ID: 42, SEQ ID: 25 and SEQ ID: 74, SEQ ID: 75 and SEQ ID: 76, SEQ ID: 77 and SEQ ID: 78, or, SEQ ID: 79 and SEQ ID: 80, or a combination thereof.

29. The method of claim 18, wherein the plurality of nucleic acid probes comprise at least one probe nucleotide sequence selected from the group consisting of SEQ ID NOS: 45-70, 85-97, 111-120 and 125-129.

30. The method of claim 18, wherein the sample is an individual sample or a pooled sample from a nasopharyngeal swab, a nasal swab, a mouth swab, a mouth wash, an aerosol, or a swab from a hard surface or a combination thereof.
